(19) United States
(12) Patent Application Publication
(10) Pub. No.: US 2025/0333738 A1
(43) Pub. Date: Oct. 30, 2025

(54) POLYNUCLEIC ACID MOLECULES FOR INHIBITING EXPRESSION OF ANGPTL3, PHARMACEUTICAL COMPOSITIONS, AND USES THEREOF

(71) Applicant: Sirius Therapeutics, Inc., San Diego, CA (US)

(72) Inventors: Curt W. Bradshaw, San Diego, CA (US); Anthony Nicholas, Oregon, WI (US)

(21) Appl. No.: 19/264,729

(22) Filed: Jul. 9, 2025

Related U.S. Application Data
(63) Continuation of application No. PCT/US2024/011097, filed on Jan. 10, 2024.
(60) Provisional application No. 63/438,498, filed on Jan. 11, 2023.

(51) Int. Cl. C12N 15/113

(52) U.S. Cl. CPC C12N 15/113; C12N 2310/14; C12N 2310/321; C12N 2310/351

(57) ABSTRACT

Disclosed herein are polynucleic acid molecules that can be utilized for suppressing the expression of Angiopoietin-like protein 3 (ANGPTL3) gene. Also, described herein are pharmaceutical compositions comprising polynucleic acid molecules targeting Angiopoietin-like protein 3 (ANGPTL3) mRNA. Further, provided herein are methods for suppressing the expression of ANGPTL3 by utilizing the polynucleic acid molecules described herein.

### CROSS-REFERENCE

This application is a continuation of International Application No. PCT/US2024/011097, filed on Jan. 10, 2024, which claims the benefit of U.S. Provisional Application No. 63/438,498, filed on Jan. 11, 2023, all of which are incorporated herein by reference in their entirety.

### SEQUENCE LISTING

The instant application contains a Sequence Listing which has been submitting electronically in XML format and is hereby incorporated by reference in its entirety. Said XML copy, created Jul. 9, 2025, is named “61382-716_301_SL.xml” and is 3,303,719 bytes in size.

### BACKGROUND OF THE DISCLOSURE

The discovery of RNA interference (RNAi) as a cellular mechanism that selectively degrades mRNAs allows for both the targeted manipulation of cellular phenotypes in cell culture and the potential for development of directed therapeutics (Behlke, 2006, Mol. Ther. 13, 644-670; Xie et al., 2006, Drug Discov. Today 11, 67-73).

Hyperlipidermia is a global condition describing elevated lipid levels within the body. Hyperlipidermia is considered a major risk factor for atherosclerosis, coronary heart disease, and other vascular disease. Angiopoietin-like protein 3 or angiopoietin-like 3 (ANGPTL3) plays an important role in lipoprotein/plasma lipid metabolism and is produced primarily by cells in the liver. Inhibition of ANGPTL3 correlates with reductions of plasma lipids, including low-density lipoprotein (LDL) cholesterol, high-density lipoprotein (HDL) cholesterol, and triglycerides.

Accordingly, there is a need for developing an effective ANGPTL3 inhibitor without cytotoxicity. The polynucleic acid molecules, conjugates thereof, and methods described herein satisfy this need and provide related advantages.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### SUMMARY OF THE DISCLOSURE

To meet the need for a more effective ANGPTL3 inhibitor, provided herein, in one aspect, are a polynucleic acid molecule for modulating an expression of angiopoietin-like protein 3 or angiopoietin-like 3 (ANGPTL3) gene, wherein the polynucleic acid molecule comprises a nucleic acid sequence in Table 1, Table 2, Table 3, or Table 4.

In one aspect, the present disclosure provides a polynucleic acid molecule for modulating an expression of Angiopoietin-like protein 3 (ANGPTL3) gene, wherein the polynucleic acid molecule comprises a nucleic acid sequence in Table 1, Table 2, Table 3, or Table 4. In some embodiments, the polynucleic acid molecule is a single-stranded nucleic acid molecule. In some embodiments, the single-stranded nucleic acid molecule comprises at least 14, 15, 16, 17, 18 consecutive nucleotides that are complementary to a nucleic acid sequence selected from SEQ ID NOs: 401-596, 819-828, and 870-875, with no more than 1, 2, 3, 4 mismatches. In some embodiments, the single-stranded nucleic acid molecule comprises a sequence that is at least 80%, at least 85%, at least 90%, or at least 95% complementary to a nucleic acid sequence selected from SEQ ID NOs: 401-596, 819-828, and 870-875.

In some embodiments, the polynucleic acid molecule is a double-stranded nucleic acid molecule comprising a passenger strand and a guide strand. In some embodiments, the passenger strand comprises a nucleic acid sequence that is at least 80%, at least 85%, at least 90%, or at least 95% identical to a nucleic acid sequence selected from SEQ ID NOs: 401-596, 819-828, and 870-875. In some embodiments, the guide strand comprises a nucleic acid sequence that is at least 80%, at least 85%, at least 90%, or at least 95% identical to a nucleic acid sequence selected from SEQ ID NOs: 1-196, 797-806, and 850-857. In some embodiments, the passenger strand comprises a nucleic acid sequence comprising at least 14, 15, 16, 17, 18, 19, or 20 consecutive sequences of a sequence selected from SEQ ID NOs: 401-596, 819-828, and 870-875 with no more than 1, 2, 3, or 4 mismatches. In some embodiments, the guide strand comprises a nucleic acid sequence comprising at least 14, 15, 16, 17, 18, 19, 20, 21, or 22 consecutive sequences of a sequence selected from SEQ ID NOs: 1-196, 797-806, and 850-857 with no more than 1, 2, 3, or 4 mismatches. In some embodiments, the passenger strand comprises a nucleic acid sequence selected from SEQ ID NOs: 401-596, 819-828, and 870-875 and the guide strand comprises a nucleic acid sequence selected from SEQ ID NOs: 1-196, 797-806, and 850-857. In some embodiments, the passenger strand comprises a nucleic acid sequence that is at least 90%, or at least 95% identical to a nucleic acid sequence selected from Table 3 (SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875). In some embodiments, the guide strand comprises a nucleic acid sequence that is at least 90%, or at least 95% identical to a nucleic acid sequence selected from Table 3 (SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857). In some embodiments, the passenger strand comprises a nucleic acid sequence selected from Table 3 (SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875) and the guide strand comprises a sequence selected from a nucleic acid sequence selected from Table 3 (SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857).

In some embodiments, the polynucleic acid molecule comprises (1) a 2′-fluoro modified nucleotide; (2) a 2′-O-methyl modified nucleotide; (3) 2′-deoxy modified nucleotide, or (4) a modified internucleotide linkage. In some embodiments, the polynucleic acid molecule comprise at least two consecutive modified internucleotide linkages at the 5′ end. In some embodiments, the guide strand comprises at least two internucleotide linkages among 3 internucleotide linkages at the 3′end substituted with modified internucleotide linkages. In some embodiments, the guide strand comprises 5′-nNfnnnNfnNfNfnnnnNfnNfnnnnnnn-3′, 5′-nNfnnnNfnnnnnnnNfnNfnnnnnnn-3′, 5′-nNfnnnnNfnnnnNfnNfrnnnnnnnn-3′, 5′-nNfnnnnNfnnnnNfnNfnNfnnnnnnn-3′, or 5′-nNfnnnnnnnnnNfnNfnNfnnnnnnn-3′, wherein “Nf” stands for a 2′-fluoro modified nucleotide, and wherein “n” stands for a 2′-O-methyl modified nucleotide. In some embodiments, the passenger strand comprises 5′-nnnnnnNfnNfnNfnnnnnnnnnn-3′, 5′-nnnnnnNfnNfNfNfnnnnnnnnnn-3′, or 5′-nnnnnnnnNfNfNfnnnnnnnnnn-3′, wherein “Nf” stands for a 2′-fluoro modified nucleotide, and wherein “n” stands for a 2′-O-methyl modified nucleotide. In some embodiments, the passenger strand comprises 5′-NfnNfnNfnNfnNfNfNfnNfnNfnNfnNfnNf-3′, wherein the guide strand comprises 5′-nNfnNfnNfnNfnNfnnnNfnNfnNfnNfnnn-3′, wherein “Nf” stands for a 2′-fluoro modified nucleotide, and wherein “n” stands for a 2′-O-methyl modified nucleotide. In some embodiments, the passenger strand comprises 5′-nnnnnnNfnNfNfNfnnnnnnnnnn-3′, wherein the guide strand comprises 5′-nNfnnnNfnNfNfnnnnNfnNfnnnnnnn-3′, wherein “Nf” stands for a 2′-fluoro modified nucleotide, and wherein “n” stands for a 2′-O-methyl modified nucleotide. In some embodiments, the passenger strand comprises 5′-nnnnnnnnNfnNfnnnnnnnnnn-3′, wherein the guide strand comprises 5′-nNfnnnnnnnnnNfnNfnnnnnnnnn-3′, wherein “Nf” stands for a 2′-fluoro modified nucleotide, and wherein “n” stands for a 2′-O-methyl modified nucleotide. In some embodiments, the passenger strand comprises 5′-nnnnnnNfnNfnNfnnnnnnnnnn-3′, wherein the guide strand comprises 5′-nNfnnnnnnnnnNfnNfnNfhnnnnnn-3′, wherein “Nf” stands for a 2′-fluoro modified nucleotide, and wherein “n” stands for a 2′-O-methyl modified nucleotide. In some embodiments, the passenger strand comprises 5′-nnnnnnNfnNfnNfnnnnnnnnnn-3′, wherein the guide strand comprises 5′-_nNfnnnnNfnnnnNfnNfnNfnnnnnnn-3′, wherein “Nf” stands for a 2′-fluoro modified nucleotide, and wherein “n” stands for a 2′-O-methyl modified nucleotide.

In some embodiments, the modified intemucleotide linkage is a phosphorothioate intemucleotide linkage. In some embodiments, the modified intemucleotide linkage comprises a stereochemically enriched phosphorothioate intemucleotide linkage.. In some instances, the guide strand comprises a nucleotide analogue selected from a group consisting of acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), and 1′,2′-Dideoxyribose-3′phosphate (dAB). In some instances, the nucleotide analogue is located at a seed region of the guide strand (positions 2-8) from the 5′ end.

In some embodiments, the passenger strand comprises a nucleic acid sequence that is at least 80%, at least 85%, at least 90%, or at least 95% identical to a nucleic acid sequence selected from SEQ ID NOs: 601-796, 830-839, and 876-881. In some embodiments, the guide strand comprises a nucleic acid sequence that is at least 80%, at least 85%, at least 90%, or at least 95% identical to a nucleic acid sequence selected from SEQ ID NOs: 201-396, 808-817, and 858-868. In some embodiments, the passenger strand comprises a nucleic acid sequence selected from SEQ ID NOs: 601-796, 830-839, and 876-881 and the guide strand comprises a nucleic acid sequence selected from SEQ ID NOs: 201-396, 808-817, and 858-868. In some embodiments, the polynucleic acid molecule is 19-25 nucleotides in length. In some embodiments, the polynucleic acid molecule is 21-23 nucleotides in length.

In one aspect, the present disclosure provides a polynucleic acid molecule for modulating expression of Angiopoietin-like protein 3 (ANGPTL3) gene, wherein the polynucleic acid molecule comprises: (a) an guide strand comprising the nucleotide sequence selected from SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857 and a passenger strand comprising the nucleotide sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875; (b) an guide strand comprising the nucleotide sequence selected from SEQ ID NOs: 205, 207, 208, 211, 218, 220, 222, 240, 243-246, 250, 252, 253, 256, 308, 309, 313, 314, 345, 346, 348, 353, 357, 358, 808-817, and 858-868, and a passenger strand comprising the nucleotide sequence selected from SEQ ID NOs: 605, 607, 608, 611, 618, 620, 622, 640, 643, 644, 645, 646, 650, 652, 653, 656, 708, 709, 713, 714, 745, 746, 748, 753, 757, 758, 830-839, and 876-881; (c)a guide strand comprising the nucleotide sequence of usUfsaguuGfguuuCfgUfgAfuuuccscsa (SEQ ID NO: 313) and a passenger strand comprising the nucleotide sequence of gsgsaaauCfaCfgAfaaccaacuaa (SEQ ID NO: 713); (d) a guide strand comprising the nucleotide sequence of usUfsagagUfauaaCfcUfuCfcauuususg (SEQ ID NO: 346) and a passenger strand comprising the nucleotide sequence of asasauggAfaGfgUfuauacucuaa (SEQ ID NO: 746); (e) a guide strand comprising the nucleotide sequence of usAfsuggaUfcaacAfuUfuUfgguugsasu (SEQ ID NO: 353) and a passenger strand comprising the nucleotide sequence of csasaccaAfaAfuGfuugauccaua (SEQ ID NO: 753); (f) a guide strand comprising the nucleotide sequence of usUfsaaggAfuuuaAfuAfcCfagauusasu (SEQ ID NO: 358) and a passenger strand comprising the nucleotide sequence of asasucugGfuAfuUfaaauccuuaa (SEQ ID NO: 758); (g) a guide strand comprising the nucleotide sequence of usAfsuuagAfuugcUfuCfaCfuauggsasg (SEQ ID NO: 308) and a passenger strand comprising the nucleotide sequence of cscsauagUfgAfaGfcaaucuaaua (SEQ ID NO: 708); (h) a guide strand comprising the nucleotide sequence of usUfsauagUfugguUfuCfgUfgauuuscsc (SEQ ID NO: 314) and a passenger strand comprising the nucleotide sequence of asasaucaCfgAfaAfccaacuauaa (SEQ ID NO: 714); (i) a guide strand comprising the nucleotide sequence of usAfsgaguAfuaacCfuUfcCfauuuusgsa (SEQ ID NO: 345) and a passenger strand comprising the nucleotide sequence of asasaaugGfaAfgGfuuauacucua (SEQ ID NO: 745); (j) a guide strand comprising the nucleotide sequence of usUfsaauuAfgauuGfcUfuCfacuausgsg (SEQ ID NO: 309) and a passenger strand comprising the nucleotide sequence of asusagugAfaGfcAfaucuaauuaa (SEQ ID NO: 709); (k) a guide strand comprising the nucleotide sequence of usAfsauuaGfauugCfuUfcAfcuaugsgsa (SEQ ID NO: 815) and a passenger strand comprising the nucleotide sequence of csasuaguGfaAfgCfaaucuaauua (SEQ ID NO: 837); (1) a guide strand comprising the nucleotide sequence of usUfsucauUfgaagUfuUfuGfugaucscsa (SEQ ID NO: 812) and a passenger strand comprising the nucleotide sequence of gsasucacAfaAfaCfuucaaugaaa (SEQ ID NO: 834); (m) a guide strand comprising the nucleotide sequence of usAfsuugcUfucacUfaUfgGfaguausasu (SEQ ID NO: 813) and a passenger strand comprising the nucleotide sequence of asusacucCfaUfaGfugaagcaaua (SEQ ID NO: 835); (n) a guide strand comprising the nucleotide sequence of usAfsgaguAfuaacCfuUfcCfauuucsgsa (SEQ ID NO: 865) and a passenger strand comprising the nucleotide sequence of gsasaaugGfaAfgGfuuauacucua (SEQ ID NO: 879); or (o) a guide strand comprising the nucleotide sequence of usAfsgaguAfuaacCfuUfcCfauuccsgsa (SEQ ID NO: 866) and a passenger strand comprising the nucleotide sequence of gsgsaaugGfaAfgGfuuauacucua (SEQ ID NO: 880), where smaller case “n” stands for 2′-O-methyl modified nucleotide, upper case followed with an “f” (i.e., “Nf”) stands for 2′-fluoro modified nucleotide, and “s” stands for 3′-phosphorothioate.

In another aspect, the present disclosure provides a polynucleic acid molecule conjugate for modulating an expression of Angiopoietin-like protein 3 (ANGPTL3) gene, wherein the polynucleic acid molecule conjugate comprises a polynucleic acid molecule described herein and an asialoglycoprotein receptor targeting moiety. In some embodiments, the polynucleic acid molecule and the asialoglycoprotein receptor targeting moiety is coupled via a linker. In some embodiments, the linker comprises formula (IV) below,

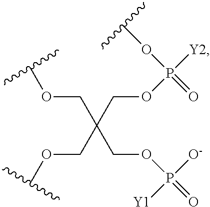

wherein at least one of Y1 and Y2 is a nucleotide in the polynucleic acid molecule. In some embodiments, the Y1 is the last nucleotide on the 3′ end of the passenger strand of the polynucleic acid molecule. In some embodiments, the Y1 and Y2 are two consecutive nucleotides in the polynucleic acid molecule. In some embodiments, the asialoglycoprotein receptor targeting moiety comprises N-Acetylgalactosamine (GalNAc). In some embodiments, the linker and the asialoglycoprotein receptor targeting moiety with the last nucleotide on the 3′ end of the passenger strand of the polynucleic acid molecule are shown in:

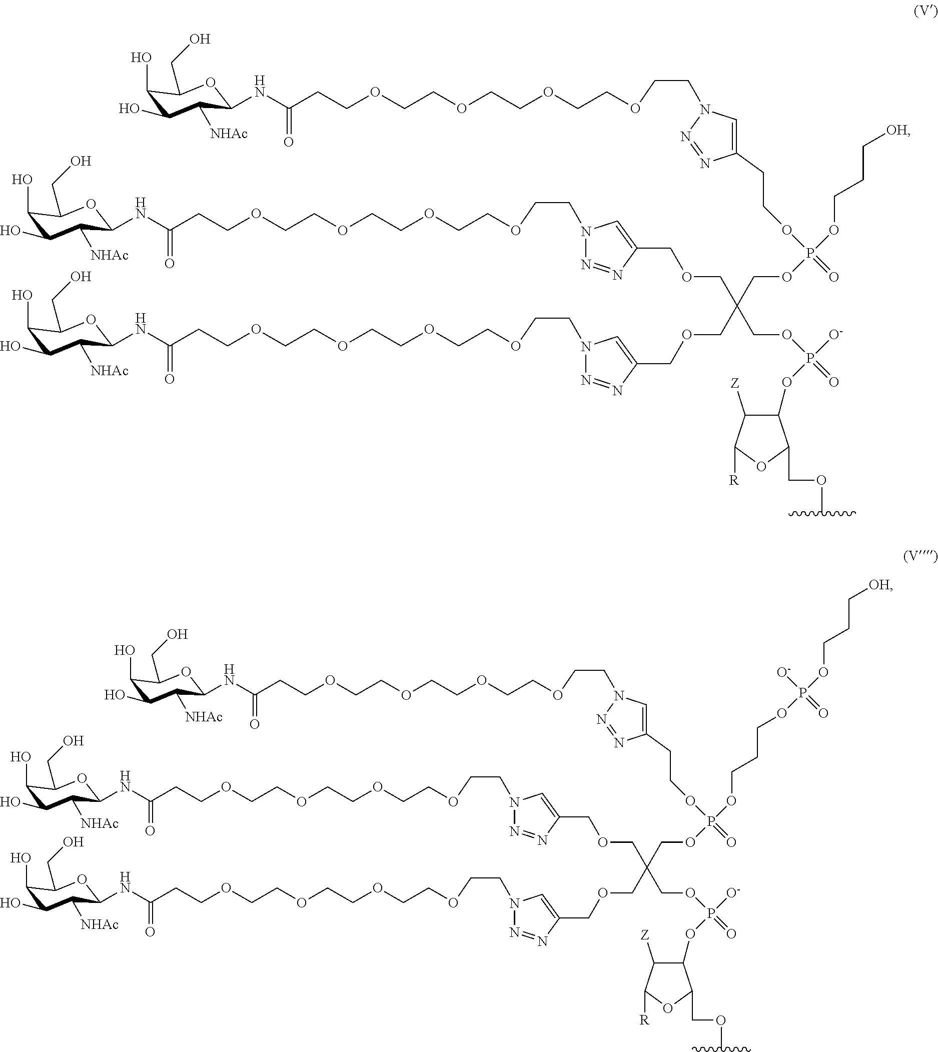

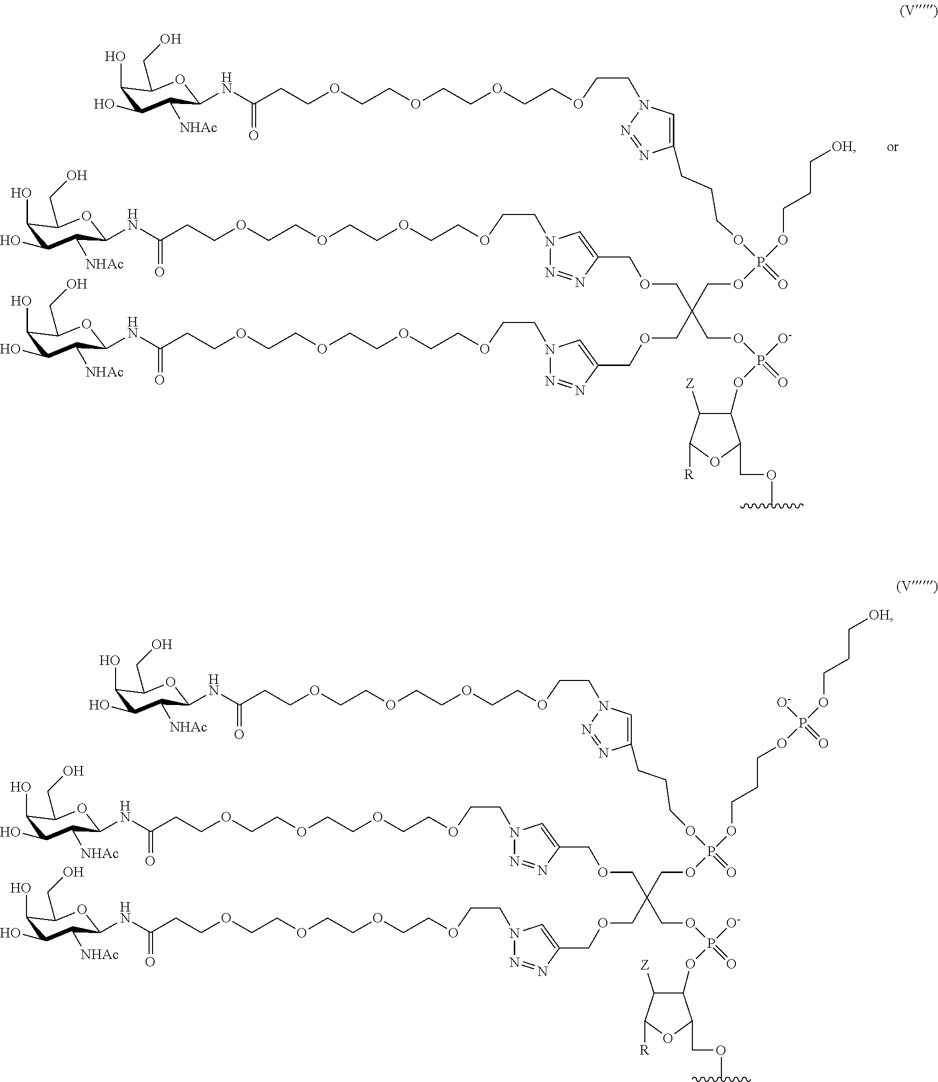

wherein Z in formula (V′), (V″ ″′), (V″″′), or (V″″″) is —H, —OH, —O-Methyl, —F, or —O— methoxyethyl, and R in formula (V′), (V″″), (V″″″), or (V″″″″) is adenine, uracil, guanine, cytosine, thymine, abasic, or others.

In one aspect, the present disclosure provides a pharmaceutical composition comprising a polynucleic acid molecule described herein, or a polynucleic acid molecule conjugate described herein, and a pharmaceutically acceptable excipient. In some embodiments, the pharmaceutical composition is formulated as a nanoparticle formulation. In some embodiments, the pharmaceutical composition is formulated for parenteral, oral, intranasal, buccal, rectal, transdermal, intravenous, subcutaneous, or intrathecal administration.

In one aspect, the present disclosure provides a method of modulating an mRNA expression of Angiopoietin-like protein 3 (ANGPTL3) gene in a subject, comprising: administering to the subject a polynucleic acid molecule described herein, a polynucleic acid molecule conjugate described herein, or a pharmaceutical composition described herein, thereby modulating the mRNA expression of ANGPTL3 gene in the subject.

In another aspect, the present disclosure provides a method of preventing, alleviating, or treating ANGPTL3-associated disease or symptom thereof in a subject in need thereof, comprising: administering to the subject a polynucleic acid molecule described herein, a polynucleic acid molecule conjugate described herein, or a pharmaceutical composition described herein, thereby preventing, alleviating, or treating ANGPTL3-associated disease or symptom thereof in the subject. In some embodiments, the ANGPTL3-associated disease or symptom thereof comprises hyperlipidermia. In some embodiments, the ANGPTL3-associated disease or symptom thereof comprises atherosclerosis, coronary heart disease, and vascular disease.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts the inhibition efficacies of selected ANGPTL3 siRNAs for in vivo testing in mice in the Example 3. The results are shown in the average % change in serum hANGPTL3 protein levels from day −4 baseline. These results correspond to the data in Table 8.

FIG. 2 depicts the inhibition efficacies of selected ANGPTL3 siRNAs for in vivo testing in mice in the Example 4. The results are shown in the average % change in serum hANGPTL3 protein levels from day −4 baseline. This results correspond to the data in Table 9.

FIG. 3 depicts the inhibition efficacies of selected ANGPTL3 siRNAs for in vivo testing in mice in the Example 5. The results are shown in the average % change in serum hANGPTL3 protein levels from day −4 baseline. These results correspond to the data in Table 10.

FIG. 4 depicts the inhibition efficacies of selected ANGPTL3 siRNAs for in vivo testing in mice in the Example 6. The results are shown in the average % change in serum hANGPTL3 protein levels relative to the vehicle control group. These results correspond to the data in Table 11.

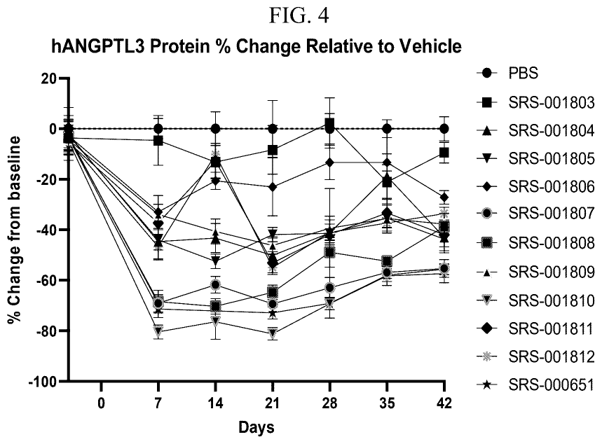

FIG. 5 depicts the inhibition efficacies of selected ANGPTL3 siRNAs for in vivo testing in cynomolgus monkeys in the Example 7. The results are shown in the average % change in serum ANGPTL3 relative to day 1. These results correspond to the data in Table 12.

FIG. 6 depicts ANGPTL3 mRNA expression level from liver tissue of cynomolgus monkeys in the Example 7. The results are shown in relative expression to the day −15.

FIG. 7 depicts the inhibition efficacies of selected ANGPTL3 siRNAs for in vivo testing in mice in the Example 8. The results are shown in the average % change in serum hANGPTL3 protein levels relative to the vehicle control group. The results correspond to the data in Table 13.

### DETAILED DESCRIPTION OF THE DISCLOSURE

Angiopoietin-like protein 3 or angiopoietin-like 3 (ANGPTL3) is a member of the angiopoietin-like protein family, which shares a structural similarity to angiopoietin protein family. ANGPTL3 is a secreted protein that is produced primarily by the liver cells and is notably expressed in kidney podocytes. ANGPTL3 regulates all 3 major lipids: LDL-cholesterol, HDL-cholesterol, and triglycerides. ANGPTL3 inhibits lipoprotein lipase (LPL) and endothelial lipase to modulate triglycerides and cholesterol metabolism.

The gene of ANGPTL3 is located on the shorter arm of chromosome 1 (1p31.3). The mRNA of ANGPTL3 (NM 014495.4) contains 2926 bp, which divides into 7 exons. ANGPTL3 is a 460-amino acid polypeptide comprising: a distinctive signal peptide sequence, a N-terminal coiled-coil domain, and a C-terminal globular fibrinogen-like domain. The N-terminal coiled-coil region (17-207 amino acid), especially at the amino acid domain 61-66, affects plasma triglyceride levels by inhibiting the catalytic activity of lipoprotein lipase (LPL). The C-terminal fibrogen-like domain (207−460 amino acid) can bind to integrin αvβ3 receptor, which affects angiogenesis. ANGPTL3 also contains a short linker region: Arg²²¹-Ala²²²-Pro²²³-Arg²²⁴, which is between N- and C-terminal domains. This liker region functions as furin cleavage sites located between amino acid residues Arg²²¹-Ala²²² and Arg²²⁴-Thr²²⁵. Similar to other members in the angiopoietin-like protein family, ANGPTL3 undergoes cleavage, resulting in separate fragments containing the N-terminal coiled-coil domain and the C-terminal fibrinogen-like domain, which appear to have different function. Both full length and truncated forms of ANGPTL3 are found in plasma. (see Tikka, A. et al., Endocrine. 2016:187-193; Wang, X. et al., JACC: Basic to Translational Science. 2019:755-762; Ono, M. et al., The Journal of Biological Chemistry. 2003:41804−41809; and NCBI Reference Sequence No: NM_014495.4).

Loss of function in the ANGPTL3 gene shows association with low levels of plasma LDL-cholesterol, HDL-cholesterol, and triglycerides. As such, targeting ANGPTL3 can achieve lipid-lowering therapy without causing severe side effect, and further provide method to reduce risk of atherosclerosis, coronary heart disease, and other vascular disease.

Described herein is a polynucleic acid molecule for modulating an expression of ANGPTL3 gene. In some aspects, the polynucleic acid molecule is a single-stranded nucleic acid molecule. In some aspects, the polynucleic acid molecule is a double-stranded nucleic acid molecule that comprises a sense strand (passenger strand) and an antisense strand (guide strand). In some aspects, the polynucleic acid molecule comprises a nucleic acid sequence in Table 1, Table 2, Table 3, or Table 4. Accordingly, provided herein are various target regions of human ANGPTL3 mRNA the polynucleic acid molecule described herein hybridizes to. In some embodiments, provided herein is the sequences of the polynucleic acid molecule described herein. In some embodiments, provided herein is the conjugates of the polynucleic acid molecule described herein. In some aspects, provided herein is the modifications of the polynucleic acid molecule described herein.

Also described herein is a method of modulating an expression of ANGPTL3 mRNA or protein in a subject. Described further herein is a method of preventing, alleviating, or treating ANGPTL3-associated disease or a symptom thereof in a subject in need thereof.

### Definitions

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B.”

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

“Percent (%) sequence identity” or “Percent (%) identity” with respect to the nucleic acid sequences identified herein is defined as the percentage of nucleic acid in a candidate sequence that are identical with the nucleic acid sequence being compared, after aligning the sequences considering any conservative substitutions as part of the sequence identity.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, and so forth. As anon-limiting example, each range discussed herein can be readily broken down into a lower third, middle third and upper third, and the like. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Unless defined otherwise, all technical and scientific terms used herein have the same meaning as commonly understood by one of ordinary skill in the art to which the polynucleic acid molecules, the polynucleic acid molecule conjugates, the pharmaceutical compositions, the methods and other aspects belong.

As used herein, the term “complementary” indicates a sufficient degree of complementarity between two nucleic acid molecules that bind stably and specifically to avoid nonspecific binding.

As used herein, the term “polynucleic acid” and the term “polynucleotide” are interchangeably used to refer a chain of nucleotides. The term “nucleotide” includes a sequence “G,” “C,” “A,” “T” and “U” each generally stand for a nucleotide that contains guanine, cytosine, adenine, thymidine and uracil as a base. In some instances, the “nucleotide” can refer to a modified nucleotide (e.g., with modified sugar moiety, modified base, modified internucleotide linkage, or combination thereof, including, but not limited to 2′-modified nucleotide, LNA, ENA, BNA, UNA, GNA etc.) In some instances, the “nucleotide” can refer to a modified nucleotide with a non-canonical base (e.g. including, but not limited to, 2-thiouridine, 2-thiothymidine, inosine, 2-aminopurine, 2,6-diaminopurine, dihydrouridine, 4-thiouridine, 4-thiothymidine, 2-thiocytidine).

As used herein, a “subject” can be any mammal, including a human and a non-human primate.

The term “condition,” as used herein, includes diseases, disorders, and susceptibilities. In some cases, the condition is an AGT related disorder or symptoms thereof.

As used herein, the term “treat,” “treating” or “treatment” of any disease or disorder refers, in one instance, to ameliorating the disease or disorder (i.e., slowing or arresting or reducing the development of the disease or at least one of the clinical symptoms thereof). In another instance, “treat”, “treating” or “treatment” refers to alleviating or ameliorating at least one physical parameter including those which may not be discernible by the patient. In yet another instance, “treat”, “treating” or “treatment” refers to modulating the disease or disorder, either physically, (e.g., stabilization of a discernible symptom), physiologically, (e.g., stabilization of a physical parameter), or both.

The terms “prevent,” “preventing,” and “prevention,” as used herein, refer to a decrease in the occurrence of pathology of a condition in a subject, who does not have, but is at risk of or susceptible to developing a disease or condition. The prevention may be complete, e.g., the total absence of pathology of a condition in a subject. The prevention may also be partial, such that the occurrence of pathology of a condition in a subject is less than that which would have occurred without the present disclosure.

“Administering” and its grammatical equivalents as used herein can refer to providing pharmaceutical compositions described herein to a subject or a patient. Conventional methods, known to those of ordinary skill in the art of medicine, can be used to administer the composition to the subject, depending upon the type of disease to be treated or the site of the disease. For example, the composition can be administered, e.g., orally, parenterally, by inhalation spray, topically, rectally, nasally, buccally, vaginally, via an implanted reservoir, or via infusion. One or more such routes can be employed.

The terms “pharmaceutical composition” and its grammatical equivalents as used herein can refer to a mixture or solution comprising a therapeutically effective amount of an active pharmaceutical ingredient together with one or more pharmaceutically acceptable excipients, carriers, and/or a therapeutic agent to be administered to a subject, e.g., a human in need thereof.

The term “pharmaceutically acceptable” and its grammatical equivalents as used herein can refer to an attribute of a material which is useful in preparing a pharmaceutical composition that is generally safe, non-toxic, and neither biologically nor otherwise undesirable and is acceptable for veterinary as well as human pharmaceutical use. “Pharmaceutically acceptable” can refer a material, such as a carrier or diluent, which does not abrogate the biological activity or properties of the compound, and is relatively nontoxic, i.e., the material may be administered to a subject without causing undesirable biological effects or interacting in a deleterious manner with any of the components of the pharmaceutical composition in which it is contained.

A “pharmaceutically acceptable excipient” refers to an excipient that can be administered to a subject, together with an agent, and which does not destroy the pharmacological activity thereof and is nontoxic when administered in doses sufficient to deliver a therapeutic amount of the agent.

The term “therapeutic agent” can refer to any agent that, when administered to a subject, has a therapeutic, diagnostic, and/or prophylactic effect and/or elicits a desired biological and/or pharmacological effect. Therapeutic agents can also be referred to as “actives” or “active agents.” Such agents include, but are not limited to, cytotoxins, radioactive ions, chemotherapeutic agents, small molecule drugs, proteins, and nucleic acids.

As used herein, the term “sense strand” can be interchangeably used with the term “passenger strand”, and the tern “antisense strand” can be interchangeably used with the term “guide strand”. In some instances, a nucleic acid sequence described herein for a sense strand and a passenger strand can be interchangeably used. Also, in some instances, a nucleic acid sequence described herein for an antisense strand and a guide strand can be interchangeably used.

As used herein, the term “consecutive sequence” refers to a sequence contains a number of consecutive nucleotides from a reference sequence. For example, if a reference sequence is N₁N₂N₃N₄N₅N₆N₇, a consecutive sequence can be N₁N₂N₃N₄ or N₃N₄N₅N₆, but a sequence of N₁N₃N₄N₅ or N₃N₄N₇ cannot be a consecutive sequence.

As used herein, the term “negative control” refers to a subject or a cell receiving no treatment or placebo.

It is appreciated that certain features of the polynucleic acid molecules, and/or polynucleic acid molecule conjugates, pharmaceutical composition comprising the polynucleic acid molecules or the polynucleic acid molecule conjugates, methods and other aspects, which are, for clarity, described in the context of separate embodiments, may also be provided in combination in a single embodiment. Conversely, various features of the polynucleic acid molecules, and/or polynucleic acid molecule conjugates, pharmaceutical composition comprising the polynucleic acid molecules or the polynucleic acid molecule conjugates, methods and other aspects, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present polynucleic acid molecules, and/or polynucleic acid molecule conjugates, pharmaceutical composition comprising the polynucleic acid molecules or the polynucleic acid molecule conjugates, methods and other aspects and are disclosed herein just as if each and every such sub-combination was individually and explicitly disclosed herein.

### Polynucleic Acid Molecules

### Target Regions of Polynucleic Acid Molecules

Described herein is a polynucleic acid molecule for modulating an expression of ANGPTL3 gene. In some instances, the polynucleic acid molecule comprises a single-stranded nucleic acid molecule that hybridizes to a certain region of mRNA. In some instances, the polynucleic acid molecule is a double-stranded nucleic acid molecule. Also described herein is a polynucleic acid molecule for modulating an expression of ANGPTL3 gene, wherein the polynucleic acid molecule is a double-stranded nucleic acid molecule, which comprises a passenger strand (a sense strand) and a guide strand (an antisense strand), and the guide strand hybridizes to a certain region of ANGPTL3 mRNA.

In some aspects, the polynucleic acid molecule described herein hybridizes to acertain region of human ANGPTL3 mRNA. In some instances, the human ANGPTL3 mRNA is as referred as NM_014495.4. In some aspects, the polynucleic acid molecule described herein hybridizes to a certain region of non-human ANGPTL3 mRNA.

In some aspects, the polynucleic acid molecule described herein hybridizes to the 5′ UTR region of human ANGPTL3 mRNA. In some aspects, the polynucleic acid molecule described herein hybridizes to the coding region of human ANGPTL3 mRNA. In some aspects, the polynucleic acid molecule described herein hybridizes to a portion of exon 1 of human ANGPTL3 mRNA. In some aspects, the polynucleic acid molecule described herein hybridizes to a portion of exon 2 of human ANGPTL3 mRNA. In some aspects, the polynucleic acid molecule described herein hybridizes to a portion of exon 3 of human ANGPTL3 mRNA. In some aspects, the polynucleic acid molecule described herein hybridizes to a portion of exon 4 of human ANGPTL3 mRNA. In some aspects, the polynucleic acid molecule described herein hybridizes to a portion of exon 5 of human ANGPTL3 mRNA. In some aspects, the polynucleic acid molecule described herein hybridizes to a portion of exon 6 of human ANGPTL3 mRNA. In some aspects, the polynucleic acid molecule described herein hybridizes to a portion of exon 7 of human ANGPTL3 mRNA. In some aspects, the polynucleic acid molecule described herein hybridizes to the 3′ UTR region of human ANGPTL3 mRNA.

In some aspects, the target region that the polynucleic acid molecule described herein hybridizes to is determined by ANGPTL3 silencing effectiveness and possible off-target effects. In some instances, the start of the target region fall between positions 1-10, 11-20, 21-30, 31-40, 41-50, 51-60, 61-70, 71-80, 81-90, or 91-100 of NM_014495.4. In some instances, the start of the target region fall between positions 101-110, 111-120, 121-130, 131-140, 141-150, 151-160, 161-170, 171-180, 181-190, or 191-200 of NM_014495.4. In some instances, the start of the target region fall between positions of 201-210, 211-220, 221-230, 231-240, 241-250, 251-260, 261-270, 271-280, 281-290, or 291-300 of NM_014495.4. In some instances, the start of the target region fall between positions 301-310, 311-320, 321-330, 331-340, 341-350, 351-360, 361-370, 371-380, 381-390, or 391-400 of NM_014495.4. In some instances, the start of the target region fall between positions 401-410, 411-420, 421-430, 431-440, 441-450, 451-460, 461-470, 471-480, 481-490, or 491-500 of NM_014495.4. In some instances, the start of the target region fall between positions 501-510, 511-520, 521-530, 531-540, 541-550, 551-560, 561-570, 571-580, 581-590, or 591-600 of NM_014495.4. In some instances, the start of the target region fall between positions 601-610, 611-620, 621-630, 631-640, 641-650, 651-660, 661-670, 671-680, 681-690, or 691-700 of NM_014495.4. In some instances, the start of the target region fall between positions 701-710, 711-720, 721-730, 731-740, 741-750, 751-760, 761-770, 771-780, 781-790, or 791-800 of NM_014495.4. In some instances, the start of the target region fall between positions 801-810, 811-820, 821-830, 831-840, 841-850, 851-860, 861-870, 871-880, 881-890, or 891-900 of NM_014495.4. In some instances, the start of the target region fall between positions 901-910, 911-920, 921-930, 931-940, 941-950, 951-960, 961-970, 971-980, 981-990, or 991-1000 of NM_014495.4. In some instances, the start of the target region fall between positions 1001-1010, 1011-1020, 1021-1030, 1031-1040, 1041-1050, 1051-1060, 1061-1070, 1071-1080, 1081-1090, or 1091-1100 of NM_014495.4. In some instances, the start of the target region fall between positions 1101-1110, 1111-1120, 1121-1130, 1131-1140, 1141-1150, 1151-1160, 1161-1170, 1171-1180, 1181-1190, or 1191-1200 of NM_014495.4. In some instances, the start of the target region fall between positions 1201-1210, 1211-1220, 1221-1230, 1231-1240, 1241-1250, 1251-1260, 1261-1270, 1271-1280, 1281-1290, or 1291-1300 of NM_014495.4. In some instances, the start of the target region fall between positions 1301-1310, 1311-1320, 1321-1330, 1331-1340, 1341-1350, 1351-1360, 1361-1370, 1371-1380, 1381-1390, or 1391-1400 of NM_014495.4. In some instances, the start of the target region fall between positions 1401-1410, 1411-1420, 1421-1430, 1431-1440, 1441-1450, 1451-1460, 1461-1470, 1471-1480, 1481-1490, or 1491-1500 of NM_014495.4. In some instances, the start of the target region fall between positions 1501-1510, 1511-1520, 1521-1530, 1531-1540, 1541-1550, 1551-1560, 1561-1570, 1571-1580, 1581-1590, or 1591-1600 of NM_014495.4. In some instances, the start of the target region fall between positions 1601-1610, 1611-1620, 1621-1630, 1631-1640, 1641-1650, 1651-1660, 1661-1670, 1671-1680, 1681-1690, or 1691-1700 of NM_014495.4. In some instances, the start of the target region fall between positions 1701-1710, 1711-1720, 1721-1730, 1731-1740, 1741-1750, 1751-1760, 1761-1770, 1771-1780, 1781-1790, or 1791-1800 of NM_014495.4. In some instances, the start of the target region fall between positions 1801-1810, 1811-1820, 1821-1830, 1831-1840, 1841-1850, 1851-1860, 1861-1870, 1871-1880, 1881-1890, or 1891-1900 of NM_014495.4. In some instances, the start of the target region fall between positions 1901-1910, 1911-1920, 1921-1930, 1931-1940, 1941-1950, 1951-1960, 1961-1970, 1971-1980, 1981-1990, or 1991-2000 of NM_014495.4. In some instances, the start of the target region fall between positions 2001-2010, 2011-2020, 2021-2030, 2031-2040, 2041-2050, 2051-2060, 2061-2070, 2071-2080, 2081-2090, or 2091-2100 of NM_014495.4. In some instances, the start of the target region fall between positions 2101-2110, 2111-2120, 2121-2130, 2131-2140, 2141-2150, 2151-2160, 2161-2170, 2171-2180, 2181-2190, or 2191-2200 of NM_014495.4. In some instances, the start of the target region fall between positions 2201-2210, 2211-2220, 2221-2230, 2231-2240, 2241-2250, 2251-2260, 2261-2270, 2271-2280, 2281-2290, or 2291-2300 of NM_014495.4. In some instances, the start of the target region fall between positions 2301-2310, 2311-2320, 2321-2330, 2331-2340, 2341-2350, 2351-2360, 2361-2370, 2371-2380, 2381-2390, or 2391-2400 of NM_014495.4. In some instances, the start of the target region fall between positions 2401-2410, 2411-2420, 2421-2430, 2431-2440, 2441-2450, 2451-2460, 2461-2470, 2471-2480, 2481-2490, or 2491-2500 of NM_014495.4. In some instances, the start of the target region fall between positions 2501-2510, 2511-2520, 2521-2530, 2531-2540, 2541-2550, 2551-2560, 2561-2570, 2571-2580, 2581-2590, or 2591-2600 of NM_014495.4. In some instances, the start of the target region fall between positions 2601-2610, 2611-2620, 2621-2630, 2631-2640, 2641-2650, 2651-2660, 2661-2670, 2671-2680, 2681-2690, or 2691-2700 of NM_014495.4. In some instances, the start of the target region fall between positions 2701-2710, 2711-2720, 2721-2730, 2731-2740, 2741-2750, 2751-2760, 2761-2770, 2771-2780, 2781-2790, or 2791-2800 of NM_014495.4. In some instances, the start of the target region fall between positions 2801-2810, 2811-2820, 2821-2830, 2831-2840, 2841-2850, 2851-2860, 2861-2870, 2871-2880, 2881-2890, or 2891-2900 of NM_014495.4. In some instances, the start of the target region fall between positions 2901-2910, 2911-2920, or 2921-2926 of NM_014495.4.

### Structure of Polynucleic Acid Molecules

### Single-Stranded Nucleic Acid Molecule

Described herein is a polynucleic acid molecule for modulating an expression of ANGPTL3 gene, wherein the polynucleic acid molecule comprises a single-stranded nucleic acid molecule that is reverse complementary to the target region of ANGPTL3 mRNA as described herein.

In some aspects, the polynucleic acid molecule described herein is not 100% complementary to the target region of ANGPTL3 mRNA. Accordingly, in some instances, the polynucleic acid molecule described herein is about 95% complementary to the target region of ANGPTL3 mRNA. In some instances, the polynucleic acid molecule described herein is about 90% complementary to the target region of ANGPTL3 mRNA. In some instances, the polynucleic acid molecule described herein is about 85% complementary to the target region of ANGPTL3 mRNA. In some instances, the polynucleic acid molecule described herein is about 80% complementary to the target region of ANGPTL3 mRNA. In some instances, the polynucleic acid molecule described herein is about 75% complementary to the target region of ANGPTL3 mRNA. In some instances, the polynucleic acid molecule described herein is about 70% complementary to the target region of ANGPTL3 mRNA.

In some aspects, the polynucleic acid molecule described herein comprises a nucleic acid sequence in Table 1, Table 2, Table 3, or Table 4. In some instances, the polynucleic acid molecule described herein comprises a nucleic acid sequence that is at least 80%, at least 85%, at least 90%, or at least 95% complementary to a nucleic acid sequence in Table 1, Table 2, Table 3, or Table 4. In some instances, the polynucleic acid molecule described herein comprises a nucleic acid sequence that is at least 80%, at least 85%, at least 90%, or at least 95% complementary to a nucleic acid sequence selected from SEQ ID NOs: 401-596, 819-828, and 870-875. In some instances, the polynucleic acid molecule described herein comprises a nucleic acid sequence that is at least 80%, at least 85%, at least 90%, or at least 95% complementary to a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875. In some instances, the polynucleic acid molecule described herein comprises a nucleic acid sequence that is at least 80%, at least 85%, at least 90%, or at least 95% complementary to a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875. In some instances, the polynucleic acid molecule described herein comprises a nucleic acid sequence that is at least 80%, at least 85%, at least 90%, or at least 95% complementary to a nucleic acid sequence selected from SEQ ID NOs: 513, 546, 553, 558, 508, 514, and 545.

In some instances, the polynucleic acid molecule described herein comprises a nucleic acid sequence that is at least 80%, at least 85%, at least 90%, or at least 95% complementary to a nucleic acid sequence in Table 1, Table 2, Table 3, or Table 4, excluding overhangs. In some instances, the polynucleic acid molecule described herein comprises a nucleic acid sequence that is at least 80%, at least 85%, at least 90%, or at least 95% complementary to a nucleic acid sequence selected from SEQ ID NOs: 401-596, 819-828, and 870-875, excluding overhangs. In some instances, the polynucleic acid molecule described herein comprises a nucleic acid sequence that is at least 80%, at least 85%, at least 90%, or at least 95% complementary to a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875, excluding overhangs. In some instances, the polynucleic acid molecule described herein comprises a nucleic acid sequence that is at least 80%, at least 85%, at least 90%, or at least 95% complementary to a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875, excluding overhangs. In some instances, the polynucleic acid molecule described herein comprises a nucleic acid sequence that is at least 80%, at least 85%, at least 90%, or at least 95% complementary to a nucleic acid sequence selected from SEQ ID NOs: 513, 546, 553, 558, 508, 514, and 545, excluding overhangs.

In yet other aspects, the polynucleic acid molecule described herein comprises a nucleic acid sequence comprising 14 consecutive nucleotides that are complementary to a nucleic acid sequence in Table 1, Table 2, Table 3, or Table 4 with no more than 1, 2, 3, or 4 mismatches. In some aspects, the polynucleic acid molecule described herein comprises a nucleic acid sequence comprising 14 consecutive (contiguous) nucleotides that are complementary to a nucleic acid sequence selected from SEQ ID NOs: 401-596, 819-828, and 870-875 with no more than 1, 2, 3, or 4 mismatches. In yet other aspects, the polynucleic acid molecule described herein comprises a nucleic acid sequence comprising 15 consecutive nucleotides that are complementary to a nucleic acid sequence in Table 1, Table 2, Table 3, or Table 4 with no more than 1, 2, 3, or 4 mismatches. In some aspects, the polynucleic acid molecule described herein comprises a nucleic acid sequence comprising 15 consecutive (contiguous) nucleotides that are complementary to a nucleic acid sequence selected from SEQ ID NOs: 401-596, 819-828, and 870-875 with no more than 1, 2, 3, or 4 mismatches. In yet other aspects, the polynucleic acid molecule described herein comprises a nucleic acid sequence comprising 16 consecutive nucleotides that are complementary to a nucleic acid sequence in Table 1, Table 2, Table 3, or Table 4 with no more than 1, 2, 3, or 4 mismatches. In some aspects, the polynucleic acid molecule described herein comprises a nucleic acid sequence comprising 16 consecutive (contiguous) nucleotides that are complementary to a nucleic acid sequence selected from SEQ ID NOs: 401-596, 819-828, and 870-875 with no more than 1, 2, 3, or 4 mismatches. In yet other aspects, the polynucleic acid molecule described herein comprises a nucleic acid sequence comprising 17 consecutive nucleotides that are complementary to a nucleic acid sequence in Table 1, Table 2, Table 3, or Table 4 with no more than 1, 2, 3, or 4 mismatches. In some aspects, the polynucleic acid molecule described herein comprises a nucleic acid sequence comprising 17 consecutive (contiguous) nucleotides that are complementary to a nucleic acid sequence selected from SEQ ID NOs: 401-596, 819-828, and 870-875 with no more than 1, 2, 3, or 4 mismatches. In yet other aspects, the polynucleic acid molecule described herein comprises a nucleic acid sequence comprising 18 consecutive nucleotides that are complementary to a nucleic acid sequence in Table 1, Table 2, Table 3, or Table 4 with no more than 1, 2, 3, or 4 mismatches. In some aspects, the polynucleic acid molecule described herein comprises a nucleic acid sequence comprising 18 consecutive (contiguous) nucleotides that are complementary to a nucleic acid sequence selected from SEQ ID NOs: 401-596, 819-828, and 870-875 with no more than 1, 2, 3, or 4 mismatches. In yet other aspects, the polynucleic acid molecule described herein comprises a nucleic acid sequence comprising 19 consecutive (contiguous) nucleotides that are complementary to a nucleic acid sequence in Table 1, Table 2, Table 3, or Table 4 with no more than 1, 2, 3, or 4 mismatches. In some aspects, the polynucleic acid molecule described herein comprises a nucleic acid sequence comprising 19 consecutive (contiguous) nucleotides that are complementary to a nucleic acid sequence selected from SEQ ID NOs: 401-596, 819-828, and 870-875 with no more than 1, 2, 3, or 4 mismatches. In yet other aspects, the polynucleic acid molecule described herein comprises a nucleic acid sequence comprising 20 consecutive (contiguous) nucleotides that are complementary to a nucleic acid sequence in Table 1, Table 2, Table 3, or Table 4 with no more than 1, 2, 3, or 4 mismatches. In some aspects, the polynucleic acid molecule described herein comprises a nucleic acid sequence comprising 20 consecutive (contiguous) nucleotides that are complementary to a nucleic acid sequence selected from SEQ ID NOs: 401-596, 819-828, and 870-875 with no more than 1, 2, 3, or 4 mismatches. In yet other aspects, the polynucleic acid molecule described herein comprises a nucleic acid sequence comprising 21 consecutive (contiguous) nucleotides that are complementary to a nucleic acid sequence in Table 1, Table 2, Table 3, or Table 4 with no more than 1, 2, 3, or 4 mismatches. In some aspects, the polynucleic acid molecule described herein comprises a nucleic acid sequence comprising 21 consecutive (contiguous) nucleotides that are complementary to a nucleic acid sequence selected from SEQ ID NOs: 401-596, 819-828, and 870-875 with no more than 1, 2, 3, or 4 mismatches. In yet other aspects, the polynucleic acid molecule described herein comprises a nucleic acid sequence comprising 22 consecutive (contiguous) nucleotides that are complementary to a nucleic acid sequence in Table 1, Table 2, Table 3, or Table 4 with no more than 1, 2, 3, or 4 mismatches. In some aspects, the polynucleic acid molecule described herein comprises a nucleic acid sequence comprising 22 consecutive (contiguous) nucleotides that are complementary to a nucleic acid sequence selected from SEQ ID NOs: 401-596, 819-828, and 870-875 with no more than 1, 2, 3, or 4 mismatches.

In some aspects, the polynucleic acid molecule described herein comprises a nucleic acid sequence comprising 14 consecutive nucleotides that are complementary to a nucleic acid sequence selected from SEQ ID NOs: 402-403, 405, 407-408, 410-411, 414-415, 418, 420-422, 432, 434, 442-446, 450-456, 460, 476-477, 496, 498, 507-509, 511, 513-514, 535, 545-546, 548-549, 553, 545-546, 548-549, 553, 557-558, 560-561, 819-828, and 870-875 with no more than 1, 2, 3, or 4 mismatches. In some aspects, the polynucleic acid molecule described herein comprises a nucleic acid sequence comprising 15 consecutive nucleotides that are complementary to a nucleic acid sequence selected from SEQ ID NOs: 402-403, 405, 407-408, 410-411, 414-415, 418, 420-422, 432, 434, 442-446, 450-456, 460, 476-477, 496, 498, 507-509, 511, 513-514, 535, 545-546, 548-549, 553, 545-546, 548-549, 553, 557-558, 560-561, 819-828, and 870-875 with no more than 1, 2, 3, or 4 mismatches. In some aspects, the polynucleic acid molecule described herein comprises a nucleic acid sequence comprising 16 consecutive nucleotides that are complementary to a nucleic acid sequence selected from SEQ ID NOs: 402-403, 405, 407-408, 410-411, 414-415, 418, 420-422, 432, 434, 442-446, 450-456, 460, 476-477, 496, 498, 507-509, 511, 513-514, 535, 545-546, 548-549, 553, 545-546, 548-549, 553, 557-558, 560-561, 819-828, and 870-875 with no more than 1, 2, 3, or 4 mismatches. In some aspects, the polynucleic acid molecule described herein comprises a nucleic acid sequence comprising 17 consecutive nucleotides that are complementary to a nucleic acid sequence selected from SEQ ID NOs: 402-403, 405, 407-408, 410-411, 414-415, 418, 420-422, 432, 434, 442-446, 450-456, 460, 476-477, 496, 498, 507-509, 511, 513-514, 535, 545-546, 548-549, 553, 545-546, 548-549, 553, 557-558, 560-561, 819-828, and 870-875 with no more than 1, 2, 3, or 4 mismatches. In some aspects, the polynucleic acid molecule described herein comprises a nucleic acid sequence comprising 18 consecutive nucleotides that are complementary to a nucleic acid sequence selected from SEQ ID NOs: 402-403, 405, 407-408, 410-411, 414-415, 418, 420-422, 432, 434, 442-446, 450-456, 460, 476-477, 496, 498, 507-509, 511, 513-514, 535, 545-546, 548-549, 553, 545-546, 548-549, 553, 557-558, 560-561, 819-828, and 870-875 with no more than 1, 2, 3, or 4 mismatches. In some aspects, the polynucleic acid molecule described herein comprises a nucleic acid sequence comprising 19 consecutive nucleotides that are complementary to a nucleic acid sequence selected from SEQ ID NOs: 402-403, 405, 407-408, 410-411, 414-415, 418, 420-422, 432, 434, 442-446, 450-456, 460, 476-477, 496, 498, 507-509, 511, 513-514, 535, 545-546, 548-549, 553, 545-546, 548-549, 553, 557-558, 560-561, 819-828, and 870-875 with no more than 1, 2, 3, or 4 mismatches. In some aspects, the polynucleic acid molecule described herein comprises a nucleic acid sequence comprising 20 consecutive nucleotides that are complementary to a nucleic acid sequence selected from SEQ ID NOs: 402-403, 405, 407-408, 410-411, 414-415, 418, 420-422, 432, 434, 442-446, 450-456, 460, 476-477, 496, 498, 507-509, 511, 513-514, 535, 545-546, 548-549, 553, 545-546, 548-549, 553, 557-558, 560-561, 819-828, and 870-875 with no more than 1, 2, 3, or 4 mismatches. In some aspects, the polynucleic acid molecule described herein comprises a nucleic acid sequence comprising 21 consecutive nucleotides that are complementary to a nucleic acid sequence selected from SEQ ID NOs: 402-403, 405, 407-408, 410-411, 414-415, 418, 420-422, 432, 434, 442-446, 450-456, 460, 476-477, 496, 498, 507-509, 511, 513-514, 535, 545-546, 548-549, 553, 545-546, 548-549, 553, 557-558, 560-561, 819-828, and 870-875 with no more than 1, 2, 3, or 4 mismatches. In some aspects, the polynucleic acid molecule described herein comprises a nucleic acid sequence comprising 22 consecutive nucleotides that are complementary to a nucleic acid sequence selected from SEQ ID NOs: 402-403, 405, 407-408, 410-411, 414-415, 418, 420-422, 432, 434, 442-446, 450-456, 460, 476-477, 496, 498, 507-509, 511, 513-514, 535, 545-546, 548-549, 553, 545-546, 548-549, 553, 557-558, 560-561, 819-828, and 870-875 with no more than 1, 2, 3, or 4 mismatches.

In some aspects, the polynucleic acid molecule described herein comprises a nucleic acid sequence comprising 14 consecutive nucleotides that are complementary to a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875 with no more than 1, 2, 3, or 4 mismatches. In some aspects, the polynucleic acid molecule described herein comprises a nucleic acid sequence comprising 15 consecutive nucleotides that are complementary to a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875 with no more than 1, 2, 3, or 4 mismatches. In some aspects, the polynucleic acid molecule described herein comprises a nucleic acid sequence comprising 16 consecutive nucleotides that are complementary to a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875 with no more than 1, 2, 3, or 4 mismatches. In some aspects, the polynucleic acid molecule described herein comprises a nucleic acid sequence comprising 17 consecutive nucleotides that are complementary to a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875 with no more than 1, 2, 3, or 4 mismatches. In some aspects, the polynucleic acid molecule described herein comprises a nucleic acid sequence comprising 18 consecutive nucleotides that are complementary to a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875 with no more than 1, 2, 3, or 4 mismatches. In some aspects, the polynucleic acid molecule described herein comprises a nucleic acid sequence comprising 19 consecutive nucleotides that are complementary to a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875 with no more than 1, 2, 3, or 4 mismatches. In some aspects, the polynucleic acid molecule described herein comprises a nucleic acid sequence comprising 20 consecutive nucleotides that are complementary to a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875 with no more than 1, 2, 3, or 4 mismatches. In some aspects, the polynucleic acid molecule described herein comprises a nucleic acid sequence comprising 21 consecutive nucleotides that are complementary to a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875 with no more than 1, 2, 3, or 4 mismatches. In some aspects, the polynucleic acid molecule described herein comprises a nucleic acid sequence comprising 22 consecutive nucleotides that are complementary to a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875 with no more than 1, 2, 3, or 4 mismatches.

In some aspects, the polynucleic acid molecule described herein comprises a nucleic acid sequence comprising 14 consecutive nucleotides that are complementary to a nucleic acid sequence selected from SEQ ID NOs: 513, 546, 553, 558, 508, 514, and 545 with no more than 1, 2, 3, or 4 mismatches. In some aspects, the polynucleic acid molecule described herein comprises a nucleic acid sequence comprising 15 consecutive nucleotides that are complementary to a nucleic acid sequence selected from SEQ ID NOs: 513, 546, 553, 558, 508, 514, and 545 with no more than 1, 2, 3, or 4 mismatches. In some aspects, the polynucleic acid molecule described herein comprises a nucleic acid sequence comprising 16 consecutive nucleotides that are complementary to a nucleic acid sequence selected from SEQ ID NOs: 513, 546, 553, 558, 508, 514, and 545 with no more than 1, 2, 3, or 4 mismatches. In some aspects, the polynucleic acid molecule described herein comprises a nucleic acid sequence comprising 17 consecutive nucleotides that are complementary to a nucleic acid sequence selected from SEQ ID NOs: 513, 546, 553, 558, 508, 514, and 545 with no more than 1, 2, 3, or 4 mismatches. In some aspects, the polynucleic acid molecule described herein comprises a nucleic acid sequence comprising 18 consecutive nucleotides that are complementary to a nucleic acid sequence selected from SEQ ID NOs: 513, 546, 553, 558, 508, 514, and 545 with no more than 1, 2, 3, or 4 mismatches. In some aspects, the polynucleic acid molecule described herein comprises a nucleic acid sequence comprising 19 consecutive nucleotides that are complementary to a nucleic acid sequence selected from SEQ ID NOs: 513, 546, 553, 558, 508, 514, and 545 with no more than 1, 2, 3, or 4 mismatches. In some aspects, the polynucleic acid molecule described herein comprises a nucleic acid sequence comprising 20 consecutive nucleotides that are complementary to a nucleic acid sequence selected from SEQ ID NOs: 513, 546, 553, 558, 508, 514, and 545 with no more than 1, 2, 3, or 4 mismatches. In some aspects, the polynucleic acid molecule described herein comprises a nucleic acid sequence comprising 21 consecutive nucleotides that are complementary to a nucleic acid sequence selected from SEQ ID NOs: 513, 546, 553, 558, 508, 514, and 545 with no more than 1, 2, 3, or 4 mismatches. In some aspects, the polynucleic acid molecule described herein comprises a nucleic acid sequence comprising 22 consecutive nucleotides that are complementary to a nucleic acid sequence selected from SEQ ID NOs: 513, 546, 553, 558, 508, 514, and 545 with no more than 1, 2, 3, or 4 mismatches.

In yet other aspects, the polynucleic acid molecule described herein comprises a nucleic acid sequence comprising 14, 15, 16, 17, 18, 19, 20, 21, or 22 consecutive nucleotides that are complementary to a nucleic acid sequence in Table 1, Table 2, Table 3, or Table 4 without overhangs with no more than 1, 2, 3, or 4 mismatches. In some aspects, the polynucleic acid molecule described herein comprises a nucleic acid sequence comprising 14, 15, 16, 17, 18, 19, 20, 21, or 22 consecutive nucleotides that are complementary to a nucleic acid sequence selected from SEQ ID NOs: 401-596, 819-828, and 870-875 without overhangs with no more than 1, 2, 3, or 4 mismatches. In some aspects, the polynucleic acid molecule described herein comprises a nucleic acid sequence comprising 14, 15, 16, 17, 18, 19, 20, 21, or 22 consecutive nucleotides that are complementary to a nucleic acid sequence selected from SEQ ID NOs: 402-403, 405, 407-408, 410-411, 414-415, 418, 420-422, 432, 434, 442-446, 450-456, 460, 476-477, 496, 498, 507-509, 511, 513-514, 535, 545-546, 548-549, 553, 545-546, 548-549, 553, 557-558, 560-561, 819-828, and 870-875 without overhangs with no more than 1, 2, 3, or 4 mismatches. In some aspects, the polynucleic acid molecule described herein comprises a nucleic acid sequence comprising 14, 15, 16, 17, 18, 19, 20, 21, or 22 consecutive nucleotides that are complementary to a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875 without overhangs with no more than 1, 2, 3, or 4 mismatches. In some aspects, the polynucleic acid molecule described herein comprises a nucleic acid sequence comprising 14, 15, 16, 17, 18, 19, 20, 21, or 22 consecutive nucleotides that are complementary to a nucleic acid sequence selected from SEQ ID NOs: 513, 546, 553, 558, 508, 514, and 545 without overhangs with no more than 1, 2, 3, or 4 mismatches.

In some aspects, the polynucleic acid molecule described herein comprises a strand of about 15-40, 16-30, 17-30, 18-30, 18-27, 18-25, 18-23, 19-23, 20-23, or 21-23 nucleotides in length. In some aspects, the polynucleic acid molecule described herein comprises a strand of about 15, 16, 17, 18, 19, 20 nucleotides in length. In some aspects, the polynucleic acid molecule described herein comprises a strand of about 21, 22, 23, 24, 25 nucleotides in length.

In some aspects, the polynucleic acid molecule described herein comprises a strand of about 26, 27, 28, 29, 30 nucleotides in length.

### Double-Stranded Nucleic Acid Molecule

Further described herein is a polynucleic acid molecule for modulating expression of ANGPTL3 gene, wherein the polynucleic acid molecule is a double-stranded molecule that comprises a passenger strand (a sense strand) and a guide strand (an antisense strand), and the guide strand is reverse complementary to the target region of ANGPTL3 mRNA as described above.

In some aspects, the guide strand described herein is 100% complementary to the target region of ANGPTL3 mRNA. In other aspects, the guide strand described herein is not 100% complementary to the target region of ANGPTL3 mRNA. Accordingly, in some instances, the guide strand described herein is about 95% complementary to the target region of ANGPTL3 mRNA. In some aspects, the guide strand described herein is about 90% complementary to the target region of ANGPTL3 mRNA. In some aspects, the guide strand described herein is about 85% complementary to the target region of ANGPTL3 mRNA. In some aspects, the guide strand described herein is about 80% complementary to the target region of ANGPTL3 mRNA. In some aspects, the guide strand described herein is about 75% complementary to the target region of ANGPTL3 mRNA. In some aspects, the guide strand described herein is about 70% complementary to the target region of ANGPTL3 mRNA.

In some aspects, the polynucleic acid molecule described herein comprises a nucleic acid sequence in Table 1, Table 2, Table 3, or Table 4. In other aspects, the polynucleic acid molecule described herein comprises a nucleic acid sequence that is at least 80%, at least 85%, at least 90%, or at least 95% identical to a sequence in Table 1, Table 2, Table 3, or Table 4. In some instances, the passenger strand described herein comprises a nucleic acid sequence that is at least 80%, at least 85%, at least 90%, or at least 95% identical to a nucleic acid sequence selected from SEQ ID NOs: 401-596, 819-828, and 870-875. In some instances, the guide strand described herein comprises a nucleic acid sequence that is at least 80%, at least 85%, at least 90%, or at least 95% identical to a nucleic acid sequence selected from SEQ ID NOs: 1-196, 797-806, and 850-857. In some instances, the passenger strand described herein comprises a nucleic acid sequence that is at least 80%, at least 85%, at least 90%, or at least 95% identical to a nucleic acid sequence selected from SEQ ID NOs: 402-403, 405, 407-408, 410-411, 414-415, 418, 420-422, 432, 434, 442-446, 450-456, 460, 476-477, 496, 498, 507-509, 511, 513-514, 535, 545-546, 548-549, 553, 545-546, 548-549, 553, 557-558, 560-561, 819-828, and 870-875. In some instances, the guide strand described herein comprises a nucleic acid sequence that is at least 80%, at least 85%, at least 90%, or at least 95% identical to a nucleic acid sequence selected from SEQ ID NOs: 2-3, 5, 7-8, 10-11, 14-15, 18, 20-22, 32, 34, 42-46, 50-56, 60, 76-77, 96, 98, 107-109, 111, 113-114, 135, 145-146, 148-149, 153, 157-158, 160-161, 797-806, and 850-857. In some instances, the passenger strand described herein comprises a nucleic acid sequence that is at least 80%, at least 85%, at least 90%, or at least 95% identical to a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875. In some instances, the guide strand described herein comprises a nucleic acid sequence that is at least 80%, at least 85%, at least 90%, or at least 95% identical to a nucleic acid sequence selected from SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857. In some instances, the passenger strand described herein comprises a nucleic acid sequence that is at least 80%, at least 85%, at least 90%, or at least 95% identical to a nucleic acid sequence selected from SEQ ID NOs: 513, 546, 553, 558, 508, 514, and 545. In some instances, the guide strand described herein comprises a nucleic acid sequence that is at least 80%, at least 85%, at least 90%, or at least 95% identical to a nucleic acid sequence selected from SEQ ID NOs: 113, 146, 153, 158, 108, 114, and 145.

In yet other aspects, the polynucleic acid molecule described herein comprises a nucleic acid sequence comprising 14 consecutive sequences out of the sequences in Table 1, Table 2, Table 3, or Table 4 with no more than 1, 2, 3, or 4 mismatches. In some aspects, the passenger strand described herein comprises a nucleic acid sequence comprising 14 consecutive sequences selected from SEQ ID NOs: 401-596, 819-828, and 870-875 with no more than 1, 2, 3, or 4 mismatches. In some aspects, the guide strand described herein comprises a nucleic acid sequence comprising 14 consecutive sequences selected from SEQ ID NOs: 1-196, 797-806, and 850-857 with no more than 1, 2, 3, or 4 mismatches. In yet other aspects, the polynucleic acid molecule described herein comprises a nucleic acid sequence comprising 15 consecutive sequences out of the sequences in Table 1, Table 2, Table 3, or Table 4 with no more than 1, 2, 3, or 4 mismatches. In some aspects, the passenger strand described herein comprises a nucleic acid sequence comprising 15 consecutive sequences selected from SEQ ID NOs: 401-596, 819-828, and 870-875 with no more than 1, 2, 3, or 4 mismatches. In some aspects, the guide strand described herein comprises a nucleic acid sequence comprising 15 consecutive sequences selected from SEQ ID NOs: 1-196, 797-806, and 850-857 with no more than 1, 2, 3, or 4 mismatches. In yet still other aspects, the polynucleic acid molecule described herein comprises a nucleic acid sequence comprising 16 consecutive sequences out of the sequences in Table 1, Table 2, Table 3, or Table 4 with no more than 1, 2, 3, or 4 mismatches. In some aspects, the passenger strand described herein comprises a nucleic acid sequence comprising 16 consecutive sequences selected from SEQ ID NOs: 401-596, 819-828, and 870-875 with no more than 1, 2, 3, or 4 mismatches. In some aspects, the guide strand described herein comprises a nucleic acid sequence comprising 16 consecutive sequences selected from SEQ ID NOs: 1-196, 797-806, and 850-857 with no more than 1, 2, 3, or 4 mismatches. In yet still other aspects, the polynucleic acid molecule described herein comprises a nucleic acid sequence comprising 17 consecutive sequences out of the sequences in Table 1, Table 2, Table 3, or Table 4 with no more than 1, 2, 3, or 4 mismatches. In some aspects, the passenger strand described herein comprises a nucleic acid sequence comprising 17 consecutive sequences selected from SEQ ID NOs: 401-596, 819-828, and 870-875 with no more than 1, 2, 3, or 4 mismatches. In some aspects, the guide strand described herein comprises a nucleic acid sequence comprising 17 consecutive sequences selected from SEQ ID NOs: 1-196, 797-806, and 850-857 with no more than 1, 2, 3, or 4 mismatches. In yet other aspects, the polynucleic acid molecule described herein comprises a nucleic acid sequence comprising 18 consecutive sequences out of the sequences in Table 1, Table 2, Table 3, or Table 4 with no more than 1, 2, 3, or 4 mismatches. In some aspects, the passenger strand described herein comprises a nucleic acid sequence comprising 18 consecutive sequences selected from SEQ ID NOs: 401-596, 819-828, and 870-875 with no more than 1, 2, 3, or 4 mismatches. In some aspects, the guide strand described herein comprises a nucleic acid sequence comprising 18 consecutive sequences selected from SEQ ID NOs: 1-196, 797-806, and 850-857 with no more than 1, 2, 3, or 4 mismatches. In yet other aspects, the polynucleic acid molecule described herein comprises a nucleic acid sequence comprising 19 consecutive sequences out of the sequences in Table 1, Table 2, Table 3, or Table 4 with no more than 1, 2, 3, or 4 mismatches. In some aspects, the passenger strand described herein comprises a nucleic acid sequence comprising 19 consecutive sequences selected from SEQ ID NOs: 401-596, 819-828, and 870-875 with no more than 1, 2, 3, or 4 mismatches. In some aspects, the guide strand described herein comprises a nucleic acid sequence comprising 19 consecutive sequences selected from SEQ ID NOs: 1-196, 797-806, and 850-857 with no more than 1, 2, 3, or 4 mismatches. In yet other aspects, the polynucleic acid molecule described herein comprises a nucleic acid sequence comprising 20 consecutive sequences out of the sequences in Table 1, Table 2, Table 3, or Table 4 with no more than 1, 2, 3, or 4 mismatches. In some aspects, the passenger strand described herein comprises a nucleic acid sequence comprising 20 consecutive sequences selected from SEQ ID NOs: 401-596, 819-828, and 870-875 with no more than 1, 2, 3, or 4 mismatches. In some aspects, the guide strand described herein comprises a nucleic acid sequence comprising 20 consecutive sequences selected from SEQ ID NOs: 1-196, 797-806, and 850-857 with no more than 1, 2, 3, or 4 mismatches. In yet other aspects, the polynucleic acid molecule described herein comprises a nucleic acid sequence comprising 21 consecutive sequences out of the sequences in Table 1, Table 2, Table 3, or Table 4 with no more than 1, 2, 3, or 4 mismatches. In some aspects, the passenger strand described herein comprises a nucleic acid sequence comprising 21 consecutive sequences selected from SEQ ID NOs: 401-596, 819-828, and 870-875 with no more than 1, 2, 3, or 4 mismatches. In some aspects, the guide strand described herein comprises a nucleic acid sequence comprising 21 consecutive sequences selected from SEQ ID NOs: 1-196, 797-806, and 850-857 with no more than 1, 2, 3, or 4 mismatches. In yet other aspects, the polynucleic acid molecule described herein comprises a nucleic acid sequence comprising 22 consecutive sequences out of the sequences in Table 1, Table 2, Table 3, or Table 4 with no more than 1, 2, 3, or 4 mismatches. In some aspects, the passenger strand described herein comprises a nucleic acid sequence comprising 22 consecutive sequences selected from SEQ ID NOs: 401-596, 819-828, and 870-875 with no more than 1, 2, 3, or 4 mismatches. In some aspects, the guide strand described herein comprises a nucleic acid sequence comprising 22 consecutive sequences selected from SEQ ID NOs: 1-196, 797-806, and 850-857 with no more than 1, 2, 3, or 4 mismatches.

In some aspects, the passenger strand described herein comprises a nucleic acid sequence comprising 15 consecutive sequences selected from SEQ ID NOs: 402-403, 405, 407-408, 410-411, 414-415, 418, 420-422, 432, 434, 442-446, 450-456, 460, 476-477, 496, 498, 507-509, 511, 513-514, 535, 545-546, 548-549, 553, 557-558, 560-561, 819-828, and 870-875 with no more than 1, 2, 3, or 4 mismatches. In some aspects, the guide strand described herein comprises a nucleic acid sequence comprising 15 consecutive sequences selected from SEQ ID NOs: 2-3, 5, 7-8, 10-11, 14-15, 18, 20-22, 32, 34, 42-46, 50-56, 60, 76-77, 96, 98, 107-109, 111, 113-114, 135, 145-146, 148-149, 153, 157-158, 160-161, 797-806, and 850-857 with no more than 1, 2, 3, or 4 mismatches. In some aspects, the passenger strand described herein comprises a nucleic acid sequence comprising 16 consecutive sequences selected from SEQ ID NOs: 402-403, 405, 407-408, 410-411, 414-415, 418, 420-422, 432, 434, 442-446, 450-456, 460, 476-477, 496, 498, 507-509, 511, 513-514, 535, 545-546, 548-549, 553, 557-558, 560-561, 819-828, and 870-875 with no more than 1, 2, 3, or 4 mismatches. In some aspects, the guide strand described herein comprises a nucleic acid sequence comprising 16 consecutive sequences selected from SEQ ID NOs: 2-3, 5, 7-8, 10-11, 14-15, 18, 20-22, 32, 34, 42-46, 50-56, 60, 76-77, 96, 98, 107-109, 111, 113-114, 135, 145-146, 148-149, 153, 157-158, 160-161, 797-806, and 850-857 with no more than 1, 2, 3, or 4 mismatches. In some aspects, the passenger strand described herein comprises a nucleic acid sequence comprising 17 consecutive sequences selected from SEQ ID NOs: 402-403, 405, 407-408, 410-411, 414-415, 418, 420-422, 432, 434, 442-446, 450-456, 460, 476-477, 496, 498, 507-509, 511, 513-514, 535, 545-546, 548-549, 553, 557-558, 560-561, 819-828, and 870-875 with no more than 1, 2, 3, or 4 mismatches. In some aspects, the guide strand described herein comprises a nucleic acid sequence comprising 17 consecutive sequences selected from SEQ ID NOs: 2-3, 5, 7-8, 10-11, 14-15, 18, 20-22, 32, 34, 42-46, 50-56, 60, 76-77, 96, 98, 107-109, 111, 113-114, 135, 145-146, 148-149, 153, 157-158, 160-161, 797-806, and 850-857 with no more than 1, 2, 3, or 4 mismatches. In some aspects, the passenger strand described herein comprises a nucleic acid sequence comprising 18 consecutive sequences selected from SEQ ID NOs: 402-403, 405, 407-408, 410-411, 414-415, 418, 420-422, 432, 434, 442-446, 450-456, 460, 476-477, 496, 498, 507-509, 511, 513-514, 535, 545-546, 548-549, 553, 557-558, 560-561, 819-828, and 870-875 with no more than 1, 2, 3, or 4 mismatches. In some aspects, the guide strand described herein comprises a nucleic acid sequence comprising 18 consecutive sequences selected from SEQ ID NOs: 2-3, 5, 7-8, 10-11, 14-15, 18, 20-22, 32, 34, 42-46, 50-56, 60, 76-77, 96, 98, 107-109, 111, 113-114, 135, 145-146, 148-149, 153, 157-158, 160-161, 797-806, and 850-857 with no more than 1, 2, 3, or 4 mismatches. In some aspects, the passenger strand described herein comprises a nucleic acid sequence comprising 19 consecutive sequences selected from SEQ ID NOs: 402-403, 405, 407-408, 410-411, 414-415, 418, 420-422, 432, 434, 442-446, 450-456, 460, 476-477, 496, 498, 507-509, 511, 513-514, 535, 545-546, 548-549, 553, 557-558, 560-561, 819-828, and 870-875 with no more than 1, 2, 3, or 4 mismatches. In some aspects, the guide strand described herein comprises a nucleic acid sequence comprising 19 consecutive sequences selected from SEQ ID NOs: 2-3, 5, 7-8, 10-11, 14-15, 18, 20-22, 32, 34, 42-46, 50-56, 60, 76-77, 96, 98, 107-109, 111, 113-114, 135, 145-146, 148-149, 153, 157-158, 160-161, 797-806, and 850-857 with no more than 1, 2, 3, or 4 mismatches. In some aspects, the passenger strand described herein comprises a nucleic acid sequence comprising 20 consecutive sequences selected from SEQ ID NOs: 402-403, 405, 407-408, 410-411, 414-415, 418, 420-422, 432, 434, 442-446, 450-456, 460, 476-477, 496, 498, 507-509, 511, 513-514, 535, 545-546, 548-549, 553, 557-558, 560-561, 819-828, and 870-875 with no more than 1, 2, 3, or 4 mismatches. In some aspects, the guide strand described herein comprises a nucleic acid sequence comprising 20 consecutive sequences selected from SEQ ID NOs: 2-3, 5, 7-8, 10-11, 14-15, 18, 20-22, 32, 34, 42-46, 50-56, 60, 76-77, 96, 98, 107-109, 111, 113-114, 135, 145-146, 148-149, 153, 157-158, 160-161, 797-806, and 850-857 with no more than 1, 2, 3, or 4 mismatches. In some aspects, the passenger strand described herein comprises a nucleic acid sequence comprising 21 consecutive sequences selected from SEQ ID NOs: 402-403, 405, 407-408, 410-411, 414-415, 418, 420-422, 432, 434, 442-446, 450-456, 460, 476-477, 496, 498, 507-509, 511, 513-514, 535, 545-546, 548-549, 553, 557-558, 560-561, 819-828, and 870-875 with no more than 1, 2, 3, or 4 mismatches. In some aspects, the guide strand described herein comprises a nucleic acid sequence comprising 21 consecutive sequences selected from SEQ ID NOs: 2-3, 5, 7-8, 10-11, 14-15, 18, 20-22, 32, 34, 42-46, 50-56, 60, 76-77, 96, 98, 107-109, 111, 113-114, 135, 145-146, 148-149, 153, 157-158, 160-161, 797-806, and 850-857 with no more than 1, 2, 3, or 4 mismatches. In some aspects, the passenger strand described herein comprises a nucleic acid sequence comprising 22 consecutive sequences selected from SEQ ID NOs: 402-403, 405, 407-408, 410-411, 414-415, 418, 420-422, 432, 434, 442-446, 450-456, 460, 476-477, 496, 498, 507-509, 511, 513-514, 535, 545-546, 548-549, 553, 557-558, 560-561, 819-828, and 870-875 with no more than 1, 2, 3, or 4 mismatches. In some aspects, the guide strand described herein comprises a nucleic acid sequence comprising 22 consecutive sequences selected from SEQ ID NOs: 2-3, 5, 7-8, 10-11, 14-15, 18, 20-22, 32, 34, 42-46, 50-56, 60, 76-77, 96, 98, 107-109, 111, 113-114, 135, 145-146, 148-149, 153, 157-158, 160-161, 797-806, and 850-857 with no more than 1, 2, 3, or 4 mismatches.

In some aspects, the passenger strand described herein comprises a nucleic acid sequence comprising 15 consecutive sequences selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875 with no more than 1, 2, 3, or 4 mismatches. In some aspects, the guide strand described herein comprises a nucleic acid sequence comprising 15 consecutive sequences selected from SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857 with no more than 1, 2, 3, or 4 mismatches. In some aspects, the passenger strand described herein comprises a nucleic acid sequence comprising 16 consecutive sequences selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875 with no more than 1, 2, 3, or 4 mismatches. In some aspects, the guide strand described herein comprises a nucleic acid sequence comprising 16 consecutive sequences selected from SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857 with no more than 1, 2, 3, or 4 mismatches. In some aspects, the passenger strand described herein comprises a nucleic acid sequence comprising 17 consecutive sequences selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875 with no more than 1, 2, 3, or 4 mismatches. In some aspects, the guide strand described herein comprises a nucleic acid sequence comprising 17 consecutive sequences selected from SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857 with no more than 1, 2, 3, or 4 mismatches. In some aspects, the passenger strand described herein comprises a nucleic acid sequence comprising 18 consecutive sequences selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875 with no more than 1, 2, 3, or 4 mismatches. In some aspects, the guide strand described herein comprises a nucleic acid sequence comprising 18 consecutive sequences selected from SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857 with no more than 1, 2, 3, or 4 mismatches. In some aspects, the passenger strand described herein comprises a nucleic acid sequence comprising 19 consecutive sequences selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875 with no more than 1, 2, 3, or 4 mismatches. In some aspects, the guide strand described herein comprises a nucleic acid sequence comprising 19 consecutive sequences selected from SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857 with no more than 1, 2, 3, or 4 mismatches. In some aspects, the passenger strand described herein comprises a nucleic acid sequence comprising 20 consecutive sequences selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875 with no more than 1, 2, 3, or 4 mismatches. In some aspects, the guide strand described herein comprises a nucleic acid sequence comprising 20 consecutive sequences selected from SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857 with no more than 1, 2, 3, or 4 mismatches. In some aspects, the passenger strand described herein comprises a nucleic acid sequence comprising 21 consecutive sequences selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875 with no more than 1, 2, 3, or 4 mismatches. In some aspects, the guide strand described herein comprises a nucleic acid sequence comprising 21 consecutive sequences selected from SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857 with no more than 1, 2, 3, or 4 mismatches. In some aspects, the passenger strand described herein comprises a nucleic acid sequence comprising 22 consecutive sequences selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875 with no more than 1, 2, 3, or 4 mismatches. In some aspects, the guide strand described herein comprises a nucleic acid sequence comprising 22 consecutive sequences selected from SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857 with no more than 1, 2, 3, or 4 mismatches.

In some aspects, the passenger strand described herein comprises a nucleic acid sequence comprising 15 consecutive sequences selected from SEQ ID NOs: 513, 546, 553, 558, 508, 514, and 545 with no more than 1, 2, 3, or 4 mismatches. In some aspects, the guide strand described herein comprises a nucleic acid sequence comprising 15 consecutive sequences selected from SEQ ID NOs: 113, 146, 153, 158, 108, 114, and 145 with no more than 1, 2, 3, or 4 mismatches.

In some aspects, the passenger strand described herein comprises a nucleic acid sequence comprising 16 consecutive sequences selected from SEQ ID NOs: 513, 546, 553, 558, 508, 514, and 545 with no more than 1, 2, 3, or 4 mismatches. In some aspects, the guide strand described herein comprises a nucleic acid sequence comprising 16 consecutive sequences selected from SEQ ID NOs: 113, 146, 153, 158, 108, 114, and 145 with no more than 1, 2, 3, or 4 mismatches.

In some aspects, the passenger strand described herein comprises a nucleic acid sequence comprising 17 consecutive sequences selected from SEQ ID NOs: 513, 546, 553, 558, 508, 514, and 545 with no more than 1, 2, 3, or 4 mismatches. In some aspects, the guide strand described herein comprises a nucleic acid sequence comprising 17 consecutive sequences selected from SEQ ID NOs: 113, 146, 153, 158, 108, 114, and 145 with no more than 1, 2, 3, or 4 mismatches.

In some aspects, the passenger strand described herein comprises a nucleic acid sequence comprising 18 consecutive sequences selected from SEQ ID NOs: 513, 546, 553, 558, 508, 514, and 545 with no more than 1, 2, 3, or 4 mismatches. In some aspects, the guide strand described herein comprises a nucleic acid sequence comprising 18 consecutive sequences selected from SEQ ID NOs: 113, 146, 153, 158, 108, 114, and 145 with no more than 1, 2, 3, or 4 mismatches.

In some aspects, the passenger strand described herein comprises a nucleic acid sequence comprising 19 consecutive sequences selected from SEQ ID NOs: 513, 546, 553, 558, 508, 514, and 545 with no more than 1, 2, 3, or 4 mismatches. In some aspects, the guide strand described herein comprises a nucleic acid sequence comprising 19 consecutive sequences selected from SEQ ID NOs: 113, 146, 153, 158, 108, 114, and 145 with no more than 1, 2, 3, or 4 mismatches. In some aspects, the passenger strand described herein comprises a nucleic acid sequence comprising 20 consecutive sequences selected from SEQ ID NOs: 513, 546, 553, 558, 508, 514, and 545 with no more than 1, 2, 3, or 4 mismatches. In some aspects, the guide strand described herein comprises a nucleic acid sequence comprising 20 consecutive sequences selected from SEQ ID NOs: 113, 146, 153, 158, 108, 114, and 145 with no more than 1, 2, 3, or 4 mismatches. In some aspects, the passenger strand described herein comprises a nucleic acid sequence comprising 21 consecutive sequences selected from SEQ ID NOs: 513, 546, 553, 558, 508, 514, and 545 with no more than 1, 2, 3, or 4 mismatches. In some aspects, the guide strand described herein comprises a nucleic acid sequence comprising 21 consecutive sequences selected from SEQ ID NOs: 5113, 146, 153, 158, 108, 114, and 145 with no more than 1, 2, 3, or 4 mismatches. In some aspects, the passenger strand described herein comprises a nucleic acid sequence comprising 22 consecutive sequences selected from SEQ ID NOs: 513, 546, 553, 558, 508, 514, and 545 with no more than 1, 2, 3, or 4 mismatches. In some aspects, the guide strand described herein comprises a nucleic acid sequence comprising 22 consecutive sequences selected from SEQ ID NOs: 113, 146, 153, 158, 108, 114, and 145 with no more than 1, 2, 3, or 4 mismatches.

In some aspects, the polynucleic acid molecule described herein comprises a passenger strand and a guide strand of about 15-30, 16-30, 17-30, 18-30, 18-27, 18-25, 18-23, 19-23, 20-23, or 21-23 nucleotides in length. In some aspects, the polynucleic acid molecule described herein comprises a passenger strand and a guide strand of about 15, 16, 17, 18, 19, 20 nucleotides in length. In some aspects, the polynucleic acid molecule described herein comprises a passenger strand and a guide strand of about 21, 22, 23, 24, 25 nucleotides in length. In some aspects, the polynucleic acid molecule described herein comprises a passenger strand and a guide strand of about 26, 27, 28, 29, 30 nucleotides in length. In some aspects, the polynucleic acid molecule described herein comprises a passenger strand of 19 nucleotides in length, and a guide strand of about 21 nucleotides in length. In some aspects, the polynucleic acid molecule described herein comprises a passenger strand of 21 nucleotides in length, and a guide strand of about 23 nucleotides in length.

In some aspects, the passenger strand and the guide strand described herein are reverse complementary to each other and form a duplex with a 3′ overhang on the guide strand. In some aspects, the passenger strand and the guide strand described herein are reverse complementary to each other and form a duplex with a 5′ overhang on the guide strand. In some aspects, the passenger strand and the guide strand described herein are reverse complementary to each other and form a duplex with a 3′ overhang on the passenger strand. In some aspects, the passenger strand and the guide strand described herein are reverse complementary to each other and form a duplex with a 5′ overhang on the passenger strand.

### Modifications of Polynucleic Acid Molecules

In some aspects, described herein is the polynucleic acid molecule described herein with modifications. In some aspects, the modifications described herein occurs one or more different structures of the polynucleic acid molecule described herein (e.g., modifications on sugar ring(s), backbone(s), base(s)). In some aspects, the modifications described herein comprise substitutions of one or more nucleotide in the polynucleic acid molecule described herein. In some aspects, different percentages of the polynucleic acid molecule described herein comprise the modifications described herein. In some aspects, different positions of the polynucleic acid molecule described herein comprise the modifications described herein. In some aspects, the modifications described herein comprise modification patterns disclosed in WO/2018/035380, which is herein incorporated by reference in its entirety.

### Types of Modifications

In some aspects, the polynucleic acid molecule described herein comprises one or more sugar-modified nucleotide. In some aspects, the sugar-modified nucleotide is a 2′-fluoro modified nucleotide. In some instances, the 2′-fluoro modified nucleotide comprises thio-modified base containing nucleotide, e.g., 2′-fluoro-2-thiouridine-3′-phosphate (U3f). In some instances, sugar-modified nucleotide includes a modification at a 2′ hydroxyl group of the ribose moiety. In some instances, the sugar-modified nucleotide includes modification with an H, OR, R, halo, SH, SR, NH2, NHR, NR2, or CN, wherein R is an alkyl moiety. In some aspects, the sugar-modified nucleotide is a 2′-O-methyl modified nucleotide or 2′-alkoxy modified nucleotide (e.g., 2′-methoxy modified nucleotide). In some instances, 2′ hydroxyl group modification includes 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA). In some instances, the alkyl moiety comprises a hetero substitution. In some instances, the carbon of the heterocyclic group is substituted by a nitrogen, oxygen or sulfur. In some aspects, the sugar-modified nucleotide is a 2′-amino modified nucleotide. In some aspects, the sugar-modified nucleotide is a 2′-azido modified nucleotide. In some aspects, the sugar-modified nucleotide is a 2′-deoxy modified nucleotide. In some aspects, the sugar-modified nucleotide is a 2′-O-methoxythyl (2′-MOE). In some aspects, the sugar-modified nucleotide is a locked nucleic acid (LNA). In some aspects, the sugar-modified nucleotide is an ethylene-bridged nucleic acid (ENA). In some aspects, the sugar-modified nucleotide is a (S)-constrained ethyl (cEt). In some aspects, the sugar-modified nucleotide is a tricyclo-DNA (tcDNA). In some aspects, the sugar-modified nucleotide is a 2′-NH2 nucleic acid. In some aspects, the polynucleic acid molecule described herein comprises a 5′-vinylphosphonate modified nucleotide. In some instances, the 5′-vinylphosphonate modified nucleotide is located at the guide strand of the double stranded polynucleic acid molecule (e.g., siRNA). In some instances, the 5′-vinylphosphonate modified nucleotide is located at the 5′ end of the guide strand. In some instances, the 5′-vinylphosphonate modified nucleotide is located at position 1 from the 5′ end of the guide strand.

In some aspects, the polynucleic acid molecule described herein comprises one or more sugarphosphate-modified nucleotide. In some aspects, the modified sugarphosphate is phosphorodiamidate morpholino (PMO). In some aspects, the modified sugarphosphate is phosphoramidate. In some instances, the heterocyclic substitution includes imidazole, and pyrrolidino. In some aspects, the modified sugarphosphate is thiophosphoramidate. In some aspects, the modified sugarphosphate is peptide nucleic acid (PNA).

In some aspects, the polynucleic acid molecule described herein comprises one or more backbone-modified nucleotide. In some aspects, the modified backbone is a methylphosphonate. In some aspects, the modified backbone is phosphorothioate. In some aspects, the modified backbone is a guanidinopropyl phosphoramidate. In some aspects, the modified backbone is a mesyl-phosphoramidate (MsPA) linkages. In some instances, the modified backbone comprises one or more of phosphorodithioates, methylphosphonates, 5′-alkylenephosphonates, 5′-methylphosphonate, 3′-alkylene phosphonates, borontrifluoridates, borano phosphate esters and selenophosphates of 3′-5′ linkage or 2′-5′ linkage, phosphotriesters, thionoalkylphosphotriesters, hydrogen phosphonate linkages, alkyl phosphonates, alkylphosphonothioates, arylphosphonothioates, phosphoroselenoates, phosphoramidates.

In some aspects, the modified nucleotide comprises a modified guanine (e.g., inosine) or one or more of any types of unnatural nucleic acids.

In some aspects, the modified backbone comprises a phosphorothioate intemucleotide linkage, and the phosphorothioate is a stereochemically enriched phosphorothioate. In certain aspects, the strand contains at least one stereochemically enriched phosphorothioate. In some aspects, the strand comprises at least 1, 2, 3 stereochemically enriched phosphorothioates. In some aspects, the strand comprises only 1, 2, 3, or 4 stereochemically enriched phosphorothioates. In further aspects, at least one (e.g., one or two) stereochemically enriched phosphorothioate is disposed between two consecutive nucleosides that are two of six 5′ end nucleosides of the strand. In yet further aspects, at least one (e.g., one or two) stereochemically enriched phosphorothioate is disposed between two consecutive nucleosides that are two of six 3′ end nucleosides of the strand. In still further aspects, one stereochemically enriched phosphorothioate is covalently bonded to the first nucleoside and the second nucleoside from the 5′ end within the strand. In some aspects, one stereochemically enriched phosphorothioate is covalently bonded to the twenty first nucleoside and the twenty second nucleoside from the 5′ end within the strand. In certain aspects, one stereochemically enriched phosphorothioate is covalently bonded to the twenty second nucleoside and the twenty third nucleoside from the 5′ end within the strand. In particular aspects, the stereochemically enriched phosphorothioate has R_{P} stereochemical identity. In certain aspects, the stereochemically enriched phosphorothioate has S_{P} stereochemical identity.

In some aspects, the polynucleic acid molecules described herein comprises one or more (e.g., from 1 to 20, from 1 to 10, or from 1 to 5) stereochemically enriched (e.g., internucleoside) phosphorothioates (e.g., having diastereomeric excess of at least 10%, at least 50%, at least 60%, at least 70%, at least 80%, or at least 90%, e.g., up to about 99%, for the P-stereogenic center). The polynucleic acid molecules described herein comprises one or more (e.g., from 1 to 20, from 1 to 10, or from 1 to 5; e.g., intemucleoside) phosphorodithioates. The phosphorodithioates may be non-P-stereogenic in the polynucleic acid molecules described herein. Phosphorothioates and phosphorodithioates may enhance the stability of the polynucleic acid molecules described herein to exonuclease activity of serum. Non-P-stereogenic phosphorodithioates may simplify the synthesis of the polynucleic acid molecule described herein by reducing the number of possible diastereomers. Typically, the phosphorothioate or phosphorodithioate may connect two contiguous nucleosides within the six 3′ end nucleosides and the six 5′ end nucleosides of the polynucleic acid molecules described herein. In some aspects, the stereochemically enriched phosphorothioate (e.g., R_{P}-enriched phosphorothioate) may be covalently bonded to the first nucleoside (e.g., the 3′-carbon atom of the first nucleoside) and the second nucleoside (e.g., the 5′-carbon atom of the second nucleoside) from the 5′ end of the guide strand. Additionally or alternatively, the stereochemically enriched phosphorothioate (e.g., S_{P}-enriched phosphorothioate) may be covalently bonded to the 21^{st} nucleoside (e.g., the 3′-carbon atom of the 21^{st} nucleoside) from the 5′ end and the 22^{nd} nucleoside (e.g., the 5′-carbon atom of the 22^{nd} nucleoside) of the guide strand. Further, additionally or alternatively, the stereochemically enriched phosphorothioate (e.g., S_{P}-enriched phosphorothioate or R_{P}-enriched phosphorothioate) may be covalently bonded to the 22^{nd} nucleoside (e.g., the 3′-carbon atom of the 22^{nd} nucleoside) and the 23^{rd} nucleoside (e.g., the 5′-carbon atom of the 23^{rd} nucleoside) from the 5′ end of the guide strand.

Combinations of a 5′ R_{P}-enriched phosphorothioate (e.g., R_{P}-enriched phosphorothioate covalently bonded to the first nucleoside (e.g., the 3′-carbon atom of the first nucleoside) and the second nucleoside (e.g., the 5′-carbon atom of the second nucleoside) from the 5′ end and a 3′ S_{P}-enriched phosphorothioate (e.g., S_{P}-enriched phosphorothioate covalently bonded to the 21^{st} nucleoside (e.g., the 3′-carbon atom of the 21^{st} nucleoside) and the 22^{nd} nucleoside (e.g., the 5′-carbon atom of the 22^{nd} nucleoside) from the 5′ end in a guide strand can produce superior efficacy and/or duration of action, e.g., as measured by the reduction in the activity of the target relative to a reference guide strand that lacks the combination of a 5′ R_{P}-enriched phosphorothioate and a 3′ S_{P}-enriched phosphorothioate, or a 5′ Rp-enriched phosphorothioate and a 3′ Sₚ and Rₚ-enriched phosphorothioate. In some embodiments, the stereochemically enriched phosphorothioate may comprise RₚRₚSₚSₚ (RₚRₚ at the positions 1 and 2 of the guide strand and SₚSₚ at the positions 21 and 22 of the guide strand) or RₚRₚSₚRₚ (RₚRₚ at the positions 1 and 2 of the guide strand and SₚRₚ at the positions 21 and 22 of the guide strand). In some aspects, the polynucleic acid molecules described herein comprises four stereochemically enriched phosphorothioates: (1) a Rp-enriched phosphorothioate covalently bonded to the 1^{st} nucleoside (e.g., the 3′-carbon atom of the 1^{st} nucleoside) and the 2^{nd} nucleoside (e.g., the 5′-carbon atom of the 2^{nd} nucleoside) from the 5′-end of the guide strand; (2) a Rp-enriched phosphorothioate covalently bonded to the 2^{nd} nucleoside (e.g., the 3′-carbon atom of the 2^{nd} nucleoside) and the 3^{rd} nucleoside (e.g., the 5′-carbon atom of the 3^{rd} nucleoside) from the 5′-end of the guide strand; (3) a Sp-enriched phosphorothioate covalently bonded to the 21^{st} nucleoside (e.g., the 3′-carbon atom of the 21^{st} nucleoside) and the 22^{th} nucleoside (e.g., the 5′-carbon atom of the 22^{th} nucleoside) from the 5′-end of the guide strand; and (4) a Sp-enriched phosphorothioate covalently bonded to the 22^{th} nucleoside (e.g., the 3′-carbon atom of the 22^{th} nucleoside) and the 23^{rd} nucleoside (e.g., the 5′-carbon atom of the 23^{rd} nucleoside) from the 5′-end of the guide strand. In some aspects, the polynucleic acid molecules described herein comprises four stereochemically enriched phosphorothioates: (1) a Rp-enriched phosphorothioate covalently bonded to the 1^{st} nucleoside (e.g., the 3′-carbon atom of the 1^{st} nucleoside) and the 2^{nd} nucleoside (e.g., the 5′-carbon atom of the 2^{nd} nucleoside) from the 5′-end of the guide strand; (2) a Rp-enriched phosphorothioate covalently bonded to the 2^{nd} nucleoside (e.g., the 3′-carbon atom of the 2^{nd} nucleoside) and the 3^{rd} nucleoside (e.g., the 5′-carbon atom of the 3^{rd} nucleoside) from the 5′-end of the guide strand; (3) a Sp-enriched phosphorothioate covalently bonded to the 21^{st} nucleoside (e.g., the 3′-carbon atom of the 21^{st} nucleoside) and the 22^{th} nucleoside (e.g., the 5′-carbon atom of the 22^{th} nucleoside) from the 5′-end of the guide strand; and (4) a Rp-enriched phosphorothioate covalently bonded to the 22^{th} nucleoside (e.g., the 3′-carbon atom of the 22^{th} nucleoside) and the 23^{rd} nucleoside (e.g., the 5′-carbon atom of the 23^{rd} nucleoside) from the 5′-end of the guide strand.

In some aspects, the polynucleic acid molecule described herein comprises one or more purine modification. In some aspects, the purine modification described herein is 2,6-diaminopurine. In some aspects, the purine modification described herein is 3-deaza-adenine. In some aspects, the purine modification described herein is 7-deaza-guanine. In some aspects, the purine modification described herein is 8-azido-adenine.

In some aspects, the polynucleic acid molecule described herein comprises one or more pyrimidine modification. In some aspects, the pyrimidine modification described herein is 2-thio-thymidine. In some aspects, the pyrimidine modification described herein is 5-carboxamide-uracil. In some aspects, the pyrimidine modification described herein is 5-methyl-cytosine. In some aspects, the pyrimidine modification described herein is 5-ethynyl uracil.

In some embodiment, the polynucleic acid molecule described herein comprises an abasic substitution. In those cases where a hybridized polynucleotide construct is contemplated for use as siRNA, a reduction of miRNA-like off-target effects is desirable. The inclusion of one or more (e.g., one or two) abasic substitutions in the hybridized polynucleotide constructs may reduce or even eliminate miRNA-like off-target effects, as the abasic substitutions lack nucleobases that are capable of engaging in base-pairing interactions and alleviate steric hindrance. Thus, the polynucleic acid molecule disclosed herein may include one or more (e.g., one or two) abasic substitutions. In some aspects, abasic substitution is at the 5^{th} nucleotide from the 5′ end of the guide strand described herein. In some aspects, abasic substitution is at the 7^{th} nucleotide from the 5′ end of the guide strand described herein.

When the polynucleic acid molecule disclosed herein includes two or more of the abasic substitutions, their structures may be same or different. In certain aspects, a passenger strand contains one abasic substitution (e.g., a guide strand may be free of abasic substitutions). In other aspects, a guide strand contains one abasic substitution (e.g., a passenger strand may be free of abasic substitutions). In yet other aspects, a guide strand contains one abasic substitution, and a passenger strand contains one abasic substitution. In further aspects, a passenger strand comprises an abasic substitution between a nucleoside number (x) and a nucleoside number (x+1), where x is an integer from 2 to 7. In yet further aspects, a guide strand includes an abasic substitution between a nucleoside number (x) and a nucleoside number (x+1), where x is an integer from 2 to 7.

The abasic substitution may be of formula (III):

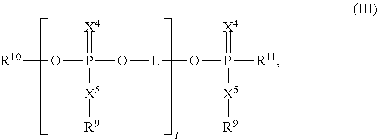

where
L is a sugar analogue, or is substituted with a heteroacyl from A, U, C, G, or is any other substituted nucleic acid (e.g., locked or unlocked nucleic acid, glycol nucleic acid, etc.);
each X⁴ is independently O or S;
each X⁵ is independently O, S, NH, or a bond;
each R⁹ is independently H, optionally substituted C₁₋₆ alkyl, optionally substituted C₂₋₆ alkenyl, optionally substituted C₂₋₆ alkynyl, optionally substituted (C₁₋₉ heterocyclyl)-C₁₋₆-alkyl, optionally substituted (C₆₋₁₀ aryl)-C₁₋₆-alkyl, optionally substituted (C₃₋₈ cycloalkyl)-C₁₋₆-alkyl, -LinkA(−T)ₚ, or a conjugation moiety;
each LinkA is independently a multivalent linker (e.g., including —C(O)—N(H)—);
each T is independently an auxiliary moiety;
R¹⁰ is a bond to a 3′-carbon atom of a nucleoside (x) in the strand;
R¹¹ is a bond to a 5′-oxygen atom of a nucleoside (x+1) in the strand;
p is an integer from 1 to 6; and t is an integer from 1 to 6.

In some aspects, the abasic substitution described herein is attached to the guide strand of the polynucleic acid molecule described herein. In particular aspects, an abasic substitution (e.g., an intemucleotide, abasic spacer of formula (III) in which t is 1) may be included in the guide strand described herein (e.g., within the seed region of the guide strand). In some aspects, an abasic substitution (e.g., an intemucleotide, abasic spacer of formula (III) in which t is 1) may be bonded to the 3′ carbon atom of the second, third, fourth, or fifth nucleoside from the 5′ end of the guide strand described herein. In certain aspects, an abasic substitution (e.g., an intemucleotide, abasic spacer of formula (III) in which t is 1) may be bonded to the 3′ carbon atom of the thirteenth, fourteenth, fifteenth, or sixteenth nucleoside from the 5′ end of the guide strand described herein. In some aspects, an abasic substitution fourth, fifth, sixth, seventh, eighth, and/or ninth nucleoside from the 5′ end of the guide strand described herein.

The polynucleic acid molecule described herein may contain a strand including a seed region including a hypoxanthine nucleobase-containing nucleoside (e.g., inosine).

In certain aspects, the hypoxanthine nucleobase-containing nucleoside is a second nucleoside from the 5′ end in the strand. In further aspects, the hypoxanthine nucleobase-containing nucleoside is a third nucleoside from the 5′ end in the strand. In yet further aspects, the hypoxanthine nucleobase-containing nucleoside is a fourth nucleoside from the 5′ end in the strand. In still further aspects, the hypoxanthine nucleobase-containing nucleoside is a fifth nucleoside from the 5′ end in the strand. In particular aspects, the hypoxanthine nucleobase-containing nucleoside is a sixth nucleoside in the strand. In particular aspects, the hypoxanthine nucleobase-containing nucleoside is a seventh nucleoside in the strand.

### Nucleotide Analogue

In some aspects, the present disclosure provides a polynucleic acid molecule incorporating a nucleotide analogue.

In some instances, modifications of the nucleotide with the nucleotide analogue described herein can alter base pairings and structural changes of the inhibitiory polynucleic acid molecule. In some intances, the nucleotide analogue can be incorporated into the polynucleic acid molecule, thereby suppressing off-target effects. In some instances, the nucleotide analogue can be incorporated into the polynucleic acid molecule, thereby improving stability and efficacy of the polynucleic acid molecule. In some instances, the nucleotide analogue described herein can be incorporated into a guide strand, a passenger strand, or a combination thereof.

In some intances, the nucleotide analogue can be placed in the polynucleic acid molecule or be a substitute for a nucleotide in the polynucleic acid molecule, thereby suppressing the off-target effects. In some instances, the nucleotide analogue can be substituted for a nucleotide in the polynucleic acid molecule, thereby improving stability and/or efficacy of the polynucleic acid molecule. In some instances, the nucleotide analogue described herein can be placed in a guide strand, a passenger strand, or both.

In some aspects, the present disclosure provides a polynucleic acid molecule comprising a passenger strand (sense strand) and a guide strand (antisense strand), wherein the guide strand comprises a nucleotide analogue as described herein. In some instances, the nucleotide analogue comprises acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), thymidine-glycol nucleic acid (GNA)S-isomer (Tgn), 2′-O-methyl-2-thiouridine-3′-phosphate (u3), 2′-fluoro-2-thiouridine-3′-phosphate (U3f), or 2-amino-2′-O-methyladenosine-3′-phosphate (al). In some instance, the nucleotide analogue is selected from a group consisting of the nucleotide analogue selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), thymidine-glycol nucleic acid (GNA)S-isomer (Tgn), 2′-O-methyl-2-thiouridine-3′-phosphate (u3), 2′-fluoro-2-thiouridine-3′-phosphate (U3), and 2-amino-2′-O-methyladenosine-3′-phosphate (al). In some instances, the nucleotide analogue is selected from a group consisting of the nucleotide analogue selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), thymidine-glycol nucleic acid (GNA)S-isomer (Tgn), 2′-O-methyl-2-thiouridine-3′-phosphate (u3), and 2′-fluoro-2-thiouridine-3′-phosphate (U3).

In some instances, the acyclic L-threoninol nucleic acid-3′-phosphate (T-T) has a generic representation shown as below, where the base can be any suitable base or modified base that can make Watson-Crick binding with the base on the opposite strand:

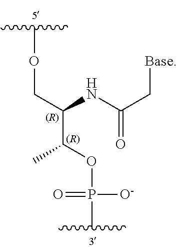

In some instances, the amidite structure of acyclic L-threoninol nucleic acid-thymine-3′-phosphate (T-T) is shown as below:

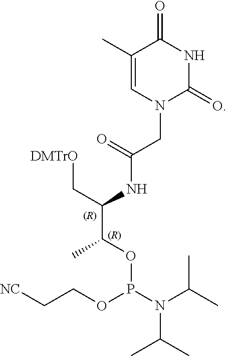

In some instances, the acyclic L-threoninol nucleic acid-thymine-3′-phosphate (T-T) is incorporated into the polynucleic acid molecule. In some instances, the acyclic L-threoninol nucleic acid-thymine-3′-phosphate (T-T) is incorporated into the siRNA. In some instances, the acyclic L-threoninol nucleic acid-thymine-3′-phosphate (T-T) is incorporated into a guide strand. In some instances, the acyclic L-threoninol nucleic acid-thymine-3′-phosphate (T-T) is incorporated into a passenger strand. In some instances, an incorporated acyclic L-threoninol nucleic acid-thymine-3′-phosphate (T-T) has structure shown as below:

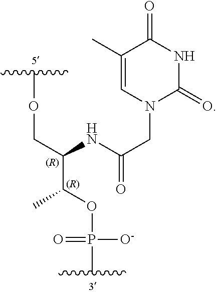

In some instances, the acyclic L-threoninol nucleic acid-thymine-3′-phosphate (T-T) is placed in the polynucleic acid molecule. In some instances, the acyclic L-threoninol nucleic acid-thymine-3′-phosphate (T-T) substitutes one or more nucleotide in siRNA. In some instances, the acyclic L-threoninol nucleic acid-thymine-3′-phosphate (T-T) substitutes one or more nucleotide in a passenger strand and/or a guide strand. In some instances, the acyclic L-threoninol nucleic acid-thymine-3′-phosphate (T-T) substitutes one or more nucleotide in a passenger strand. In some instances, the acyclic L-threoninol nucleic acid-thymine-3′-phosphate (T-T) substitutes one or more nucleotide in a guide strand.

In some instances, the amidite structure of acyclic L-threoninol nucleic acid-adenine-3′-phosphate (T-A) is shown as below:

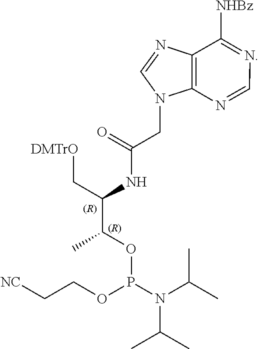

In some instances, the acyclic L-threoninol nucleic acid-adenine-3′-phosphate (T-A) is incorporated into the polynucleic acid molecule. In some instances, the acyclic L-threoninol nucleic acid-adenine-3′-phosphate (T-A) is incorporated into the siRNA. In some instances, the acyclic L-threoninol nucleic acid-adenine-3′-phosphate (T-A)is incorporated into a guide strand. In some instances, the acyclic L-threoninol nucleic acid-adenine-3′-phosphate (T-A) is incorporated into a passenger strand. In some instances, an incorporated acyclic L-threoninol nucleic acid-adenine-3′-phosphate (T-A) has structure shown as below:

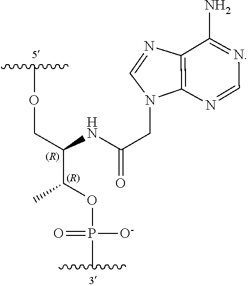

In some instances, the acyclic L-threoninol nucleic acid-adenine-3′-phosphate (T-A) is placed in the polynucleic acid molecule. In some instances, the acyclic L-threoninol nucleic acid-adenine-3′-phosphate (T-A) substitutes one or more nucleotide in siRNA. In some instances, the acyclic L-threoninol nucleic acid-adenine-3′-phosphate (T-A) substitutes one or more nucleotide in a passenger strand and/or a guide strand. In some instances, the acyclic L-threoninol nucleic acid-adenine-3′-phosphate (T-A) substitutes one or more nucleotide in a passenger strand. In some instances, the acyclic L-threoninol nucleic acid-adenine-3′-phosphate (T-A) substitutes one or more the nucleotide in a guide strand.

In some instances, the amidite structure of acyclic N-Acetyl L-threoninol abasic nucleic acid-3′-phosphate (T-NAc) is shown as below:

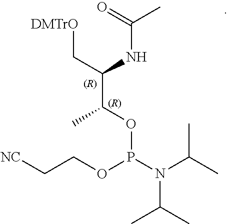

In some instances, the acyclic N-Acetyl L-threoninol abasic nucleic acid-3′-phosphate (T-NAc) is incorporated into the polynucleic acid molecule. In some instances, the acyclic N-Acetyl L-threoninol abasic nucleic acid-3′-phosphate (T-NAc) is incorporated into the siRNA. In some instances, the acyclic N-Acetyl L-threoninol abasic nucleic acid-3′-phosphate (T-NAc) is incorporated into a guide strand. In some instances, the acyclic N-Acetyl L-threoninol abasic nucleic acid-3′-phosphate (T-NAc) is incorporated into a passenger strand. In some instances, an incorporated acyclic N-Acetyl L-threoninol abasic nucleic acid-3′-phosphate (T-NAc) has structure shown as below:

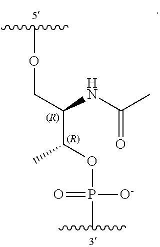

In some instances, the acyclic N-Acetyl L-threoninol abasic nucleic acid-3′-phosphate (T-NAc) is placed in the polynucleic acid molecule. In some instances, the acyclic N-Acetyl L-threoninol abasic nucleic acid-3′-phosphate (T-NAc) substitutes one or more nucleotide in siRNA. In some instances, the acyclic N-Acetyl L-threoninol abasic nucleic acid-3′-phosphate (T-NAc) substitutes one or more nucleotide in a passenger strand and/or a guide strand. In some instances, acyclic N-Acetyl L-threoninol abasic nucleic acid-3′-phosphate (T-NAc) substitutes one or more nucleotide in a passenger strand. In some instances, the acyclic N-Acetyl L-threoninol abasic nucleic acid-3′-phosphate (T-NAc) substitutes one or more nucleotide in a guide strand.

In some instances, the amidite structure of 1′,2′-Dideoxyribose-3′-phosphate (dAB) is shown as below:

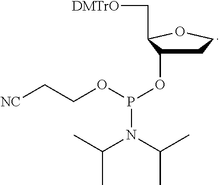

In some instances, the 1′,2′-Dideoxyribose-3′-phosphate (dAB) is incorporated into the polynucleic acid molecule. In some instances, the 1′,2′-Dideoxyribose-3′-phosphate (dAB) is incorporated into the siRNA. In some instances, the 1′,2′-Dideoxyribose-3′-phosphate (dAB) is incorporated into a guide strand. In some instances, the 1′,2′-Dideoxyribose-3′-phosphate (dAB) is incorporated into a passenger strand. In some instances, an incorporated 1′,2′-Dideoxyribose-3′-phosphate (dAB) has structure shown as below:

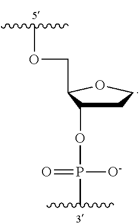

In some instances, the 1′,2′-Dideoxyribose-3′-phosphate (dAB) is placed in the polynucleic acid molecule. In some instances, the 1′,2′-Dideoxyribose-3′-phosphate (dAB) substitutes one or more nucleotide in siRNA. In some instances, the 1′,2′-Dideoxyribose-3′-phosphate (dAB) substitutes one or more nucleotide in a passenger strand and/or a guide strand. In some instances, the 1′,2′-Dideoxyribose-3′-phosphate (dAB) substitutes one or more nucleotide in a passenger strand. In some instances, the 1′,2′-Dideoxyribose-3′-phosphate (dAB) substitutes one or more nucleotide in a guide strand.

In some instances, the amidite structure of thymidine-glycol nucleic acid (GNA)S-isomer (Tgn) is shown as below:

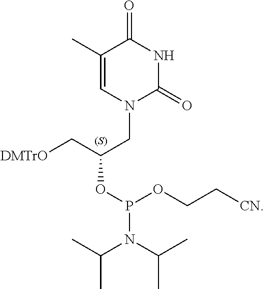

In some instances, the thymidine-glycol nucleic acid (GNA)S-isomer (Tgn) is incorporated into the polynucleic acid molecule. In some instances, the thymidine-glycol nucleic acid (GNA)S-isomer (Tgn) is incorporated into the siRNA. In some instances, the thymidine-glycol nucleic acid (GNA)S-isomer (Tgn) is incorporated into a guide strand. In some instances, the thymidine-glycol nucleic acid (GNA)S-isomer (Tgn) is incorporated into a passenger strand. In some instances, an incorporated thymidine-glycol nucleic acid (GNA)S-isomer (Tgn) has structure shown as below:

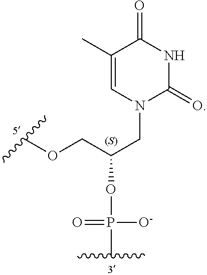

In some instances, the thymidine-glycol nucleic acid (GNA)S-isomer (Tgn) is placed in the polynucleic acid molecule. In some instances, the thymidine-glycol nucleic acid (GNA)S-isomer (Tgn) substitutes one or more nucleotide in siRNA. In some instances, the thymidine-glycol nucleic acid (GNA)S-isomer (Tgn) substitutes one or more nucleotide in a passenger strand and/or a guide strand. In some instances, the thymidine-glycol nucleic acid (GNA)S-isomer (Tgn) substitutes one or more nucleotide in a passenger strand. In some instances, the thymidine-glycol nucleic acid (GNA)S-isomer (Tgn) substitutes one or more nucleotide in a guide strand.

In some instances, the amidite structure of 2′-O-methyl-2-thiouridine-3′-phosphate (u3) is shown as below:

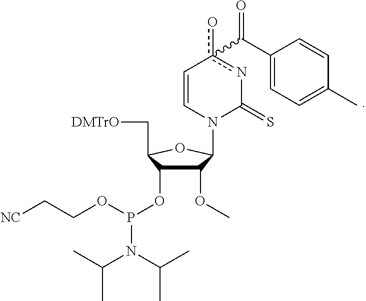

In some instances, the 2′-O-methyl-2-thiouridine-3′-phosphate (u3) is incorporated into the polynucleic acid molecule. In some instances, the 2′-O-methyl-2-thiouridine-3′-phosphate (u3) is incorporated into the siRNA. In some instances, the 2′-O-methyl-2-thiouridine-3′-phosphate (u3) is incorporated into a guide strand. In some instances, the 2′-O-methyl-2-thiouridine-3′-phosphate (u3) is incorporated into a passenger strand. In some instances, an incorporated 2′-O-methyl-2-thiouridine-3′-phosphate (u3) has structure shown as below:

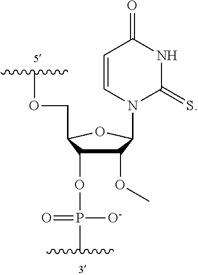

In some instances, the 2′-O-methyl-2-thiouridine-3′-phosphate (u3) is placed in the polynucleic acid molecule. In some instances, the 2′-O-methyl-2-thiouridine-3′-phosphate (u3) substitutes one or more nucleotide in siRNA. In some instances, the 2′-O-methyl-2-thiouridine-3′-phosphate (u3) substitutes one or more nucleotide in a passenger strand and/or a guide strand. In some instances, the 2′-O-methyl-2-thiouridine-3′-phosphate (u3) substitutes one or more nucleotide in a passenger strand. In some instances, the 2′-O-methyl-2-thiouridine-3′-phosphate (u3) substitutes one or more nucleotide in a guide strand.

In some instances, the amidite structure of 2′-fluoro-2-thiouridine-3′-phosphate (U3) is shown as below:

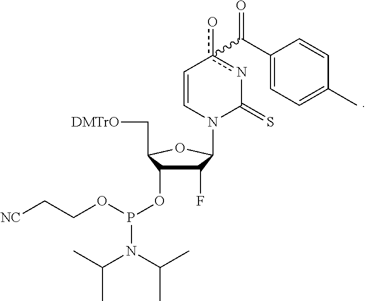

In some instances, the 2′-fluoro-2-thiouridine-3′-phosphate (U3) is incorporated into the polynucleic acid molecule. In some instances, the 2′-fluoro-2-thiouridine-3′-phosphate (U3f) is incorporated into the siRNA. In some instances, the 2′-fluoro-2-thiouridine-3′-phosphate (U3f) is incorporated into a guide strand. In some instances, the 2′-fluoro-2-thiouridine-3′-phosphate (U3f) is incorporated into a passenger strand. In some instances, an incorporated 2′-fluoro-2-thiouridine-3′-phosphate (U3f) has structure shown as below:

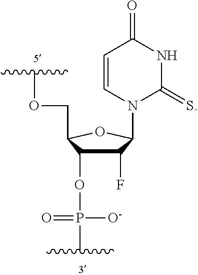

In some instances, the 2′-fluoro-2-thiouridine-3′-phosphate (U3f) is placed in the polynucleic acid molecule. In some instances, the 2′-fluoro-2-thiouridine-3′-phosphate (U3f) substitutes one or more nucleotide in siRNA. In some instances, the 2′-fluoro-2-thiouridine-3′-phosphate (U3) substitutes one or more nucleotide in a passenger strand and/or a guide strand. In some instances, the 2′-fluoro-2-thiouridine-3′-phosphate (U3) substitutes one or more the nucleotide in a passenger strand. In some instances, the 2′-fluoro-2-thiouridine-3′-phosphate (U3f) substitutes one or more the nucleotide in a guide strand.

In some instances, the amidite structure of 2-amino-2′-O-methyladenosine-3′-phosphate (al) is shown as below:

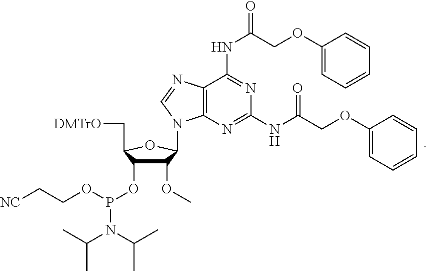

In some instances, the 2-amino-2′-O-methyladenosine-3′-phosphate (al) is incorporated into the polynucleic acid molecule. In some instances, the 2-amino-2′-O-methyladenosine-3′-phosphate (al) is incorporated into the siRNA. In some instances, the 2-amino-2′-O-methyladenosine-3′-phosphate (al) is incorporated into a guide strand. In some instances, the 2-amino-2′-O-methyladenosine-3′-phosphate (al) is incorporated into a passenger strand. In some instances, an incorporated 2-amino-2′-O-methyladenosine-3′-phosphate (al) has structure shown as below:

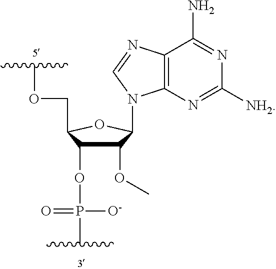

In some instances, the 2-amino-2′-O-methyladenosine-3′-phosphate (al) is placed in the polynucleic acid molecule. In some instances, the 2-amino-2′-O-methyladenosine-3′-phosphate (al) substitutes one or more nucleotide in siRNA. In some instances, the 2-amino-2′-O-methyladenosine-3′-phosphate (al) substitutes one or more nucleotide in a passenger strand and/or a guide strand. In some instances, the 2-amino-2′-O-methyladenosine-3′-phosphate (al) substitutes one or more nucleotide in a passenger strand. In some instances, the 2-amino-2′-O-methyladenosine-3′-phosphate (al) substitutes one or more nucleotide in a guide strand.

In some instances, the nucleotide analogue comprises hypoxanthine nucleobase-containing nucleoside (e.g., inosine).

### The Amount and Location of Modifications

In some aspects, the polynucleic acid molecule described herein comprises one or more type of modifications as described above. Accordingly, in some aspects, about 10% of the nucleotides from the polynucleic acid molecule described herein are modified with one or more type of modifications as described above. In other aspects, about 20% of the nucleotides from the polynucleic acid molecule described herein are modified with one or more type of modifications as described above. In other aspects, about 30% of the nucleotides from the polynucleic acid molecule described herein are modified with one or more type of modifications as described above. In other aspects, about 40% of the nucleotides from the polynucleic acid molecule described herein are modified with one or more type of modifications as described above. In other aspects, about 50% of the nucleotides from the polynucleic acid molecule described herein are modified with one or more type of modifications as described above. In other aspects, about 60% of the nucleotides from the polynucleic acid molecule described herein are modified with one or more type of modifications as described above. In other aspects, about 70% of the nucleotides from the polynucleic acid molecule described herein are modified with one or more type of modifications as described above. In other aspects, about 80% of the nucleotides from the polynucleic acid molecule described herein are modified with one or more type of modifications as described above. In other aspects, about 90% of the nucleotides from the polynucleic acid molecule described herein are modified with one or more type of modifications as described above. In other aspects, 100% of the nucleotides from the polynucleic acid molecule described herein are modified with one or more type of modifications as described above.

In some aspects, the one or more types of modifications described herein occurs at different positions within the polynucleic acid molecule described herein. In some aspects, the one or more types of modifications described herein occurs in the seed region within the polynucleic acid molecule described herein. In some aspects, the one or more types of modifications described herein occurs at 3′ end of the polynucleic acid molecule described herein. In some aspects, the one or more types of modifications described herein occurs at 5′ end of the polynucleic acid molecule described herein. In some aspects, the one or more types of modifications described herein occurs dispersedly within the polynucleic acid molecule described herein. In some aspects, the one or more types of modifications described herein occurs in clusters within the polynucleic acid molecule described herein.

In some aspects, the polynucleic acid molecule is modified with a nucleotide analogue described herein by incorporating the nucleotide analogue in a seed region of the guide strand (positions 2-8 from 5′ end of the guide strand). As described herein, “seed region” refers to a region on the polynucleic acid molecule that comprises sequence that is essential for the binding of the polynucleic acid molecule described herein to the target RNA, e.g., mRNA.

In some instances, the polynucleic acid molecule is modified with a nucleotide analogue described herein at positions 3-8, positions 4-8, positions 5-8, positions 6-8, or positions 7-8 from the 5′ end of the guide strand. In some instances, the polynucleic acid molecule is modified with a nucleotide analogue described herein at position 2, position 3, position 4, position 5, position 6, position 7, position 8, from the 5′ end of the guide strand or combination thereof. In some instances, the polynucleic acid molecule is modified with one, two, three, four, five, six, or seven nucleotide analogues. In some instances, two or more consecutive positions in the guide strand of the polynucleic acid molecule are modified with nucleotide analogues. In some instances, two or more nucleotide analogue modifications can be placed in the polynucleic acid molecule at alternative positions (e.g., positions 3 and 5, positions 4 and 6, positions 5 and 7, etc.).

In some instances, the polynucleic acid molecule comprises a polynucleic acid molecule. In some instances, the polynucleic acid molecule is an siRNA comprising a guide strand and a passenger strand. In some instances, the nucleotide analogue is located at the seed region of the guide strand at positions 2-8 from the 5′ end. In some instances, the nucleotide analogue is located at at positions 3-8 from the 5′ end. In some instances, the nucleotide analogue is located at positions 4-8 from the 5′ end. In some instances, the nucleotide analogue is located at positions 5-8 from the 5′ end. In some instances, the nucleotide analogue is located at positions 6-8 from the 5′ end. the nucleotide analogue is located at positions 6-7 from the 5′ end. In some instances, the nucleotide analogue is located at positions 7-8 from the 5′ end.

In some instances, the nucleotide analogue is located at any one of positions 2-8 from the 5′ end of the guide strand. In some instances, the nucleotide analogue is located at any one of positions 3-8 from the 5′ end of the guide strand. In some instances, the nucleotide analogue is located at any one of positions 4-8 from the 5′ end of the guide strand. In some instances, the nucleotide analogue is located at any one of positions 5-8 from the 5′ end of the guide strand. In some instances, the nucleotide analogue is located at any one of positions 6-8 from the 5′ end of the guide strand. In some instances, the nucleotide analogue is located at any one of positions 6-7 from the 5′ end of the guide strand. In some instances, the nucleotide analogue is located at any one of positions 7-8 from the 5′ end of the guide strand.

In some instances, the nucleotide analogue is located at position 1 from the 5′ end of the guide strand. In some instances, the nucleotide analogue is located at position 2 from the 5′ end of the guide strand. In some instances, the nucleotide analogue is located at position 3 from the 5′ end of the guide strand. In some instances, the nucleotide analogue is located at position 4 from the 5′ end of the guide strand. In some instances, the nucleotide analogue is located at position 5 from the 5′ end of the guide strand. In some instances, the nucleotide analogue is located at position 6 from the 5′ end of the guide strand. In some instances, the nucleotide analogue is located at position 7 from the 5′ end of the guide strand. In some instances, the nucleotide analogue is located at position 8 from the 5′ end of the guide strand. In some instances, the nucleotide analogue is located at position 9 from the 5′ end of the guide strand. In some instances, the nucleotide analogue is located at position 10 from the 5′ end of the guide strand. In some instances, the nucleotide analogue is located at position 11 from the 5′ end of the guide strand. In some instances, the nucleotide analogue is located at position 12 from the 5′ end of the guide strand. In some instances, the nucleotide analogue is located at position 13 from the 5′ end of the guide strand. In some instances, the nucleotide analogue is located at position 14 from the 5′ end of the guide strand.

In some instances, the nucleotide analogue is located at position 3 from the 5′ end of the guide strand. In some instances, the nucleotide analogue is located at position 3 from the 5′ end of the guide strand, and the guide strand further comprises 2-thiouridine-3′-phosphate nucleotide. In some instances, the nucleotide analogue located at position 3 from the 5′ end of the guide strand, and the guide strand further comprises 2′-O-methyl-2-thiouridine-3′-phosphate (u3). In some instances, the nucleotide analogue is located at position 3 from the 5′ end of the guide strand, and the guide strand further comprises at least one, at least two, at least three, or at least four 2′-F modified nucleotides. In some instances, the nucleotide analogue is located at position 3 from the 5′ end of the guide strand, and the guide strand further comprises 2′-F modified nucleotides at at least one of positions 2, 7, 12, 14, and 16 from the 5′ end. In some instances, the nucleotide analogue is located at position 3 from the 5′ end of the guide strand, and the guide strand further comprises 2′-F modified nucleotides at positions 2, 7, 12, 14, and 16 from the 5′ end.

### Specific Modification Patterns

In some aspects, described herein is a specific modification pattern for the polynucleic acid molecule which is a double-stranded nucleic acid molecule comprising a passenger strand and a guide strand. In some aspects, the guide strand comprises a 2′-fluoro modified nucleotide in position 2. In some aspects, the guide strand comprises a 2′-fluoro modified nucleotide in position 14. In some aspects, the guide strand comprises 2′-fluoro modified nucleotides in positions 2 and 14. In some aspects, the guide strand comprises a 2′-fluoro modified nucleotide in position 12. In some aspects, the guide strand comprises a 2′-fluoro modified nucleotide in position 16. In other aspects, the guide strand comprises a 2′-fluoro modified nucleotide in position 6. In other aspects, the guide strand comprises a 2′-fluoro modified nucleotide in position 7. In other aspects, the guide strand comprises a 2′-fluoro modified nucleotide in position 8. In other aspects, the guide strand comprises a 2′-fluoro modified nucleotide in position 9. In other aspects, the guide strand comprises a 2′-fluoro modified nucleotide in position 4.

In some aspects, described herein is a specific modification pattern for the polynucleic acid molecule which is a double-stranded nucleic acid molecule comprising a passenger strand and a guide strand. In some aspects, the passenger strand comprises a 2′-fluoro modified nucleotide in position 9. In some aspects, the passenger strand comprises a 2′-fluoro modified nucleotide in position 11. In some aspects, the passenger strand comprises 2′-fluoro modified nucleotides in positions 9 and 11. In some aspects, the passenger strand comprises a 2′-fluoro modified nucleotide in position 7. In some aspects, the passenger strand comprises a 2′-fluoro modified nucleotide in position 10. In some aspects, the passenger strand comprises 2′-fluoro modified nucleotides in positions 9, 11, and 7. the passenger strand comprises 2′-fluoro modified nucleotides in positions 9 and 11, and 10. the passenger strand comprises 2′-fluoro modified nucleotides in positions 9 and 7. the passenger strand comprises 2′-fluoro modified nucleotides in positions 9 and 10. the passenger strand comprises 2′-fluoro modified nucleotides in positions 9, 11, 7, and 10. In other aspects, the passenger strand comprises a 2′-fluoro modified nucleotide in position 8. In other aspects, the passenger strand comprises a 2′-fluoro modified nucleotide in position 12. In other aspects, the passenger strand comprises a 2′-fluoro modified nucleotide in position 16.

In some aspects, the passenger strand and guide strand of the polynucleic acid molecule comprises any combination of two or more 2′-fluoro modified nucleotides at the positions described in the above two paragraphs.

In some aspects, the guide strand comprises 5′-nNfnnnNfnNfNfnnnnNfnNfnnnnnnn-3′. In some aspects, the guide strand comprises 5′-nNfnnnNfnnnnnnnNfnNfnnnnnnn-3′. In some aspects, the guide strand comprises 5′-nNfnnnnNfnnnnNfnNfnnnnnnnn-3′. In the modification patterns described above, “Nf” stands for a 2′-fluoro modified nucleotide, and wherein “n” stands for a 2′-O-methyl modified nucleotide.

In some aspects, the passenger strand comprises 5′-nnnnnnNfnNfnNfnnnnnnnnn-3′. In some aspects, the passenger strand comprises 5′-nnnnnnNfnNfNfNfmnnnnnnnnn-3′. In some aspects, the passenger strand comprises 5′-nnnnnnnnNfNfNfnnnnnnnnnn-3′. In the modification patterns described above, “Nf” stands for a 2′-fluoro modified nucleotide, and wherein “n” stands for a 2′-O-methyl modified nucleotide.

In some aspects, described herein is a specific modification pattern for the polynucleic acid molecule which is a double-stranded nucleic acid molecule comprising a passenger strand and a guide strand, wherein the passenger strand comprises about twelve 2′-fluoro modified nucleotides and about nine 2′-O-methyl modified nucleotides, and wherein the guide strand comprises about nine 2′-fluoro modified nucleotides and about fourteen 2′-O-methyl modified nucleotides.

In some aspects, described herein is a specific modification pattern, wherein the passenger strand is fully modified and comprises twelve 2′-fluoro modified nucleotides, nine 2′-O-methyl modified nucleotides, and wherein the guide strand is fully modified and comprises nine 2′-fluoro modified nucleotides and fourteen 2′-O-methyl modified nucleotides.

In some aspects, described herein is a specific modification pattern, wherein the passenger strand comprises 5′-NfnNfnNfnNfnNfNfNfnNfnNfnNfnNfnNf-3′, wherein the guide strand comprises 5′-nNfnNfnNfnNfnNfinnNfnNfnNfnNfnnn-3′, wherein “Nf” stands for a 2′-fluoro modified nucleotide, and wherein “n” stands for a 2′-O-methyl modified nucleotide.

In some aspects, described herein is a specific modification pattern, wherein the passenger strand comprises 5′-NfnNfnNfnNfnNfNfNfnNfnNfnNfnNfnNf-3′, wherein the guide strand comprises 5′-nNfnNfnNfnNfhNfhnnNfnNfnNfnNfnnn-3′, wherein the passenger strand and/or guide strand comprises one or more phosphorothioate linkage, wherein “Nf” stands for a 2′-fluoro modified nucleotide, and wherein “n” stands for a 2′-O-methyl modified nucleotide. In other aspects, described herein is a specific modification pattern, wherein the passenger strand comprises 5′-NfnNfnNfnNfnNfNfNfnNfnNfhNfhNfhNf-3′, wherein the guide strand comprises 5′-nNfnNfnNfnNfnNfnmNfnNfnNfnNfnnn-3′, wherein the passenger strand comprises two phosphorothioate linkages, wherein the guide comprises four phosphorothioate linkages, wherein “Nf” stands for a 2′-fluoro modified nucleotide, and wherein “n” stands for a 2′-O-methyl modified nucleotide.

In some aspects, described herein is a specific modification pattern, wherein the passenger strand comprises 5′-nsnsnnnnNfnNfNfnnnnnnnnnn-3′, wherein the guide strand comprises 5′-nsNfsnnnnNfnnnnNfnNfnNfnnnnnsnsn-3′, wherein the passenger comprises two phosphorothioate linkages, wherein “s” stands for phosphorothioate linkages, wherein “Nf” stands for a 2′-fluoro modified nucleotide, and wherein “n” stands for a 2′-O-methyl modified nucleotide.

In some aspects, described herein is a specific modification pattern, wherein the passenger strand and/or guide strand is modified as Type I in Table 6.

TABLE 6
Nucleotide Modification Patterns

| Pattern Name | Pattern |
|---|---|
| Type I for passenger strand | 5′-NfsnsNfnNfnNfnNfNfNfnNfnNfnNfnNfnNf-3′ |
| Type I for guide strand | 5′-nsNfsnNfnNfnNfnNfnnnNfnNfnNfnNfnsnsn-3′ |
| Type II for passenger strand | 5′-nsnsnnnnNfnNfNfNfnnnnnnnnnn-3′ |
| Type II for guide strand | 5′-nsNfsnnnNfnNfNfnnnnNfnNfnnnnnsnsn-3′ |
| Type III for passenger | 5′-nsnsnnnnnnNfnNfnnnnnnnnnn-3′ |
| strand | |
| Type III for guide strand | 5′-nsNfsnnnnnnnnnNfnNfnnnnnnnsnsn-3′ |
| Type IV for passenger | 5′-nsnsnnnnNfnNfnNfnnnnnnnnnn-3′ |
| strand | |
| Type IV for guide strand | 5′-nsNfsnnnnnnnnnNfnNfnNfnnnnnsnsn-3′ |
| Type V for passenger strand | 5′-nsnsnnnnNfnNfnNfnnnnnnnnnn-3′ |
| Type V for guide strand | 5′-nsNfsnnnnNfnnnnNfnNfnNfnnnnnsnsn-3′ |
| Type VI for passenger | 5′-nnnnnnNfnNfnNfnnnnnnnnnn-invdN-invdN-3′ |
| strand | |

Note:
“Nf” stands for a 2′-fluoro modified nucleotide, “n” stands for a 2′-O-methyl modified nucleotide, “s” stands for a 3′-phosphorothioate, “invdN” stands for an inverted deoxy-nucleotide.

In some aspects, the polynucleic acid molecule provided herein comprises a passenger strand from a nucleic acid sequence selected from SEQ ID NOs: 401-596, 819-828, and 870-875 and a guide strand comprises a nucleic acid sequence selected from SEQ ID NOs: 1-196, 797-806, and 850-857. In other aspects, the polynucleic acid molecule provided herein comprises a passenger strand from a nucleic acid sequence selected from SEQ ID NOs: 401-596, 819-828, and 870-875, a guide strand comprises a nucleic acid sequence selected from SEQ ID NOs: 1-196, 797-806, and 850-857, and wherein the passenger strand and/or guide strand is modified in Type I modification pattern specified in Table 6. In some aspects, the polynucleic acid molecule provided herein comprises a passenger strand from a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875, and a guide strand comprises a nucleic acid sequence selected from SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857.

In other aspects, the polynucleic acid molecule provided herein comprises a passenger strand from a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875, a guide strand comprises a nucleic acid sequence selected from SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857, and wherein the passenger strand and/or guide strand is modified in Type I modification pattern specified in Table 6.

In some aspects, the polynucleic acid molecule provided herein comprises a passenger strand from a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875, and a guide strand comprises a nucleic acid sequence selected from SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857. In other aspects, the polynucleic acid molecule provided herein comprises a passenger strand from a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875, and a guide strand comprises a nucleic acid sequence selected from SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857, and wherein the passenger strand and/or guide strand is modified in Type I modification pattern specified in Table 6.

In some aspects, the polynucleic acid molecule provided herein comprises a passenger strand from a nucleic acid sequence selected from SEQ ID NOs: 513, 546, 553, 558, 508, 514, and 545, and a guide strand comprises a nucleic acid sequence selected from SEQ ID NOs: 113, 146, 153, 158, 108, 114, and 145. In other aspects, the polynucleic acid molecule provided herein comprises a passenger strand from a nucleic acid sequence selected from SEQ ID NOs: 513, 546, 553, 558, 508, 514, and 545, and a guide strand comprises a nucleic acid sequence selected from SEQ ID NOs: 113, 146, 153, 158, 108, 114, and 145, and wherein the passenger strand and/or guide strand is modified in Type I modification pattern specified in Table 6.

In some aspects, described herein is a specific modification pattern, wherein the passenger strand comprises about four 2′-fluoro modified nucleotides and about seventeen 2′-O-methyl modified nucleotides, and wherein the guide strand comprises about six 2′-fluoro modified nucleotides and about seventeen 2′-O-methyl modified nucleotides.

In some aspects, described herein is a specific modification pattern, wherein the passenger strand is fully modified and comprises four 2′-fluoro modified nucleotides, seventeen 2′-O-methyl modified nucleotides, and wherein the guide strand is fully modified and comprises six 2′-fluoro modified nucleotides and seventeen 2′-O-methyl modified nucleotides.

In some aspects, described herein is a specific modification pattern, wherein the passenger strand comprises 5′-nnnnnnNfnNfNfNfnnnnnnnnnn-3′, wherein the guide strand comprises 5′-nNfnnnNfnNfNfnnnnNfnNfhnnnnnn-3′, wherein “Nf” stands for a 2′-fluoro modified nucleotide, and wherein “n” stands for a 2′-O-methyl modified nucleotide.

In some aspects, described herein is a specific modification pattern, wherein the passenger strand comprises 5′-nnnnnnNfnNfNfNfnnnnnnnnnn-3′, wherein the guide strand comprises 5′-nNfnnnNfnNfNfnnnnNfnNfinnnnnn-3′, wherein the passenger strand and/or guide strand comprises one or more phosphorothioate linkage, wherein “Nf” stands for a 2′-fluoro modified nucleotide, and wherein “n” stands for a 2′-O-methyl modified nucleotide. In other aspects, described herein is a specific modification pattern, wherein the passenger strand comprises 5′-nnnnnnNfnNfNfNfnnnnnnnnnn-3′, wherein the guide strand comprises 5′-nNfnnnNfnNfNfnnnnNfnNfnnnnnnn-3′, wherein the passenger strand comprises two phosphorothioate linkages, wherein the guide comprises four phosphorothioate linkages, wherein “Nf” stands for a 2′-fluoro modified nucleotide, and wherein “n” stands for a 2′-O-methyl modified nucleotide.

In some aspects, described herein is a specific modification pattern, wherein the passenger strand and/or guide strand is modified as Type II in Table 6.

In some aspects, the polynucleic acid molecule provided herein comprises a passenger strand comprising a nucleic acid sequence selected from SEQ ID NOs: 401-596, 819-828, and 870-875, and/or a guide strand comprising a nucleic acid sequence selected from SEQ ID NOs: 1-196, 797-806, and 850-857, and wherein the passenger strand and/or guide strand is modified in Type II modification pattern specified in Table 6. In other aspects, the polynucleic acid molecule provided herein comprises a passenger strand comprising a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875, and/or a guide strand comprising a nucleic acid sequence selected from SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857, and wherein the passenger strand and/or guide strand is modified in Type II modification pattern specified in Table 6. In other aspects, the polynucleic acid molecule provided herein comprises a passenger strand comprising a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875, and/or a guide strand comprising a nucleic acid sequence selected from SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857, and wherein the passenger strand and/or guide strand is modified in Type II modification pattern specified in Table 6. In other aspects, the polynucleic acid molecule provided herein comprises a passenger strand comprising a nucleic acid sequence selected from SEQ ID NOs: 405, 408, 418, 422, 440, 444-446, 450, 452-453, 456, 513, 546, 553, 509. 558, 508, 514, and 545, and/or a guide strand comprising a nucleic acid sequence selected from SEQ ID NOs: 113, 146, 153, 158, 108, 114, and 145, and wherein the passenger strand and/or guide strand is modified in Type II modification pattern specified in Table 6.

In some aspects, described herein is a specific modification pattern, wherein the passenger strand comprises about two 2′-fluoro modified nucleotides and about nineteen 2′-O-methyl modified nucleotides, and wherein the guide strand comprises about three 2′-fluoro modified nucleotides and about twenty 2′-O-methyl modified nucleotides.

In some aspects, described herein is a specific modification pattern, wherein the passenger strand is fully modified and comprises two 2′-fluoro modified nucleotides and nineteen 2′-O-methyl modified nucleotides, and wherein the guide strand is fully modified and comprises three 2′-fluoro modified nucleotides and twenty 2′-O-methyl modified nucleotides.

In some aspects, described herein is a specific modification pattern, wherein the passenger strand comprises 5′-nnnnnnnnNfnNfnnnnnnnnnn-3′, wherein the guide strand comprises 5′-nNfnnnnnnnnnNfnNfnnnnnnnnn-3′, wherein “Nf” stands for a 2′-fluoro modified nucleotide, and wherein “n” stands for a 2′-O-methyl modified nucleotide.

In some aspects, described herein is a specific modification pattern, wherein the passenger strand comprises 5′-nnnnnnnnNfnNfnnnnnnnnnn-3′, wherein the guide strand comprises 5′-nNfnnnnnnnnnNfnNfnnnnnnnnn-3′, wherein the passenger strand and/or guide strand comprises one or more phosphorothioate linkage, wherein “Nf” stands for a 2′-fluoro modified nucleotide, and wherein “n” stands for a 2′-O-methyl modified nucleotide. In other aspects, described herein is a specific modification pattern, wherein the passenger strand comprises 5′-nnnnnnnnNfnNfnnnnnnnnnn-3′, wherein the guide strand comprises 5′-nNfnnnnnnnnnNfnNfnnnnnnnnn-3′, wherein the passenger comprises two phosphorothioate linkages, wherein the guide comprises four phosphorothioate linkages, wherein “Nf” stands for a 2′-fluoro modified nucleotide, and wherein “n” stands for a 2′-O-methyl modified nucleotide.

In some aspects, described herein is a specific modification pattern, wherein the passenger strand and/or guide strand is modified as Type III in Table 6.

In some aspects, the polynucleic acid molecule provided herein comprises a passenger strand comprising a nucleic acid sequence selected from SEQ ID NOs: 401-596, 819-828, and 870-875, and/or a guide strand comprising a nucleic acid sequence selected from SEQ ID NOs: 1-196, 797-806, and 850-857, and wherein the passenger strand and/or guide strand is modified in Type III modification pattern specified in Table 6. In other aspects, the polynucleic acid molecule provided herein comprises a passenger strand comprising a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875, and/or a guide strand comprising a nucleic acid sequence selected from SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857, and wherein the passenger strand and/or guide strand is modified in Type III modification pattern specified in Table 6. In other aspects, the polynucleic acid molecule provided herein comprises a passenger strand comprising a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875, and/or a guide strand comprising a nucleic acid sequence selected from SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857, and wherein the passenger strand and/or guide strand is modified in Type III modification pattern specified in Table 6. In other aspects, the polynucleic acid molecule provided herein comprises a passenger strand comprising a nucleic acid sequence selected from SEQ ID NOs: 513, 546, 553, 558, 508, 514, and 545, and/or a guide strand comprising a nucleic acid sequence selected from SEQ ID NOs: 113, 146, 153, 158, 108, 114, and 145, and wherein the passenger strand and/or guide strand is modified in Type III modification pattern specified in Table 6.

In some aspects, described herein is a specific modification pattern, wherein the passenger strand comprises about three 2′-fluoro modified nucleotides and about eighteen 2′-O-methyl modified nucleotides, and wherein the guide strand comprises about four 2′-fluoro modified nucleotides and about nineteen 2′-O-methyl modified nucleotides.

In some aspects, described herein is a specific modification pattern, wherein the passenger strand is fully modified and comprises three 2′-fluoro modified nucleotides, eighteen 2′-O-methyl modified nucleotides, and wherein the guide strand is fully modified and comprises four 2′-fluoro modified nucleotides, nineteen 2′-O-methyl modified nucleotides.

In some aspects, described herein is a specific modification pattern, wherein the passenger strand comprises 5′-nnnnnnNfnNfnNfnnnnnnnnnn-3′, wherein the guide strand comprises 5′-nNfnnnnnnnnnNfnNfnNfnnnnnnn-3′, wherein “Nf” stands for a 2′-fluoro modified nucleotide, and wherein “n” stands for a 2′-O-methyl modified nucleotide.

In some aspects, described herein is a specific modification pattern, wherein the passenger strand comprises 5′-nnnnnnNfnNfnNfnnnnnnnnnn-3′, wherein the guide strand comprises 5′-nNfnnnnnnnnnNfnNfnNfnnnnnnn-3′, wherein the passenger strand and/or guide strand comprises one or more phosphorothioate linkage, wherein “Nf” stands for a 2′-fluoro modified nucleotide, and wherein “n” stands for a 2′-O-methyl modified nucleotide. In other aspects, described herein is a specific modification pattern, wherein the passenger strand comprises 5′-nnnnnnNfnNfnNfnnnnnnnnnn-3′, wherein the guide strand comprises 5′-nNfnnnnnnnnnNfnNfnNfnnnnnnn-3′, wherein the passenger comprises two phosphorothioate linkages, wherein the guide comprises four phosphorothioate linkages, wherein “Nf” stands for a 2′-fluoro modified nucleotide, and wherein “n” stands for a 2′-O-methyl modified nucleotide.

In some aspects, described herein is a specific modification pattern, wherein the passenger strand and/or guide strand is modified as Type IV in Table 6.

In some aspects, the polynucleic acid molecule provided herein comprises a passenger strand comprising a nucleic acid sequence selected from SEQ ID NOs: 401-596, 819-828, and 870-875, and/or a guide strand comprising a nucleic acid sequence selected from SEQ ID NOs: 1-196, 797-806, and 850-857, and wherein the passenger strand and/or guide strand is modified in Type IV modification pattern specified in Table 6. In other aspects, the polynucleic acid molecule provided herein comprises a passenger strand comprising a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875, and/or a guide strand comprising a nucleic acid sequence selected from SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857, and wherein the passenger strand and/or guide strand is modified in Type IV modification pattern specified in Table 6. In other aspects, the polynucleic acid molecule provided herein comprises a passenger strand comprising a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875, and/or a guide strand comprising a nucleic acid sequence selected from SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857, and wherein the passenger strand and/or guide strand is modified in Type IV modification pattern specified in Table 6. In other aspects, the polynucleic acid molecule provided herein comprises a passenger strand comprising a nucleic acid sequence selected from SEQ ID NOs: 513, 546, 553, 558, 508, 514, and 545, and/or a guide strand comprising a nucleic acid sequence selected from SEQ ID NOs: 113, 146, 153, 158, 108, 114, and 145, and wherein the passenger strand and/or guide strand is modified in Type IV modification pattern specified in Table 6.

In some aspects, described herein is a specific modification pattern, wherein the passenger strand comprises about three 2′-fluoro modified nucleotides and about eighteen 2′-O-methyl modified nucleotides, and wherein the guide strand comprises about five 2′-fluoro modified nucleotides and about eighteen 2′-O-methyl modified nucleotides.

In some aspects, described herein is a specific modification pattern, wherein the passenger strand is fully modified and comprises three 2′-fluoro modified nucleotides and eighteen 2′-O-methyl modified nucleotides, and wherein the guide strand is fully modified and comprises five 2′-fluoro modified nucleotides, eighteen 2′-O-methyl modified nucleotides.

In some aspects, described herein is a specific modification pattern, wherein the passenger strand comprises 5′-nnnnnnNfnNfnNfnnnnnnnnnn-3′, wherein the guide strand comprises 5′-_nNfnnnnNfnnnnNfnNfnNfnnnnnnn-3′, wherein “Nf” stands for a 2′-fluoro modified nucleotide, and wherein “n” stands for a 2′-O-methyl modified nucleotide.

In some aspects, described herein is a specific modification pattern, wherein the passenger strand comprises 5′-nnnnnnNfnNfnNfnnnnnnnnnn-3′, wherein the guide strand comprises 5′-nNfnnnnNfnnnnNfnNfnNfnnnnnnn-3′, wherein the passenger strand and/or guide strand comprises one or more phosphorothioate linkage, wherein “Nf” stands for a 2′-fluoro modified nucleotide, and wherein “n” stands for a 2′-O-methyl modified nucleotide. In other aspects, described herein is a specific modification pattern, wherein the passenger strand comprises 5′-nnnnnnNfnNfnNfnnnnnnnnnn-3′, wherein the guide strand comprises 5′-nNfnnnnNfhnnnNfnNfnNfnnnnnnn-3′, wherein the passenger strand comprises two phosphorothioate linkages, wherein the guide comprises four phosphorothioate linkages, wherein “Nf” stands for a 2′-fluoro modified nucleotide, and wherein “n” stands for a 2′-O-methyl modified nucleotide.

In some aspects, described herein is a specific modification pattern, wherein the passenger strand and/or guide strand is modified as Type V in Table 6.

In some aspects, the polynucleic acid molecule provided herein comprises a passenger strand comprising a nucleic acid sequence selected from SEQ ID NOs: 401-596, 819-828, and 870-875, and/or a guide strand comprising a nucleic acid sequence selected from SEQ ID NOs: 1-196, 797-806, and 850-857, and wherein the passenger strand and/or guide strand is modified in Type V modification pattern specified in Table 6. In other aspects, the polynucleic acid molecule provided herein comprises a passenger strand comprising a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875, and/or a guide strand comprising a nucleic acid sequence selected from SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857, and wherein the passenger strand and/or guide strand is modified in Type V modification pattern specified in Table 6. In other aspects, the polynucleic acid molecule provided herein comprises a passenger strand comprising a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875, and/or a guide strand comprising a nucleic acid sequence selected from SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857, and wherein the passenger strand and/or guide strand is modified in Type V modification pattern specified in Table 6. In other aspects, the polynucleic acid molecule provided herein comprises a passenger strand comprising a nucleic acid sequence selected from SEQ ID NOs: 513, 546, 553, 558, 508, 514, and 545, and/or a guide strand comprising a nucleic acid sequence selected from SEQ ID NOs: 113, 146, 153, 158, 108, 114, and 145, and wherein the passenger strand and/or guide strand is modified in Type V modification pattern specified in Table 6.

In some aspects, described herein is a specific modification pattern, wherein the passenger strand comprises about three 2′-fluoro modified nucleotides and about eighteen 2′-O-methyl modified nucleotides, with one or more inverted deoxy-nucleotides on the 3′ end as an overhang.

In some aspects, described herein is a specific modification pattern, wherein the passenger strand is fully modified and comprises three 2′-fluoro modified nucleotides and eighteen 2′-O-methyl modified nucleotides, with two inverted deoxy-nucleotides on the 3′ end as an overhang.

In some aspects, described herein is a specific modification pattern, wherein the passenger strand comprises 5′-nnnnnnNfnNfnNfnnnnnnnnnn-invdN-invdN-3′, wherein “Nf” stands for a 2′-fluoro modified nucleotide, wherein “n” stands for a 2′-O-methyl modified nucleotide, and “invdN” stands for an inverted deoxy-nucleotide. In some instances, the invdN is an inverted deoxyl-thymine. In some aspects, the linker conjugated with one or more targeting moieties as shown in Formula (IV″) or (IV″′) is added to the first nucleic acid on the 5′ end. In some aspects, the linker conjugated with one or more GalNAc as shown in Formula (V″) or (V″′) is added to the first nucleic acid on the 5′ end. In some aspects, the modification pattern comprises one or more phosphorothioate linkages. In some aspects, the modification pattern is shown in Formula (VII). In some aspects, the 5′ end modification known in the art is applied to the one or more inverted nucleotides.

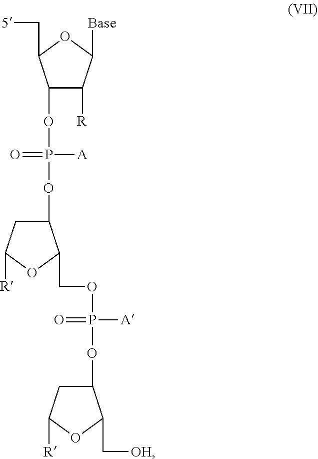

wherein R is a moiety that corresponds to the sugar modification described herein, in some instances, R is —O-methyl; wherein R′ is thymine, abasic, or others; wherein A is —O or —S; and wherein A′ is —O or —S.

In some aspects, the polynucleic acid molecule provided herein comprises a passenger strand comprising a nucleic acid sequence selected from SEQ ID NOs: 401-596, 819-828, and 870-875, and/or a guide strand comprising a nucleic acid sequence selected from SEQ ID NOs: 1-196, 797-806, and 850-857, and wherein the passenger strand is modified in Type VI modification pattern specified in Table 6 or as described in the preceding paragraph. In other aspects, the polynucleic acid molecule provided herein comprises a passenger strand comprising a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875, and/or a guide strand comprising a nucleic acid sequence selected from SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857, and wherein the passenger strand is modified in Type VI modification pattern specified in Table 6 or as described in the preceding paragraph. In other aspects, the polynucleic acid molecule provided herein comprises a passenger strand comprising a nucleic acid sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875, and/or a guide strand comprising a nucleic acid sequence selected from SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857, and wherein the passenger strand is modified in Type VI modification pattern specified in Table 6 or as described in the preceding paragraph. In other aspects, the polynucleic acid molecule provided herein comprises a passenger strand comprising a nucleic acid sequence selected from SEQ ID NOs: 513, 546, 553, 558, 508, 514, and 545, and/or a guide strand comprising a nucleic acid sequence selected from SEQ ID NOs: 113, 146, 153, 158, 108, 114, and 145, and wherein the passenger strand is modified in Type VI modification pattern specified in Table 6 or as described in the preceding paragraph.

In some aspects, described herein is a specific modification pattern, wherein the passenger strand comprises about three 2′-fluoro modified nucleotides and about eighteen 2′-O-methyl modified nucleotides, and wherein the guide strand comprises about four 2′-fluoro modified nucleotides and about nineteen 2′-O-methyl modified nucleotides.

In some aspects, described herein is a specific modification pattern, wherein the passenger strand is fully modified and comprises three 2′-fluoro modified nucleotides, eighteen 2′-O-methyl modified nucleotides, and wherein the guide strand is fully modified and comprises four 2′-fluoro modified nucleotides, nineteen 2′-O-methyl modified nucleotides.

In some aspects, described herein is a specific modification pattern, wherein the passenger strand comprises 5′-nsnsnnnnNfnNfnNfnnnnnnnnnn-3′, wherein the guide strand comprises 5′-nsNfsnnnnNfnnnnNfnNfnNfnnnnnsnsn-3′, wherein “Nf” stands for a 2′-fluoro modified nucleotide, and wherein “n” stands for a 2′-O-methyl modified nucleotide.

In some aspects, described herein is a specific modification pattern, wherein the passenger strand comprises 5′-nsnsnnnnNfnNfnNfnnnnnnnnnn-3′, wherein the guide strand comprises 5′-nsNfsnnnnNfnnnnNfnNfnNfnnnnnsnsn-3′, wherein the passenger strand and/or guide strand comprises one or more phosphorothioate linkage, wherein “Nf” stands for a 2′-fluoro modified nucleotide, and wherein “n” stands for a 2′-O-methyl modified nucleotide. In other aspects, described herein is a specific modification pattern, wherein the passenger strand comprises 5′-nsnsnnnnNfnNfnNfmrnnnnnnnn-3′, wherein the guide strand comprises 5′-nsNfsnnnnNfhnnnNfnNfnNfnnnnsnsn-3′, wherein the passenger comprises two phosphorothioate linkages, wherein the guide comprises four phosphorothioate linkages, wherein “Nf” stands for a 2′-fluoro modified nucleotide, and wherein “n” stands for a 2′-O-methyl modified nucleotide.

Described herein is a polynucleic acid molecule, whose passenger strand comprises a nucleic acid sequence that is at least 80% identical to a nucleic acid sequence selected from SEQ ID NOs: 601-796, 830-839, and 876-881. Described herein is a polynucleic acid molecule, whose passenger strand comprises a nucleic acid sequence that is at least 85% identical to a nucleic acid sequence selected from SEQ ID NOs: 601-796, 830-839, and 876-881. Described herein is a polynucleic acid molecule, whose passenger strand comprises a nucleic acid sequence that is at least 90% identical to a nucleic acid sequence selected from SEQ ID NOs: 601-796, 830-839, and 876-881. Described herein is a polynucleic acid molecule, whose passenger strand comprises a nucleic acid sequence that is at least 95% identical to a nucleic acid sequence selected from SEQ ID NOs: 601-796, 830-839, and 876-881.

Described herein is a polynucleic acid molecule, which guide strand comprises a nucleic acid sequence that is at least 80% identical to a nucleic acid sequence selected from SEQ ID NOs: 201-396, 808-817, and 858-868. Described herein is a polynucleic acid molecule, which guide strand comprises a nucleic acid sequence that is at least 85% identical to a nucleic acid sequence selected from SEQ ID NOs: 201-396, 808-817, and 858-868. Described herein is a polynucleic acid molecule, which guide strand comprises a nucleic acid sequence that is at least 90% identical to a nucleic acid sequence selected from SEQ ID NOs: 201-396, 808-817, and 858-868. Described herein is a polynucleic acid molecule, which guide strand comprises a nucleic acid sequence that is at least 95% identical to a nucleic acid sequence selected from SEQ ID NOs: 201-396, 808-817, and 858-868.

A polynucleic acid molecule for modulating expression of ANGPTL3 gene, wherein polynucleic acid molecule comprises a guide strand comprising the nucleotide sequence selected from SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857 and a passenger strand comprising the nucleotide sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875.

A polynucleic acid molecule for modulating expression of ANGPTL3 gene, wherein polynucleic acid molecule comprises a guide strand comprising the nucleotide sequence selected from SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857 and a passenger strand comprising the nucleotide sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875.

A polynucleic acid molecule for modulating expression of ANGPTL3 gene, wherein polynucleic acid molecule comprises a guide strand comprising the nucleotide sequence selected from SEQ ID NOs: 113, 146, 153, 158, 108, 114, and 145 and a passenger strand comprising the nucleotide sequence selected from SEQ ID NOs: 513, 546, 553, 558, 508, 514, and 545.

A polynucleic acid molecule for modulating expression of ANGPTL3 gene, wherein polynucleic acid molecule comprises a guide strand comprising the nucleotide sequence selected from SEQ ID NOs: 205, 207, 208, 211, 218, 220, 222, 240, 243-246, 250, 252, 253, 256, 308, 309, 313, 314, 345, 346, 348, 353, 357, 358, 808-817, and 858-868 and a passenger strand comprising the nucleotide sequence selected from SEQ ID NOs: 605, 607, 608, 611, 618, 620, 622, 640, 643, 644, 645, 646, 650, 652, 653, 656, 708, 709, 713, 714, 745, 746, 748, 753, 757, 758, 830-839, and 876-881.

A polynucleic acid molecule for modulating expression of ANGPTL3 gene, wherein polynucleic acid molecule comprises a guide strand comprising the nucleotide sequence selected from SEQ ID NOs: 205, 207, 208, 211, 218, 220, 222, 240, 243-246, 250, 252, 253, 256, 308, 309, 313, 314, 345, 346, 348, 353, 357, 358, 808-817, and 858-868, and a passenger strand comprising the nucleotide sequence selected from SEQ ID NOs: 605, 607, 608, 611, 618, 620, 622, 640, 643, 644, 645, 646, 650, 652, 653, 656, 708, 709, 713, 714, 745, 746, 748, 753, 757, 758, 830-839, and 876-881.

A polynucleic acid molecule for modulating expression of ANGPTL3 gene, wherein polynucleic acid molecule comprises a guide strand comprising the nucleotide sequence selected from SEQ ID NOs: 313, 346, 353, 358, 308, 314, and 345, and a passenger strand comprising the nucleotide sequence selected from SEQ ID NOs: 713, 746, 753, 758, 708, 714, and 745.

A polynucleic acid molecule for modulating expression of ANGPTL3 gene, wherein polynucleic acid molecule comprises a guide strand comprising the nucleotide sequence of usUfsaguuGfguuuCfgUfgAfuuuccscsa (SEQ ID NO: 313) and a passenger strand comprising the nucleotide sequence of gsgsaaauCfaCfgAfaaccaacuaa (SEQ ID NO: 713), where smaller case “n” stands for 2′-O-methyl modified nucleotide, upper case followed with an “f” (i.e., “Nf”) stands for 2′-fluoro modified nucleotide, and “s” stands for 3′-phosphorothioate.

A polynucleic acid molecule for modulating expression of ANGPTL3 gene, wherein polynucleic acid molecule comprises a guide strand comprising the nucleotide sequence of usUfsagagUfauaaCfcUfuCfcauuususg (SEQ ID NO: 346) and a passenger strand comprising the nucleotide sequence of asasauggAfaGfgUfuauacucuaa (SEQ ID NO: 746), where smaller case “n” stands for 2′-O-methyl modified nucleotide, upper case followed with an “f” (i.e., “Nf”) stands for 2′-fluoro modified nucleotide, and “s” stands for 3′-phosphorothioate.

A polynucleic acid molecule for modulating expression of ANGPTL3 gene, wherein polynucleic acid molecule comprises a guide strand comprising the nucleotide sequence of usAfsuggaUfcaacAfuUfuUfgguugsasu (SEQ ID NO: 353) and a passenger strand comprising the nucleotide sequence of csasaccaAfaAfuGfuugauccaua (SEQ ID NO: 753), where smaller case “n” stands for 2′-O-methyl modified nucleotide, upper case followed with an “f” (i.e., “Nf”) stands for 2′-fluoro modified nucleotide, and “s” stands for 3′-phosphorothioate.

A polynucleic acid molecule for modulating expression of ANGPTL3 gene, wherein polynucleic acid molecule comprises a guide strand comprising the nucleotide sequence of usUfsaaggAfuuuaAfuAfcCfagauusasu (SEQ ID NO: 358) and a passenger strand comprising the nucleotide sequence of asasucugGfuAfuUfaaauccuuaa (SEQ ID NO: 758), where smaller case “n” stands for 2′-O-methyl modified nucleotide, upper case followed with an “f” (i.e., “Nf”) stands for 2′-fluoro modified nucleotide, and “s” stands for 3′-phosphorothioate.

A polynucleic acid molecule for modulating expression of ANGPTL3 gene, wherein polynucleic acid molecule comprises a guide strand comprising the nucleotide sequence of usAfsuuagAfuugcUfuCfaCfuauggsasg (SEQ ID NO: 308) and a passenger strand comprising the nucleotide sequence of cscsauagUfgAfaGfcaaucuaaua (SEQ ID NO: 708), where smaller case “n” stands for 2′-O-methyl modified nucleotide, upper case followed with an “f” (i.e., “Nf”) stands for 2′-fluoro modified nucleotide, and “s” stands for 3′-phosphorothioate.

A polynucleic acid molecule for modulating expression of ANGPTL3 gene, wherein polynucleic acid molecule comprises a guide strand comprising the nucleotide sequence of usUfsauagUfugguUfuCfgUfgauuuscsc (SEQ ID NO: 314) and a passenger strand comprising the nucleotide sequence of asasaucaCfgAfaAfccaacuauaa (SEQ ID NO: 714), where smaller case “n” stands for 2′-O-methyl modified nucleotide, upper case followed with an “f” (i.e., “Nf”) stands for 2′-fluoro modified nucleotide, and “s” stands for 3′-phosphorothioate.

A polynucleic acid molecule for modulating expression of ANGPTL3 gene, wherein polynucleic acid molecule comprises a guide strand comprising the nucleotide sequence of usAfsgaguAfuaacCfuUfcCfauuuusgsa (SEQ ID NO: 345) and a passenger strand comprising the nucleotide sequence of asasaaugGfaAfgGfuuauacucua (SEQ ID NO: 745), where smaller case “n” stands for 2′-O-methyl modified nucleotide, upper case followed with an “f” (i.e., “Nf”) stands for 2′-fluoro modified nucleotide, and “s” stands for 3′-phosphorothioate.

A polynucleic acid molecule for modulating expression of ANGPTL3 gene, wherein polynucleic acid molecule comprises a guide strand comprising the nucleotide sequence of usUfsaauuAfgauuGfcUfuCfacuausgsg (SEQ ID NO: 309) and a passenger strand comprising the nucleotide sequence of asusagugAfaGfcAfaucuaauuaa (SEQ ID NO: 709), where smaller case “n” stands for 2′-O-methyl modified nucleotide, upper case followed with an “f” (i.e., “Nf”) stands for 2′-fluoro modified nucleotide, and “s” stands for 3′-phosphorothioate.

A polynucleic acid molecule for modulating expression of ANGPTL3 gene, wherein polynucleic acid molecule comprises a guide strand comprising the nucleotide sequence of usAfsauuaGfauugCfuUfcAfcuaugsgsa (SEQ ID NO: 815) and a passenger strand comprising the nucleotide sequence of csasuaguGfaAfgCfaaucuaauua (SEQ ID NO: 837), where smaller case “n” stands for 2′-O-methyl modified nucleotide, upper case followed with an “f” (i.e., “Nf”) stands for 2′-fluoro modified nucleotide, and “s” stands for 3′-phosphorothioate.

A polynucleic acid molecule for modulating expression of ANGPTL3 gene, wherein polynucleic acid molecule comprises a guide strand comprising the nucleotide sequence of usUfsucauUfgaagUfuUfuGfugaucscsa (SEQ ID NO: 812) and a passenger strand comprising the nucleotide sequence of gsasucacAfaAfaCfuucaaugaaa (SEQ ID NO: 834), where smaller case “n” stands for 2′-O-methyl modified nucleotide, upper case followed with an “f” (i.e., “Nf”) stands for 2′-fluoro modified nucleotide, and “s” stands for 3′-phosphorothioate.

A polynucleic acid molecule for modulating expression of ANGPTL3 gene, wherein polynucleic acid molecule comprises a guide strand comprising the nucleotide sequence of usAfsuugcUfucacUfaUfgGfaguausasu (SEQ ID NO: 813) and a passenger strand comprising the nucleotide sequence of asusacucCfaUfaGfugaagcaaua (SEQ ID NO: 835), where smaller case “n” stands for 2′-O-methyl modified nucleotide, upper case followed with an “f” (i.e., “Nf”) stands for 2′-fluoro modified nucleotide, and “s” stands for 3′-phosphorothioate.

A polynucleic acid molecule for modulating expression of ANGPTL3 gene, wherein polynucleic acid molecule comprises a guide strand comprising the nucleotide sequence of usAfsgaguAfuaacCfuUfcCfauuucsgsa (SEQ ID NO: 865) and a passenger strand comprising the nucleotide sequence of gsasaaugGfaAfgGfuuauacucua (SEQ ID NO: 879), where smaller case “n” stands for 2′-O-methyl modified nucleotide, upper case followed with an “f” (i.e., “Nf”) stands for 2′-fluoro modified nucleotide, and “s” stands for 3′-phosphorothioate.

A polynucleic acid molecule for modulating expression of ANGPTL3 gene, wherein polynucleic acid molecule comprises a guide strand comprising the nucleotide sequence of usAfsgaguAfuaacCfuUfcCfauuccsgsa (SEQ ID NO: 866) and a passenger strand comprising the nucleotide sequence of gsgsaaugGfaAfgGfuuauacucua (SEQ ID NO: 880), where smaller case “n” stands for 2′-O-methyl modified nucleotide, upper case followed with an “f” (i.e., “Nf”) stands for 2′-fluoro modified nucleotide, and “s” stands for 3′-phosphorothioate.

In one aspect, described herein is a specific modification motif or pattern for the double-stranded inhibitiory polynucleic acid molecule comprising a passenger strand and a guide strand.

In some aspects, the guide strand comprises a nucleotide analogue selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), thymidine-glycol nucleic acid (GNA)S-isomer (Tgn), 2′-O-methyl-2-thiouridine-3′-phosphate (u3), 2′-fluoro-2-thiouridine-3′-phosphate (U3f), or 2-amino-2′-O-methyladenosine-3′-phosphate (al). In some instance, the nucleotide analogue is selected from a group consisting of the nucleotide analogue selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), thymidine-glycol nucleic acid (GNA)S-isomer (Tgn), 2′-O-methyl-2-thiouridine-3′-phosphate (u3), 2′-fluoro-2-thiouridine-3′-phosphate (U3), and 2-amino-2′-O-methyladenosine-3′-phosphate (al). In some instances, the nucleotide analogue is selected from a group consisting of the nucleotide analogue selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), thymidine-glycol nucleic acid (GNA)S-isomer (Tgn), 2′-O-methyl-2-thiouridine-3′-phosphate (u3), and 2′-fluoro-2-thiouridine-3′-phosphate (U3). In some instances, the nucleotide analogue is selected from a group consisting of the nucleotide analogue selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), and 1′,2′-Dideoxyribose-3′phosphate (dAB). In some instances, the guide strand comprises a nucleotide analogue selected from a group consisting of acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), and 1′,2′-Dideoxyribose-3′phosphate (dAB).

In some aspects, the guide strand comprises a nucleotide analogue at one of positions 2-8 from the 5′ end, and further comprises a 2′-fluoro modified nucleotide at position 2 from the 5′ end. In some aspects, the guide strand comprises a nucleotide analogue at one of positions 2-8 from the 5′ end, and further comprises a 2′-fluoro modified nucleotide at position 7 from the 5′ end. In some aspects, the guide strand comprises a nucleotide analogue at one of positions 2-8 from the 5′ end, and further comprises 2′-fluoro modified nucleotides at position 12 from the 5′ end. In some aspects, the guide strand comprises a nucleotide analogue at one of positions 2-8 from the 5′ end, and further comprises a 2′-fluoro modified nucleotide at position 14 from the 5′ end. In some aspects, the guide strand comprises a nucleotide analogue at one of positions 2-8 from the 5′ end, and further comprises a 2′-fluoro modified nucleotide at position 16 from the 5′ end. In other aspects, the guide strand comprises a nucleotide analogue at one of positions 2-8 from the 5′ end, and further comprises a 2′-fluoro modified nucleotide at positions 2, 12, 14, 16, or combination thereof from the 5′ end. In other aspects, the guide strand comprises a nucleotide analogue at one of positions 2-8 from the 5′ end, and further comprises a 2′-fluoro modified nucleotide at positions 2, 7, 12, 14, 16, or combination thereof from the 5′ end. In other aspects, the guide strand comprises a nucleotide analogue at one of positions 2-8 from the 5′ end, and further comprises a 2′-fluoro modified nucleotide at at least three of positions 2, 12, 14, and 16 from the 5′ end. In other aspects, the guide strand comprises a nucleotide analogue at one of positions 2-8 from the 5′ end, and further comprises a 2′-fluoro modified nucleotide at at least three of positions 2, 7, 12, 14, and 16 from the 5′ end.

In some instances, the nucleotide analogue is located at position 6 from the 5′ end of the guide strand. In some instances, the nucleotide analogue is located at position 6 from the 5′ end of the guide strand, and the guide strand further comprises 2′-fluoro (2′-F) modified nucleotides at at least one of positions 2, 7, 12, 14, and 16 from the 5′ end. In some instances, the nucleotide analogue is located at position 6 from the 5′ end of the guide strand, and the guide strand further comprises 2′-fluoro (2′-F) modified nucleotides at at least two of positions 2, 7, 12, 14, and 16 from the 5′ end. In some instances, the nucleotide analogue is located at position 6 from the 5′ end of the guide strand, and the guide strand further comprises 2′-fluoro (2′-F) modified nucleotides at at least three of positions 2, 7, 12, 14, and 16 from the 5′ end. In some instances, the nucleotide analogue is located at position 6 from the 5′ end of the guide strand, and the guide strand further comprises 2′-fluoro (2′-F) modified nucleotides at positions 2, 7, 12, 14, and 16 from the 5′ end.

In some instances, the nucleotide analogue is located at position 7 from the 5′ end of the guide strand. In some instances, the nucleotide analogue is located at position 7 from the 5′ end of the guide strand, and the guide strand further comprises 2′-fluoro (2′-F) modified nucleotides at at least one of positions 2, 6, 8, 12, 14, and 16 from the 5′ end. In some instances, the nucleotide analogue is located at position 7 from the 5′ end of the guide strand, and the guide strand further comprises 2′-fluoro (2′-F) modified nucleotides at at least one of positions 2, 12, 14, and 16 from the 5′ end. In some instances, the nucleotide analogue is located at position 7 from the 5′ end of the guide strand, and the guide strand further comprises 2′-fluoro (2′-F) modified nucleotides at at least two of positions 2, 12, 14, and 16 from the 5′ end. In some instances, the nucleotide analogue is located at position 7 from the 5′ end of the guide strand, and the guide strand further comprises 2′-fluoro (2′-F) modified nucleotides at at least three of positions 2, 12, 14, and 16 from the 5′ end. In some instances, the nucleotide analogue is located at position 7 from the 5′ end of the guide strand, and the guide strand further comprises 2′-fluoro (2′-F) modified nucleotides at positions 2, 12, 14, and 16 from the 5′ end. In some instances, the nucleotide analogue is located at position 7 from the 5′ end of the guide strand, and the guide strand comprises 2′-F modified nucleotides at positions 2, 6, 12, 14, and 16 from the 5′ end. In some instances, the nucleotide analogue is located at position 7 from the 5′ end of the guide strand, and the guide strand comprises 2′-F modified nucleotides at positions 2, 8, 12, 14, and 16 from the 5′ end.

In some instances, the nucleotide analogue is located at position 8 from the 5′ end of the guide strand. In some instances, the nucleotide analogue is located at position 8 from the 5′ end of the guide strand, and the guide strand further comprises 2′-fluoro (2′-F) modified nucleotides at at least one of positions 2, 7, 12, 14, and 16 from the 5′ end. In some instances, the nucleotide analogue is located at position 8 from the 5′ end of the guide strand, and the guide strand further comprises 2′-fluoro (2′-F) modified nucleotides at at least two of positions 2, 7, 12, 14, and 16 from the 5′ end. In some instances, the nucleotide analogue is located at position 8 from the 5′ end of the guide strand, and the guide strand further comprises 2′-fluoro (2′-F) modified nucleotides at at least three of positions 2, 7, 12, 14, and 16 from the 5′ end. In some instances, the nucleotide analogue is located at position 8 from the 5′ end of the guide strand, and the guide strand further comprises 2′-fluoro (2′-F) modified nucleotides at positions 2, 7, 12, 14, and 16 from the 5′ end.

In some instances, the nucleotides of the guide strand comprises DNA or RNA. As described herein, in some instances, the DNA nucleotide comprises an unmodified DNA comprising: an unmodified adenine nucleotide (A), an unmodified guanine nucleotide (G), an unmodified thymine nucleotide (T), or an unmodified cytosine nucleotide (C). As described herein, in some instances, the RNA comprises an unmodified RNA comprising: an unmodified adenine nucleotide (A), an unmodified guanine nucleotide (G), an unmodified uracil nucleotide (U), or an unmodified cytosine nucleotide (C).

In some instances, the nucleotides of the guide strand comprises the DNA, RNA, nucleotide analogue, 2′-F modified nucleotide, or 2′-O-alkyl modified nucleotide. In some instances, the 2′-O-alkly modified nucleotide comprises 2′-O-methyl modified nucleotides. In some instances, the nucleotides of the guide strand that are not the nucleotide analogue or 2′-F modified nucleotide are selected from DNA nucleotide, RNA nucleotide, and 2′-O-alkly modified nucleotide. In some instances, the nucleotides of the guide strand that are not the nucleotide analogue or 2′-F modified nucleotide are 2′-O-methyl modified nucleotides.

In some instances, the guide strand comprises at least one, at least two, at least three, at least four, at least five, at least six, at least seven, or at least eight phosphorothioate modified internucleotide linkages. In some instances, the guide strand comprises at least two, at least three, or at least four phosphorothioate modified internucleotide linkages. In some instances, the guide strand comprises at most one, at most two, at most three, at most four, at most five, at most six, at most seven, or at most eight phosphorothioate modified internucleotide linkages. In some instances, the guide strand comprises one, two, three, four, five, six, seven, or eight phosphorothioate modified internucleotide linkages. In some instances, the guide strand comprises from 1 to 8, from 2 to 8, from 3 to 8, from 4 to 8, from 5 to 8, or from 6 to 8 phosphorothioate modified intemucleotide linkages. In some instances, the guide strand comprises from 1 to 4, from 2 to 4, or from 3 to 4 phosphorothioate modified intemucleotide linkages.

In some instances, the guide strand comprises one phosphorothioate modified intemucleotide linkages at the 5′ end and one phosphorothioate modified intemucleotide linkages at the 3′ end. In some instances, the guide strand comprises two phosphorothioate modified intemucleotide linkages at the 5′ end and two phosphorothioate modified intemucleotide linkages at the 3′ end. In some instances, the guide strand comprises three phosphorothioate modified intemucleotide linkages at the 5′ end and three phosphorothioate modified intemucleotide linkages at the 3′ end. In some instances, the guide strand comprises four phosphorothioate modified intemucleotide linkages at the 5′ end and four phosphorothioate modified intemucleotide linkages at the 3′ end.

In some instances, the guide strand comprises 2′-O-methyl modified nucleotide, 2′-F modified nucleotide, and/or nucleotide analogue described herein. In some instances, the guide strand comprises a nucleic acid sequence of 5′-mFmmmXFmmmmFmFmFmmmmmmm-3′, wherein m is 2′-O-methyl modified nucleotide, F is 2′-fluoro (2′-F) modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), thymidine-glycol nucleic acid (GNA)S-isomer (Tgn), 2′-O-methyl-2-thiouridine-3′-phosphate (u3), 2′-fluoro-2-thiouridine-3′-phosphate (U3f), and 2-amino-2′-O-methyladenosine-3′-phosphate (al). In some instances, the guide strand comprises a nucleic acid sequence of 5′-mFmmmXFmmmmFmFmFmmmmmmm-3′, wherein m is 2′-O-methyl modified nucleotide, F is 2′-fluoro (2′-F) modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), and thymidine-glycol nucleic acid (GNA)S-isomer (Tgn).

In some instances, the guide strand comprises a nucleic acid sequence of 5′-mFmmmmXmmmmFmFmFmmmmmmm-3′, wherein m is 2′-O-methyl modified nucleotide, F is 2′-F modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), thymidine-glycol nucleic acid (GNA)S-isomer (Tgn), 2′-O-methyl-2-thiouridine-3′-phosphate (u3), 2′-fluoro-2-thiouridine-3′-phosphate (U3f), and 2-amino-2′-O-methyladenosine-3′-phosphate (al). In some instances, the guide strand comprises a nucleic acid sequence of 5′-mFmmmmXmmmmFmFmFmmmmmmm-3′, wherein m is 2′-O-methyl modified nucleotide, F is 2′-F modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), and thymidine-glycol nucleic acid (GNA)S-isomer (Tgn).

In some instances, the guide strand comprises a nucleic acid sequence of 5′-mFmmmFXmmmmFmFmFmmmmmmm-3′, wherein m is 2′-O-methyl modified nucleotide, F is 2′-F modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), thymidine-glycol nucleic acid (GNA)S-isomer (Tgn), 2′-O-methyl-2-thiouridine-3′-phosphate (u3), 2′-fluoro-2-thiouridine-3′-phosphate (U3f), and 2-amino-2′-O-methyladenosine-3′-phosphate (al). In some instances, the guide strand comprises a nucleic acid sequence of 5′-mFmmmFXmmmmFmFmFmmmmmmm-3′, wherein m is 2′-O-methyl modified nucleotide, F is 2′-F modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), and thymidine-glycol nucleic acid (GNA)S-isomer (Tgn).

In some instances, the guide strand comprises a nucleic acid sequence of 5′-mFmmmmXFmmmFmFmFmmmmmmm-3′, wherein m is 2′-O-methyl modified nucleotide, F is 2′-F modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), thymidine-glycol nucleic acid (GNA)S-isomer (Tgn), 2′-O-methyl-2-thiouridine-3′-phosphate (u3), 2′-fluoro-2-thiouridine-3′-phosphate (U3f), and 2-amino-2′-O-methyladenosine-3′-phosphate (al). In some instances, the guide strand comprises a nucleic acid sequence of 5′-mFmmmmXFmmmFmFmFmmmmmmm-3′, wherein m is 2′-O-methyl modified nucleotide, F is 2′-F modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), and thymidine-glycol nucleic acid (GNA)S-isomer (Tgn).

In some instances, the guide strand comprises a nucleic acid sequence of 5′-mFmmmmFXmmmFmFmFmmmmmmm-3′, wherein m is 2′-O-methyl modified nucleotide, F is 2′-F modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), thymidine-glycol nucleic acid (GNA)S-isomer (Tgn), 2′-O-methyl-2-thiouridine-3′-phosphate (u3), 2′-fluoro-2-thiouridine-3′-phosphate (U3f), and 2-amino-2′-O-methyladenosine-3′-phosphate (al). In some instances, the guide strand comprises a nucleic acid sequence of 5′-mFmmmmFXmmmFmFmFmmmmmmm-3′, wherein m is 2′-O-methyl modified nucleotide, F is 2′-F modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), and thymidine-glycol nucleic acid (GNA)S-isomer (Tgn).

In some instances, the guide strand comprises 2′-O-methyl modified nucleotide, 2′-F modified nucleotide, nucleotide analogue described herein, or phophorothioate intemucleotide linkage. In some instances, the guide strand comprises one or more phosphorothioate modified intemucleotide linkages at the 5′ end and one or more phosphorothioate modified intemucleotide linkages at the 3′ end. In some instances, the guide strand comprises two phosphorothioate modified intemucleotide linkages at the 5′ end and two phosphorothioate modified intemucleotide linkages at the 3′ end. In some instances, the guide strand comprises a nucleic acid sequence of 5′-msFsmmmXFmmmmFmFmFmmmmmsmsm-3′, wherein m is 2′-O-methyl modified nucleotide, F is 2′-F modified nucleotide, s is phophorothioate intemucleotide linkage, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), thymidine-glycol nucleic acid (GNA)S-isomer (Tgn), 2′-O-methyl-2-thiouridine-3′-phosphate (u3), 2′-fluoro-2-thiouridine-3′-phosphate (U3f), and 2-amino-2′-O-methyladenosine-3′-phosphate (al).

In some instances, the guide strand comprises a nucleic acid sequence of 5′-msFsmmmXFmmmmFmFmFmmmmmsmsm-3′, wherein m is 2′-O-methyl modified nucleotide, F is 2′-F modified nucleotide, s is phophorothioate intemucleotide linkage, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), and thymidine-glycol nucleic acid (GNA)S-isomer (Tgn).

In some instances, the guide strand comprises a nucleic acid sequence of 5′-msFsmmmmXmmmmFmFmFmmmmmsmsm-3′, wherein m is 2′-O-methyl modified nucleotide, F is 2′-F modified nucleotide, s is phophorothioate intemucleotide linkage, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), thymidine-glycol nucleic acid (GNA)S-isomer (Tgn), 2′-O-methyl-2-thiouridine-3′-phosphate (u3), 2′-fluoro-2-thiouridine-3′-phosphate (U3), and 2-amino-2′-O-methyladenosine-3′-phosphate (al). In some instances, the guide strand comprises a nucleic acid sequence of 5′-msFsmmmmXmmmmFmFmFmmmmmsmsm-3′, wherein m is 2′-O-methyl modified nucleotide, F is 2′-F modified nucleotide, s is phophorothioate intemucleotide linkage, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), and thymidine-glycol nucleic acid (GNA)S-isomer (Tgn).

In some instances, the guide strand comprises a nucleic acid sequence of 5′-msFsmmmmFXmmmFmFmFmmmmmsmsm-3′, wherein m is 2′-O-methyl modified nucleotide, F is 2′-F modified nucleotide, s is phophorothioate intemucleotide linkage, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), thymidine-glycol nucleic acid (GNA)S-isomer (Tgn), 2′-O-methyl-2-thiouridine-3′-phosphate (u3), 2′-fluoro-2-thiouridine-3′-phosphate (U3), and 2-amino-2′-O-methyladenosine-3′-phosphate (al). In some instances, the guide strand comprises a nucleic acid sequence of 5′-msFsmmmmFXmmmFmFmFmmmmmsmsm-3′, wherein m is 2′-O-methyl modified nucleotide, F is 2′-F modified nucleotide, s is phophorothioate intemucleotide linkage, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), and thymidine-glycol nucleic acid (GNA)S-isomer (Tgn).

In some instances, the nucleotide analogue is located at at least one of positions 2-12 from the 5′ end of the guide strand, and the nucleotide analogue comprises 2′-O-methyl-2-thiouridine-3′-phosphate (u3) or 2′-fluoro-2-thiouridine-3′-phosphate (U3f). In some instances, the nucleotide analogue that is located at position 12 of the 5′ end of the guide strand, and the nucleotide analogue consists of 2′-fluoro-2-thiouridine-3′-phosphate (U3). In some instances, the nucleotide analogue is located at position 3 of the 5′ end of the guide strand, and the nucleotide analogue comprises 2′-O-methyl-2-thiouridine-3′-phosphate (u3). In some instances, the nucleotide analogue is located at position 3 of the 5′ end of the guide strand, and the nucleotide analogue consists of 2′-O-methyl-2-thiouridine-3′-phosphate (u3). In some instances, the guide strand comprises a nucleic acid sequence of 5′-mFmmmmFXmmmF′mFmFmmmmmmm-3′, where m is 2′-O-methyl modified nucleotide, F is 2′-F modified nucleotide, X is selected from acyclic L-threoninol nucleic acid-thymine-3′-phosphate (T-T), acyclic N-acetyl L-threoninol abasic nucleic acid-3′phosphate (T-NAc), 1′,2′-Dideoxyribose-3′-phosphate (dAB), and thymidine-glycol nucleic acid (GNA)S-isomer (Tgn), and F′ is 2′-fluoro-2-thiouridine-3′-phosphate (U3f). In some instances, the guide strand comprises a nucleic acid sequence of 5′-msFsmmmmFXmmmF′mFmFmmmmmsmsm-3′, where m is 2′-O-methyl modified nucleotide, F is 2′-F modified nucleotide, s is phophorothioate intemucleotide linkage, X is selected from acyclic L-threoninol nucleic acid-thymine-3′-phosphate (T-T), acyclic N-acetyl L-threoninol abasic nucleic acid-3′phosphate (T-NAc), 1′,2′-Dideoxyribose-3′-phosphate (dAB), and thymidine-glycol nucleic acid (GNA)S-isomer (Tgn), and F′ is 2′-fluoro-2-thiouridine-3′-phosphate (U3f). In some instances, the guide strand comprises a nucleic acid sequence of 5′-mFXmmmFmmmmFmFmFmmmmmmm-3′, where m is 2′-O-methyl modified nucleotide, F is 2′-F modified nucleotide, and X is 2′-O-methyl-2-thiouridine-3′-phosphate (u3). In some instances, the guide strand comprises a nucleic acid sequence of 5′-msFsXmmmFmmmmFmFmFmmmmmsmsm-3′, where m is 2′-O-methyl modified nucleotide, F is 2′-F modified nucleotide, s is phophorothioate intemucleotide linkage, and X is 2′-O-methyl-2-thiouridine-3′-phosphate (u3).

In some aspects, described herein is a specific modification motif of pattern for the double-stranded polynucleic acid molecule comprising a passenger strand and an guide strand. In some aspects, described herein is a specific modification pattern of the passenger strand.

In some instances, the passenger strand comprises one or more nucleotide analogue. In some instances, the passenger strand comprises one or more nucleotide analogue at locations opposite to the seed region of the guide strand. In some instances, the passenger strand comprises one or more nucleotide analogue at locations 12-22 from the 5′ end. In some instances, the passenger strand comprises one or more nucleotide analogue at locations 2-10 from the 3′ end. In some instances, the passenger strand comprises one or more 2-amino-2′-O-methyladenosine-3′-phosphate (al). In some instances, the passenger strand comprises 2-amino-2′-O-methyladenosine-3′-phosphate (al) at location 11, 12, 13, 14, or 15 from the 5′ end. In some instances, the passenger strand comprises 2-amino-2′-O-methyladenosine-3′-phosphate (al) at location 13 from the 5′ end. In some instances, the passenger strand comprises 2-amino-2′-O-methyladenosine-3′-phosphate (al) at location 14 from the 5′ end. In some instances, the passenger strand comprises 2-amino-2′-O-methyladenosine-3′-phosphate (al) at location 15 from the 5′ end.

In some instances, the passenger strand comprises at least one, at least two, at least three, at least four, at least five, at least six, at least seven, or at least eight 2′-fluoro (2′-F) modified nucleotides. In some instances, the passenger strand comprises at least two, at least three, or at least four 2′-F modified nucleotides. In some instances, the passenger strand comprises at most one, at most two, at most three, at most four, at most five, at most six, at most seven, or at most eight 2′-fluoro (2′-F) modified nucleotides. In some instances, the passenger strand comprises from one to eight, from two to eight, from three to eight, from four to eight, from five to eight, from six to eight, or from seven to eight 2′-fluoro (2′-F) modified nucleotides. In some instances, the passenger strand comprises one, two, three, four, five, six, seven, or eight 2′-fluoro (2′-F) modified nucleotides. In some instances, the passenger strand comprises one 2′-F modified nucleotides. In some instances, the passenger strand comprises two 2′-F modified nucleotides. In some instances, the passenger strand comprises three 2′-F modified nucleotides. In some instances, the passenger strand comprises four 2′-F modified nucleotides. In some instances, the passenger strand comprises five 2′-F modified nucleotides.

In some aspects, the passenger strand comprises a 2′-fluoro modified nucleotide at position 7 from the 5′ end. In some aspects, the passenger strand comprises a 2′-fluoro modified nucleotide at position 9 from the 5′ end. In some aspects, the passenger strand comprises 2′-fluoro modified nucleotides at position 11 from the 5′ end. In some aspects, the passenger strand comprises a 2′-fluoro modified nucleotide at at least one of positions 7, 9, and 11 from the 5′ end. In some aspects, the passenger strand comprises a 2′-fluoro modified nucleotide at positions 7, 9, 11, or combination thereof from the 5′ end. In some aspects, the passenger strand comprises a 2′-fluoro modified nucleotide at positions 7, 9, and 11 from the 5′ end.

In some instances, the nucleotides of the passenger strand comprises DNA nucleotide or RNA nucleotide. As described herein, in some instances, the DNA nucleotide comprises an unmodified DNA nucleotide comprising: an unmodified adenine nucleotide (A), an unmodified guanine nucleotide (G), an unmodified thymine nucleotide (T), or an unmodified cytosine nucleotide (C). As described herein, in some instances, the RNA nucleotide comprises an unmodified RNA nucleotide comprising: an unmodified adenine nucleotide (A), an unmodified guanine nucleotide (G), an unmodified uracil nucleotide (U), or an unmodified cytosine nucleotide (C).

In some instances, the nucleotides of the passenger strand comprises the DNA nucleotide, RNA nucleotide, nucleotide analogue, 2′-F modified nucleotide, or 2′-O-alkyl modified nucleotide. In some instances, the 2′-O-alkly modified nucleotide comprises 2′-O-methyl modified nucleotides. In some instances, the nucleotides of the passenger strand that are not the nucleotide analogue or 2′-F modified nucleotide are selected from 2′-O-alkyl modified nucleotide, 2′-alkoxy modified nucleotide, 2′-alkyl modified nucleotide, 2′-halo modified nucleotide, DNA nucleotide, RNA nucleotide, ENA, BNA, LNA, and UNA. In some instances, the nucleotides in the passenger strand that are not 2′-F modified nucleotide are 2′-O-methyl modified nucleotides.

In some instances, the passenger strand comprises at least one, at least two, at least three, at least four, at least five, at least six, at least seven, or at least eight phosphorothioate modified intemucleotide linkages. In some instances, the passenger strand comprises at least two, at least three, or at least four phosphorothioate modified intemucleotide linkages. In some instances, the passenger strand comprises at least one phosphorothioate modified intemucleotide linkage. In some instances, the passenger strand comprises at most one, at most two, at most three, at most four, at most five, at most six, at most seven, or at most eight phosphorothioate modified intemucleotide linkages. In some instances, the passenger strand comprises one, two, three, four, five, six, seven, or eight phosphorothioate modified intemucleotide linkages. In some instances, the passenger strand comprises from 1 to 8, from 2 to 8, from 3 to 8, from 4 to 8, from 5 to 8, or from 6 to 8 phosphorothioate modified intemucleotide linkages. In some instances, the passenger strand comprises from 1 to 4, from 2 to 4, or from 3 to 4 phosphorothioate modified intemucleotide linkages.

In some instances, the passenger strand comprises at least one, at least two, at least three, or at least four phosphorothioate modified intemucleotide linkage at the 5′ end. In some instances, the passenger strand comprises at least one phosphorothioate modified intemucleotide linkage at the 5′ end. In some instances, the passenger strand comprises at least one, at least two, at least three, or at least four phosphorothioate modified intemucleotide linkage at the 3′ end. In some instances, the passenger strand comprises at least one phosphorothioate modified intemucleotide linkage at the 3′ end.

In some instances, the passenger strand comprises one phosphorothioate modified intemucleotide linkage at the 5′ end. In some instances, the passenger strand comprises two phosphorothioate modified intemucleotide linkage at the 5′ end. In some instances, the passenger strand comprises three phosphorothioate modified intemucleotide linkage at the 5′ end. In some instances, the passenger strand comprises four phosphorothioate modified intemucleotide linkage at the 5′ end.

In some instances, the passenger strand comprises one phosphorothioate modified intemucleotide linkage at the 3′ end. In some instances, the passenger strand comprises two phosphorothioate modified intemucleotide linkage at the 3′ end. In some instances, the passenger strand comprises three phosphorothioate modified intemucleotide linkage at the 3′ end. In some instances, the passenger strand comprises four phosphorothioate modified intemucleotide linkage at the 3′ end.

In some instances, the passenger strand comprises one phosphorothioate modified intemucleotide linkages at the 5 end and one phosphorothioate modified intemucleotide linkages at the 3′ end. In some instances, the passenger strand comprises two phosphorothioate modified intemucleotide linkages at the 5′ end and two phosphorothioate modified intemucleotide linkages at the 3′ end. In some instances, the passenger strand comprises three phosphorothioate modified intemucleotide linkages at the 5′ end and three phosphorothioate modified intemucleotide linkages at the 3′ end. In some instances, the passenger strand comprises four phosphorothioate modified intemucleotide linkages at the 5′ end and four phosphorothioate modified intemucleotide linkages at the 3′ end.

In some aspects, described herein is a specific modification pattern for a double-stranded polynucleic nucleic acid molecule comprising a passenger strand and an guide strand. In some instances, the guide strand comprises a nucleic acid sequence of mFmmmXFmmmmFmFmFmmmmmmm, and the passenger strand comprises mmmmmmFmFmFmmmmmmmmmm, wherein m is 2′-O-methyl modified nucleotide, F is 2′-F modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), thymidine-glycol nucleic acid (GNA)S-isomer (Tgn), 2′-O-methyl-2-thiouridine-3′-phosphate (u3), 2′-fluoro-2-thiouridine-3′-phosphate (U3f), or 2-amino-2′-O-methyladenosine-3′-phosphate (al). In some instances, the guide strand comprises a nucleic acid sequence of 5′-mFmmmXFmmmmFmFmFmmmmmmm-3′, and the passenger strand comprises mmmmmmFmFmFmmmmmmmmmm, wherein m is 2′-O-methyl modified nucleotide, F is 2′-F modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), and thymidine-glycol nucleic acid (GNA)S-isomer (Tgn). In some instances, the guide strand comprises a nucleic acid sequence of 5′-mFmmmXFmmmmFmFmFmmmmmmm-3′, and the passenger strand comprises 5′-mmmmmmFmFmFmmX′mmmmmmm-3′, wherein m is 2′-O-methyl modified nucleotide, F is 2′-F modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), and thymidine-glycol nucleic acid (GNA)S-isomer (Tgn), and X′ is 2-amino-2′-O-methyladenosine-3′-phosphate (al). In some instances, the guide strand comprises a nucleic acid sequence of 5′-mFmmmXFmmmmFmFmFmmmmmmm-3′, and the passenger strand comprises 5′-mmmmmmFmFmFmmmX′mmmmmm-3′, wherein m is 2′-O-methyl modified nucleotide, F is 2′-F modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), and thymidine-glycol nucleic acid (GNA)S-isomer (Tgn), and X′ is 2-amino-2′-O-methyladenosine-3′-phosphate (al).

In some instances, the guide strand comprises a nucleic acid sequence of 5′-msFsmmmXFmmmmFmFmFmmmmmsmsm-3′, and the passenger strand comprises 5′-msmsmmmmFmFmFmmmmmmmmmm-3′, wherein m is 2′-O-methyl modified nucleotide, F is 2′-F modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), thymidine-glycol nucleic acid (GNA)S-isomer (Tgn), 2′-O-methyl-2-thiouridine-3′-phosphate (u3), 2′-fluoro-2-thiouridine-3′-phosphate (U3f), or 2-amino-2′-O-methyladenosine-3′-phosphate (al). In some instances, the guide strand comprises a nucleic acid sequence of 5′-msFsmmmXFmmmmFmFmFmmmmmsmsm-3′, and the passenger strand comprises 5′-msmsmmmmFmFmFmmmmmmmmmm-3′, wherein m is 2′-O-methyl modified nucleotide, F is 2′-F modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), and thymidine-glycol nucleic acid (GNA)S-isomer (Tgn). In some instances, the guide strand comprises a nucleic acid sequence of 5′-msFsmmmXFmmmmFmFmFmmmmmsmsm-3′, and the passenger strand comprises 5′-msmsmmmmFmFmFmmX′mmmmmmm-3′, wherein m is 2′-O-methyl modified nucleotide, F is 2′-F modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), and thymidine-glycol nucleic acid (GNA)S-isomer (Tgn), and X′ is 2-amino-2′-O-methyladenosine-3′-phosphate (al). In some instances, the guide strand comprises a nucleic acid sequence of 5′-msFsmmmXFmmmmFmFmFmmmmmsmsm-3′, and the passenger strand comprises 5′-msmsmmmmFmFmFmmmX′mmmmmm-3′, wherein m is 2′-O-methyl modified nucleotide, F is 2′-F modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), and thymidine-glycol nucleic acid (GNA)S-isomer (Tgn), and X′ is 2-amino-2′-O-methyladenosine-3′-phosphate (al).

In some instances, the guide strand comprises a nucleic acid sequence of 5′-mFmmmmXmmmmFmFmFmmmmmmm-3′, and the passenger strand comprises 5′-mmmmmmFmFmFmmmmmmmmmm-3′, wherein m is 2′-O-methyl modified nucleotide, F is 2′-F modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), thymidine-glycol nucleic acid (GNA)S-isomer (Tgn), 2′-O-methyl-2-thiouridine-3′-phosphate (u3), 2′-fluoro-2-thiouridine-3′-phosphate (U3f), or 2-amino-2′-O-methyladenosine-3′-phosphate (al). In some instances, the guide strand comprises a nucleic acid sequence of 5′-mFmmmmXmmmmFmFmFmmmmmmm-3′, and the passenger strand comprises 5′-mmmmmmFmFmFmmmmmmmmmm-3′, wherein m is 2′-O-methyl modified nucleotide, F is 2′-F modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), and thymidine-glycol nucleic acid (GNA)S-isomer (Tgn). In some instances, the guide strand comprises a nucleic acid sequence of 5′-mFmmmmXmmmmFmFmFmmmmmmm-3′, and the passenger strand comprises 5′-msmsmmmmFmFmFmmX′mmmmmmm-3′, wherein m is 2′-O-methyl modified nucleotide, F is 2′-F modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), and thymidine-glycol nucleic acid (GNA)S-isomer (Tgn), and X′ is 2-amino-2′-O-methyladenosine-3′-phosphate (al). In some instances, the guide strand comprises a nucleic acid sequence of 5′-mFmmmmXmmmmFmFmFmmmmmmm-3′, and the passenger strand comprises 5′-msmsmmmmFmFmFmmmX′mmmmmm-3′, wherein m is 2′-O-methyl modified nucleotide, F is 2′-F modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), and thymidine-glycol nucleic acid (GNA)S-isomer (Tgn), and X′ is 2-amino-2′-O-methyladenosine-3′-phosphate (al).

In some instances, the guide strand comprises a nucleic acid sequence of 5′-msFsmmmmXmmmmFmFmFmmmmmsmsm-3′, and the passenger strand comprises 5′-msmsmmmmFmFmFmmmmmmmmmm-3′, wherein m is 2′-O-methyl modified nucleotide, F is 2′-F modified nucleotide, s is phosphorothioate intemucleotide linkage, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), thymidine-glycol nucleic acid (GNA)S-isomer (Tgn), 2′-O-methyl-2-thiouridine-3′-phosphate (u3), 2′-fluoro-2-thiouridine-3′-phosphate (U3f), or 2-amino-2′-O-methyladenosine-3′-phosphate (al). In some instances, the guide strand comprises a nucleic acid sequence of 5′-msFsmmmmXmmmmFmFmFmmmmmsmsm-3′, and the passenger strand comprises 5′-msmsmmmmFmFmFmmmmmmmmmm-3′, wherein m is 2′-O-methyl modified nucleotide, F is 2′-F modified nucleotide, s is phosphorothioate internucleotide linkage, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), and thymidine-glycol nucleic acid (GNA)S-isomer (Tgn). In some instances, the guide strand comprises a nucleic acid sequence of 5′-msFsmmmmXmmmmFmFmFmmmmmsmsm-3′, and the passenger strand comprises 5′-msmsmmmmFmFmFmmX′mmmmmmm-3′, wherein m is 2′-O-methyl modified nucleotide, F is 2′-F modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), and thymidine-glycol nucleic acid (GNA)S-isomer (Tgn), and X′ is 2-amino-2′-O-methyladenosine-3′-phosphate (al). In some instances, the guide strand comprises a nucleic acid sequence of 5′-msFsmmmmXmmmmFmFmFmmmmmsmsm-3′, and the passenger strand comprises 5′-msmsmmmmFmFmFmmmX′mmmmmm-3′, wherein m is 2′-O-methyl modified nucleotide, F is 2′-F modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), and thymidine-glycol nucleic acid (GNA)S-isomer (Tgn), and X′ is 2-amino-2′-O-methyladenosine-3′-phosphate (al).

In some instances, the guide strand comprises a nucleic acid sequence of 5′-mFmmmmFXmmmFmFmFmmmmmmm-3′, and the passenger strand comprises 5′-mmmmmmFmFmFmmmmmmmmmm-3′, wherein m is 2′-O-methyl modified nucleotide, F is 2′-F modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), thymidine-glycol nucleic acid (GNA)S-isomer (Tgn), 2′-O-methyl-2-thiouridine-3′-phosphate (u3), 2′-fluoro-2-thiouridine-3′-phosphate (U3f), or 2-amino-2′-O-methyladenosine-3′-phosphate (al). In some instances, the guide strand comprises a nucleic acid sequence of 5′-mFmmmmFXmmmFmFmFmmmmmmm-3′, and the passenger strand comprises 5′-mmmmmmFmFmFmmmmmmmmmm-3′, wherein m is 2′-O-methyl modified nucleotide, F is 2′-F modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), and thymidine-glycol nucleic acid (GNA)S-isomer (Tgn). In some instances, the guide strand comprises a nucleic acid sequence of 5′-mFmmmmFXmmmFmFmFmmmmmmm-3′, and the passenger strand comprises 5′-msmsmmmmFmFmFmmX′mmmmmmm-3′, wherein m is 2′-O-methyl modified nucleotide, F is 2′-F modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), and thymidine-glycol nucleic acid (GNA)S-isomer (Tgn), and X′ is 2-amino-2′-O-methyladenosine-3′-phosphate (al). In some instances, the guide strand comprises a nucleic acid sequence of 5′-mFmmmmFXmmmFmFmFmmmmmmm-3′, and the passenger strand comprises 5′-msmsmmmmFmFmFmmmX′mmmmmm-3′, wherein m is 2′-O-methyl modified nucleotide, F is 2′-F modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), and thymidine-glycol nucleic acid (GNA)S-isomer (Tgn), and X′ is 2-amino-2′-O-methyladenosine-3′-phosphate (al).

In some instances, the guide strand comprises a nucleic acid sequence of 5′-msFsmmmmFXmmmFmFmFmmmmmsmsm-3′, and the passenger strand comprises 5′-msmsmmmmFmFmFmmmmmmmmmm-3′, wherein m is 2′-O-methyl modified nucleotide, F is 2′-F modified nucleotide, s is phosphorothioate internucleotide linkage, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), thymidine-glycol nucleic acid (GNA)S-isomer (Tgn), 2′-O-methyl-2-thiouridine-3′-phosphate (u3), 2′-fluoro-2-thiouridine-3′-phosphate (U3f), or 2-amino-2′-O-methyladenosine-3′-phosphate (al). In some instances, the guide strand comprises a nucleic acid sequence of 5′-msFsmmmmFXmmmFmFmFmmmmmsmsm-3′, and the passenger strand comprises 5′-msmsmmmmFmFmFmmmmmmmmmm-3′, wherein m is 2′-O-methyl modified nucleotide, F is 2′-F modified nucleotide, s is phosphorothioate internucleotide linkage, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), and thymidine-glycol nucleic acid (GNA)S-isomer (Tgn). In some instances, the guide strand comprises a nucleic acid sequence of 5′-msFsmmmmFXmmmFmFmFmmmmmsmsm-3′, and the passenger strand comprises 5′-msmsmmmmFmFmFmmX′mmmmmmm-3′, wherein m is 2′-O-methyl modified nucleotide, F is 2′-F modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), and thymidine-glycol nucleic acid (GNA)S-isomer (Tgn), and X′ is 2-amino-2′-O-methyladenosine-3′-phosphate (al). In some instances, the guide strand comprises a nucleic acid sequence of 5′-msFsmmmmFXmmmFmFmFmmmmmsmsm-3′, and the passenger strand comprises 5′-msmsmmmmFmFmFmmmX′mmmmmm-3′, wherein m is 2′-O-methyl modified nucleotide, F is 2′-F modified nucleotide, and X is selected from acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), 1′,2′-Dideoxyribose-3′-phosphate (dAB), and thymidine-glycol nucleic acid (GNA)S-isomer (Tgn), and X′ is 2-amino-2′-O-methyladenosine-3′-phosphate (al).

### Conjugation

### Targeting Moiety

In certain aspects, the polynucleic acid molecule described herein is coupled or conjugated with one or more targeting moieties to form a polynucleotide-targeting moiety conjugate molecule. In some instances, a targeting moiety is selected based on its ability to target the conjugate molecule described herein to a desired cell population, tissue, or an organ selectively or preferably. In some instances, the targeting moiety targets the cell, tissue, or an organ that expresses the corresponding binding partner (e.g., either the corresponding receptor or ligand) of the targeting moiety. For example, the polynucleic acid molecule conjugated with N-acetyl galactosamine (GalNAc) can target hepatocytes expressing asialoglycoprotein (ASGP-R). A targeting moiety (i.e., an intracellular targeting moiety) that targets a desired site within the cell (e.g., endoplasmic reticulum, Golgi apparatus, nucleus, or mitochondria) may be included in the hybridized polynucleotide constructs disclosed herein. Non-limiting examples of the intracellular targeting moieties are provided in WO 2015/069932 and in WO 2015/188197; the disclosure of the intracellular targeting moieties in WO 2015/069932 and in WO 2015/188197 is incorporated herein by reference.

The polynucleic acid molecule described herein, thus, may include one or more targeting moieties selected from the group consisting of intracellular targeting moieties, extracellular targeting moieties, and combinations thereof. Thus, the inclusion of one or more targeting moieties (e.g., extracellular targeting moieties including targeting moieties independently selected from the group consisting of folate, mannose, N-acetyl galactosamine, and prostate specific membrane antigen) and one or more intracellular targeting moiety (e.g., a moiety targeting endoplasmic reticulum, Golgi apparatus, nucleus, or mitochondria) in the polynucleic acid molecule described herein can facilitate the delivery of the polynucleotides to a specific site within the specific cell population. In some aspects, the targeting moiety contains one or more mannose carbohydrates. Mannose targets the mannose receptor, which is a 175 KDa membrane-associated receptor that is expressed on sinusoidal liver cells and antigen presenting cells (e.g., macrophages and dendritic cells). It is a highly effective endocytotic/recycling receptor that binds and internalizes mannosylated pathogens and proteins (Lennartz et. al. J. Biol. Chem. 262:9942-9944,1987; Taylor et. al. J. Biol. Chem. 265:12156-62, 1990).

Some of the targeting moieties are described herein. In some aspects, the targeting moiety contains or specifically binds to a protein selected from the group including insulin, insulin-like growth factor receptor 1 (IGF1R), IGF2R, insulin-like growth factor (IGF; e.g., IGF 1 or 2), mesenchymal epithelial transition factor receptor (c-met; also known as hepatocyte growth factor receptor (HGFR)), hepatocyte growth factor (HGF), epidermal growth factor receptor (EGFR), epidermal growth factor (EGF), heregulin, fibroblast growth factor receptor (FGFR), platelet-derived growth factor receptor (PDGFR), platelet-derived growth factor (PDGF), vascular endothelial growth factor receptor (VEGFR), vascular endothelial growth factor (VEGF), tumor necrosis factor receptor (TNFR), tumor necrosis factor alpha (TNF-α), TNF-P, folate receptor (FOLR), folate, transferrin, transferrin receptor (TfR), mesothelin, Fc receptor, c-kit receptor, c-kit, an integrin (e.g., an α4 integrin or a β-1 integrin), P-selectin, sphingosine-1-phosphate receptor-1 (S1PR), hyaluronate receptor, leukocyte function antigen-1 (LFA-1), CD4, CD11, CD18, CD20, CD25, CD27, CD52, CD70, CD80, CD85, CD95 (Fas receptor), CD106 (vascular cell adhesion molecule 1 (VCAM1), CD166 (activated leukocyte cell adhesion molecule (ALCAM)), CD178 (Fas ligand), CD253 (TNF-related apoptosis-inducing ligand (TRAIL)), ICOS ligand, CCR2, CXCR3, CCR5, CXCL12 (stromal cell-derived factor 1 (SDF-1)), interleukin 1 (IL-1), IL-Ira, IL-2, IL-3, IL-4, IL-6, IL-7, IL-8, CTLA-4, MART-1, gp100, MAGE-1, ephrin (Eph) receptor, mucosal addressin cell adhesion molecule 1 (MAdCAM-1), carcinoembryonic antigen (CEA), LewisY, MUC-1, epithelial cell adhesion molecule (EpCAM), cancer antigen 125 (CA125), prostate specific membrane antigen (PSMA), TAG-72 antigen, and fragments thereof. In further aspects, the targeting moiety contains erythroblastic leukemia viral oncogene homolog (ErbB) receptor (e.g., ErbB1 receptor; ErbB2 receptor; ErbB3 receptor; and ErbB4 receptor). In some aspects, the targeting moiety contains one or more (e.g., from 1 to 6)N-acetyl galactosamines (GalNAc). In certain aspects, the targeting moiety contains one or more (e.g., from 1 to 6) mannoses. In other aspects, the targeting moiety contains a folate ligand. The folate ligand has the structure:

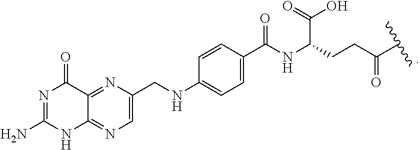 [SMILES: *C(=O)CC[C@H](NC(=O)c1ccc(NCc2cnc3[nH]c(N)nc(=O)c3n2)cc1)C(=O)O]

Certain targeting moieties may include bombesin, gastrin, gastrin-releasing peptide, tumor growth factors (TGF) (e.g., TGF-α or TGF-β), or vaccinia virus growth factor (VVGF). Non-peptidyl targeting moieties can also be used in the targeting moieties and may include, for example, steroids, carbohydrates, vitamins, and lectins. Some targeting moieties may include a polypeptide, such as somatostatin or somatostatin analog (e.g., octreotide or lanreotide), bombesin, or an antibody or antigen-binding fragment thereof. Antibodies may be of any recognized class or subclass, e.g., IgG, IgA, IgM, IgD, or IgE. Typical are those antibodies which fall within the IgG class. The antibodies can be derived from any species according techniques known in the art. Typically, however, the antibody is of human, murine, or rabbit origin. In addition, the antibody may be polyclonal or monoclonal, but is typically monoclonal.

Human or chimeric (e.g., humanized) antibodies may be used in targeting moieties. Targeting moieties may include an antigen-binding fragment of an antibody. Such antibody fragments may include, for example, the Fab′, F(ab′)2, Fv, or Fab fragments, single domain antibody, ScFv, or other antigen-binding fragments. Fc fragments may also be employed in targeting moieties. Such antibody fragments can be prepared, for example, by proteolytic enzyme digestion, for example, by pepsin or papain digestion, reductive alkylation, or recombinant techniques. The materials and methods for preparing antibody fragments are well-known to those skilled in the art. See, e.g., Parham, J. Immunology, 131:2895, 1983; Lamoyi et al., J.

### Immunological Methods, 56:235, 1983.

Other peptides for use as a targeting auxiliary moiety in polynucleic acid molecule described herein can be selected from KiSS peptides and analogs, urotensin II peptides and analogs, GnRH I and II peptides and analogs, depreotide, vapreotide, vasoactive intestinal peptide (VIP), cholecystokinin (CCK), RGD-containing peptides, melanocyte-stimulating hormone (MSH) peptide, neurotensin, calcitonin, glutathione, YIGSR (leukocyte-avid peptides, e.g., P483H, which contains the heparin-binding region of platelet factor-4 (PF-4) and a lysine-rich sequence), atrial natriuretic peptide (ANP), β-amyloid peptides, delta-opioid antagonists (such as ITIPP(psi)), annexin-V, endothelin, leukotriene B4 (LTB4), chemotactic peptides (e.g., N-formyl-methionyl-leucyl-phenylalanine-lysine (fMLFK), GP IIb/IIIa receptor antagonists (e.g., DMP444), human neutrophil elastase inhibitor (EPI-HNE-2 and EPI-HNE-4), plasmin inhibitor, antimicrobial peptides, apticide (P280 and P274), thrombospondin receptor (including analogs such as TP-1300), bitistatin, pituitary adenylyl cyclase type I receptor (PAC1), fibrin α-chain, peptides derived from phage display libraries, and conservative substitutions thereof.

One or more (e.g., from 1 to 6) targeting moieties can be linked to MOIETY or to X2 in formula (V′, V″, or V″′) through -LinkA-.

In some aspects, the targeting moiety includes one or more (e.g., from 1 to 6 or from 1 to 3) asialoglycoprotein receptor ligands (e.g., GalNAc). In some aspects, an asialoglycoprotein receptor ligand (e.g., GalNAc) is attached to -LinkA- through an anomeric carbon (e.g., where the anomeric carbon is the carbon atom in an acetal or a hemiaminal). In some aspects, an asialoglycoprotein receptor ligand (e.g., GalNAc) comprises an anomeric carbon bonded to trivalent, tetravalent linker, pentavalent, or hexavalent linker, wherein the anomeric carbon is part of a hemiaminal group. An asialoglycoprotein receptor ligand (e.g., GalNAc) attached to a linker through a hemiaminal may produce a hybridized polynucleotide construct having superior efficacy in gene silencing as compared to hybridized polynucleotide constructs having the asialoglycoprotein receptor ligand (e.g., GalNAc) attached to a linker through an acetal.

In some aspects, the linker and three asialoglycoprotein receptor targeting moieties, each of which comprises GalNAc, are as shown in Formula (V). In some instances, the conjugate described herein only comprises one asialoglycoprotein receptor targeting moiety, so the conjugate comprises a structure of Formula (V) with any two of the targeting moieties removed. In some instances, the conjugate described herein only comprises two asialoglycoprotein receptor targeting moieties, so the conjugate described herein comprises a structure of Formula (V) with any one of the targeting moieties removed.

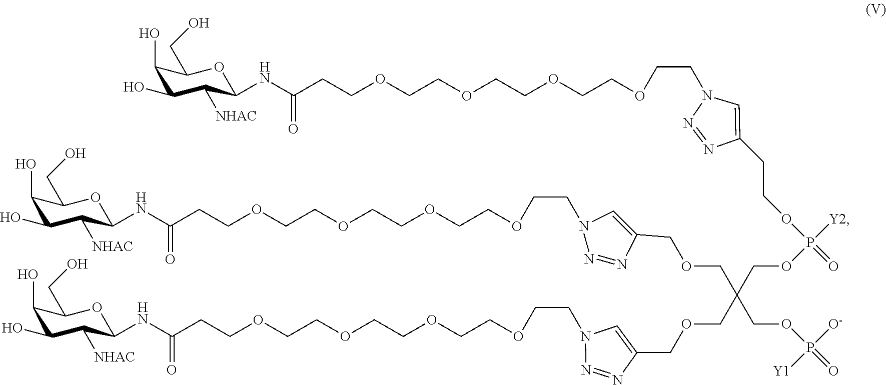

wherein one of Y1 and Y2 is nucleotide, or wherein both Y1 and Y2 are nucleotides and Y1 and Y2 are consecutive or neighboring nucleotides from the polynucleic acid molecule described herein.

In some aspects, the linker and the targeting moieties described herein are conjugated to 3′ end of the passenger strand (e.g., as shown in Formula (V′)). In some aspects, the linker and the targeting moieties described herein are conjugated to 5′ end of the passenger strand (e.g., as shown in Formula (V″) or (V″′)). In some aspects, the linker and the targeting moieties described herein are conjugated to 3′ end of the guide strand(e.g., as shown in Formula (V′)). In some aspects, the linker and the targeting moieties described herein are conjugated to 5′ end of the guide strand (e.g., as shown in Formula (V″) or (V″′)).

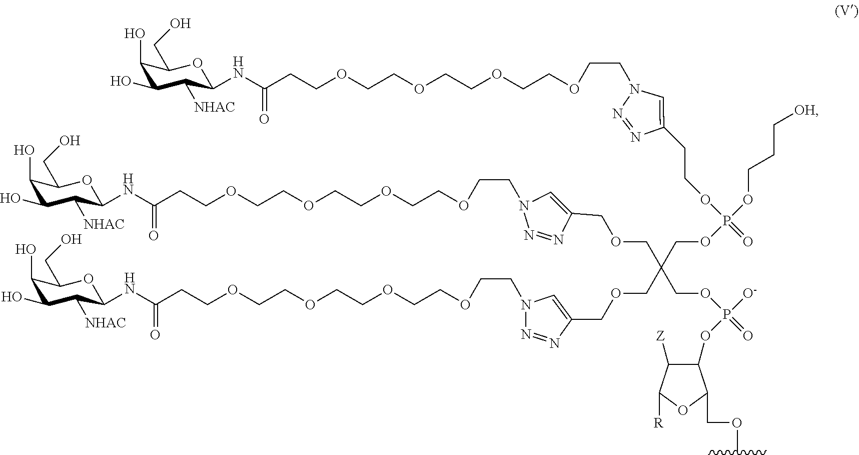

wherein Z in formula (V′) corresponds to one of the sugar modifications described herein (e.g., —H, —OH, —O-Methyl, —F, or —O-methoxyethyl), and R in formula (V′) is adenine, uracil, guanine, cytosine, thymine, abasic, or others.

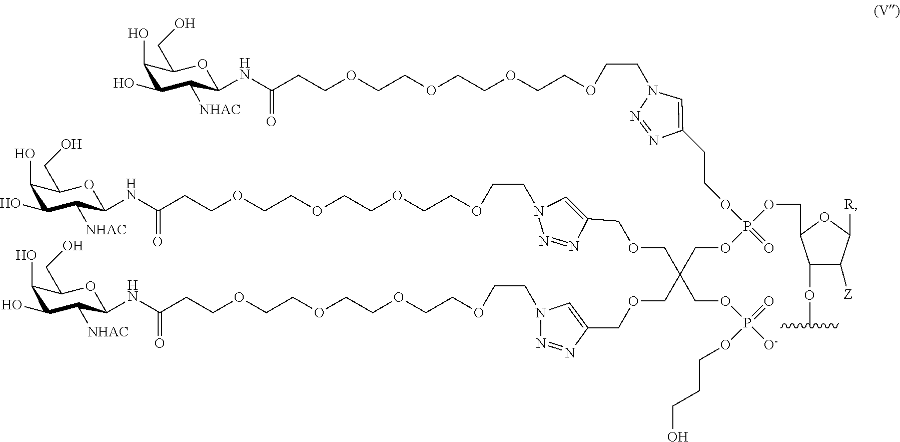

wherein Z in formula (V″) is a moiety that corresponds to one of the sugar modifications described herein (e.g., —H, —OH, —O-Methyl, —F, or —O-methoxyethyl) and R in formula (V″) is adenine, uracil, guanine, cytosine, thymine, abasic, or others.

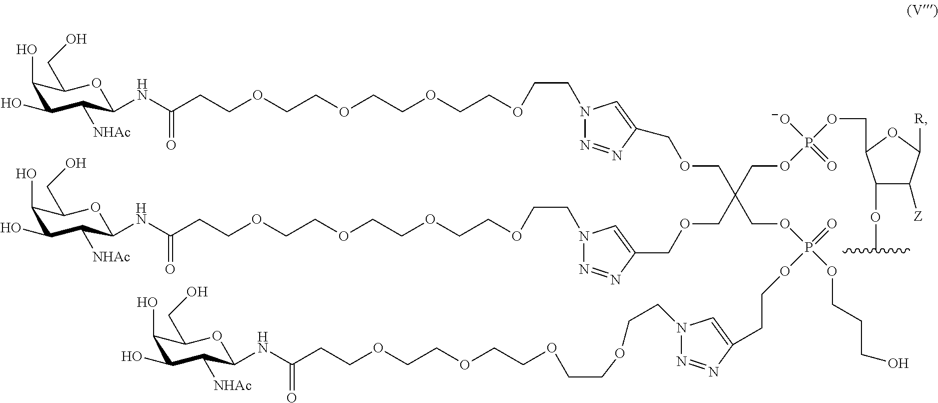

wherein Z in formula (V″′) is a moiety that corresponds to one of the sugar modifications described herein (e.g., —H, —OH, —O-Methyl, —F, or —O-methoxyethyl) and R in formula (V″′) is adenine, uracil, guanine, cytosine, thymine, abasic, or others.

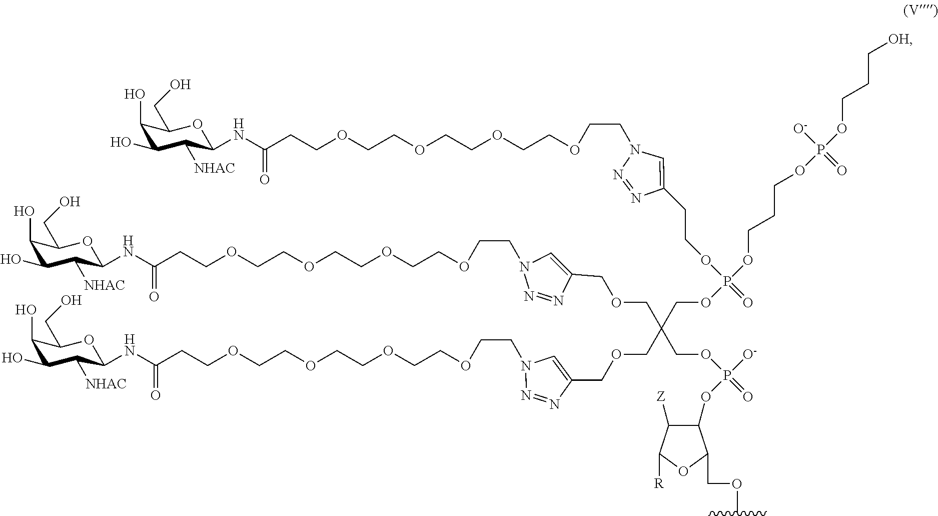

wherein Z in formula (V″″) is a moiety that corresponds to one of the sugar modifications described herein (e.g., —H, —OH, —O-Methyl, —F, or —O-methoxyethyl) and R in formula (V″″) is adenine, uracil, guanine, cytosine, thymine, abasic, or others.

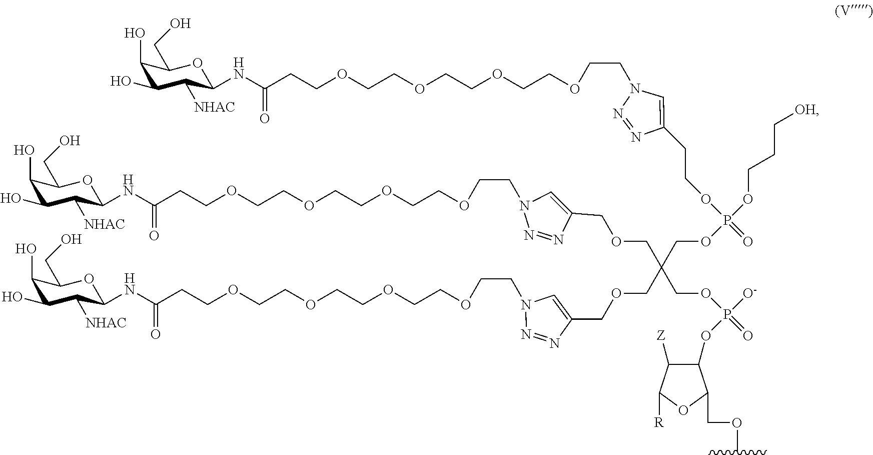

wherein Z in formula (V″″′) is a moiety that corresponds to one of the sugar modifications described herein (e.g., —H, —OH, —O-Methyl, —F, or —O-methoxyethyl) and R in formula (V″″′) is adenine, uracil, guanine, cytosine, thymine abasic or others.

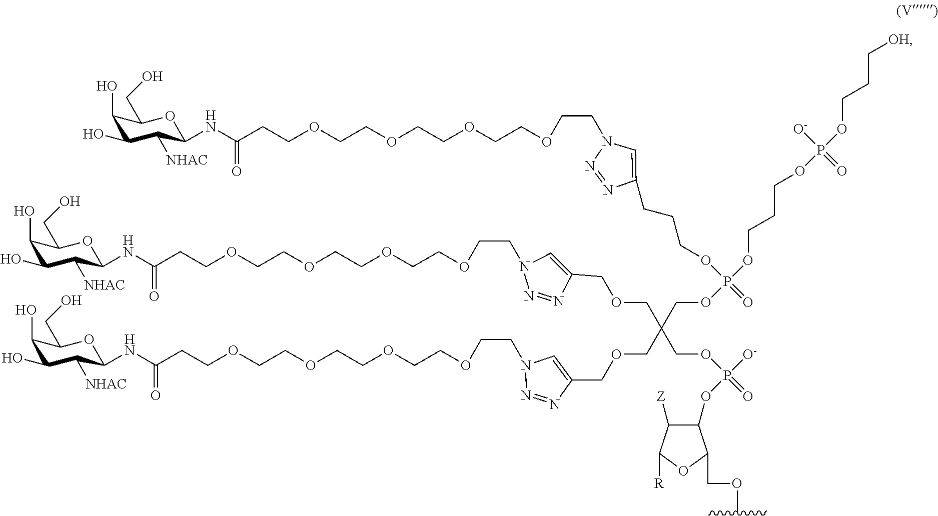

wherein Z in formula (V″″″) is a moiety that corresponds to one of the sugar modifications described herein (e.g., —H, —OH, —O-Methyl, —F, or —O-methoxyethyl) and R in formula (V″″″) is adenine, uracil, guanine, cytosine, thymine, abasic, or others.

In some instances, the 3′ end of passenger strand (sense strand) from Table 1, Table 2, Table 3, or Table 4 is conjugated with X2-GalNAc (see Formula (V), (V′), (V″), (V″′), (V″″), (V″″′), or (V″″″)). In some instances, the 5′ end of passenger strand (sense strand) from Table 1, Table 2, Table 3, or Table 4 is conjugated with X2-GalNAc (see Formula (V), (V′), (V″), (V″′), (V″″), (V″″′), or (V″″″)). In some instances, a nucleic acid within passenger strand (sense strand) (not at the 5′ or 3′ end) from Table 1, Table 2, Table 3, or Table 4 is conjugated with X2-GalNAc (see Formula (V), (V′), (V″), (V″′), (V″″), (V″′″), or (V″″″)). In some instances, the 3′ end of guide strand (antisense strand) from Table 1, Table 2, Table 3, or Table 4 is conjugated with X2-GalNAc (see Formula (V), (V′), (V″), (V″′), (V″″), (V″″′), or (V″″″)). In some instances, the 5′ end of guide strand (antisense strand) from Table 1, Table 2, Table 3, or Table 4 is conjugated with X2-GalNAc (see Formula (V), (V′), (V″), (V″′), (V″″), (V″″′), or (V″″″)). In some instances, a nucleic acid within guide strand (antisense strand) (not at the 5′ or 3′ end) from Table 1, Table 2, Table 3, or Table 4 is conjugated with X2-GalNAc (see Formula (V), (V′), (V″), (V″′), (V″″), (V″′″), or (V″″″)).

One or more endosomal escape moieties (e.g., from 1 to 6 or from 1 to 3) can be attached to a polynucleotide construct or a hybridized polynucleotide construct disclosed herein as an auxiliary moiety. Exemplary endosomal escape moieties include chemotherapeutics (e.g., quinolones such as chloroquine); fusogenic lipids (e.g., dioleoylphosphatidyl-ethanolamine (DOPE)); and polymers such as polyethylenimine (PEI); poly(beta-amino ester)s; polypeptides, such as polyarginines (e.g., octaarginine) and polylysines (e.g., octalysine); proton sponges, viral capsids, and peptide transduction domains as described herein. For example, fusogenic peptides can be derived from the M2 protein of influenza A viruses; peptide analogs of the influenza virus hemagglutinin; the HEF protein of the influenza C virus; the transmembrane glycoprotein of filoviruses; the transmembrane glycoprotein of the rabies virus; the transmembrane glycoprotein (G) of the vesicular stomatitis virus; the fusion protein of the Sendai virus; the transmembrane glycoprotein of the Semliki forest virus; the fusion protein of the human respiratory syncytial virus (RSV); the fusion protein of the measles virus; the fusion protein of the Newcastle disease virus; the fusion protein of the visna virus; the fusion protein of murine leukemia virus; the fusion protein of the HTL virus; and the fusion protein of the simian immunodeficiency virus (SIV). Other moieties that can be employed to facilitate endosomal escape are described in Dominska et al., Journal of Cell Science, 123(8):1183-1189, 2010. Specific examples of endosomal escape moieties including moieties suitable for conjugation to the hybridized polynucleotide constructs disclosed herein are provided, e.g., in WO 2015/188197; the disclosure of these endosomal escape moieties is incorporated by reference herein.

One or more endosomal escape moieties (e.g., from 1 to 6 or from 1 to 3) can be attached to a MOIETY or X2 in formula (V′, V″, V″′, V″″, V″″′, or V″″″) through -LinkA-, as described herein.

One or more cell penetrating peptides (CPP) (e.g., from 1 to 6 or from 1 to 3) can be attached to a polynucleotide construct or a hybridized polynucleotide construct disclosed herein as an auxiliary moiety. The CPP can be linked to the hybridized polynucleotide bioreversibly through a disulfide linkage, as disclosed herein. Thus, upon delivery to a cell, the CPP can be cleaved intracellularly, e.g., by an intracellular enzyme (e.g., protein disulfide isomerase, thioredoxin, or a thioesterase) and thereby release the polynucleotide.

CPPs are known in the art (e.g., TAT or Arg8) (Snyder and Dowdy, 2005, Expert Opin. Drug Deliv. 2, 43-51). Specific examples of CPPs including moieties suitable for conjugation to the hybridized polynucleotide constructs disclosed herein are provided, e.g., in WO 2015/188197; the disclosure of these CPPs is incorporated by reference herein.

CPPs are positively charged peptides that are capable of facilitating the delivery of biological cargo to a cell. It is believed that the cationic charge of the CPPs is essential for their function. Moreover, the transduction of these proteins does not appear to be affected by cell type, and these proteins can efficiently transduce nearly all cells in culture with no apparent toxicity (Nagahara et al., Nat. Med. 4:1449-52, 1998). In addition to full-length proteins, CPPs have also been used successfully to induce the intracellular uptake of DNA (Abu-Amer, supra), antisense polynucleotides (Astriab-Fisher et al., Pharm. Res, 19:744-54, 2002), small molecules (Polyakov et al., Bioconjug. Chem. 11:762-71, 2000) and even inorganic 40 nm iron particles (Dodd et al., J. Immunol. Methods 256:89-105, 2001; Wunderbaldinger et al., Bioconjug. Chem. 13:264-8, 2002; Lewin et al., Nat. Biotechnol. 18:410-4, 2000; Josephson et al., Bioconjug. Chem. 10:186-91, 1999) suggesting that there is considerable flexibility in particle size in this process.

In one embodiment, a CPP useful in the methods and compositions as described herein includes a peptide featuring substantial alpha-helicity. It has been discovered that transfection is optimized when the CPP exhibits significant alpha-helicity. In another embodiment, the CPP includes a sequence containing basic amino acid residues that are substantially aligned along at least one face of the peptide. A CPP described herein may be a naturally occurring peptide or a synthetic peptide.

One or more cell penetrating peptides (e.g., from 1 to 6 or from 1 to 3) can be attached to a MOIETY or X2 in formula (V′, V″, V″′, V″″, V″″′, or V″″″) through -LinkA-, as described herein.

The polynucleotide constructs and the hybridized polynucleotide constructs disclosed herein can also include covalently attached neutral polymer-based auxiliary moieties. Neutral polymers include poly(C1-6 alkylene oxide), e.g., poly(ethylene glycol) and poly(propylene glycol) and copolymers thereof, e.g., di- and triblock copolymers. Other examples of polymers include esterified poly(acrylic acid), esterified poly(glutamic acid), esterified poly(aspartic acid), poly(vinyl alcohol), poly(ethylene-co-vinyl alcohol), poly(N-vinyl pyrrolidone), poly(ethyloxazoline), poly(alkylacrylates), poly(acrylamide), poly(N-alkylacrylamides), poly(N-acryloylmorpholine), poly(lactic acid), poly(glycolic acid), poly(dioxanone), poly(caprolactone), styrene-maleic acid anhydride copolymer, poly(L-lactide-co-glycolide) copolymer, divinyl ether-maleic anhydride copolymer, N-(2-hydroxypropyl)methacrylamide copolymer (HMPA), polyurethane, N-isopropylacrylamide polymers, and poly(N,N-dialkylacrylamides). Exemplary polymer auxiliary moieties may have molecular weights of less than 100, 300, 500, 1000, or 5000 Da (e.g., greater than 100 Da). Other polymers are known in the art.

One or more polymers (e.g., from 1 to 6 or from 1 to 3) can be attached to a MOIETY or X2 in formula (V′, V″, V″′, V″″, V″″′, or V″″″) through -LinkA-, as described herein.

### Conjugation Linkers

In some aspects, the polynucleic acid molecules described herein comprises a passenger strand or guide strand bonded to at least one group of formula (I)

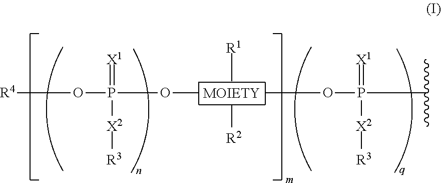

or a salt thereof, or a stereoisomer thereof,
where
each X¹ is independently O or S;
each X² is independently O, S, NH, or a bond;
MOIETY is optionally substituted C₂₋₁₀ alkane-tetrayl or a group -M¹-M²-M³-, wherein each M¹ and each M³ is independently absent or optionally substituted C₁₋₆ alkylene, and M² is optionally substituted C₃₋₉ heterocycle-tetrayl, optionally substituted C₆₋₁₀ arene-tetrayl, or optionally substituted C₃₋₈ cycloalkane-tetrayl;
each R¹ and each R² is independently H, optionally substituted C₁₋₁₆ alkyl, optionally substituted C₂₋₁₆ heteroalkyl, a conjugation moiety, or -LinkA(-T)ₚ, provided that at least one R¹ or at least one R² is a conjugation moiety or -LinkA(-T)ₚ;
each R³ is independently H, optionally substituted C₁₋₁₆ alkyl, optionally substituted C₂₋₁₆ heteroalkyl, optionally substituted C₂₋₁₆ alkenyl, optionally substituted C₂₋₁₆ alkynyl, optionally substituted (C₁₋₉ heterocyclyl)-C₁₋₆-alkyl, optionally substituted (C₆₋₁₀ aryl)-C₁₋₆-alkyl, optionally substituted (C₃₋₈ cycloalkyl)-C₁₋₆-alkyl, a conjugation moiety, or -LinkA(-T)ₚ;
R⁴ is H, optionally substituted C₁₋₆ alkyl, -LinkA(-T)ₚ, or -Sol;
each LinkA is independently a multivalent linker (e.g., including —C(O)—N(H)— (e.g., at least one multivalent linker including —C(O)—N(H)— bonded to T));
each T is independently an auxiliary moiety;
Sol is solid support;
m is an integer from 1 to 6;
each n is independently 0 or 1;
each p is independently an integer from 1 to 6; and q is an integer from 0 to 3.

The at least one group of formula (I) may be bonded to a 5′ end, 3′ end, internucleoside phosphate, internucleoside phosphorothioate, or internucleoside phosphorodithioate of the polynucleotide. When the at least one group of formula (I) is bonded to the internucleoside phosphate, internucleoside phosphorothioate, or internucleoside phosphorodithioate, q is 0. The polynucleotide construct contains no more than one Sol.

Group -LinkA- can include from 0 to 3 multivalent monomers (e.g., optionally substituted C1-6 alkane-triyl, optionally substituted C1-6 alkane-tetrayl, or trivalent nitrogen atom) and one or more divalent monomers (e.g., from 1 to 40), where each divalent monomer is independently optionally substituted C1-6 alkylene; optionally substituted C2-6 alkenylene; optionally substituted C2-6 alkynylene; optionally substituted C3-8 cycloalkylene; optionally substituted C3-8 cycloalkenylene; optionally substituted C6-14 arylene; optionally substituted C1-9 heteroarylene having 1 to 4 heteroatoms selected from N, O, and S; optionally substituted C1-9 heterocyclylene having 1 to 4 heteroatoms selected from N, O, and S; imino; optionally substituted N; O; or S(O)m, wherein m is 0, 1, or 2. In some aspects, each monomer is independently optionally substituted C1-6 alkylene; optionally substituted C3-8 cycloalkylene; optionally substituted C3-8 cycloalkenylene; optionally substituted C6-14 arylene; optionally substituted C1-9 heteroarylene having 1 to 4 heteroatoms selected from N, O, and S; optionally substituted C1-9 heterocyclylene having 1 to 4 heteroatoms selected from N, O, and S; imino; optionally substituted N; O; or S(O)m, where m is 0, 1, or 2 (e.g., m is 2). In certain aspects, each monomer is independently optionally substituted C1-6 alkylene; optionally substituted C3-8 cycloalkylene; optionally substituted C3-8 cycloalkenylene; optionally substituted C6-14 arylene; optionally substituted C1-9 heteroarylene having 1 to 4 heteroatoms selected from N, O, and S; optionally substituted C1-9 heterocyclylene having 1 to 4 heteroatoms selected from N, O, and S; optionally substituted N; O; or S(O)m, where m is 0, 1, or 2 (e.g., m is 2). The non-bioreversible linker connecting the auxiliary moiety to the conjugating moiety or to the reaction product thereof can include from 2 to 500 (e.g., from 2 to 300 or from 2 to 200) of such monomers. Group -LinkA- may include a poly(alkylene oxide) (e.g., polyethylene oxide, polypropylene oxide, poly(trimethylene oxide), polybutylene oxide, poly(tetramethylene oxide), and diblock or triblock co-polymers thereof). In some aspects, the non-bioreversible linker includes polyethylene oxide (e.g., poly(ethylene oxide) having a molecular weight of less than 1 kDa).

Group -LinkA(-T)p in formula (I) may be prepared by a process described in the sections below. In some instances, -LinkA(-T)p is of formula (II):

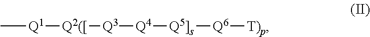

where
each s is independently an integer from 0 to 20 (e.g., from 0 to 10), where the repeating units are the same or different;
Q¹ is a conjugation linker (e.g., [-Q³-Q⁴-Q⁵]ₛ-Q^{C}-, where Q^{C} is optionally substituted C₂₋₁₂ heteroalkylene (e.g., a heteroalkylene containing—C(O)—N(H)—, —N(H)—C(O)—, —S(O)₂—N(H)—, or —N(H)—S(O)₂—), optionally substituted C₁₋₁₂ thioheterocyclvlene e.g.

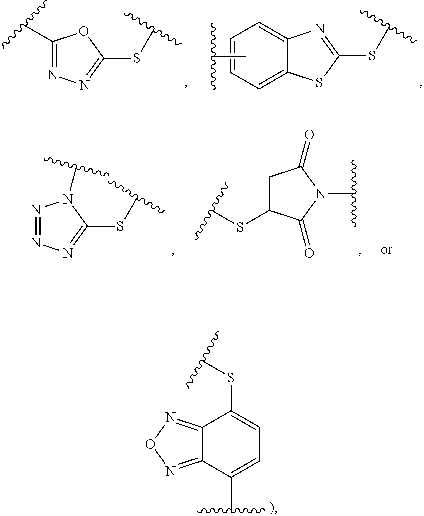

optionally substituted C₁₋₁₂ heterocyclylene (e.g., 1,2,3-triazole-1,4-diyl or

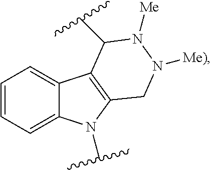

cyclobut-3-ene-1,2-dione-3,4-diyl, or pyrid-2-yl hydrazone);
Q² is a linear group (e.g., [-Q³-Q⁴-Q⁵]ₛ-), if p is 1, or a branched group (e.g., [-Q³-Q⁴-Q⁵]ₛ-Q⁷([Q³-Q⁴-Q⁵]ₛ-(Q⁷)ₚ₁)ₚ₂, where p1 is 0 or 1, p2 is 0, 1, 2, or 3), if p is an integer from 2 to 6;
each Q³ and each Q⁶ is independently absent, —CO—, —NH—, —O—, —S—, —SO₂—, —OC(O)—, —COO—, —NHC(O)—, —C(O)NH—, —CH₂—, —CH₂NH—, —NHCH₂—, —CH₂O—, or —OCH₂—; each Q⁴ is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, optionally substituted C₆₋₁₀ arylene, optionally substituted C₁₋₉ heteroarylene, or optionally substituted C₁₋₉ heterocyclylene;
each Q⁵ is independently absent, —CO—, —NH—, —O—, —S—, —SO₂—, —CH₂—, —C(O)O—, —OC(O)—,
   —C(O)NH—, —NH—C(O)—, —NH—CH(R^{a})—C(O)—, or —C(O)—CH(R^{a})—NH—;
each Q⁷ is independently optionally substituted C₁₋₆ alkane-triyl, optionally substituted C₁₋₆ alkane-tetrayl, optionally substituted C₂₋₆ heteroalkane-triyl, or optionally substituted C₂₋₆ heteroalkane-tetrayl; and
each R^{a} is independently H or an amino acid side chain;
provided that at least one of Q³, Q⁴, and Q⁵ is present.

In some aspects, each Q⁴ is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, or optionally substituted C₁₋₉ heterocyclylene. In certain aspects, s is 1, 2, 3, 4, 5, 6, 7, 8, 9, 10, 11, 12, 13, 14, 15, 16, 17, 18, 19, or 20.

Thus, in formula (II), LinkA may include a single branching point, if each p1 is 0, or multiple branching points, if at least one p1 is 1.

In formula (II), Q¹ may be —O-Q^{L}-Q^{C}-, where Q^{L} is optionally substituted C₂₋₁₂ heteroalkylene, optionally substituted C₁₋₁₂ alkylene, or -(optionally substituted C₁₋₆ alkylene)-(optionally substituted C₆₋₁₀ arylene)-. In some aspects, Q^{L} is optionally substituted C₂₋₁₂ heteroalkylene or optionally substituted C₁₋₁₂ alkylene. In formula (II), Q^{C} may be:

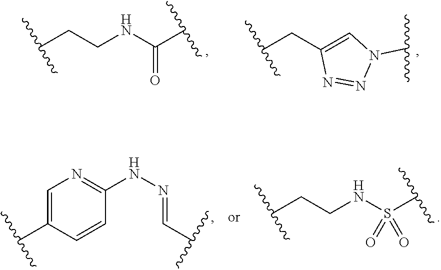 [SMILES: *CCNC(*)=O ; *Cc1cn(*)nn1 ; */C=N/Nc1ccc(*)cn1 ; *CCNS(*)(=O)=O]

In formula (II), Q² may be a linear group of formula [-Q³-Q⁴-Q⁵]ₛ-where Q³, Q⁴, and Q⁵ are as defined for formula (II). Alternatively, Q² may be a branched group [-Q³_Q⁴-Q⁵]ₛ-Q⁷([-Q³-Q⁴-, Q⁵]ₛ-Q⁷)ₚ₁)ₚ₂, where each Q⁷ is independently optionally substituted C₁₋₆ alkane-triyl, optionally substituted C₁₋₆ alkane-tetrayl, optionally substituted C₂₋₆ heteroalkane-triyl, or optionally substituted C₂₋₆ heteroalkane-tetrayl;
where
p1 is 0 or 1;
p2 is 0, 1, 2, or 3;
where,
when p1 is 0, LinkA is a trivalent or tetravalent linker, and,
when p1 is 1, LinkA is a tetravalent, pentavalent, or hexavalent linker.

In certain aspects, p1 is 0.

In some aspects, Q⁷ is:

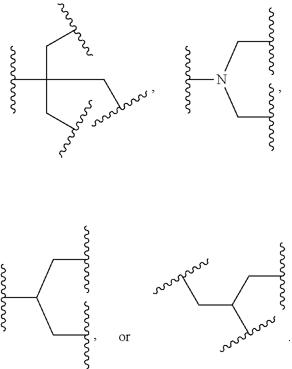 [SMILES: *CC(*)(C*)C* ; *CN(*)C* ; *CC(*)C* ; *CC(*)C*]

Compounds that may be used in the preparation of group -LinkA(-T)p in formula (I) are described herein as well as in WO 2015/188197. Non-limiting examples of -LinkA include:

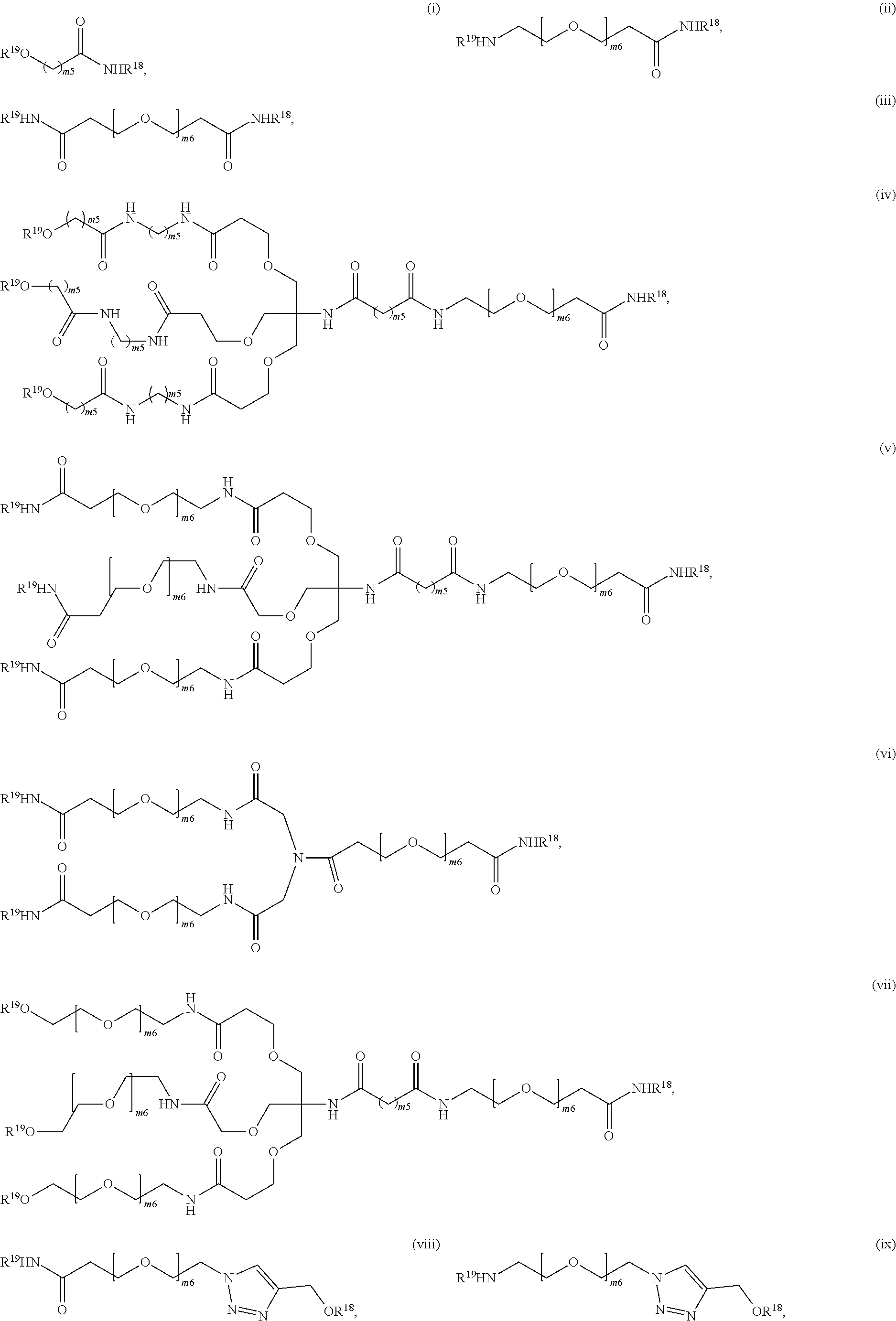

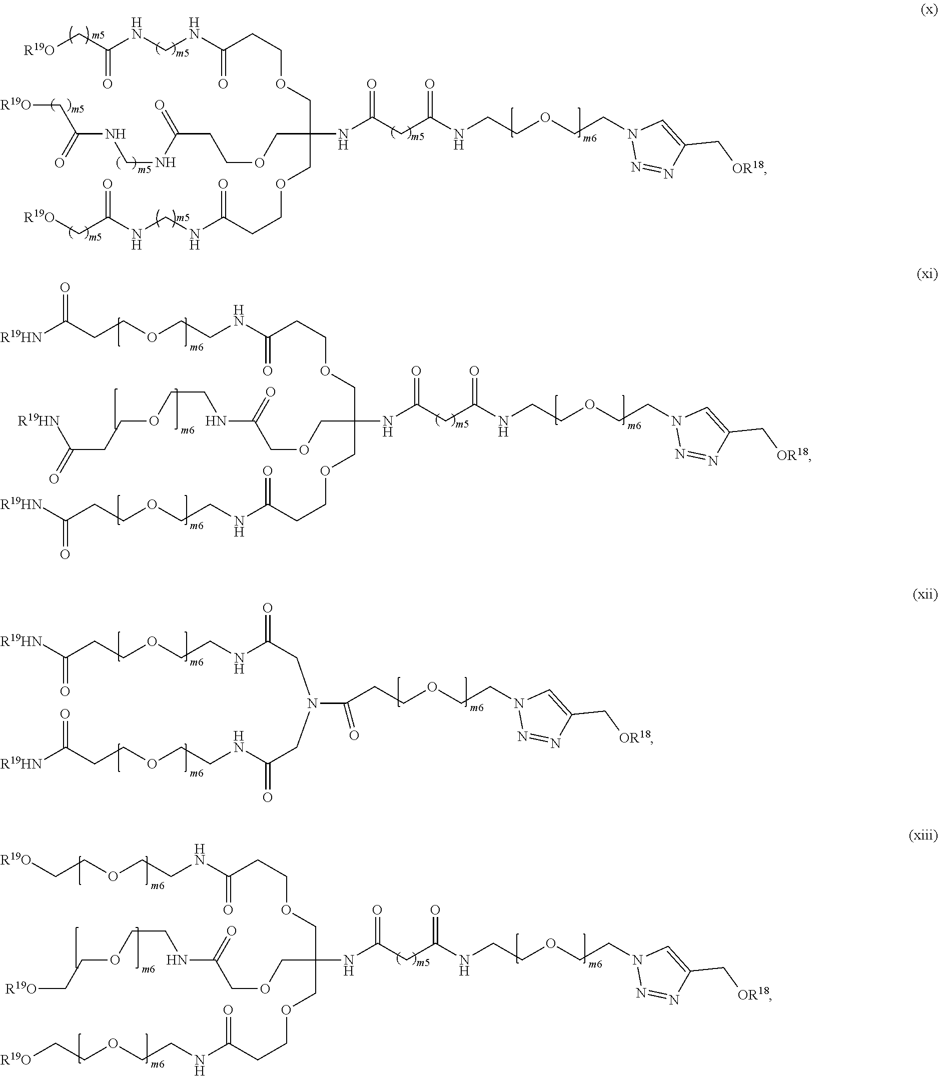

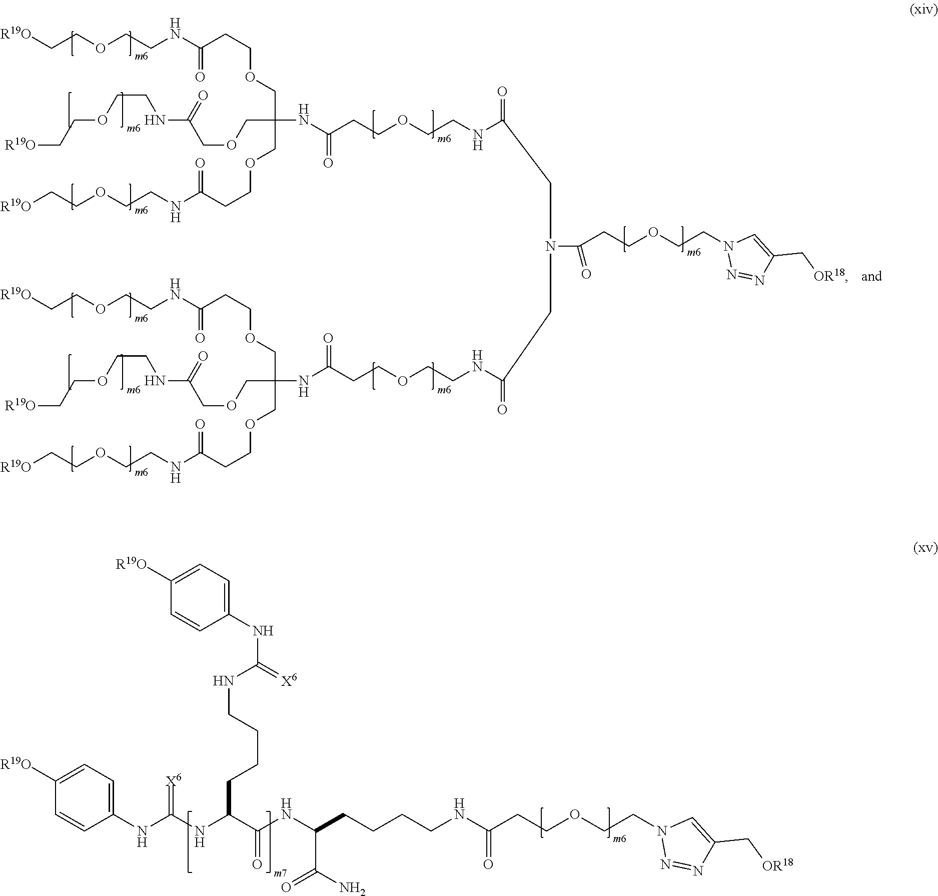

where
R¹⁸ is a bond to MOIETY,
each R¹⁹ is independently a bond to auxiliary moiety,
each m5 is independently an integer from 1 to 20,
each m6 is independently an integer from 1 to 10,
m7 is an integer from 1 to 6, and
each X⁶ is independently O or S.

In formula (II), when the conjugation linker is of formula [-Q³-Q⁴-Q⁵]ₛ-Q^{C}-, -Q²([-Q³-Q⁴-Q⁵]ₛ-Q⁶-T)ₚ may be:

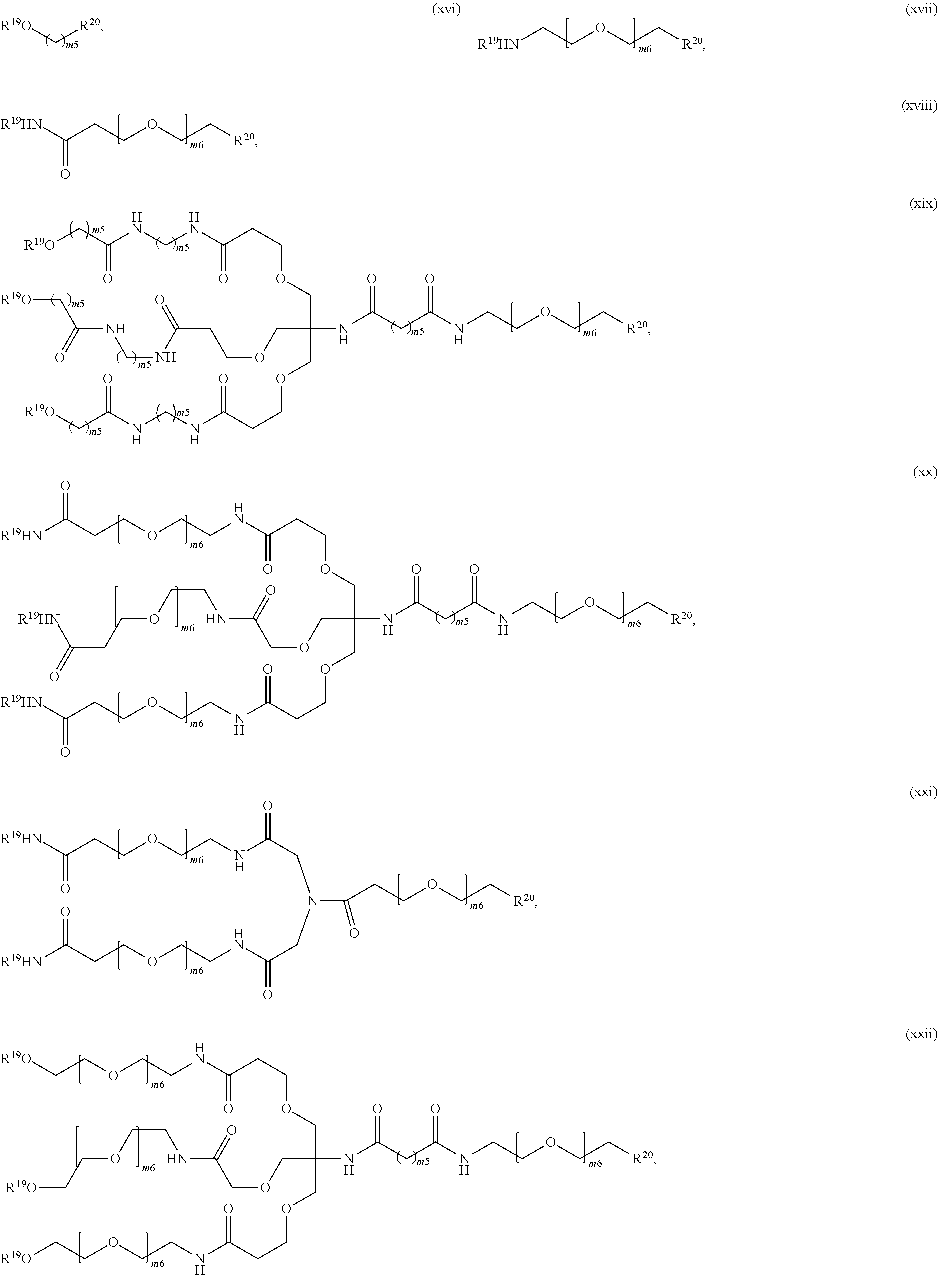

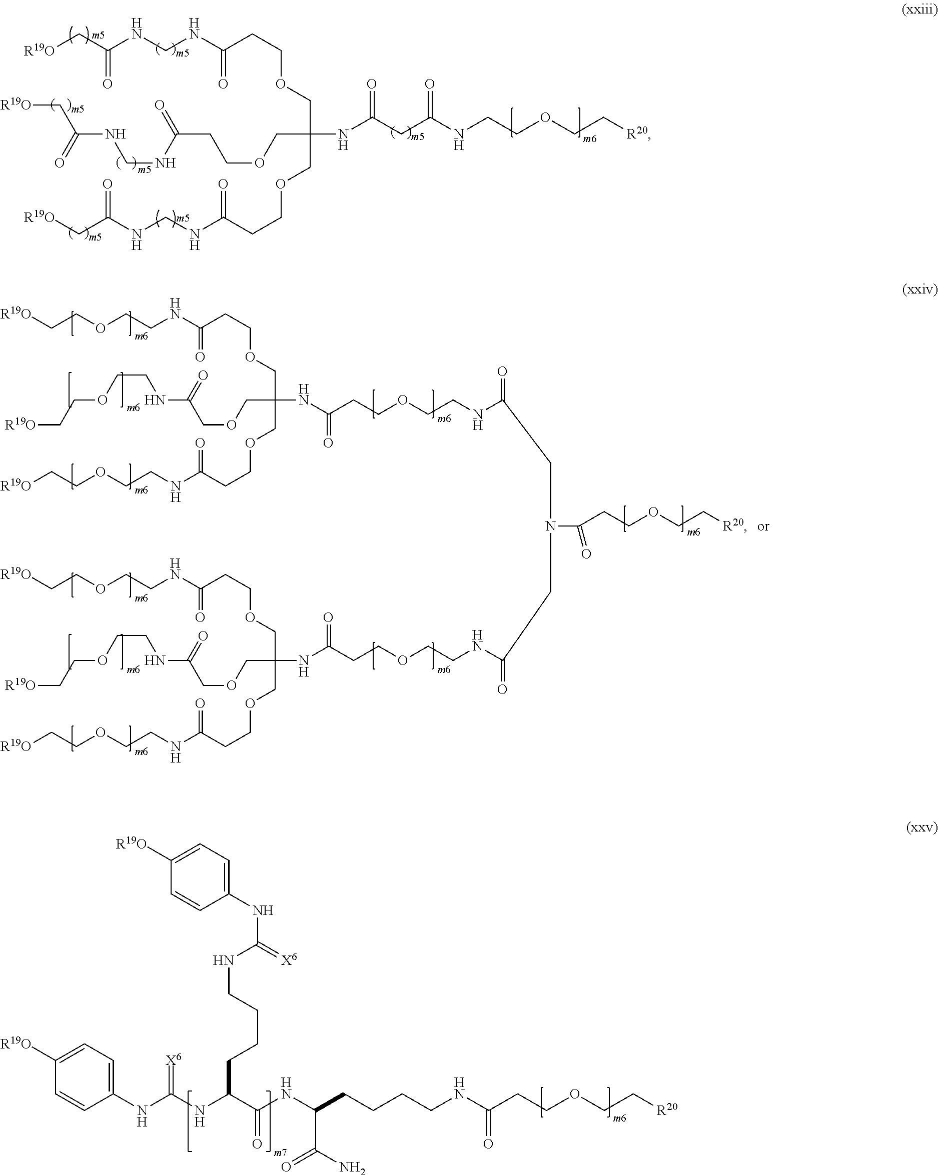

where
R²⁰ is a bond to Q^{C} in Q¹,
each R¹⁹ is independently a bond to an auxiliary moiety,
each m5 is independently an integer from 1 to 20,
each m6 is independently an integer from 1 to 10,
m7 is an integer from 1 to 6, and
each X⁶ is independently O or S.

In some aspects, the linker described herein is cleavable. In some aspects, the linker described herein is non-cleavable.

In some aspects, the polynucleic acid molecule described herein comprises a passenger strand or a guide strand bonded to at least one group of formula (IV),

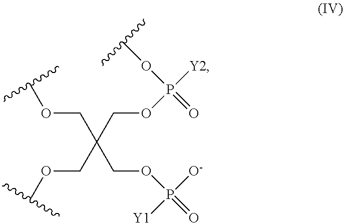

wherein at least one of Y1 or Y2 is a nucleotide from the polynucleic acid molecule. In some instances, the linker comprises formula (IV). In some instances, the linker and the asialoglycoprotein receptor targeting moiety with the last nucleotide on the 3′ end of the passenger strand of the polynucleic acid molecule are shown in (V′), (V″ ″), (V″″′), or (V″″″) as described herein.

In some instances, the Y1 is the last nucleotide on the 3′ end or the first nucleotide on the 5′ end of one of the strands of the polynucleic acid molecule. In some instances, the Y1 is the last nucleotide on the 3′ end or the first nucleotide on the 5′ end of the passenger strand of the polynucleic acid molecule. In some instances, the Y1 is the last nucleotide on the 3′ end or the first nucleotide on the 5′ end of the passenger strand of the polynucleic acid molecule, and the Y2 is a 3-hydroxy-propoxy group. In some instances, the Y2 is the first nucleotide on the 5′ end or the last nucleotide on the 3′ end of one of the strands of the polynucleic acid molecule. In some instances, the Y2 is the first nucleotide on the 5′ end or the last nucleotide on the 3′ end of the passenger strand of the polynucleic acid molecule. In some instances, the Y2 is the first nucleotide on the 5′ end or the last nucleotide on the 3′ end of the passenger strand of the polynucleic acid molecule, and the Y1 is a 3-hydroxy-propoxy group. In other instances, the Y1 and Y2 are two consecutive nucleotides in one of the strands of the polynucleic acid molecule.

In some aspects, the targeting moiety described herein is conjugated to 3′ end of the passenger strand (e.g., formula (IV′)). In some aspects, the targeting moiety described herein is conjugated to 5′ end of the passenger strand (e.g., formula (IV″) or (IV″′)). In some aspects, the targeting moiety described herein is conjugated to 3′ end of the guide strand (e.g., formula (IV′)). In some aspects, the targeting moiety described herein is conjugated to 5′ end of the guide strand (e.g., formula (IV″) or (IV″′)).

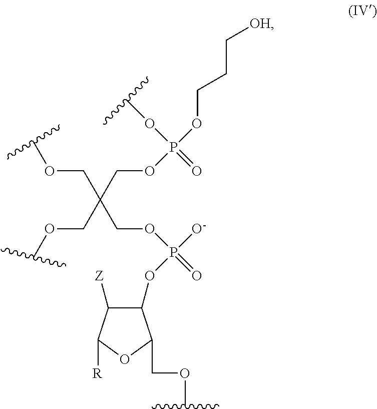

wherein Z in formula (IV′) is a moiety that corresponds to one of the sugar modifications described herein (e.g., —H, —OH, —O-Methyl, —F, or —O-methoxyethyl) and R in formula (IV′) is adenine, uracil, guanine, cytosine, thymine, abasic, or others.

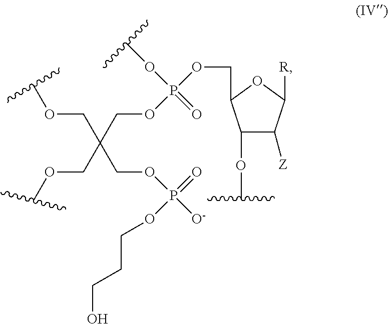

wherein Z in formula (IV″) is a moiety that corresponds to one of the sugar modifications described herein (e.g., —H, —OH, —O-Methyl, —F, or —O-methoxyethyl) and R in formula (IV″) is adenine, uracil, guanine, cytosine, thymine, abasic, or others.

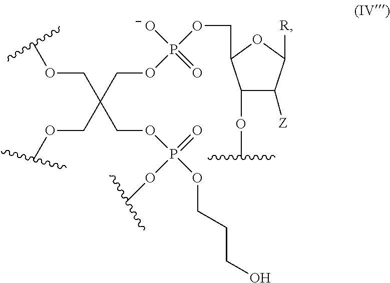

wherein Z in formula (IV″′) is a moiety that corresponds to one of the sugar modifications described herein (e.g., —H, —OH, —O-Methyl, —F, or —O-methoxyethyl) and R in formula (IV″′) is adenine, uracil, guanine, cytosine, thymine, abasic, or others.

In some aspects, the linker conjugated with one or more targeting moieties as shown in Formula (IV″) or (IV″′) is added to the first nucleotide on the 5′ end. In some aspects, the linker conjugated with one or more GalNAc as shown in Formula (V″) or (V″′) is added to the first nucleotide on the 5′ end. In some aspects, the modification pattern comprises one or more phosphorothioate modified internucleotide linkages. In some aspects, the modification pattern is shown in Formula (VII). In some aspects, the 5′ end modification known in the art is applied to the one or more inverted nucleotides.

### Pharmaceutical Compositions

Delivery of the polynucleic acid molecules described herein can be achieved by contacting a cell with the construct using a variety of methods. In particular aspects, the polynucleic acid molecule described herein is formulated with various excipients, vehicles, and carriers, as described more fully elsewhere herein.

A pharmaceutical composition described herein can be prepared to include a hybridized polynucleotide construct disclosed herein, into a form suitable for administration to a subject using carriers, excipients, and vehicles. Frequently used excipients include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, anti-oxidants, chelating agents, and inert gases. Other pharmaceutically acceptable vehicles include aqueous solutions, non-toxic excipients, including salts, preservatives, buffers and the like, as described, for instance, in Remington: The Science and Practice of Pharmacy, 21st Ed., Gennaro, Ed., Lippencott Williams & Wilkins (2005), and The United States Pharmacopeia: The National Formulary (USP 36 NF31), published in 2013. The pH and exact concentration of the various components of the pharmaceutical composition are adjusted according to routine skills in the art. See Goodman and Gilman's, The Pharmacological Basis for Therapeutics.

The pharmaceutical compositions described herein may be administered locally or systemically. The therapeutically effective amounts will vary according to factors, such as the degree of infection in a subject, the age, sex, and weight of the individual. Dosage regimes can be adjusted to provide the optimum therapeutic response. For example, several divided doses can be administered daily or the dose can be proportionally reduced as indicated by the exigencies of the therapeutic situation.

The pharmaceutical composition can be administered in a convenient manner, such as by injection (e.g., subcutaneous, intravenous, intraorbital, and the like), oral administration, ophthalmic application, inhalation, topical application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. The composition will typically be sterile and fluid to the extent that easy syringability exists. Typically the composition will be stable under the conditions of manufacture and storage and preserved against the contaminating action of microorganisms, such as bacteria and fungi. The vehicle can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants.

Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols, such as mannitol, sorbitol, or sodium chloride are used in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

It is especially advantageous to formulate parenteral compositions in dosage unit form for ease of administration and uniformity of dosage. Dosage unit form as used herein, refers to physically discrete units suited as unitary dosages for the subject to be treated; each unit containing a predetermined quantity of pharmaceutical composition is calculated to produce the desired therapeutic effect in association with the required pharmaceutical vehicle. The specification for the dosage unit forms are related to the characteristics of the pharmaceutical composition and the particular therapeutic effect to be achieve. The principal pharmaceutical composition is compounded for convenient and effective administration in effective amounts with a suitable pharmaceutically acceptable vehicle in an acceptable dosage unit. In the case of compositions containing supplementary active ingredients, the dosages are determined by reference to the usual dose and manner of administration of the ingredients.

The pharmaceutical composition can be orally administered, for example, in a carrier, e.g., in an enteric-coated unit dosage form. The pharmaceutical composition and other ingredients can also be enclosed in a hard or soft-shell gelatin capsule or compressed into tablets. For oral therapeutic administration, the pharmaceutical composition can be incorporated with excipients and used in the form of ingestible tablets, troches, capsules, pills, wafers, and the like. Such compositions and preparations should contain at least 1% by weight of active compound. The percentage of the compositions and preparations can, of course, be varied and can conveniently be between about 5% to about 80% of the weight of the unit. The tablets, troches, pills, capsules, and the like can also contain the following: a binder, such as gum tragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Any material used in preparing any dosage unit form should be of pharmaceutically acceptable purity and substantially non-toxic in the amounts employed. In addition, the pharmaceutical composition can be incorporated into sustained-release preparations and formulations.

The pharmaceutical composition described herein may comprise one or more permeation enhancer that facilitates bioavailability of the polynucleic acid molecule described herein. WO 2000/67798, Muranishi, 1990, Crit. Rev. Ther. Drug Carrier Systems, 7, 1, Lee et al., 1991, Crit. Rev. Ther. Drug Carrier Systems, 8, 91 are herein incorporated by reference in its entirety. In some aspects, the permeation enhancer is intestinal. In some aspects, the permeation enhancer is transdermal. In some aspects, the permeation enhancer is to facilitate crossing the brain-blood barrier. In some aspects, the permeation enhancer improves the permeability in the oral, nasal, buccal, pulmonary, vaginal, or corneal delivery model. In some aspects, the permeation enhancer is a fatty acid or a derivative thereof. In some aspects, the permeation enhancer is a surfactant or a derivative thereof. In some aspects, the permeation enhancer is a bile salt or a derivative thereof. In some aspects, the permeation enhancer is a chelating agent or a derivative thereof. In some aspects, the permeation enhancer is a non-chelating non-surfactant or a derivative thereof. In some aspects, the permeation enhancer is an ester or a derivative thereof. In some aspects, the permeation enhancer is an ether or a derivative thereof. In some aspects, the permeation enhancer is arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof. In one specific aspect, the permeation enhancer is sodium caprate (C10).

In some aspects, the permeation enhancer is chenodeoxycholic acid (CDCA), ursodeoxychenodeoxycholic acid (UDCA), cholic acid, dehydrocholic acid, deoxycholic acid, glucholic acid, glycholic acid, glycodeoxycholic acid, taurocholic acid taurodeoxycholic acid, sodium tauro-24,25-dihydro-fusidate or sodium glycodihydrofusidate. In some aspects, the permeation enhancer is polyoxyethylene-9-lauryl ether, or polyoxyethylene-20-cetyl ether.

For the polynucleic acid molecule described herein, suitable pharmaceutically acceptable salts include (i) salts formed with cations such as sodium, potassium, ammonium, magnesium, calcium, polyamines such as spermine and spermidine, etc.; (ii) acid addition salts formed with inorganic acids, for example hydrochloric acid, hydrobromic acid, sulfuric acid, phosphoric acid, and the like; and (iii) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like.

While the hybridized polynucleotide constructs described herein may not require the use of excipients for delivery to the target cell, the use of excipients may be advantageous in some aspects. Thus, for delivery to the target cell, the hybridized polynucleic acid molecule described herein can non-covalently bind an excipient to form a complex. The excipient can be used to alter biodistribution after delivery, to enhance uptake, to increase half-life or stability of the strands in the hybridized polynucleotide constructs (e.g., improve nuclease resistance), and/or to increase targeting to a particular cell or tissue type.

Exemplary excipients include a condensing agent (e.g., an agent capable of attracting or binding a nucleic acid through ionic or electrostatic interactions); a fusogenic agent (e.g., an agent capable of fusing and/or being transported through a cell membrane); a protein to target a particular cell or tissue type (e.g., thyrotropin, melanotropin, lectin, glycoprotein, surfactant protein A, or any other protein); a lipid; a lipopolysaccharide; a lipid micelle or a liposome (e.g., formed from phospholipids, such as phosphotidylcholine, fatty acids, glycolipids, ceramides, glycerides, cholesterols, or any combination thereof); a nanoparticle (e.g., silica, lipid, carbohydrate, or other pharmaceutically-acceptable polymer nanoparticle); a polyplex formed from cationic polymers and an anionic agent (e.g., a CRO), where exemplary cationic polymers include polyamines (e.g., polylysine, polyarginine, polyamidoamine, and polyethylene imine); cholesterol; a dendrimer (e.g., a polyamidoamine (PAMAM) dendrimer); a serum protein (e.g., human serum albumin (HSA) or low-density lipoprotein (LDL)); a carbohydrate (e.g., dextran, pullulan, chitin, chitosan, inulin, cyclodextrin, or hyaluronic acid); a lipid; a synthetic polymer, (e.g., polylysine (PLL), polyethylenimine, poly-L-aspartic acid, poly-L-glutamic acid, styrene-maleic acid anhydride copolymer, poly(L-lactide-co-glycolic) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacrylic acid), N-isopropylacrylamide polymer, pseudopeptide-polyamine, peptidomimetic polyamine, or polyamine); a cationic moiety (e.g., cationic lipid, cationic porphyrin, quaternary salt of a polyamine, or alpha helical peptide); a multivalent sugar (e.g., multivalent lactose, multivalent galactose, N-acetyl-galactosamine, N-acetyl-glucosamine, multivalent mannose, or multivalent fucose); a vitamin (e.g., vitamin A, vitamin E, vitamin K, vitamin B, folic acid, vitamin B12, riboflavin, biotin, or pyridoxal); a cofactor; or a drug to disrupt cellular cytoskeleton to increase uptake (e.g., taxol, vincristine, vinblastine, cytochalasin, nocodazole, japlakinolide, latrunculin A, phalloidin, swinholide A, indanocine, or myoservin).

Other therapeutic agents as described herein may be included in a pharmaceutical composition described herein in combination with a polynucleic acid molecule described herein.

### Methods of Treatment

In some aspects, described herein is a method of modulating mRNA expression of ANGPTL3 gene in a subject, comprising: administering to the subject a polynucleic acid molecule described herein, a polynucleic acid molecule conjugate described herein, or a pharmaceutical composition described herein, thereby modulating the mRNA expression of ANGPTL3 gene in the subject. In some aspects, described herein is a method of preventing, alleviating, or treating ANGPTL3-associated disease or symptom thereof in a subject in need thereof, comprising: administering to the subject a polynucleic acid molecule described herein, a polynucleic acid molecule conjugate described herein, or a pharmaceutical composition described herein.

In some aspects, the method described herein reduces expression of ANGPTL3 gene in a subject by about or at least 10% compared to a negative control. In some aspects, the method described herein reduces expression of ANGPTL3 gene in a subject by about or at least 20% compared to a negative control. In some aspects, the method described herein reduces expression of ANGPTL3 gene in a subject by about or at least 30% compared to a negative control. In some aspects, the method described herein reduces expression of ANGPTL3 gene in a subject by about or at least 40% compared to a negative control. In some aspects, the method described herein reduces expression of ANGPTL3 gene in a subject by about or at least 50% compared to a negative control. In some aspects, the method described herein reduces expression of ANGPTL3 gene in a subject by about or at least 60% compared to a negative control. In some aspects, the method described herein reduces expression of ANGPTL3 gene in a subject by about or at least 70% compared to a negative control. In some aspects, the method described herein reduces expression of ANGPTL3 gene in a subject by about or at least 80% compared to a negative control. In some aspects, the method described herein reduces expression of ANGPTL3 gene in a subject by about or at least 90% compared to a negative control. In some aspects, the method described herein reduces expression of ANGPTL3 gene in a subject by about 100% compared to a negative control.

In some aspects, described herein is a method of modulating ANGPTL3 protein level in a subject in need thereof, comprising administering to the subject a polynucleic acid molecule described herein, a polynucleic acid molecule conjugate described herein, or a pharmaceutical composition described herein, wherein the polynucleic acid molecule described herein, the polynucleic acid molecule conjugate described herein, or the pharmaceutical composition described herein modulates the ANGPTL3 protein level in the subject. In some aspects, described herein is a method of preventing, alleviating, or treating ANGPTL3-associated disease or symptom thereof in a subject in need thereof, comprising: administering to the subject a polynucleic acid molecule described herein, a polynucleic acid molecule conjugate described herein, or a pharmaceutical composition described herein, wherein the polynucleic acid molecule described herein, the polynucleic acid molecule conjugate described herein, or the pharmaceutical composition described herein modulates the ANGPTL3 protein level in the subject.

In some another aspects, the method described herein reduces ANGPTL3 activity level in a subject by about or at least 10% compared to a negative control. In some aspects, the method described herein reduces ANGPTL3 level in a subject by about or at least 20% compared to a negative control. In some aspects, the method described herein reduces ANGPTL3 level in a subject by about or at least 30% compared to a negative control. In some aspects, the method described herein reduces ANGPTL3 level in a subject by about or at least 40% compared to a negative control. In some aspects, the method described herein reduces ANGPTL3 level in a subject by about or at least 50% compared to a negative control. In some aspects, the method described herein reduces ANGPTL3 level in a subject by about or at least 60% compared to a negative control. In some aspects, the method described herein reduces ANGPTL3 level in a subject by about or at least 70% compared to a negative control. In some aspects, the method described herein reduces ANGPTL3 level in a subject by about or at least 80% compared to a negative control. In some aspects, the method described herein reduces ANGPTL3 level in a subject by about or at least 90% compared to a negative control. In some aspects, the method described herein reduces ANGPTL3 level in a subject by about 100% compared to a negative control.

In some aspects, provided herein includes methods of preventing, alleviating, or treating ANGPLT3-related or associated disease or a symptom thereof in a subject. In some aspects, the ANGPLT3-related or associated disease or a symptom comprises hyperlipedemia, coronary heart disease, vascular disease, severe hypertriglyceridemia, familial chylomicronemia syndrome (FCS), high fasting triglycerides on restricted low-fat diet, overweight or obese, type 2 diabetes, dyslipidaemia, cardiovascular disease,atherosclerosis, stroke, acute pancreatitis, atherosclerotic cardiovascular disease, atrial fibrillation, myocardial infarction, calcific aortic valve stenosis, suffers from cardiac arrest, or peripheral arterial disease.

### EXAMPLES

These examples are provided for illustrative purposes only and not to limit the scope of the claims provided herein. For all of the sequences presented below, oligonucleotide structure representation reads from left to right (5′ to 3′). Monomer codes present in the oligonucleotide code are linked by 5′-3′ phosphodiester bonds unless specified (succeeded by 3′ intemucleotide linkage reading left to right). Abbreviations of nucleotide monomers used in oligonucleotide structure representation are as follows. “A” stands for Adenosine-3′-phosphate; “a” stands for 2′-O-methyladenosine-3′-phosphate; “Af” stands for 2′-fluoroadenosine-3′-phosphate; “dA” stands for 2′-deoxyadenosine-3′-phosphate; “al” refers to 2-Amino-2′-O-methyladenosine-3′-phosphate; “C” stands for Cytidine-3′-phosphate; “c” stands for 2′-O-methylcytidine-3′-phosphate; “Cf” stands for 2′-fluorocytidine-3′-phosphate; “dC” stands for 2′-deoxycytidine-3′-phosphate; “G” stands for Guanosine-3′-phosphate; “g” stands for 2′-O-methylguanosine-3′-phosphate; “Gf” stands for 2′-fluoroguanosine-3′-phosphate; “dG” stands for 2′-deoxyguanosine-3′-phosphate; “U” stands for Uridine-3′-phosphate; “u” stands for 2′-O-methyluridine-3′-phosphate; “Uf” stands for 2′-fluorouridine-3′-phosphate; “u3” refers to 2′-O-methyl-2-thiouridine-3′-phosphate; “U3f” refers to 2′-fluoro-2-thiouridine-3′-phosphate; “dU” stands for 2′-deoxyuridine-3′-phosphate; “T” stands for 5-methyluridine-3′-phosphate; “t” stands for 2′-O-methyl-5-methyluridine-3′-phosphate; “Tf” stands for 2′-fluoro-5-methyluridine-3′-phosphate; “dT” stands for thymidine-3′-phosphate; “s” stands for 3′-phosphorothioate; “(T-T)” refers to acyclic L-threoninol nucleic acid-thymine-3′-phosphate; “(T-A)” refers to acyclic L-threoninol nucleic acid-adenine-3′-phosphate; “(T-NAc)” refers to acyclic N-acetyl L-threoninol abasic nucleic acid-3′phosphate; “(dAB)” refers to 1′,2′-Dideoxyribose-3′-phosphate; and “(Tgn)” refers to thymidine-glycol nucleic acid (GNA)S-isomer.

### Example 1—In Vitro Efficacy of siRNAs Targeting ANGPTL3

A panel of siRNAs were generated (shown in Table 1), and each passenger strand (sense strand) was conjugated with a triantennary GalNAc moiety. The siRNA-GalNAc conjugates were evaluated in vitro in primary human hepatocytes.

Cryopreserved primary human hepatocytes (PHHs) were thawed and plated on collagen-coated 96-well plates at a density of 9×10⁴ cells per well. Hepatocytes were treated by incubating with the siRNAs shown in Table 1 with each passenger strand (sense strand) conjugated with a triantennary GalNAc moiety in the absence of transfection reagents (free uptake) for 48 hours. Cells were treated with the siRNAs at a concentrations of 10 μM or 0.5 μM. Untreated PHHs were used as a negative control. An siRNA targeting an unrelated gene (Ahsa1) was also used as a negative control. At the end of the incubation period, the cells were lysed, the mRNA was isolated, and the relative expression of the target gene was measured by branched DNA (bDNA) assay and normalized to a house-keeping gene, human GAPDH, using standard protocols. 4 replicates were run per dose level per each siRNA. The result of in vitro potency of the siRNAs are showed in Table 5.

### Example 2—Drug Response Curves for Selected ANGPTL3 siRNAs

A selected group of siRNAs targeting ANGPTL3 shown in Table 2 were used, and each passenger strand (sense strand) was conjugated with a triantennary GalNAc moiety. For dose response curve determination, primary human hepatocytes are seeded into 96-well plates at appropriate density. The siRNA-GalNAc conjugates were evaluated at 10 different doses in primary human hepatocytes, using a similar experimental setup in Example 1. The corresponding drug response curves are depicted in Table 7.

### Example 3—In Vivo Testing of ANGPTL3 siRNA in Mice

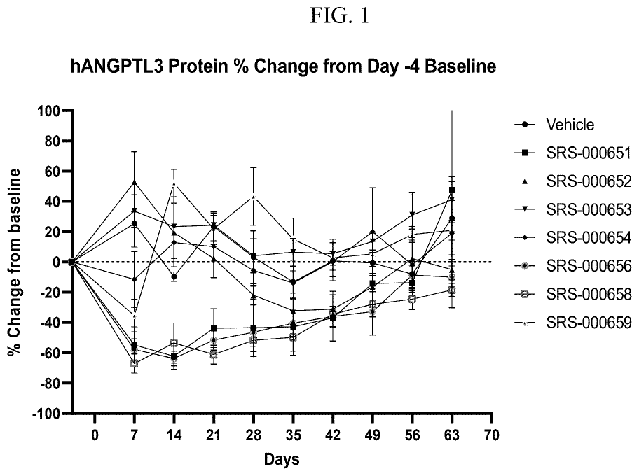

Selected siRNAs shown in Table 3 or PBS were administered in a single subcutaneous dose into transgenic ANGPTL3 mice (n=6 per group) available commercially from Shanghai Model Organisms Center, Inc (C57BL/6-Angptl3^{em2(hANGPTL3)Smoc}, cat #NM-HU-210036). The 3′ end of the passenger strand (sense strand) of each siRNA is conjugated with a triantennary GalNAc moiety (X2-GalNAc as in Formula (V′)). Selected siRNAs were administered at a single subcutaneous dose level of 1 mg/kg on day 0. Serum hANGPTL3 protein levels were measured by ELISA (R&D Systems, cat #DANL30) on days −4 (predose) and days 7, 14, 21, 28, 35, 42, 49, 56, and 63. For each individual, the % change in serum ANGPTL3 relative to the day −4 predose value was calulated. The group average % change in serum hANGPTL3 protein levels from day −4 baseline and standard error are reported in Table 8. Results are plotted and shown in FIG. 1.

Example 3: Study Design

| | ANGPTL3 | | | | |
|---|---|---|---|---|---|
| | Target | Group | | Dose | Blood |
| Duplex ID | Position | # | n/group | Level | Collection |
| PBS/Vehicle | — | 1 | 6 | Single | Days −4 |
| SRS-000651 | 269 | 2 | 6 | 1 | (predose), 7, |
| SRS-000652 | 412 | 3 | 6 | mg/kg | 14, 21, 28, 35, |
| SRS-000653 | 485 | 4 | 6 | SQ | 42, 49, 56, 63 |
| SRS-000654 | 520 | 5 | 6 | on | |
| SRS-000656 | 1101 | 6 | 6 | Day 0 | |
| SRS-000658 | 1357 | 7 | 6 | | |
| SRS-000659 | 1363 | 8 | 6 | | |

### Example 4—In Vivo Testing of ANGPTL3 siRNA in Mice

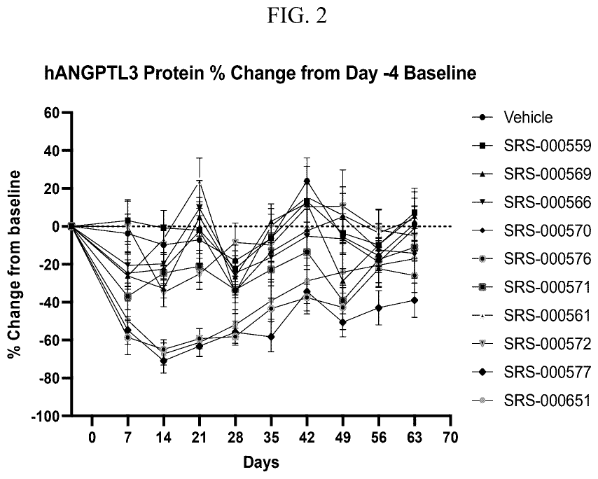

Selected siRNAs shown in Table 3 or PBS were administered in a single subcutaneous dose into transgenic ANGPTL3 mice (n=5 per group) available commercially from Shanghai Model Organisms Center, Inc (C57BL/6-Angptl3^{em2(hANGPTL3)Smoc}, cat #NM-HU-210036). The 3′ end of the passenger strand (sense strand) of each siRNA is conjugated with a triantennary GalNAc moiety (X2-GalNAc as in Formula (V′)). Selected siRNAs were administered at a single subcutaneous dose level of 1 mg/kg on day 0. Serum hANGPTL3 protein levels were measured by ELISA (R&D Systems, cat #DANL30) on days −4 (predose) and days 7, 14, 21, 28, 35, 42, 49, 56, and 63. For each individual, the % change in serum ANGPTL3 relative to the day −4 predose value was calulated. The group average % change in serum hANGPTL3 protein levels from day −4 baseline and standard error are reported in Table 8. Results are plotted and shown in FIG. 1. The average 0% change in serum hANGPTL3 protein levels from day−4 baseline and standard error are reported in Table 9. Results are plotted and shown in FIG. 2.

Example 4: Study Design

| | ANGPTL3 | | | | |
|---|---|---|---|---|---|
| | Target | Group | | Dose | Blood |
| Duplex ID | Position | # | n/group | Level | Collection |
| PBS/Vehicle | — | 1 | 5 | Single | Days −4 |
| SRS-000559 | 56 | 2 | 5 | 1 | (predose), 7, |
| SRS-000569 | 354 | 3 | 5 | mg/kg | 14, 21, 28, 35, |
| SRS-000566 | 165 | 4 | 5 | SQ | 42, 49, 56, 63 |
| SRS-000570 | 396 | 5 | 5 | on | |
| SRS-000576 | 1358 | 6 | 5 | Day 0 | |
| SRS-000571 | 473 | 7 | 5 | | |
| SRS-000561 | 72 | 8 | 5 | | |
| SRS-000572 | 555 | 9 | 5 | | |
| SRS-000577 | 1384 | 10 | 5 | | |
| SRS-000651 | 269 | 11 | 5 | | |

### Example 5—In Vivo Testing of ANGPTL3 siRNA in Mice

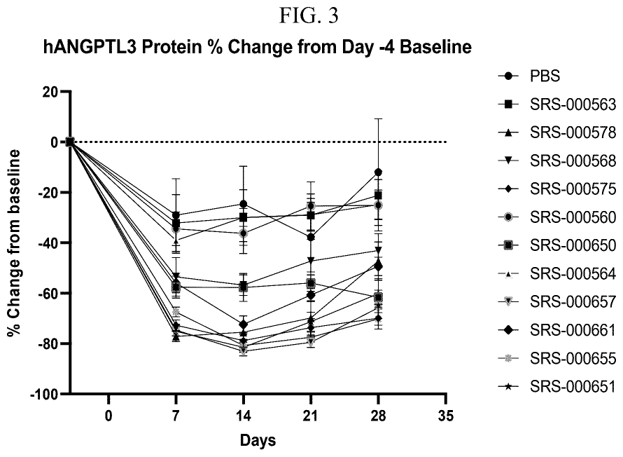

Selected siRNAs shown in Table 3 or PBS were administered in a single subcutaneous dose into transgenic ANGPTL3 mice (n=5 per group) available commercially from Shanghai Model Organisms Center, Inc (C57BL/6 Angptl3^{em2(hANGPTL3)Smoc}, cat #NM-HU-210036). The 3′ end of the passenger strand (sense strand) of each siRNA is conjugated with a triantennary GalNAc moiety (X2-GaNAc as in Formula (V′)). Selected siRNAs were administered at a single subcutaneous dose level of 1 mg/kg on day 0. Serum hANGPTL3 protein levels were measured by ELISA (R&D Systems, cat #DANL30) on days −4 (predose) and days 7, 14, 21, and 28. For each individual, the 00 change in serum ANGPTL3 relative to the day −4 predose value was calulated. The average 0 change in serum hANGPTL3 protein levels from day −4 baseline and standard error are reported in Table 10. Results are plotted and shown in FIG. 3.

### Example 5: Study Design

Example 5: Study Design

| | ANGPTL3 | | | | |
|---|---|---|---|---|---|
| | Target | Group | | Dose | Blood |
| Duplex ID | Position | # | n/group | Level | Collection |
| PBS/Vehicle | NA | 1 | 5 | Single | Days −4 |
| SRS-000563 | 159 | 2 | 5 | 1 | (predose), |
| SRS-000578 | 1496 | 3 | 5 | mg/kg | 7, 14, 21, |
| SRS-000568 | 283 | 4 | 5 | SQ | 28 |
| SRS-000575 | 1014 | 5 | 5 | on | |
| SRS-000560 | 71 | 6 | 5 | Day 0 | |
| SRS-000650 | 151 | 7 | 5 | | |
| SRS-000564 | 161 | 8 | 5 | | |
| SRS-000657 | 1103 | 9 | 5 | | |
| SRS-000661 | 1494 | 10 | 5 | | |
| SRS-000655 | 1012 | 11 | 5 | | |
| SRS-000651 | 269 | 12 | 5 | | |

### Example 6—In Vivo Testing of ANGPTL3 siRNA in Mice

Selected siRNAs shown in Table 3 or PBS were administered in a single subcutaneous dose into transgenic ANGPTL3 mice (n=5 per group) available commercially from Shanghai Model Organisms Center, Inc (C57BL/6-Angptl3^{em2(hANGPTL3)Smoc}, cat #NM-HU-210036). The 3′ end of the passenger strand (sense strand) of each siRNA is conjugated with a triantennary GalNAc moiety (X2-GalNAc as in Formula (V′)). Selected siRNAs were administered at a single subcutaneous dose level of 1 mg/kg on day 0. Serum hANGPTL3 protein levels were measured by ELISA (R&D Systems, cat #DANL30) on days −4 (predose) and days 7, 14, 21, 28, 35, and 42. For each individual, the % change in serum ANGPTL3 relative to the PBS group mean per timepoint was calculated. The average % change in serum hANGPTL3 protein levels relative to the PBS group and standard error are reported in Table 11. Results are plotted and

Example 6: Study Design

| | ANGPTL3 | | | | |
|---|---|---|---|---|---|
| | Target | Group | | Dose | Blood |
| Duplex ID | Position | # | n/group | Level | Collection |
| PBS/Vehicle | NA | 1 | 5 | Single | Days −4 |
| SRS-001803 | 54 | 2 | 5 | 1 | (predose), 7, |
| SRS-001804 | 158 | 3 | 5 | mg/kg | 14, 21, 28, 35, |
| SRS-001805 | 267 | 4 | 5 | SQ | 42 |
| SRS-001806 | 281 | 5 | 5 | on | |
| SRS-001807 | 919 | 6 | 5 | Day 0 | |
| SRS-001808 | 1007 | 7 | 5 | | |
| SRS-001809 | 1008 | 8 | 5 | | |
| SRS-001810 | 1013 | 9 | 5 | | |
| SRS-001811 | 1099 | 10 | 5 | | |
| SRS-001812 | 1102 | 11 | 5 | | |
| SRS-000651 | 269 | 12 | 5 | | |

### Example 7—In Vivo Testing of ANGPTL3 siRNA in Non-Human Primates (NHP)

Sequences of siRNAs used for the non-human primate study are specified in Table 4 The 3′ end of each passenger strand (sense strand) is conjugated with a GalNAc via X2 (as in Formula (V′)). SRS-001704 was used as a benchmark or a reference in this example.

Information regarding SRS-001704 is shown below:

Guide/antisense structure code:
(SEQ ID NO: 886)
5′-usAfscsUfgAfuCfaAfaUfaUfgUfuGfaGfsc-3′;
Guide/antisense base sequence:
(SEQ ID NO: 885)
5′-UACUGAUCAAAUAUGUUGAGC-3′;

Passenger/sense structure code: 5′-s(invAb)sgcucaacaUfAfUfuugaucaguas(invAb)-3′ (SEQ ID NO: 888), and a GalNAc was conjugated to the 5′ end of the passenger strand via formula V″′; and

Passenger/sense base sequence:
(SEQ ID NO: 887)
5′-GCUCAACAUAUUUGAUCAGUA-3′.

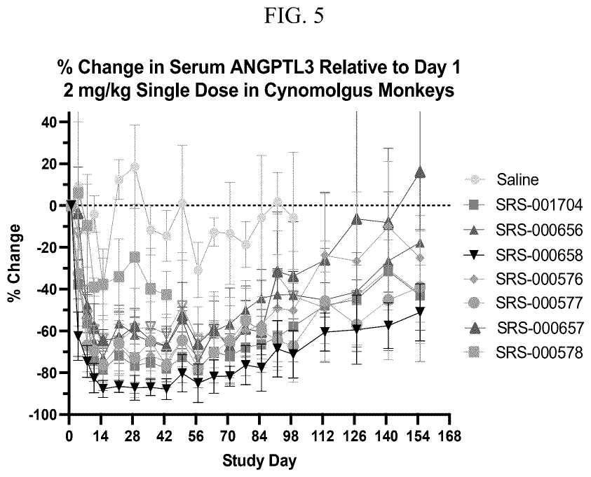

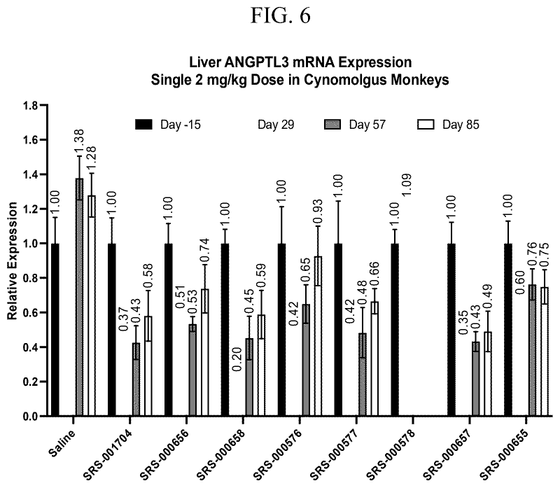

On day 1, male cynomolgus monkeys (n=4 per treatment group/siRNA) were administered a single 2 mg/kg subcutaneous injection of ANGPTL3 siRNA constructs as shown in Table 4, or sterile istonic saline. Blood samples were collected pre-dose, and on days 4, 8, 11, 15, 22, 29, 36, 43, 50, 57, 64, 71, 78,85, 92, 99, 113, 127, 141, and 155, and processed to serum. ANGPTL3 circulating protein levels in all serum samples were analyzed using an ANGPTL3 ELISA assay (Thermo Fisher, catalog #: EH29RBX5). Results are expressed as the group mean of each individual's % change in circulating ANGPTL3 protein relative to the day 1 pre-dose timepoint and shown in Table 12 and FIG. 5. To assess depth of ANGPTL3 mRNA silencing in liver tissue, ultrasound guided liver biopsies were collected pre-dose (day −15) and on days 29, 57, and 85. At each timepoint, qPCR was performed on all samples to measure ANGPTL3 mRNA, normalizing to ACTB housekeeping gene. ANGPTL3 mRNA expression relative to the day −15 timepoint are plotted in FIG. 6.

### Example 8—In Vivo Testing of ANGPTL3 siRNA in Mice

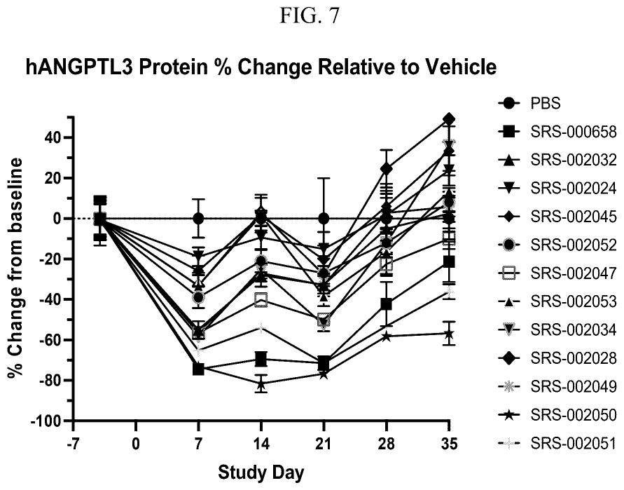

Selected siRNAs shown in Table 3 or PBS were administered in a single subcutaneous dose into transgenic ANGPTL3 mice (n=5 per group) available commercially from Shanghai Model Organisms Center, Inc (C57BL/6-Angptl3^{em2(hANGPTL3)Smoc}, cat #NM-HU-210036). The 3′ end of the passenger strand (sense strand) of each siRNA is conjugated with a triantennary GalNAc moiety (X2-GalNAc as in Formula (V′)). Selected siRNAs were administered at a single subcutaneous dose level of 1 mg/kg on day 0. Serum hANGPTL3 protein levels were measured by ELISA (R&D Systems, cat #DANL30) on Days −4 (predose) and days 7, 14, 21, 28,and 35. For each individual, the % change in serum ANGPTL3 relative to the PBS group mean per timepoint was calculated. The average % change in serum hANGPTL3 protein levels relative to the PBS group and standard error are reported in Table 13. Results are plotted and shown in FIG. 7.

While preferred aspects of the present disclosure have been shown and described herein, it will be obvious to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

TABLE 1
siRNAs Evaluated at 10 and 0.5 μM in vitro in primary human hepatocytes

| Duplex | Target | SEQ ID | Guide/Antisense Structure | SEQ ID | Guide/Antisense Base | SEQ ID | Passenger/Sense | SEQ ID | Passenger/Sense Base |
|---|---|---|---|---|---|---|---|---|---|
| ID | Position | NO. | Code (5′-3′) | NO. | Sequence (5′-3′) | NO. | Structure Code (5′-3′) | NO. | Sequence (5′-3′) |
| SRS- | 44 | 201 | usGfscuuaAfuuguGfaAfcAf | 1 | UGCUUAAUUGUGAACAU | 601 | asasaaauGfuUfcAfcaau | 401 | AAAAAUGUUCACAAU |
| 000362 | | | uuuuusasu | | UUUUAU | | uaagca | | UAAGCA |
| SRS- | 47 | 202 | usGfsgagcUfuaauUfgUfgAf | 2 | UGGAGCUUAAUUGUGAA | 602 | asasuguuCfaCfaAfuuaa | 402 | AAUGUUCACAAUUAA |
| 000363 | | | acauususu | | CAUUUU | | gcucca | | GCUCCA |
| SRS- | 49 | 203 | usAfsaggaGfcuuaAfuUfgUf | 3 | UAAGGAGCUUAAUUGUG | 603 | usgsuucaCfaAfuUfaagc | 403 | UGUUCACAAUUAAGC |
| 000364 | | | gaacasusu | | AACAUU | | uccuua | | UCCUUA |
| SRS- | 52 | 204 | usAfsagaaGfgagcUfuAfaUf | 4 | UAAGAAGGAGCUUAAUU | 604 | uscsacaaUfuAfaGfcucc | 404 | UCACAAUUAAGCUCCU |
| 000365 | | | ugugasasc | | GUGAAC | | uucuua | | UCUUA |
| SRS- | 56 | 205 | usUfsaaaaAfgaagGfaGfcUf | 5 | UUAAAAAGAAGGAGCUU | 605 | asasuuaaGfcUfcCfuucu | 405 | AAUUAAGCUCCUUCUU |
| 000366 | | | uaauusgsu | | AAUUGU | | uuuuaa | | UUUAA |
| SRS- | 57 | 206 | usAfsuaaaAfagaaGfgAfgCf | 6 | UAUAAAAAGAAGGAGCU | 606 | asusuaagCfuCfcUfucuu | 406 | AUUAAGCUCCUUCUUU |
| 000367 | | | uuaaususg | | UAAUUG | | uuuaua | | UUAUA |
| SRS- | 71 | 207 | usUfsaacuAfgaggAfaCfaAf | 7 | UUAACUAGAGGAACAAU | 607 | ususuuauUfgUfuCfcuc | 407 | UUUUAUUGUUCCUCU |
| 000368 | | | uaaaasasg | | AAAAAG | | uaguuaa | | AGUUAA |
| SRS- | 72 | 208 | usAfsuaacUfagagGfaAfcAf | 8 | UAUAACUAGAGGAACAA | 608 | ususuauuGfuUfcCfucu | 408 | UUUAUUGUUCCUCUA |
| 000369 | | | auaaasasa | | UAAAAA | | aguuaua | | GUUAUA |
| SRS- | 76 | 209 | usGfsgaaaUfaacuAfgAfgGf | 9 | UGGAAAUAACUAGAGGA | 609 | ususguucCfuCfuAfguu | 409 | UUGUUCCUCUAGUUA |
| 000370 | | | aacaasusa | | ACAAUA | | auuucca | | UUUCCA |
| SRS- | 149 | 210 | usCfsuaacAfuagcAfaAfuCf | 10 | UCUAACAUAGCAAAUCU | 610 | asuscaag AfuUfuGfcua | 410 | AUCAAGAUUUGCUAU |
| 000371 | | | uugaususu | | UGAUUU | | uguuaga | | GUUAGA |
| SRS- | 151 | 211 | usGfsucuaAfcauaGfcAfaAf | 11 | UGUCUAACAUAGCAAAU | 611 | csasagauUfuGfcUfaug | 411 | CAAGAUUUGCUAUGU |
| 000372 | | | ucuugsasu | | CUUGAU | | uuagaca | | UAGACA |
| SRS- | 152 | 212 | usCfsgucuAfacauAfgCfaAf | 12 | UCGUCUAACAUAGCAAA | 612 | asasgauuUfgCfuAfugu | 412 | AAGAUUUGCUAUGUU |
| 000373 | | | aucuusgsa | | UCUUGA | | uagacga | | AGACGA |
| SRS- | 153 | 213 | usUfscgucUfaacaUfaGfcAf | 13 | UUCGUCUAACAUAGCAA | 613 | asgsauuuGfcUfaUfguu | 413 | AGAUUUGCUAUGUUA |
| 000374 | | | aaucususg | | AUCUUG | | agacgaa | | GACGAA |
| SRS- | 154 | 214 | usAfsucguCfuaacAfuAfgCf | 14 | UAUCGUCUAACAUAGCA | 614 | gsasuuugCfuAfuGfuua | 414 | GAUUUGCUAUGUUAG |
| 000375 | | | aaaucsusu | | AAUCUU | | gacgaua | | ACGAUA |
| SRS- | 155 | 215 | usCfsaucgUfcuaaCfaUfaGf | 15 | UCAUCGUCUAACAUAGC | 615 | asusuugc UfaUfgUfuag | 415 | AUUUGCUAUGUUAGA |
| 000376 | | | caaauscsu | | AAAUCU | | acgauga | | CGAUGA |
| SRS- | 156 | 216 | usAfscaucGfucuaAfcAfuAf | 16 | UACAUCGUCUAACAUAG | 616 | ususugcuAfuGfuUfaga | 416 | UUUGCUAUGUUAGAC |
| 000377 | | | gcaaasusc | | CAAAUC | | cgaugua | | GAUGUA |
| SRS- | 157 | 217 | usUfsacauCfgucuAfaCfaUf | 17 | UUACAUCGUCUAACAUA | 617 | ususgcuaUfgUfuAfgac | 417 | UUGCUAUGUUAGACG |
| 000378 | | | agcaasasu | | GCAAAU | | gauguaa | | AUGUAA |
| SRS- | 158 | 218 | usUfsuuacAfucguCfuAfaCf | 18 | UUUUACAUCGUCUAACA | 618 | gscsuaugUfuAfgAfcga | 418 | GCUAUGUUAGACGAU |
| 000379 | | | auagcsasa | | UAGCAA | | uguaaaa | | GUAAAA |
| SRS- | 160 | 219 | usUfsuuuaCfaucgUfcUfaAf | 19 | UUUUUACAUCGUCUAAC | 619 | csusauguUfaGfaCfgau | 419 | CUAUGUUAGACGAUG |
| 000380 | | | cauagscsa | | AUAGCA | | guaaaaa | | UAAAAA |
| SRS- | 161 | 220 | usUfsuuuuAfcaucGfuCfuAf | 20 | UUUUUUACAUCGUCUAA | 620 | usasuguuAfgAfcGfaug | 420 | UAUGUUAGACGAUGU |
| 000381 | | | acauasgsc | | CAUAGC | | uaaaaaa | | AAAAAA |
| SRS- | 163 | 221 | usAfsauuuUfuacaUfcGfuCf | 21 | UAAUUUUUACAUCGUCU | 621 | usgsuuagAfcGfaUfgua | 421 | UGUUAGACGAUGUAA |
| 000382 | | | uaacasusa | | AACAUA | | aaaauua | | AAAUUA |
| SRS- | 165 | 222 | usAfsaaauUfuuuaCfaUfcGf | 22 | UAAAAUUUUUACAUCGU | 622 | ususagac GfaUfgUfaaaa | 422 | UUAGACGAUGUAAAA |
| 000383 | | | ucuaascsa | | CUAACA | | auuuua | | AUUUUA |
| SRS- | 178 | 223 | usGfsaggcCfauugGfcUfaAf | 23 | UGAGGCCAUUGGCUAAA | 623 | asasauuuUfaGfcCfaaug | 423 | AAAUUUUAGCCAAUG |
| 000384 | | | | | AUUUUU | | gccuca | | GCCUCA |
| SRS- | 179 | 224 | usGfsgaggCfcauuGfgCfuAf | 24 | UGGAGGCCAUUGGCUAA | 624 | asasuuuuAfgCfcAfaug | 424 | AAUUUUAGCCAAUGG |
| 000385 | | | aaauususu | | AAUUUU | | gccucca | | CCUCCA |
| SRS- | 183 | 225 | usUfsgaagGfaggcCfaUfuGf | 25 | UUGAAGGAGGCCAUUGG | 625 | ususagccAfaUfgGfccuc | 425 | UUAGCCAAUGGCCUCC |
| 000386 | | | gcuaasasa | | CUAAAA | | cuucaa | | UUCAA |
| SRS- | 187 | 226 | usCfsaacuGfaaggAfgGfcCf | 26 | UCAACUGAAGGAGGCCA | 626 | cscsaaugGfcCfuCfcuuc | 426 | CCAAUGGCCUCCUUCA |
| 000387 | | | auuggscsu | | UUGGCU | | aguuga | | GUUGA |
| SRS- | 188 | 227 | usCfscaacUfgaagGfaGfgCf | 27 | UCCAACUGAAGGAGGCC | 627 | csasauggCfcUfcCfuuca | 427 | CAAUGGCCUCCUUCAG |
| 000388 | | | cauugsgsc | | AUUGGC | | guugga | | UUGGA |
| SRS- | 189 | 228 | usCfsccaaCfugaaGfgAfgGf | 28 | UCCCAACUGAAGGAGGC | 628 | asasuggcCfuCfcUfucag | 428 | AAUGGCCUCCUUCAGU |
| 000389 | | | ccauusgsg | | CAUUGG | | uuggga | | UGGGA |
| SRS- | 195 | 229 | usCfscaugUfcccaAfcUfgAf | 29 | UCCAUGUCCCAACUGAA | 629 | csusccuuCfaGfuUfggg | 429 | CUCCUUCAGUUGGGAC |
| 000390 | | | aggagsgsc | | GGAGGC | | acaugga | | AUGGA |
| SRS- | 198 | 230 | usAfsgaccAfugucCfcAfaCf | 30 | UAGACCAUGUCCCAACU | 630 | csusucag UfuGfgGfaca | 430 | CUUCAGUUGGGACAU |
| 000391 | | | ugaagsgsa | | GAAGGA | | uggucua | | GGUCUA |
| SRS- | 202 | 231 | usUfsuuaaGfaccaUfgUfcCf | 31 | UUUUAAGACCAUGUCCC | 631 | asgsuuggGfaCfaUfggu | 431 | AGUUGGGACAUGGUC |
| 000392 | | | caacusgsa | | AACUGA | | cuuaaaa | | UUAAAA |
| SRS- | 204 | 232 | usUfscuuuAfagacCfaUfgUf | 32 | UUCUUUAAGACCAUGUC | 632 | ususgggaCfaUfgGfucu | 432 | UUGGGACAUGGUCUU |
| 000393 | | | cccaascsu | | CCAACU | | uaaagaa | | AAAGAA |
| SRS- | 206 | 233 | usAfsgucuUfuaagAfcCfaUf | 33 | UAGUCUUUAAGACCAUG | 633 | gsgsgacaUfgGfuCfuua | 433 | GGGACAUGGUCUUAA |
| 000394 | | | gucccsasa | | UCCCAA | | aagacua | | AGACUA |
| SRS- | 207 | 234 | usAfsaguc UfuuaaGfaCfcAf | 34 | UAAGUCUUUAAGACCAU | 634 | gsgsacauGfgUfcUfuaaa | 434 | GGACAUGGUCUUAAA |
| 000395 | | | uguccscsa | | GUCCCA | | gacuua | | GACUUA |
| SRS- | 208 | 235 | usAfsaaguCfuuuaAfgAfcCf | 35 | UAAAGUCUUUAAGACCA | 635 | gsascaugGfuCfuUfaaag | 435 | GACAUGGUCUUAAAG |
| 000396 | | | augucscsc | | UGUCCC | | acuuua | | ACUUUA |
| SRS- | 209 | 236 | usCfsaaag UfcuuuAfaGfaCf | 36 | UCAAAGUCUUUAAGACC | 636 | ascsauggUfcUfuAfaaga | 436 | ACAUGGUCUUAAAGA |
| 000397 | | | cauguscsc | | AUGUCC | | cuuuga | | CUUUGA |
| SRS- | 210 | 237 | usAfscaaaGfucuuUfaAfgAf | 37 | UACAAAGUCUUUAAGAC | 637 | csasugguCfuUfaAfagac | 437 | CAUGGUCUUAAAGAC |
| 000398 | | | ccaugsusc | | CAUGUC | | uuugua | | UUUGUA |
| SRS- | 217 | 238 | usCfsuuauGfgacaAfaGfuCf | 38 | UCUUAUGGACAAAGUCU | 638 | ususaaagAfcUfuUfguc | 438 | UUAAAGACUUUGUCC |
| 000399 | | | uuuaasgsa | | UUAAGA | | cauaaga | | AUAAGA |
| SRS- | 237 | 239 | usUfscauuAfauuuGfgCfcCf | 39 | UUCAUUAAUUUGGCCCU | 639 | ascsgaagGfgCfcAfaauu | 439 | ACGAAGGGCCAAAUU |
| 000400 | | | uucguscsu | | UCGUCU | | aaugaa | | AAUGAA |
| SRS- | 269 | 240 | usAfscugaUfcaaaUfaUfgUf | 40 | UACUGAUCAAAUAUGUU | 640 | ascsucaaCfaUfaUfuuga | 440 | ACUCAACAUAUUUGA |
| 000401 | | | ugagususu | | GAGUUU | | ucagua | | UCAGUA |
| SRS- | 274 | 241 | usAfsaaagAfcugaUfcAfaAf | 41 | UAAAAGACUGAUCAAAU | 641 | ascsauauUfuGfaUfcagu | 441 | ACAUAUUUGAUCAGU |
| 000402 | | | uaugususg | | AUGUUG | | cuuuua | | CUUUUA |
| SRS- | 282 | 242 | usAfsgaucAfuaaaAfaGfaCf | 42 | UAGAUCAUAAAAAGACU | 642 | gsasucag UfcUfuUfuua | 442 | GAUCAGUCUUUUUAU |
| 000403 | | | ugaucsasa | | GAUCAA | | ugaucua | | GAUCUA |
| SRS- | 283 | 243 | usUfsagauCfauaaAfaAfgAf | 43 | UUAGAUCAUAAAAAGAC | 643 | asuscaguCfuUfuUfuau | 443 | AUCAGUCUUUUUAUG |
| 000404 | | | cugauscsa | | UGAUCA | | gaucuaa | | AUCUAA |
| SRS- | 354 | 244 | usUfsugacUfuguaGfuUfuAf | 44 | UUUGACUUGUAGUUUAU | 644 | ascsauauAfaAfcUfacaa | 444 | ACAUAUAAACUACAA |
| 000405 | | | uaugusasg | | AUGUAG | | gucaaa | | GUCAAA |
| SRS- | 396 | 245 | usUfsuugaGfuugaGfuUfcAf | 45 | UUUUGAGUUGAGUUCAA | 645 | uscsacuuGfaAfcUfcaac | 445 | UCACUUGAACUCAACU |
| 000406 | | | agugascsa | | GUGACA | | ucaaaa | | CAAAA |
| SRS- | 412 | 246 | usUfsaggaGfgcuuUfcAfaGf | 46 | UUAGGAGGCUUUCAAGU | 646 | csasaaacUfuGfaAfagcc | 446 | CAAAACUUGAAAGCCU |
| 000407 | | | uuuugsasg | | UUUGAG | | uccuaa | | CCUAA |
| SRS- | 462 | 247 | usAfsguugCfucuuCfuAfaAf | 47 | UAGUUGCUCUUCUAAAU | 647 | asasauauUfuAfgAfaga | 447 | AAAUAUUUAGAAGAG |
| 000408 | | | uauuuscsa | | AUUUCA | | gcaacua | | CAACUA |
| SRS- | 470 | 248 | usAfsguuaGfuuagUfuGfcUf | 48 | UAGUUAGUUAGUUGCUC | 648 | asgsaagaGfcAfaCfuaac | 448 | AGAAGAGCAACUAAC |
| 000409 | | | cuucusasa | | UUCUAA | | uaacua | | UAACUA |
| SRS- | 472 | 249 | usUfsaaguUfaguuAfgUfuGf | 49 | UUAAGUUAGUUAGUUGC | 649 | asasgagc AfaCfuAfacua | 449 | AAGAGCAACUAACUA |
| 000410 | | | cucuuscsu | | UCUUCU | | acuuaa | | ACUUAA |
| SRS- | 473 | 250 | usUfsuaagUfuaguUfaGfuUf | 50 | UUUAAGUUAGUUAGUUG | 650 | asgsagcaAfcUfaAfcuaa | 450 | AGAGCAACUAACUAAC |
| 000411 | | | gcucususc | | CUCUUC | | cuuaaa | | UUAAA |
| SRS- | 474 | 251 | usAfsuuaaGfuuagUfuAfgUf | 51 | UAUUAAGUUAGUUAGUU | 651 | gsasgcaaCfuAfaCfuaac | 451 | GAGCAACUAACUAACU |
| 000412 | | | ugcucsusu | | GCUCUU | | uuaaua | | UAAUA |
| SRS- | 485 | 252 | usGfsuugaUfuuugAfaUfuAf | 52 | UGUUGAUUUUGAAUUAA | 652 | usasacuuAfaUfuCfaaaa | 452 | UAACUUAAUUCAAAA |
| 000413 | | | aguuasgsu | | GUUAGU | | ucaaca | | UCAACA |
| SRS- | 520 | 253 | usAfsagugAfaguuAfcUfuCf | 53 | UAAGUGAAGUUACUUCU | 653 | ascsccagAfaGfuAfacuu | 453 | ACCCAGAAGUAACUUC |
| 000414 | | | ugggusgsu | | GGGUGU | | cacuua | | ACUUA |
| SRS- | 521 | 254 | usUfsaaguGfaaguUfaCfuUf | 54 | UUAAGUGAAGUUACUUC | 654 | cscscagaAfgUfaAfcuuc | 454 | CCCAGAAGUAACUUCA |
| 000415 | | | cugggsusg | | UGGGUG | | acuuaa | | CUUAA |
| SRS- | 553 | 255 | usGfsaugcUfauuaUfcUfuGf | 55 | UGAUGCUAUUAUCUUGU | 655 | asasaaacAfaGfaUfaaua | 455 | AAAAACAAGAUAAUA |
| 000416 | | | | | UUUUCU | | gcauca | | GCAUCA |
| SRS- | 555 | 256 | usUfsugauGfcuauUfaUfcUf | 56 | UUUGAUGCUAUUAUCUU | 656 | asasacaaGfaUfaAfuagc | 456 | AAACAAGAUAAUAGC |
| 000417 | | | uguuususu | | GUUUUU | | aucaaa | | AUCAAA |
| SRS- | 560 | 257 | usGfsgucuUfugauGfcUfaUf | 57 | UGGUCUUUGAUGCUAUU | 657 | asgsauaaUfaGfcAfucaa | 457 | AGAUAAUAGCAUCAA |
| 000418 | | | uaucususg | | AUCUUG | | agacca | | AGACCA |
| SRS- | 562 | 258 | usAfsagguCfuuugAfuGfcUf | 58 | UAAGGUCUUUGAUGCUA | 658 | asusaauaGfcAfuCfaaag | 458 | AUAAUAGCAUCAAAG |
| 000419 | | | auuauscsu | | UUAUCU | | accuua | | ACCUUA |
| SRS- | 609 | 259 | usCfsuaugCfuguuGfgUfuUf | 59 | UCUAUGCUGUUGGUUUA | 659 | csasauuaAfaCfcAfacag | 459 | CAAUUAAACCAACAGC |
| 000420 | | | aauugsusu | | AUUGUU | | cauaga | | AUAGA |
| SRS- | 615 | 260 | usAfsuuugAfcuauGfcUfgUf | 60 | UAUUUGACUAUGCUGUU | 660 | asasccaaCfaGfcAfuagu | 460 | AACCAACAGCAUAGUC |
| 000421 | | | ugguususa | | GGUUUA | | caaaua | | AAAUA |
| SRS- | 703 | 261 | usAfsguucUfugguGfcUfcUf | 61 | UAGUUCUUGGUGCUCUU | 661 | asgsccaaGfaGfcAfccaa | 461 | AGCCAAGAGCACCAAG |
| 000422 | | | uggcususg | | GGCUUG | | gaacua | | AACUA |
| SRS- | 832 | 262 | usUfsugagAfguugCfuGfgGf | 62 | UUUGAGAGUUGCUGGGU | 662 | uscsagacCfcAfgCfaacu | 462 | UCAGACCCAGCAACUC |
| 000423 | | | ucugasusg | | CUGAUG | | cucaaa | | UCAAA |
| SRS- | 833 | 263 | usCfsuugaGfaguuGfcUfgGf | 63 | UCUUGAGAGUUGCUGGG | 663 | csasgaccCfaGfcAfacuc | 463 | CAGACCCAGCAACUCU |
| 000424 | | | gucugsasu | | UCUGAU | | ucaaga | | CAAGA |
| SRS- | 834 | 264 | usAfscuugAfgaguUfgCfuGf | 64 | UACUUGAGAGUUGCUGG | 664 | asgsacccAfgCfaAfcucu | 464 | AGACCCAGCAACUCUC |
| 000425 | | | ggucusgsa | | GUCUGA | | caagua | | AAGUA |
| SRS- | 840 | 265 | usUfsgaaaAfacuuGfaGfaGf | 65 | UUGAAAAACUUGAGAGU | 665 | asgscaacUfcUfcAfaguu | 465 | AGCAACUCUCAAGUUU |
| 000426 | | | uugcusgsg | | UGCUGG | | uuucaa | | UUCAA |
| SRS- | 874 | 266 | usCfscaugGfacuaCfcUfgAf | 66 | UCCAUGGACUACCUGAU | 666 | ususauauCfaGfgUfagu | 466 | UUAUAUCAGGUAGUC |
| 000427 | | | uauaascsa | | AUAACA | | ccaugga | | CAUGGA |
| SRS- | 877 | 267 | usUfsguccAfuggaCfuAfcCf | 67 | UUGUCCAUGGACUACCU | 667 | usasucag GfuAfgUfcca | 467 | UAUCAGGUAGUCCAU |
| 000428 | | | ugauasusa | | GAUAUA | | uggacaa | | GGACAA |
| SRS- | 878 | 268 | usAfsugucCfauggAfcUfaCf | 68 | UAUGUCCAUGGACUACC | 668 | asuscaggUfaGfuCfcaug | 468 | AUCAGGUAGUCCAUG |
| 000429 | | | cugausasu | | UGAUAU | | gacaua | | GACAUA |
| SRS- | 879 | 269 | usAfsauguCfcaugGfaCfuAf | 69 | UAAUGUCCAUGGACUAC | 669 | uscsagguAfgUfcCfaug | 469 | UCAGGUAGUCCAUGG |
| 000430 | | | ccugasusa | | CUGAUA | | gacauua | | ACAUUA |
| SRS- | 885 | 270 | usUfsgaauUfaaugUfcCfaUf | 70 | UUGAAUUAAUGUCCAUG | 670 | asgsuccaUfgGfaCfauua | 470 | AGUCCAUGGACAUUA |
| 000431 | | | ggacusasc | | GACUAC | | auucaa | | AUUCAA |
| SRS- | 886 | 271 | usUfsugaaUfuaauGfuCfcAf | 71 | UUUGAAUUAAUGUCCAU | 671 | gsusccauGfgAfcAfuua | 471 | GUCCAUGGACAUUAA |
| 000432 | | | uggacsusa | | GGACUA | | auucaaa | | UUCAAA |
| SRS- | 887 | 272 | usGfsuugaAfuuaaUfgUfcCf | 72 | UGUUGAAUUAAUGUCCA | 672 | uscscaugGfaCfaUfuaau | 472 | UCCAUGGACAUUAAU |
| 000238 | | | auggascsu | | UGGACU | | ucaaca | | UCAACA |
| SRS- | 892 | 273 | usUfscgauGfuugaAfuUfaAf | 73 | UUCGAUGUUGAAUUAAU | 673 | gsgsacauUfaAfuUfcaac | 473 | GGACAUUAAUUCAAC |
| 000433 | | | uguccsasu | | GUCCAU | | aucgaa | | AUCGAA |
| SRS- | 894 | 274 | usAfsuucgAfuguuGfaAfuUf | 74 | UAUUCGAUGUUGAAUUA | 674 | ascsauuaAfuUfcAfacau | 474 | ACAUUAAUUCAACAUC |
| 000434 | | | aauguscsc | | AUGUCC | | cgaaua | | GAAUA |
| SRS- | 895 | 275 | usUfsauucGfauguUfgAfaUf | 75 | UUAUUCGAUGUUGAAUU | 675 | csasuuaaUfuCfaAfcauc | 475 | CAUUAAUUCAACAUCG |
| 000435 | | | uaaugsusc | | AAUGUC | | gaauaa | | AAUAA |
| SRS- | 897 | 276 | usUfscuauUfcgauGfuUfgAf | 76 | UUCUAUUCGAUGUUGAA | 676 | ususaauuCfaAfcAfucga | 476 | UUAAUUCAACAUCGA |
| 000436 | | | auuaasusg | | UUAAUG | | auagaa | | AUAGAA |
| SRS- | 898 | 277 | usAfsucuaUfucgaUfgUfuGf | 77 | UAUCUAUUCGAUGUUGA | 677 | usasauuc AfaCfaUfcgaa | 477 | UAAUUCAACAUCGAA |
| 000437 | | | aauuasasu | | AUUAAU | | uagaua | | UAGAUA |
| SRS- | 899 | 278 | usCfsaucuAfuucgAfuGfuUf | 78 | UCAUCUAUUCGAUGUUG | 678 | asasuucaAfcAfuCfgaau | 478 | AAUUCAACAUCGAAU |
| 000438 | | | gaauusasa | | AAUUAA | | agauga | | AGAUGA |
| SRS- | 902 | 279 | usAfsuccaUfcuauUfcGfaUf | 79 | UAUCCAUCUAUUCGAUG | 679 | uscsaacaUfcGfaAfuaga | 479 | UCAACAUCGAAUAGA |
| 000439 | | | guugasasu | | UUGAAU | | uggaua | | UGGAUA |
| SRS- | 903 | 280 | usGfsaucc AfucuaUfuCfgAf | 80 | UGAUCCAUCUAUUCGAU | 680 | csasacauCfgAfaUfagau | 480 | CAACAUCGAAUAGAU |
| 000440 | | | uguugsasa | | GUUGAA | | ggauca | | GGAUCA |
| SRS- | 904 | 281 | usUfsgaucCfaucuAfuUfcGf | 81 | UUGAUCCAUCUAUUCGA | 681 | asascaucGfaAfuAfgaug | 481 | AACAUCGAAUAGAUG |
| 000441 | | | auguusgsa | | UGUUGA | | gaucaa | | GAUCAA |
| SRS- | 905 | 282 | usGfsugauCfcaucUfaUfuCf | 82 | UGUGAUCCAUCUAUUCG | 682 | ascsaucgAfaUfaGfaugg | 482 | ACAUCGAAUAGAUGG |
| 000442 | | | gaugususg | | AUGUUG | | aucaca | | AUCACA |
| SRS- | 908 | 283 | usUfsuuguGfauccAfuCfuAf | 83 | UUUUGUGAUCCAUCUAU | 683 | uscsgaauAfgAfuGfgau | 483 | UCGAAUAGAUGGAUC |
| 000443 | | | uucgasusg | | UCGAUG | | cacaaaa | | ACAAAA |
| SRS- | 912 | 284 | usAfsaguuUfugugAfuCfcAf | 84 | UAAGUUUUGUGAUCCAU | 684 | asusagauGfgAfuCfacaa | 484 | AUAGAUGGAUCACAA |
| 000444 | | | ucuaususc | | CUAUUC | | aacuua | | AACUUA |
| SRS- | 915 | 285 | usUfsugaaGfuuuuGfuGfaUf | 85 | UUUGAAGUUUUGUGAUC | 685 | gsasuggaUfcAfcAfaaac | 485 | GAUGGAUCACAAAAC |
| 000445 | | | ccaucsusa | | CAUCUA | | uucaaa | | UUCAAA |
| SRS- | 923 | 286 | usAfscguuUfcauuGfaAfgUf | 86 | UACGUUUCAUUGAAGUU | 686 | ascsaaaaCfuUfcAfauga | 486 | ACAAAACUUCAAUGA |
| 000446 | | | uuugusgsa | | UUGUGA | | aacgua | | AACGUA |
| SRS- | 925 | 287 | usCfscacgUfuucaUfuGfaAf | 87 | UCCACGUUUCAUUGAAG | 687 | asasaacuUfcAfaUfgaaa | 487 | AAAACUUCAAUGAAA |
| 000447 | | | guuuusgsu | | UUUUGU | | cgugga | | CGUGGA |
| SRS- | 926 | 288 | usCfsccacGfuuucAfuUfgAf | 88 | UCCCACGUUUCAUUGAA | 688 | asasacuuCfaAfuGfaaac | 488 | AAACUUCAAUGAAAC |
| 000448 | | | aguuususg | | GUUUUG | | guggga | | GUGGGA |
| SRS- | 929 | 289 | usUfscuccCfacguUfuCfaUf | 89 | UUCUCCCACGUUUCAUU | 689 | csusucaaUfgAfaAfcgu | 489 | CUUCAAUGAAACGUG |
| 000449 | | | ugaagsusu | | GAAGUU | | gggagaa | | GGAGAA |
| SRS- | 930 | 290 | usUfsucucCfcacgUfuUfcAf | 90 | UUUCUCCCACGUUUCAU | 690 | ususcaauGfaAfaCfgug | 490 | UUCAAUGAAACGUGG |
| 000450 | | | uugaasgsu | | UGAAGU | | ggagaaa | | GAGAAA |
| SRS- | 931 | 291 | usGfsuucuCfccacGfuUfuCf | 91 | UGUUCUCCCACGUUUCA | 691 | uscsaaugAfaAfcGfugg | 491 | UCAAUGAAACGUGGG |
| 000451 | | | auugasasg | | UUGAAG | | gagaaca | | AGAACA |
| SRS- | 933 | 292 | usUfsaguuCfucccAfcGfuUf | 92 | UUAGUUCUCCCACGUUU | 692 | asasugaaAfcGfuGfgga | 492 | AAUGAAACGUGGGAG |
| 000452 | | | ucauusgsa | | CAUUGA | | gaacuaa | | AACUAA |
| SRS- | 934 | 293 | usGfsuaguUfcuccCfaCfgUf | 93 | UGUAGUUCUCCCACGUU | 693 | asusgaaaCfgUfgGfgaga | 493 | AUGAAACGUGGGAGA |
| 000453 | | | uucaususg | | UCAUUG | | acuaca | | ACUACA |
| SRS- | 936 | 294 | usUfsuguaGfuucuCfcCfaCf | 94 | UUUGUAGUUCUCCCACG | 694 | gsasaacgUfgGfgAfgaac | 494 | GAAACGUGGGAGAAC |
| 000454 | | | guuucsasu | | UUUCAU | | uacaaa | | UACAAA |
| SRS- | 937 | 295 | usUfsuuguAfguucUfcCfcAf | 95 | UUUUGUAGUUCUCCCAC | 695 | asasacguGfgGfaGfaacu | 495 | AAACGUGGGAGAACU |
| 000455 | | | cguuuscsa | | GUUUCA | | acaaaa | | ACAAAA |
| SRS- | 942 | 296 | usCfscauaUfuuguAfgUfuCf | 96 | UCCAUAUUUGUAGUUCU | 696 | usgsggagAfaCfuAfcaaa | 496 | UGGGAGAACUACAAA |
| 000456 | | | ucccascsg | | CCCACG | | uaugga | | UAUGGA |
| SRS- | 965 | 297 | usAfsuucuCfcaucAfaGfcCf | 97 | UAUUCUCCAUCAAGCCU | 697 | usgsggagGfcUfuGfaug | 497 | UGGGAGGCUUGAUGG |
| 000457 | | | ucccasasa | | CCCAAA | | gagaaua | | AGAAUA |
| SRS- | 966 | 298 | usAfsauuc UfccauCfaAfgCf | 98 | UAAUUCUCCAUCAAGCC | 698 | gsgsgaggCfuUfgAfugg | 498 | GGGAGGCUUGAUGGA |
| 000458 | | | cucccsasa | | UCCCAA | | agaauua | | GAAUUA |
| SRS- | 992 | 299 | usAfsguauAfucuuCfuCfuAf | 99 | UAGUAUAUCUUCUCUAG | 699 | gsgsgccuAfgAfgAfaga | 499 | GGGCCUAGAGAAGAU |
| 000459 | | | ggcccsasa | | GCCCAA | | uauacua | | AUACUA |
| SRS- | 994 | 300 | usGfsgaguAfuaucUfuCfuCf | 100 | UGGAGUAUAUCUUCUCU | 700 | gscscuagAfgAfaGfaua | 500 | GCCUAGAGAAGAUAU |
| 000460 | | | uaggcscsc | | AGGCCC | | uacucca | | ACUCCA |
| SRS- | 998 | 301 | usCfsuaugGfaguaUfaUfcUf | 101 | UCUAUGGAGUAUAUCUU | 701 | asgsagaaGfaUfaUfacuc | 501 | AGAGAAGAUAUACUC |
| 000461 | | | ucucusasg | | CUCUAG | | cauaga | | CAUAGA |
| SRS- | 999 | 302 | usAfscuauGfgaguAfuAfuCf | 102 | UACUAUGGAGUAUAUCU | 702 | gsasgaagAfuAfuAfcuc | 502 | GAGAAGAUAUACUCC |
| 000462 | | | uucucsusa | | UCUCUA | | cauagua | | AUAGUA |
| SRS- | 1000 | 303 | usCfsacuaUfggagUfaUfaUf | 103 | UCACUAUGGAGUAUAUC | 703 | asgsaagaUfaUfaCfucca | 503 | AGAAGAUAUACUCCA |
| 000463 | | | cuucuscsu | | UUCUCU | | uaguga | | UAGUGA |
| SRS- | 1001 | 304 | usUfscacuAfuggaGfuAfuAf | 104 | UUCACUAUGGAGUAUAU | 704 | gsasagauAfuAfcUfccau | 504 | GAAGAUAUACUCCAU |
| 000464 | | | ucuucsusc | | CUUCUC | | agugaa | | AGUGAA |
| SRS- | 1002 | 305 | usUfsucacUfauggAfgUfaUf | 105 | UUUCACUAUGGAGUAUA | 705 | asasgauaUfaCfuCfcaua | 505 | AAGAUAUACUCCAUA |
| 000465 | | | aucuuscsu | | UCUUCU | | gugaaa | | GUGAAA |
| SRS- | 1006 | 306 | usUfsugcuUfcacuAfuGfgAf | 106 | UUUGCUUCACUAUGGAG | 706 | usasuacuCfcAfuAfgug | 506 | UAUACUCCAUAGUGA |
| 000466 | | | guauasusc | | UAUAUC | | aagcaaa | | AGCAAA |
| SRS- | 1007 | 307 | usAfsuugc UfucacUfaUfgGf | 107 | UAUUGCUUCACUAUGGA | 707 | asusacucCfaUfaGfugaa | 507 | AUACUCCAUAGUGAA |
| 000467 | | | aguausasu | | GUAUAU | | gcaaua | | GCAAUA |
| SRS- | 1012 | 308 | usAfsuuagAfuugcUfuCfaCf | 108 | UAUUAGAUUGCUUCACU | 708 | cscsauagUfgAfaGfcaau | 508 | CCAUAGUGAAGCAAUC |
| 000468 | | | uauggsasg | | AUGGAG | | cuaaua | | UAAUA |
| SRS- | 1014 | 309 | usUfsaauuAfgauuGfcUfuCf | 109 | UUAAUUAGAUUGCUUCA | 709 | asusagugAfaGfcAfauc | 509 | AUAGUGAAGCAAUCU |
| 000469 | | | acuausgsg | | CUAUGG | | uaauuaa | | AAUUAA |
| SRS- | 1092 | 310 | usUfscgugAfuuucCfcAfaGf | 110 | UUCGUGAUUUCCCAAGU | 710 | ususuuac UfuGfgGfaaa | 510 | UUUUACUUGGGAAAU |
| 000470 | | | uaaaasasg | | AAAAAG | | ucacgaa | | CACGAA |
| SRS- | 1098 | 311 | usUfsugguUfucguGfaUfuUf | 111 | UUUGGUUUCGUGAUUUC | 711 | ususgggaAfaUfcAfcga | 511 | UUGGGAAAUCACGAA |
| 000471 | | | cccaasgsu | | CCAAGU | | aaccaaa | | ACCAAA |
| SRS- | 1100 | 312 | usAfsguugGfuuucGfuGfaUf | 112 | UAGUUGGUUUCGUGAUU | 712 | gsgsgaaaUfcAfcGfaaac | 512 | GGGAAAUCACGAAACC |
| 000472 | | | uucccsasa | | UCCCAA | | caacua | | AACUA |
| SRS- | 1101 | 313 | usUfsaguuGfguuuCfgUfgAf | 113 | UUAGUUGGUUUCGUGAU | 713 | gsgsaaauCfaCfgAfaacc | 513 | GGAAAUCACGAAACCA |
| 000473 | | | uuuccscsa | | UUCCCA | | aacuaa | | ACUAA |
| SRS- | 1103 | 314 | usUfsauagUfugguUfuCfgUf | 114 | UUAUAGUUGGUUUCGUG | 714 | asasaucaCfgAfaAfccaa | 514 | AAAUCACGAAACCAAC |
| 000474 | | | gauuuscsc | | AUUUCC | | cuauaa | | UAUAA |
| SRS- | 1104 | 315 | usGfsuauaGfuuggUfuUfcGf | 115 | UGUAUAGUUGGUUUCGU | 715 | asasucacGfaAfaCfcaac | 515 | AAUCACGAAACCAACU |
| 000475 | | | ugauususc | | GAUUUC | | uauaca | | AUACA |
| SRS- | 1105 | 316 | usCfsguauAfguugGfuUfuCf | 116 | UCGUAUAGUUGGUUUCG | 716 | asuscacgAfaAfcCfaacu | 516 | AUCACGAAACCAACUA |
| 000476 | | | gugaususu | | UGAUUU | | auacga | | UACGA |
| SRS- | 1106 | 317 | usGfscguaUfaguuGfgUfuUf | 117 | UGCGUAUAGUUGGUUUC | 717 | uscsacgaAfaCfcAfacua | 517 | UCACGAAACCAACUAU |
| 000477 | | | cgugasusu | | GUGAUU | | uacgca | | ACGCA |
| SRS- | 1107 | 318 | usAfsgcguAfuaguUfgGfuUf | 118 | UAGCGUAUAGUUGGUUU | 718 | csascgaaAfcCfaAfcuau | 518 | CACGAAACCAACUAUA |
| 000478 | | | ucgugsasu | | CGUGAU | | acgcua | | CGCUA |
| SRS- | 1110 | 319 | usUfsguagCfguauAfgUfuGf | 119 | UUGUAGCGUAUAGUUGG | 719 | gsasaaccAfaCfuAfuacg | 519 | GAAACCAACUAUACGC |
| 000479 | | | guuucsgsu | | UUUCGU | | cuacaa | | UACAA |
| SRS- | 1136 | 320 | usGfsgacaUfugccAfgUfaAf | 120 | UGGACAUUGCCAGUAAU | 720 | usgscgauUfaCfuGfgcaa | 520 | UGCGAUUACUGGCAA |
| 000480 | | | ucgcasasc | | CGCAAC | | ugucca | | UGUCCA |
| SRS- | 1137 | 321 | usGfsggacAfuugcCfaGfuAf | 121 | UGGGACAUUGCCAGUAA | 721 | gscsgauuAfcUfgGfcaa | 521 | GCGAUUACUGGCAAU |
| 000481 | | | aucgcsasa | | UCGCAA | | uguccca | | GUCCCA |
| SRS- | 1138 | 322 | usGfsgggaCfauugCfcAfgUf | 122 | UGGGGACAUUGCCAGUA | 722 | csgsauuaCfuGfgCfaaug | 522 | CGAUUACUGGCAAUG |
| 000482 | | | aaucgscsa | | AUCGCA | | ucccca | | UCCCCA |
| SRS- | 1139 | 323 | usUfsggggAfcauuGfcCfaGf | 123 | UUGGGGACAUUGCCAGU | 723 | gsasuuacUfgGfcAfaug | 523 | GAUUACUGGCAAUGU |
| 000483 | | | uaaucsgsc | | AAUCGC | | uccccaa | | CCCCAA |
| SRS- | 1141 | 324 | usAfsuuggGfgacaUfuGfcCf | 124 | UAUUGGGGACAUUGCCA | 724 | ususacugGfcAfaUfguc | 524 | UUACUGGCAAUGUCCC |
| 000484 | | | aguaasusc | | GUAAUC | | cocaaua | | CAAUA |
| SRS- | 1147 | 325 | usGfsauugCfauugGfgGfaCf | 125 | UGAUUGCAUUGGGGACA | 725 | gscsaaugUfcCfcCfaaug | 525 | GCAAUGUCCCCAAUGC |
| 000485 | | | auugcscsa | | UUGCCA | | caauca | | AAUCA |
| SRS- | 1148 | 326 | usGfsgauuGfcauuGfgGfgAf | 126 | UGGAUUGCAUUGGGGAC | 726 | csasauguCfcCfcAfaugc | 526 | CAAUGUCCCCAAUGCA |
| 000486 | | | cauugscsc | | AUUGCC | | aaucca | | AUCCA |
| SRS- | 1150 | 327 | usCfsgggaUfugcaUfuGfgGf | 127 | UCGGGAUUGCAUUGGGG | 727 | asusguccCfcAfaUfgcaa | 527 | AUGUCCCCAAUGCAAU |
| 000487 | | | gacaususg | | ACAUUG | | ucccga | | CCCGA |
| SRS- | 1154 | 328 | usUfsuuccGfggauUfgCfaUf | 128 | UUUUCCGGGAUUGCAUU | 728 | cscsccaaUfgCfaAfuccc | 528 | CCCCAAUGCAAUCCCG |
| 000488 | | | uggggsasc | | GGGGAC | | ggaaaa | | GAAAA |
| SRS- | 1157 | 329 | usUfsguuuUfccggGfaUfuGf | 129 | UUGUUUUCCGGGAUUGC | 729 | csasaugcAfaUfcCfcgga | 529 | CAAUGCAAUCCCGGAA |
| 000489 | | | cauugsgsg | | AUUGGG | | aaacaa | | AACAA |
| SRS- | 1158 | 330 | usUfsuguuUfuccgGfgAfuUf | 130 | UUUGUUUUCCGGGAUUG | 730 | asasugcaAfuCfcCfggaa | 530 | AAUGCAAUCCCGGAAA |
| 000490 | | | gcauusgsg | | CAUUGG | | aacaaa | | ACAAA |
| SRS- | 1159 | 331 | usUfsuuguUfuuccGfgGfaUf | 131 | UUUUGUUUUCCGGGAUU | 731 | asusgcaaUfcCfcGfgaaa | 531 | AUGCAAUCCCGGAAAA |
| 000491 | | | ugcaususg | | GCAUUG | | acaaaa | | CAAAA |
| SRS- | 1160 | 332 | usCfsuuugUfuuucCfgGfgAf | 132 | UCUUUGUUUUCCGGGAU | 732 | usgscaauCfcCfgGfaaaa | 532 | UGCAAUCCCGGAAAAC |
| 000492 | | | uugcasusu | | UGCAUU | | caaaga | | AAAGA |
| SRS- | 1161 | 333 | usUfscuuuGfuuuuCfcGfgGf | 133 | UUCUUUGUUUUCCGGGA | 733 | gscsaaucCfcGfgAfaaac | 533 | GCAAUCCCGGAAAACA |
| 000493 | | | auugcsasu | | UUGCAU | | aaagaa | | AAGAA |
| SRS- | 1162 | 334 | usAfsucuuUfguuuUfcCfgGf | 134 | UAUCUUUGUUUUCCGGG | 734 | csasauccCfgGfaAfaaca | 534 | CAAUCCCGGAAAACAA |
| 000494 | | | gauugscsa | | AUUGCA | | aagaua | | AGAUA |
| SRS- | 1179 | 335 | usCfsaaguAfgaaaAfcAfcCf | 135 | UCAAGUAGAAAACACCA | 735 | gsasuuugGfuGfuUfuuc | 535 | GAUUUGGUGUUUUCU |
| 000495 | | | aaaucsusu | | AAUCUU | | uacuuga | | ACUUGA |
| SRS- | 1180 | 336 | usCfscaagUfagaaAfaCfaCfc | 136 | UCCAAGUAGAAAACACC | 736 | asusuuggUfgUfuUfucu | 536 | AUUUGGUGUUUUCUA |
| 000496 | | | aaauscsu | | AAAUCU | | acuugga | | CUUGGA |
| SRS- | 1181 | 337 | usCfsccaaGfuagaAfaAfcAf | 137 | UCCCAAGUAGAAAACAC | 737 | ususugguGfuUfuUfcua | 537 | UUUGGUGUUUUCUAC |
| 000497 | | | ccaaasusc | | CAAAUC | | cuuggga | | UUGGGA |
| SRS- | 1186 | 338 | usGfsugauCfccaaGfuAfgAf | 138 | UGUGAUCCCAAGUAGAA | 738 | usgsuuuuCfuAfcUfugg | 538 | UGUUUUCUACUUGGG |
| 000498 | | | aaacascsc | | AACACC | | gaucaca | | AUCACA |
| SRS- | 1189 | 339 | usUfsuuguGfauccCfaAfgUf | 139 | UUUUGUGAUCCCAAGUA | 739 | ususucuaCfuUfgGfgau | 539 | UUUCUACUUGGGAUC |
| 000499 | | | agaaasasc | | GAAAAC | | cacaaaa | | ACAAAA |
| SRS- | 1190 | 340 | usCfsuuugUfgaucCfcAfaGf | 140 | UCUUUGUGAUCCCAAGU | 740 | ususcuacUfuGfgGfauc | 540 | UUCUACUUGGGAUCAC |
| 000500 | | | uagaasasa | | AGAAAA | | acaaaga | | AAAGA |
| SRS- | 1192 | 341 | usUfsgcuuUfgugaUfcCfcAf | 141 | UUGCUUUGUGAUCCCAA | 741 | csusacuuGfgGfaUfcaca | 541 | CUACUUGGGAUCACAA |
| 000501 | | | aguagsasa | | GUAGAA | | aagcaa | | AGCAA |
| SRS- | 1193 | 342 | usUfsugcuUfugugAfuCfcCf | 142 | UUUGCUUUGUGAUCCCA | 742 | usascuugGfgAfuCfacaa | 542 | UACUUGGGAUCACAA |
| 000502 | | | aaguasgsa | | AGUAGA | | agcaaa | | AGCAAA |
| SRS- | 1287 | 343 | usGfsguuuGfuuauAfuUfuA | 143 | UGGUUUGUUAUAUUUAC | 743 | asasugguAfaAfuAfuaa | 543 | AAUGGUAAAUAUAAC |
| 000503 | | | fccauususa | | CAUUUA | | caaacca | | AAACCA |
| SRS- | 1349 | 344 | usAfsccuuCfcauuUfuGfaGf | 144 | UACCUUCCAUUUUGAGA | 744 | gsasagucUfcAfaAfaug | 544 | GAAGUCUCAAAAUGG |
| 000504 | | | acuucscsa | | CUUCCA | | gaaggua | | AAGGUA |
| SRS- | 1357 | 345 | usAfsgaguAfuaacCfuUfcCf | 145 | UAGAGUAUAACCUUCCA | 745 | asasaaugGfaAfgGfuua | 545 | AAAAUGGAAGGUUAU |
| 000505 | | | auuuusgsa | | UUUUGA | | uacucua | | ACUCUA |
| SRS- | 1358 | 346 | usUfsagagUfauaaCfcUfuCf | 146 | UUAGAGUAUAACCUUCC | 746 | asasauggAfaGfgUfuau | 546 | AAAUGGAAGGUUAUA |
| 000506 | | | cauuususg | | AUUUUG | | acucuaa | | CUCUAA |
| SRS- | 1359 | 347 | usAfsuagaGfuauaAfcCfuUf | 147 | UAUAGAGUAUAACCUUC | 747 | asasuggaAfgGfuUfaua | 547 | AAUGGAAGGUUAUAC |
| 000507 | | | ccauususu | | CAUUUU | | cucuaua | | UCUAUA |
| SRS- | 1363 | 348 | usUfsuuuaUfagagUfaUfaAf | 148 | UUUUUAUAGAGUAUAAC | 748 | gsasagguUfaUfaCfucua | 548 | GAAGGUUAUACUCUA |
| 000508 | | | ccuucscsa | | CUUCCA | | uaaaaa | | UAAAAA |
| SRS- | 1365 | 349 | usGfsauuuUfauagAfgUfaUf | 149 | UGAUUUUAUAGAGUAUA | 749 | asgsguuaUfaCfuCfuaua | 549 | AGGUUAUACUCUAUA |
| 000509 | | | aaccususc | | ACCUUC | | aaauca | | AAAUCA |
| SRS- | 1381 | 350 | usGfsaucaAfcauuUfuGfgUf | 150 | UGAUCAACAUUUUGGUU | 750 | asasucaaCfcAfaAfaugu | 550 | AAUCAACCAAAAUGU |
| 000510 | | | ugauususu | | GAUUUU | | ugauca | | UGAUCA |
| SRS- | 1382 | 351 | usGfsgaucAfacauUfuUfgGf | 151 | UGGAUCAACAUUUUGGU | 751 | asuscaacCfaAfaAfuguu | 551 | AUCAACCAAAAUGUU |
| 000511 | | | uugaususu | | UGAUUU | | gaucca | | GAUCCA |
| SRS- | 1383 | 352 | usUfsggauCfaacaUfuUfuGf | 152 | UUGGAUCAACAUUUUGG | 752 | uscsaaccAfaAfaUfguug | 552 | UCAACCAAAAUGUUG |
| 000512 | | | guugasusu | | UUGAUU | | auccaa | | AUCCAA |
| SRS- | 1384 | 353 | usAfsuggaUfcaacAfuUfuUf | 153 | UAUGGAUCAACAUUUUG | 753 | csasaccaAfaAfuGfuuga | 553 | CAACCAAAAUGUUGA |
| 000513 | | | gguugsasu | | GUUGAU | | uccaua | | UCCAUA |
| SRS- | 1386 | 354 | usGfsgaugGfaucaAfcAfuUf | 154 | UGGAUGGAUCAACAUUU | 754 | ascscaaaAfuGfuUfgauc | 554 | ACCAAAAUGUUGAUCC |
| 000514 | | | uuggususg | | UGGUUG | | caucca | | AUCCA |
| SRS- | 1387 | 355 | usUfsggauGfgaucAfaCfaUf | 155 | UUGGAUGGAUCAACAUU | 755 | cscsaaaaUfgUfuGfaucc | 555 | CCAAAAUGUUGAUCCA |
| 000515 | | | uuuggsusu | | UUGGUU | | auccaa | | UCCAA |
| SRS- | 1467 | 356 | usCfsauuaAfcuugGfaAfuGf | 156 | UCAUUAACUUGGAAUGA | 756 | asasccucAfuUfcCfaagu | 556 | AACCUCAUUCCAAGUU |
| 000516 | | | agguusasa | | GGUUAA | | uaauga | | AAUGA |
| SRS- | 1494 | 357 | usAfsggauUfuaauAfcCfaGf | 157 | UAGGAUUUAAUACCAGA | 757 | asusaaucUfgGfuAfuua | 557 | AUAAUCUGGUAUUAA |
| 000517 | | | auuaususa | | UUAUUA | | aauccua | | AUCCUA |
| SRS- | 1496 | 358 | usUfsaaggAfuuuaAfuAfcCf | 158 | UUAAGGAUUUAAUACCA | 758 | asasucugGfuAfuUfaaa | 558 | AAUCUGGUAUUAAAU |
| 000518 | | | agauusasu | | GAUUAU | | uccuuaa | | CCUUAA |
| SRS- | 1498 | 359 | usCfsuuaaGfgauuUfaAfuAf | 159 | UCUUAAGGAUUUAAUAC | 759 | uscsugguAfuUfaAfauc | 559 | UCUGGUAUUAAAUCC |
| 000519 | | | ccagasusu | | CAGAUU | | cuuaaga | | UUAAGA |
| SRS- | 1563 | 360 | usUfsuguaUfguuuAfaUfcUf | 160 | UUUGUAUGUUUAAUCUU | 760 | asusuuaaGfaUfuAfaaca | 560 | AUUUAAGAUUAAACA |
| 000520 | | | uaaausasg | | AAAUAG | | uacaaa | | UACAAA |
| SRS- | 1566 | 361 | usUfsgauuGfuaugUfuUfaAf | 161 | UUGAUUGUAUGUUUAAU | 761 | usasagauUfaAfaCfauac | 561 | UAAGAUUAAACAUAC |
| 000521 | | | ucuuasasa | | CUUAAA | | aaucaa | | AAUCAA |
| SRS- | 1583 | 362 | usUfsauuc UfuuaaGfgUfuAf | 162 | UUAUUCUUUAAGGUUAU | 762 | uscsacauAfaCfcUfuaaa | 562 | UCACAUAACCUUAAAG |
| 000522 | | | ugugasusu | | GUGAUU | | gaauaa | | AAUAA |
| SRS- | 1654 | 363 | usAfsaauuGfauucCfcAfcAf | 163 | UAAAUUGAUUCCCACAU | 763 | usgsugauGfuGfgGfaau | 563 | UGUGAUGUGGGAAUC |
| 000523 | | | ucacasasa | | CACAAA | | caauuua | | AAUUUA |
| SRS- | 1662 | 364 | usAfsccauCfuaaaAfuUfgAf | 164 | UACCAUCUAAAAUUGAU | 764 | gsgsgaauCfaAfuUfuua | 564 | GGGAAUCAAUUUUAG |
| 000524 | | | uucccsasc | | UCCCAC | | gauggua | | AUGGUA |
| SRS- | 1761 | 365 | usUfsauuaGfcucaUfaUfgAf | 165 | UUAUUAGCUCAUAUGAU | 765 | asgsgcauCfaUfaUfgagc | 565 | AGGCAUCAUAUGAGC |
| 000525 | | | ugccususu | | GCCUUU | | uaauaa | | UAAUAA |
| SRS- | 1765 | 366 | usGfsugauAfuuagCfuCfaUf | 166 | UGUGAUAUUAGCUCAUA | 766 | asuscauaUfgAfgCfuaau | 566 | AUCAUAUGAGCUAAU |
| 000526 | | | augausgsc | | UGAUGC | | aucaca | | AUCACA |
| SRS- | 1766 | 367 | usUfsgugaUfauuaGfcUfcAf | 167 | UUGUGAUAUUAGCUCAU | 767 | uscsauauGfaGfcUfaaua | 567 | UCAUAUGAGCUAAUA |
| 000527 | | | uaugasusg | | AUGAUG | | ucacaa | | UCACAA |
| SRS- | 1816 | 368 | usGfsucaaGfuuuaGfaGfuUf | 168 | UGUCAAGUUUAGAGUUU | 768 | gsusuaaaAfcUfcUfaaac | 568 | GUUAAAACUCUAAAC |
| 000528 | | | uuaacsasa | | UAACAA | | uugaca | | UUGACA |
| SRS- | 1818 | 369 | usUfsagucAfaguuUfaGfaGf | 169 | UUAGUCAAGUUUAGAGU | 769 | usasaaac UfcUfaAfacuu | 569 | UAAAACUCUAAACUU |
| 000529 | | | uuuuasasc | | UUUAAC | | gacuaa | | GACUAA |
| SRS- | 1822 | 370 | usUfsauuuAfgucaAfgUfuUf | 170 | UUAUUUAGUCAAGUUUA | 770 | ascsucuaAfaCfuUfgacu | 570 | ACUCUAAACUUGACUA |
| 000530 | | | agagususu | | GAGUUU | | aaauaa | | AAUAA |
| SRS- | 1900 | 371 | usAfscauaCfucugUfgCfuGf | 171 | UACAUACUCUGUGCUGA | 771 | asuscgucAfgCfaCfagag | 571 | AUCGUCAGCACAGAGU |
| 000531 | | | acgaususu | | CGAUUU | | uaugua | | AUGUA |
| SRS- | 2074 | 372 | usGfsucaaUfgugaCfuUfaGf | 172 | UGUCAAUGUGACUUAGU | 772 | gsascuacUfaAfgUfcaca | 572 | GACUACUAAGUCACAU |
| 000532 | | | uagucsasu | | AGUCAU | | uugaca | | UGACA |
| SRS- | 2098 | 373 | usGfsguauAfgugaUfaCfcUf | 173 | UGGUAUAGUGAUACCUC | 773 | ascsaugaGfgUfaUfcacu | 573 | ACAUGAGGUAUCACU |
| 000533 | | | caugususa | | AUGUUA | | auacca | | AUACCA |
| SRS- | 2231 | 374 | usUfsaaacUfuuaaCfuCfgAf | 174 | UUAAACUUUAACUCGAU | 774 | usgsgcauCfgAfgUfuaa | 574 | UGGCAUCGAGUUAAA |
| 000534 | | | ugccascsa | | GCCACA | | aguuuaa | | GUUUAA |
| SRS- | 2232 | 375 | usAfsuaaaCfuuuaAfcUfcGf | 175 | UAUAAACUUUAACUCGA | 775 | gsgscaucGfaGfuUfaaag | 575 | GGCAUCGAGUUAAAG |
| 000535 | | | augccsasc | | UGCCAC | | uuuaua | | UUUAUA |
| SRS- | 2242 | 376 | usUfsagggGfaaauAfuAfaAf | 176 | UUAGGGGAAAUAUAAAC | 776 | usasaaguUfuAfuAfuuu | 576 | UAAAGUUUAUAUUUC |
| 000536 | | | cuuuasasc | | UUUAAC | | ccccuaa | | CCCUAA |
| SRS- | 2560 | 377 | usCfscuuuAfacaaAfuGfgGf | 177 | UCCUUUAACAAAUGGGU | 777 | ususaaacCfcAfuUfugu | 577 | UUAAACCCAUUUGUU |
| 000537 | | | uuuaasusu | | UUAAUU | | uaaagga | | AAAGGA |
| SRS- | 2568 | 378 | usCfsacuaUfauccUfuUfaAf | 178 | UCACUAUAUCCUUUAAC | 778 | asusuuguUfaAfaGfgau | 578 | AUUUGUUAAAGGAUA |
| 000538 | | | caaausgsg | | AAAUGG | | auaguga | | UAGUGA |
| SRS- | 2569 | 379 | usGfscacuAfuaucCfuUfuAf | 179 | UGCACUAUAUCCUUUAA | 779 | ususuguuAfaAfgGfaua | 579 | UUUGUUAAAGGAUAU |
| 000539 | | | acaaasusg | | CAAAUG | | uagugca | | AGUGCA |
| SRS- | 2571 | 380 | usGfsggcaCfuauaUfcCfuUf | 180 | UGGGCACUAUAUCCUUU | 780 | usgsuuaaAfgGfaUfaua | 580 | UGUUAAAGGAUAUAG |
| 000540 | | | uaacasasa | | AACAAA | | gugccca | | UGCCCA |
| SRS- | 2572 | 381 | usUfsgggcAfcuauAfuCfcUf | 181 | UUGGGCACUAUAUCCUU | 781 | gsusuaaaGfgAfuAfuag | 581 | GUUAAAGGAUAUAGU |
| 000541 | | | uuaacsasa | | UAACAA | | ugcccaa | | GCCCAA |
| SRS- | 2576 | 382 | usAfsacuuGfggcaCfuAfuAf | 182 | UAACUUGGGCACUAUAU | 782 | asasggauAfuAfgUfgcc | 582 | AAGGAUAUAGUGCCC |
| 000542 | | | uccuususa | | CCUUUA | | caaguua | | AAGUUA |
| SRS- | 2577 | 383 | usUfsaacuUfgggcAfcUfaUf | 183 | UUAACUUGGGCACUAUA | 783 | asgsgauaUfaGfuGfccca | 583 | AGGAUAUAGUGCCCA |
| 000543 | | | auccususu | | UCCUUU | | aguuaa | | AGUUAA |
| SRS- | 2578 | 384 | usAfsuaacUfugggCfaCfuAf | 184 | UAUAACUUGGGCACUAU | 784 | gsgsauauAfgUfgCfccaa | 584 | GGAUAUAGUGCCCAA |
| 000544 | | | uauccsusu | | AUCCUU | | guuaua | | GUUAUA |
| SRS- | 2579 | 385 | usUfsauaaCfuuggGfcAfcUf | 185 | UUAUAACUUGGGCACUA | 785 | gsasuauaGfuGfcCfcaag | 585 | GAUAUAGUGCCCAAG |
| 000545 | | | auaucscsu | | UAUCCU | | uuauaa | | UUAUAA |
| SRS- | 2598 | 386 | usUfsgacaAfagguAfgGfuCf | 186 | UUGACAAAGGUAGGUCA | 786 | asusggugAfcCfuAfccu | 586 | AUGGUGACCUACCUUU |
| 000546 | | | accausasu | | CCAUAU | | uugucaa | | GUCAA |
| SRS- | 2599 | 387 | usUfsugacAfaaggUfaGfgUf | 187 | UUUGACAAAGGUAGGUC | 787 | usgsgugaCfcUfaCfcuu | 587 | UGGUGACCUACCUUUG |
| 000547 | | | caccasusa | | ACCAUA | | ugucaaa | | UCAAA |
| SRS- | 2600 | 388 | usAfsuugaCfaaagGfuAfgGf | 188 | UAUUGACAAAGGUAGGU | 788 | gsgsugacCfuAfcCfuuu | 588 | GGUGACCUACCUUUGU |
| 000548 | | | ucaccsasu | | CACCAU | | gucaaua | | CAAUA |
| SRS- | 2640 | 389 | usGfsacauGfuauaUfuGfgAf | 189 | UGACAUGUAUAUUGGAU | 789 | asasuuauCfcAfaUfauac | 589 | AAUUAUCCAAUAUAC |
| 000549 | | | uaauususg | | AAUUUG | | auguca | | AUGUCA |
| SRS- | 2772 | 390 | usUfsagucUfguggUfcCfuGf | 190 | UUAGUCUGUGGUCCUGA | 790 | asusuuuc AfgGfaCfcaca | 590 | AUUUUCAGGACCACAG |
| 000550 | | | aaaaususa | | AAAUUA | | gacuaa | | ACUAA |
| SRS- | 2775 | 391 | usGfscuuaGfucugUfgGfuCf | 191 | UGCUUAGUCUGUGGUCC | 791 | ususcaggAfcCfaCfagac | 591 | UUCAGGACCACAGACU |
| 000551 | | | cugaasasa | | UGAAAA | | uaagca | | AAGCA |
| SRS- | 2778 | 392 | usAfscagcUfuaguCfuGfuGf | 192 | UACAGCUUAGUCUGUGG | 792 | asgsgaccAfcAfgAfcuaa | 592 | AGGACCACAGACUAAG |
| 000552 | | | guccusgsa | | UCCUGA | | gcugua | | CUGUA |
| SRS- | 2813 | 393 | usGfsuauuCfuggcCfcUfaAf | 193 | UGUAUUCUGGCCCUAAA | 793 | ususuuuuUfaGfgGfcca | 593 | UUUUUUUAGGGCCAG |
| 000553 | | | aaaaasusc | | AAAAUC | | gaauaca | | AAUACA |
| SRS- | 2816 | 394 | usUfsugguAfuucuGfgCfcCf | 194 | UUUGGUAUUCUGGCCCU | 794 | ususuuagGfgCfcAfgaa | 594 | UUUUAGGGCCAGAAU |
| 000554 | | | uaaaasasa | | AAAAAA | | uaccaaa | | ACCAAA |
| SRS- | 2817 | 395 | usUfsuuggUfauucUfgGfcCf | 195 | UUUUGGUAUUCUGGCCC | 795 | ususuaggGfcCfaGfaaua | 595 | UUUAGGGCCAGAAUA |
| 000555 | | | cuaaasasa | | UAAAAA | | ccaaaa | | CCAAAA |
| SRS- | 2892 | 396 | usGfsaagcAfacucAfuAfuAf | 196 | UGAAGCAACUCAUAUAU | 796 | ususuaauAfuAfuGfagu | 596 | UUUAAUAUAUGAGUU |
| 000556 | | | uuaaasusa | | UAAAUA | | ugcuuca | | GCUUCA |

Note:
the target position is relative to human transcript NM_014495.4; the corresponding sequence of a certain SEQ ID NO. is located on its right column of the same row. “A″ refers to adenosine-3′-phosphate; “a″ refers to 2′-O-methyladenosine-3′-phosphate; “Af” refers to 2′-fluoroadenosine-3′-phosphate; “dA″ refers to 2′-deoxyadenosine-3-phosphate; “C″ refers to cytidine-3′-phosphate; “c″ refers to 2′-O-methylcytidine-3′-phosphate; “Cf″ refers to 2′-fluorocytidine-3′-phosphate; “dC″ refers to 2′-deoxycytidine-3′-phosphate; “G″ refers to guanosine-3′-phosphate; “g″ refers to 2′-O-methylguanosine-3′-phosphate; “Gf″ refers to 2′-fluoroguanosine-3′-phosphate; “dG″ refers to 2′-deoxyguanosine-3′-phosphate; “U″ refers to uridine-3′-phosphate; “u″ refers to 2′-O-methyluridine-3′-phosphate; “Uf″ refers to 2′-fluorouridine-3′-phosphate; “dU″ refers to 2′-deoxyuridine-3′-phosphate; “T″ refers to 5-methyluridine-3′-phosphate; “t″ refers to 2′-O-methyl-5-methyluridine-3′-phosphate; “Tf″ refers to 2′-fluoro-5-methyluridine-3′-phosphate; “dT″ refers to thymidine-3′-phosphate; “s″ refers to 3′-phosphorothioate; The “Target Position in NM_014495.4″ is defined as the 5′ position of a 21-mer target site in human transcript (NCBI Reference Sequence No: NM_014495.4).
Note that the passenger structure (sense structure) code or passenger strand (sense strand) sequence is coupled to a targeting moiety (e.g., GalNAc or galactose) at the 3′ end of the passenger strand.

TABLE 2
siRNAs Evaluated at 10 pt Dose Response in vitro in primary human hepatocytes

| | | SEQ | Guide/Antisense | SEQ | Guide/Antisense | SEQ | Passenger/Sense | SEQ | Passenger/Sense |
|---|---|---|---|---|---|---|---|---|---|
| | Target | ID | Structure Code | ID | Base Sequence | ID | Structure Code | ID | Base Sequence |
| Duplex ID | Position | NO. | (5′-3′) | NO. | (5′-3′) | NO. | (5′-3′) | NO. | (5′-3′) |
| SRS-000363 | 47 | 202 | usGfsgagcUfuaauUfgUf | 2 | UGGAGCUUAAUUGU | 602 | asasuguuCfaCfaAfu | 402 | AAUGUUCACAAUU |
| | | | gAfacauususu | | GAACAUUUU | | uaagcucca | | AAGCUCCA |
| SRS-000364 | 49 | 203 | usAfsaggaGfcuuaAfuUf | 3 | UAAGGAGCUUAAUU | 603 | usgsuucaCfaAfuUfa | 403 | UGUUCACAAUUAA |
| | | | gUfgaacasusu | | GUGAACAUU | | agcuccuua | | GCUCCUUA |
| SRS-000366 | 56 | 205 | usUfsaaaaAfgaagGfaGf | 5 | UUAAAAAGAAGGAG | 605 | asasuuaaGfcUfcCfuu | 405 | AAUUAAGCUCCUU |
| | | | cUfuaauusgsu | | CUUAAUUGU | | cuuuuuaa | | CUUUUUAA |
| SRS-000368 | 71 | 207 | usUfsaacuAfgaggAfaCf | 7 | UUAACUAGAGGAAC | 607 | ususuuauUfgUfuCfc | 407 | UUUUAUUGUUCCU |
| | | | aAfuaaaasasg | | AAUAAAAAG | | ucuaguuaa | | CUAGUUAA |
| SRS-000369 | 72 | 208 | usAfsuaacUfagagGfaAf | 8 | UAUAACUAGAGGAA | 608 | ususuauuGfuUfcCfu | 408 | UUUAUUGUUCCUC |
| | | | cAfauaaasasa | | CAAUAAAAA | | cuaguuaua | | UAGUUAUA |
| SRS-000371 | 149 | 210 | usCfsuaacAfuagcAfaAf | 10 | UCUAACAUAGCAAA | 610 | asuscaagAfuUfuGfc | 410 | AUCAAGAUUUGCU |
| | | | uCfuugaususu | | UCUUGAUUU | | uauguuaga | | AUGUUAGA |
| SRS-000372 | 151 | 211 | usGfsucuaAfcauaGfcAf | 11 | UGUCUAACAUAGCA | 611 | csasagauUfuGfcUfa | 411 | CAAGAUUUGCUAU |
| | | | aAfucuugsasu | | AAUCUUGAU | | uguuagaca | | GUUAGACA |
| SRS-000375 | 154 | 214 | usAfsucguCfuaacAfuAf | 14 | UAUCGUCUAACAUA | 614 | gsasuuugCfuAfuGfu | 414 | GAUUUGCUAUGUU |
| | | | gCfaaaucsusu | | GCAAAUCUU | | uagacgaua | | AGACGAUA |
| SRS-000376 | 155 | 215 | usCfsaucgUfcuaaCfaUf | 15 | UCAUCGUCUAACAU | 615 | asusuugcUfaUfgUfu | 415 | AUUUGCUAUGUUA |
| | | | aGfcaaauscsu | | AGCAAAUCU | | agacgauga | | GACGAUGA |
| SRS-000379 | 159 | 218 | usUfsuuacAfucguCfuAf | 18 | UUUUACAUCGUCUA | 618 | gscsuaugUfuAfgAfc | 418 | GCUAUGUUAGACG |
| | | | aCfauagcsasa | | ACAUAGCAA | | gauguaaaa | | AUGUAAAA |
| SRS-000381 | 161 | 220 | usUfsuuuuAfcaucGfuCf | 20 | UUUUUUACAUCGUC | 620 | usasuguuAfgAfcGfa | 420 | UAUGUUAGACGAU |
| | | | uAfacauasgsc | | UAACAUAGC | | uguaaaaaa | | GUAAAAAA |
| SRS-000382 | 163 | 221 | usAfsauuuUfuacaUfcGf | 21 | UAAUUUUUACAUCG | 621 | usgsuuagAfcGfaUfg | 421 | UGUUAGACGAUGU |
| | | | uCfuaacasusa | | UCUAACAUA | | uaaaaauua | | AAAAAUUA |
| SRS-000383 | 165 | 222 | usAfsaaauUfuuuaCfaUf | 22 | UAAAAUUUUUACAU | 622 | ususagacGfaUfgUfa | 422 | UUAGACGAUGUAA |
| | | | cGfucuaascsa | | CGUCUAACA | | aaaauuuua | | AAAUUUUA |
| SRS-000393 | 204 | 232 | usUfscuuuAfagacCfaUf | 32 | UUCUUUAAGACCAU | 632 | ususgggaCfaUfgGfu | 432 | UUGGGACAUGGUC |
| | | | gUfcccaascsu | | GUCCCAACU | | cuuaaagaa | | UUAAAGAA |
| SRS-000395 | 207 | 234 | usAfsagucUfuuaaGfaCf | 34 | UAAGUCUUUAAGAC | 634 | gsgsacauGfgUfcUfu | 434 | GGACAUGGUCUUA |
| | | | cAfuguccscsa | | CAUGUCCCA | | aaagacuua | | AAGACUUA |
| SRS-000403 | 282 | 242 | usAfsgaucAfuaaaAfaGf | 42 | UAGAUCAUAAAAAG | 642 | gsasucagUfcUfuUfu | 442 | GAUCAGUCUUUUU |
| | | | aCfugaucsasa | | ACUGAUCAA | | uaugaucua | | AUGAUCUA |
| SRS-000404 | 283 | 243 | usUfsagauCfauaaAfaAf | 43 | UUAGAUCAUAAAAA | 643 | asuscaguCfuUfuUfu | 443 | AUCAGUCUUUUUA |
| | | | gAfcugauscsa | | GACUGAUCA | | augaucuaa | | UGAUCUAA |
| SRS-000405 | 354 | 244 | usUfsugacUfuguaGfuU | 44 | UUUGACUUGUAGUU | 644 | ascsauauAfaAfcUfac | 444 | ACAUAUAAACUAC |
| | | | fuAfuaugusasg | | UAUAUGUAG | | aagucaaa | | AAGUCAAA |
| SRS-000406 | 396 | 245 | usUfsuugaGfuugaGfuU | 45 | UUUUGAGUUGAGUU | 645 | uscsacuuGfaAfcUfca | 445 | UCACUUGAACUCA |
| | | | fcAfagugascsa | | CAAGUGACA | | acucaaaa | | ACUCAAAA |
| SRS-000407 | 412 | 246 | usUfsaggaGfgcuuUfcAf | 46 | UUAGGAGGCUUUCA | 646 | csasaaacUfuGfaAfag | 446 | CAAAACUUGAAAG |
| | | | aGfuuuugsasg | | AGUUUUGAG | | ccuccuaa | | CCUCCUAA |
| SRS-000411 | 473 | 250 | usUfsuaagUfuaguUfaGf | 50 | UUUAAGUUAGUUAG | 650 | asgsagcaAfcUfaAfcu | 450 | AGAGCAACUAACU |
| | | | uUfgcucususc | | UUGCUCUUC | | aacuuaaa | | AACUUAAA |
| SRS-000412 | 474 | 251 | usAfsuuaaGfuuagUfuA | 51 | UAUUAAGUUAGUUA | 651 | gsasgcaaCfuAfaCfua | 451 | GAGCAACUAACUA |
| | | | fgUfugcucsusu | | GUUGCUCUU | | acuuaaua | | ACUUAAUA |
| SRS-000413 | 485 | 252 | usGfsuugaUfuuugAfaU | 52 | UGUUGAUUUUGAAU | 652 | usasacuuAfaUfuCfaa | 452 | UAACUUAAUUCAA |
| | | | fuAfaguuasgsu | | UAAGUUAGU | | aaucaaca | | AAUCAACA |
| SRS-000414 | 520 | 253 | usAfsagugAfaguuAfcU | 53 | UAAGUGAAGUUACU | 653 | ascsccagAfaGfuAfac | 453 | ACCCAGAAGUAAC |
| | | | fuCfugggusgsu | | UCUGGGUGU | | uucacuua | | UUCACUUA |
| SRS-000415 | 521 | 254 | usUfsaaguGfaaguUfaCf | 54 | UUAAGUGAAGUUAC | 654 | cscscagaAfgUfaAfcu | 454 | CCCAGAAGUAACU |
| | | | uUfcugggsusg | | UUCUGGGUG | | ucacuuaa | | UCACUUAA |
| SRS-000416 | 553 | 255 | usGfsaugcUfauuaUfcUf | 55 | UGAUGCUAUUAUCU | 655 | asasaaacAfaGfaUfaa | 455 | AAAAACAAGAUAA |
| | | | uGfuuuuuscsu | | UGUUUUUCU | | uagcauca | | UAGCAUCA |
| SRS-000417 | 555 | 256 | usUfsugauGfcuauUfaUf | 56 | UUUGAUGCUAUUAU | 656 | asasacaaGfaUfaAfua | 456 | AAACAAGAUAAUA |
| | | | cUfuguuususu | | CUUGUUUUU | | gcaucaaa | | GCAUCAAA |
| SRS-000421 | 615 | 260 | usAfsuuugAfcuauGfcU | 60 | UAUUUGACUAUGCU | 660 | asasccaaCfaGfcAfua | 460 | AACCAACAGCAUA |
| | | | fgUfugguususa | | GUUGGUUUA | | gucaaaua | | GUCAAAUA |
| SRS-000436 | 897 | 276 | usUfscuauUfcgauGfuUf | 76 | UUCUAUUCGAUGUU | 676 | ususaauuCfaAfcAfu | 476 | UUAAUUCAACAUC |
| | | | gAfauuaasusg | | GAAUUAAUG | | cgaauagaa | | GAAUAGAA |
| SRS-000437 | 898 | 277 | usAfsucuaUfucgaUfgUf | 77 | UAUCUAUUCGAUGU | 677 | usasauucAfaCfaUfcg | 477 | UAAUUCAACAUCG |
| | | | uGfaauuasasu | | UGAAUUAAU | | aauagaua | | AAUAGAUA |
| SRS-000456 | 942 | 296 | usCfscauaUfuuguAfgUf | 96 | UCCAUAUUUGUAGU | 696 | usgsggagAfaCfuAfc | 496 | UGGGAGAACUACA |
| | | | uCfucccascsg | | UCUCCCACG | | aaauaugga | | AAUAUGGA |
| SRS-000458 | 966 | 298 | usAfsauucUfccauCfaAf | 98 | UAAUUCUCCAUCAA | 698 | gsgsgaggCfuUfgAfu | 498 | GGGAGGCUUGAUG |
| | | | gCfcucccsasa | | GCCUCCCAA | | ggagaauua | | GAGAAUUA |
| SRS-000467 | 1007 | 307 | usAfsuugcUfucacUfaUf | 107 | UAUUGCUUCACUAU | 707 | asusacucCfaUfaGfug | 507 | AUACUCCAUAGUG |
| | | | gGfaguausasu | | GGAGUAUAU | | aagcaaua | | AAGCAAUA |
| SRS-000468 | 1012 | 308 | usAfsuuagAfuugcUfuC | 108 | UAUUAGAUUGCUUC | 708 | cscsauagUfgAfaGfca | 508 | CCAUAGUGAAGCA |
| | | | faCfuauggsasg | | ACUAUGGAG | | aucuaaua | | AUCUAAUA |
| SRS-000469 | 1014 | 309 | usUfsaauuAfgauuGfcUf | 109 | UUAAUUAGAUUGCU | 709 | asusagugAfaGfcAfa | 509 | AUAGUGAAGCAAU |
| | | | uCfacuausgsg | | UCACUAUGG | | ucuaauuaa | | CUAAUUAA |
| SRS-000471 | 1098 | 311 | usUfsugguUfucguGfaU | 111 | UUUGGUUUCGUGAU | 711 | ususgggaAfaUfcAfc | 511 | UUGGGAAAUCACG |
| | | | fuUfcccaasgsu | | UUCCCAAGU | | gaaaccaaa | | AAACCAAA |
| SRS-000473 | 1101 | 313 | usUfsaguuGfguuuCfgU | 113 | UUAGUUGGUUUCGU | 713 | gsgsaaauCfaCfgAfaa | 513 | GGAAAUCACGAAA |
| | | | fgAfuuuccscsa | | GAUUUCCCA | | ccaacuaa | | CCAACUAA |
| SRS-000474 | 1103 | 314 | usUfsauagUfugguUfuC | 114 | UUAUAGUUGGUUUC | 714 | asasaucaCfgAfaAfcc | 514 | AAAUCACGAAACC |
| | | | fgUfgauuuscsc | | GUGAUUUCC | | aacuauaa | | AACUAUAA |
| SRS-000495 | 1179 | 335 | usCfsaaguAfgaaaAfcAf | 135 | UCAAGUAGAAAACA | 735 | gsasuuugGfuGfuUfu | 535 | GAUUUGGUGUUUU |
| | | | cCfaaaucsusu | | CCAAAUCUU | | ucuacuuga | | CUACUUGA |
| SRS-000505 | 1357 | 345 | usAfsgaguAfuaacCfuUf | 145 | UAGAGUAUAACCUU | 745 | asasaaugGfaAfgGfu | 545 | AAAAUGGAAGGUU |
| | | | cCfauuuusgsa | | CCAUUUUGA | | uauacucua | | AUACUCUA |
| SRS-000506 | 1358 | 346 | usUfsagagUfauaaCfcUf | 146 | UUAGAGUAUAACCU | 746 | asasauggAfaGfgUfu | 546 | AAAUGGAAGGUUA |
| | | | uCfcauuususg | | UCCAUUUUG | | auacucuaa | | UACUCUAA |
| SRS-000508 | 1363 | 348 | usUfsuuuaUfagagUfaUf | 148 | UUUUUAUAGAGUAU | 748 | gsasagguUfaUfaCfu | 548 | GAAGGUUAUACUC |
| | | | aAfccuucscsa | | AACCUUCCA | | cuauaaaaa | | UAUAAAAA |
| SRS-000509 | 1365 | 349 | usGfsauuuUfauagAfgU | 149 | UGAUUUUAUAGAGU | 749 | asgsguuaUfaCfuCfu | 549 | AGGUUAUACUCUA |
| | | | faUfaaccususc | | AUAACCUUC | | auaaaauca | | UAAAAUCA |
| SRS-000513 | 1384 | 353 | usAfsuggaUfcaacAfuUf | 153 | UAUGGAUCAACAUU | 753 | csasaccaAfaAfuGfuu | 553 | CAACCAAAAUGUU |
| | | | uUfgguugsasu | | UUGGUUGAU | | gauccaua | | GAUCCAUA |
| SRS-000517 | 1494 | 357 | usAfsggauUfuaauAfcCf | 157 | UAGGAUUUAAUACC | 757 | asusaaucUfgGfuAfu | 557 | AUAAUCUGGUAUU |
| | | | aGfauuaususa | | AGAUUAUUA | | uaaauccua | | AAAUCCUA |
| SRS-000518 | 1496 | 358 | usUfsaaggAfuuuaAfuA | 158 | UUAAGGAUUUAAUA | 758 | asasucugGfuAfuUfa | 558 | AAUCUGGUAUUAA |
| | | | fcCfagauusasu | | CCAGAUUAU | | aauccuuaa | | AUCCUUAA |
| SRS-000520 | 1563 | 360 | usUfsuguaUfguuuAfaU | 160 | UUUGUAUGUUUAAU | 760 | asusuuaaGfaUfuAfa | 560 | AUUUAAGAUUAAA |
| | | | fcUfuaaausasg | | CUUAAAUAG | | acauacaaa | | CAUACAAA |
| SRS-000521 | 1566 | 361 | usUfsgauuGfuaugUfuU | 161 | UUGAUUGUAUGUUU | 761 | usasagauUfaAfaCfau | 561 | UAAGAUUAAACAU |
| | | | faAfucuuasasa | | AAUCUUAAA | | acaaucaa | | ACAAUCAA |

Note:
the target position is relative to human transcript NM_014495.4; the corresponding sequence of a certain SEQ ID NO. is located on its right column of the same row. “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “dU” refers to 2′-deoxyuridine-3′-phosphate; “T“ refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to thymidine-3′-phosphate; “s” refers to 3′-phosphorothioate; The “Target Position in NM_014495.4” is defined as the 5′ position of a 21-mer target site in human transcript (NCBI Reference Sequence No: NM_014495.4).
Note that the passenger structure (sense structure) code or passenger strand (sense strand) sequence is coupled to a targeting moiety (e.g., GalNAc or galactose) at the 3′ end of the passenger strand.

TABLE 3
siRNAs Selected for In Vivo Humanized Mouse Model Screening

| | | SEQ | | SEQ | Guide/Antisense | SEQ | | SEQ | Passenger/Sense |
|---|---|---|---|---|---|---|---|---|---|
| Duplex | Target | ID | Guide/Antisense Structure Code | ID | Base Sequence | ID | Passenger/Sense Structure | ID | Base Sequence |
| ID | Position | NO. | (5′-3′) | NO. | (5′-3′) | NO. | Code (5′-3′) | NO. | (5′-3′) |
| SRS- | 56 | 205 | usUfsaaaaAfgaagGfaGfcUfuaauus | 5 | UUAAAAAGAAGGAGCUUAAU | 605 | asasuuaaGfcUfcCfuucuuuuuaa | 405 | AAUUAAGCUCCUUCUUUUU |
| 000559 | | | gsu | | UGU | | | | AA |
| SRS- | 71 | 207 | usUfsaacuAfgaggAfaCfaAfuaaaas | 7 | UUAACUAGAGGAACAAUAAA | 607 | ususuuauUfgUfuCfcucuaguua | 407 | UUUUAUUGUUCCUCUAGUU |
| 000560 | | | asg | | AAG | | a | | AA |
| SRS- | 72 | 208 | usAfsuaacUfagagGfaAfcAfauaaas | 8 | UAUAACUAGAGGAACAAUAA | 608 | ususuauuGfuUfcCfucuaguuau | 408 | UUUAUUGUUCCUCUAGUUA |
| 000561 | | | asa | | AAA | | a | | UA |
| SRS- | 151 | 211 | usGfsucuaAfcauaGfcAfaAfucuug | 11 | UGUCUAACAUAGCAAAUCUU | 611 | csasagauUfuGfcUfauguuagaca | 411 | CAAGAUUUGCUAUGUUAGA |
| 000650 | | | sasu | | GAU | | | | CA |
| SRS- | 159 | 218 | usUfsuuacAfucguCfuAfaCfauagcs | 18 | UUUUACAUCGUCUAACAUAG | 618 | gscsuaugUfuAfgAfcgauguaaa | 418 | GCUAUGUUAGACGAUGUAA |
| 000563 | | | asa | | CAA | | a | | AA |
| SRS- | 161 | 220 | usUfsuuuuAfcaucGfuCfuAfacaua | 20 | UUUUUUACAUCGUCUAACAU | 620 | usasuguuAfgAfcGfauguaaaaa | 420 | UAUGUUAGACGAUGUAAA |
| 000564 | | | sgsc | | AGC | | a | | AAA |
| SRS- | 165 | 222 | usAfsaaauUfuuuaCfaUfcGfucuaas | 22 | UAAAAUUUUUACAUCGUCUA | 622 | ususagacGfaUfgUfaaaaauuuua | 422 | UUAGACGAUGUAAAAAUU |
| 000566 | | | csa | | ACA | | | | UUA |
| SRS- | 269 | 240 | usAfscugaUfcaaaUfaUfgUfugagu | 40 | UACUGAUCAAAUAUGUUGAG | 640 | ascsucaaCfaUfaUfuugaucagua | 440 | ACUCAACAUAUUUGAUCAG |
| 000651 | | | susu | | UUU | | | | UA |
| SRS- | 283 | 243 | usUfsagauCfauaaAfaAfgAfcugaus | 43 | UUAGAUCAUAAAAAGACUGA | 643 | asuscaguCfuUfuUfuaugaucua | 443 | AUCAGUCUUUUUAUGAUCU |
| 000568 | | | csa | | UCA | | a | | AA |
| SRS- | 354 | 244 | usUfsugacUfuguaGfuUfuAfuaugu | 44 | UUUGACUUGUAGUUUAUAUG | 644 | ascsauauAfaAfcUfacaagucaaa | 444 | ACAUAUAAACUACAAGUCA |
| 000569 | | | sasg | | UAG | | | | AA |
| SRS- | 396 | 245 | usUfsuugaGfuugaGfuUfcAfaguga | 45 | UUUUGAGUUGAGUUCAAGUG | 645 | uscsacuuGfaAfcUfcaacucaaaa | 445 | UCACUUGAACUCAACUCAA |
| 000570 | | | scsa | | ACA | | | | AA |
| SRS- | 412 | 246 | usUfsaggaGfgcuuUfcAfaGfuuuug | 46 | UUAGGAGGCUUUCAAGUUUU | 646 | csasaaacUfuGfaAfagccuccuaa | 446 | CAAAACUUGAAAGCCUCCU |
| 000652 | | | sasg | | GAG | | | | AA |
| SRS- | 473 | 250 | usUfsuaagUfuaguUfaGfuUfgcucu | 50 | UUUAAGUUAGUUAGUUGCUC | 650 | asgsagcaAfcUfaAfcuaacuuaaa | 450 | AGAGCAACUAACUAACUUA |
| 000571 | | | susc | | UUC | | | | AA |
| SRS- | 485 | 252 | usGfsuugaUfuuugAfaUfuAfaguua | 52 | UGUUGAUUUUGAAUUAAGUU | 652 | usasacuuAfaUfuCfaaaaucaaca | 452 | UAACUUAAUUCAAAAUCAA |
| 000653 | | | sgsu | | AGU | | | | CA |
| SRS- | 520 | 253 | usAfsagugAfaguuAfcUfuCfugggu | 53 | UAAGUGAAGUUACUUCUGGG | 653 | ascsccagAfaGfuAfacuucacuua | 453 | ACCCAGAAGUAACUUCACU |
| 000654 | | | sgsu | | UGU | | | | UA |
| SRS- | 555 | 256 | usUfsugauGfcuauUfaUfcUfuguuu | 56 | UUUGAUGCUAUUAUCUUGUU | 656 | asasacaaGfaUfaAfuagcaucaaa | 456 | AAACAAGAUAAUAGCAUCA |
| 000572 | | | susu | | UUU | | | | AA |
| SRS- | 1012 | 308 | usAfsuuagAfuugcUfuCfaCfuaugg | 108 | UAUUAGAUUGCUUCACUAUG | 708 | cscsauagUfgAfaGfcaaucuaaua | 508 | CCAUAGUGAAGCAAUCUAA |
| 000655 | | | sasg | | GAG | | | | UA |
| SRS- | 1014 | 309 | usUfsaauuAfgauuGfcUfuCfacuau | 109 | UUAAUUAGAUUGCUUCACUA | 709 | asusagugAfaGfcAfaucuaauuaa | 509 | AUAGUGAAGCAAUCUAAUU |
| 000575 | | | sgsg | | UGG | | | | AA |
| SRS- | 1101 | 313 | usUfsaguuGfguuuCfgUfgAfuuucc | 113 | UUAGUUGGUUUCGUGAUUUC | 713 | gsgsaaauCfaCfgAfaaccaacuaa | 513 | GGAAAUCACGAAACCAACU |
| 000656 | | | scsa | | CCA | | | | AA |
| SRS- | 1103 | 314 | usUfsauagUfugguUfuCfgUfgauuu | 114 | UUAUAGUUGGUUUCGUGAUU | 714 | asasaucaCfgAfaAfccaacuauaa | 514 | AAAUCACGAAACCAACUAU |
| 000657 | | | scsc | | UCC | | | | AA |
| SRS- | 1357 | 345 | usAfsgaguAfuaacCfuUfcCfauuuu | 145 | UAGAGUAUAACCUUCCAUUU | 745 | asasaaugGfaAfgGfuuauacucua | 545 | AAAAUGGAAGGUUAUACUC |
| 000658 | | | sgsa | | UGA | | | | UA |
| SRS- | 1358 | 346 | usUfsagagUfauaaCfcUfuCfcauuus | 146 | UUAGAGUAUAACCUUCCAUU | 746 | asasauggAfaGfgUfuauacucuaa | 546 | AAAUGGAAGGUUAUACUCU |
| 000576 | | | usg | | UUG | | | | AA |
| SRS- | 1363 | 348 | usUfsuuuaUfagagUfaUfaAfecuuc | 148 | UUUUUAUAGAGUAUAACCUU | 748 | gsasagguUfaUfaCfucuauaaaaa | 548 | GAAGGUUAUACUCUAUAAA |
| 000659 | | | scsa | | CCA | | | | AA |
| SRS- | 1384 | 353 | usAfsuggaUfcaacAfuUfuUfgguug | 153 | UAUGGAUCAACAUUUUGGUU | 753 | csasaccaAfaAfuGfuugauccaua | 553 | CAACCAAAAUGUUGAUCCA |
| 000577 | | | sasu | | GAU | | | | UA |
| SRS- | 1494 | 357 | usAfsggauUfuaauAfcCfaGfauuau | 157 | UAGGAUUUAAUACCAGAUUA | 757 | asusaaucUfgGfuAfuuaaauccua | 557 | AUAAUCUGGUAUUAAAUCC |
| 000661 | | | susa | | UUA | | | | UA |
| SRS- | 1496 | 358 | usUfsaaggAfuuuaAfuAfcCfagauu | 158 | UUAAGGAUUUAAUACCAGAU | 758 | asasucugGfuAfuUfaaauccuuaa | 558 | AAUCUGGUAUUAAAUCCUU |
| 000578 | | | sasu | | UAU | | | | AA |
| SRS- | 54 | 808 | usAfsaaagAfaggaGfcUfuAfauugu | 797 | UAAAAGAAGGAGCUUAAUUG | 830 | ascsaauuAfaGfcUfccuucuuuua | 819 | ACAAUUAAGCUCCUUCUUU |
| 001803 | | | sgsa | | UGA | | | | UA |
| SRS- | 158 | 809 | usUfsuacaUfcgucUfaAfcAfuagcas | 798 | UUUACAUCGUCUAACAUAGC | 831 | usgscuauGfuUfaGfacgauguaa | 820 | UGCUAUGUUAGACGAUGUA |
| 001804 | | | asa | | AAA | | a | | AA |
| SRS- | 267 | 810 | usUfsgaucAfaauaUfgUfuGfaguuu | 799 | UUGAUCAAAUAUGUUGAGUU | 832 | asasacucAfaCfaUfauuugaucaa | 821 | AAACUCAACAUAUUUGAUC |
| 001805 | | | susu | | UUU | | | | AA |
| SRS- | 281 | 811 | usGfsaucaUfaaaaAfgAfcUfgaucas | 800 | UGAUCAUAAAAAGACUGAUC | 833 | usgsaucaGfuCfuUfuuuaugauc | 822 | UGAUCAGUCUUUUUAUGAU |
| 001806 | | | asa | | AAA | | a | | CA |
| SRS- | 919 | 812 | usUfsucauUfgaagUfuUfuGfugauc | 801 | UUUCAUUGAAGUUUUGUGAU | 834 | gsasucacAfaAfaCfuucaaugaaa | 823 | GAUCACAAAACUUCAAUGA |
| 001807 | | | scsa | | CCA | | | | AA |
| SRS- | 1007 | 813 | usAfsuugcUfucacUfaUfgGfaguau | 802 | UAUUGCUUCACUAUGGAGUA | 835 | asusacucCfaUfaGfugaagcaaua | 824 | AUACUCCAUAGUGAAGCAA |
| 001808 | | | sasu | | UAU | | | | UA |
| SRS- | 1008 | 814 | usGfsauugCfuucaCfuAfuGfgagua | 803 | UGAUUGCUUCACUAUGGAGU | 836 | usascuccAfuAfgUfgaagcaauca | 825 | UACUCCAUAGUGAAGCAAU |
| 001809 | | | susa | | AUA | | | | CA |
| SRS- | 1013 | 815 | usAfsauuaGfauugCfuUfcAfcuaug | 804 | UAAUUAGAUUGCUUCACUAU | 837 | csasuaguGfaAfgCfaaucuaauua | 826 | CAUAGUGAAGCAAUCUAAU |
| 001810 | | | sgsa | | GGA | | | | UA |
| SRS- | 1099 | 816 | usGfsuuggUfuucgUfgAfuUfuccca | 805 | UGUUGGUUUCGUGAUUUCCC | 838 | usgsggaaAfuCfaCfgaaaccaaca | 827 | UGGGAAAUCACGAAACCAA |
| 001811 | | | sasg | | AAG | | | | CA |
| SRS- | 1102 | 817 | usAfsuaguUfgguuUfcGfuGfauuuc | 806 | UAUAGUUGGUUUCGUGAUUU | 839 | gsasaaucAfcGfaAfaccaacuaua | 828 | GAAAUCACGAAACCAACUA |
| 001812 | | | SCSC | | CCC | | | | UA |
| SRS- | 1357 | 858 | usAfsgagu(T-NAc) | 850 | UAGAGUUAACCUUCCAUUUU | 881 | asasaaugGfaAfgGfuuaaacucua | 870 | AAAAUGGAAGGUUAAACUC |
| 002024 | | | uaacCfuUfcCfauuuusgsa | | GA | | | | UA |
| SRS- | 1357 | 859 | usAfsgagu(dAB)uaacCfuUfcCfau | 850 | UAGAGUUAACCUUCCAUUUU | 881 | asasaaugGfaAfgGfuuaaacucua | 870 | AAAAUGGAAGGUUAAACUC |
| 002028 | | | uuusgsa | | GA | | | | UA |
| SRS- | 1357 | 860 | usAfsgag(T-NAc) | 851 | UAGAGAUAACCUUCCAUUUU | 876 | asasaaugGfaAfgGfuuaugcucu | 871 | AAAAUGGAAGGUUAUGCUC |
| 002032 | | | AfuaacCfuUfcCfauuuusgsa | | GA | | a | | UA |
| SRS- | 1357 | 861 | usAfsgag(dAB)AfuaacCfuUfcCfa | 851 | UAGAGAUAACCUUCCAUUUU | 876 | asasaaugGfaAfgGfuuaugcucu | 871 | AAAAUGGAAGGUUAUGCUC |
| 002034 | | | uuuusgsa | | GA | | a | | UA |
| SRS- | 1357 | 862 | usAfsgaguAf(T-NAc) | 852 | UAGAGUAAACCUUCCAUUUU | 877 | asasaaugGfaAfgGfuuguacucu | 872 | AAAAUGGAAGGUUGUACUC |
| 002045 | | | aacCfuUfcCfauuuusgsa | | GA | | a | | UA |
| SRS- | 1357 | 863 | usAfsgagu(T-A) | 853 | UAGAGUAUAACCUUCCAUUU | 878 | asasaaugGfaAfgGfuuauacucua | 873 | AAAAUGGAAGGUUAUACUC |
| 002047 | | | uaacCfuUfcCfauuuusgsa | | UGA | | | | UA |
| SRS- | 1357 | 864 | usAfsgaguAf(dAB)aacCfuUfcCfa | 852 | UAGAGUAAACCUUCCAUUUU | 877 | asasaaugGfaAfgGfuuguacucu | 872 | AAAAUGGAAGGUUGUACUC |
| 002049 | | | uuuusgsa | | GA | | a | | UA |
| SRS- | 1357 | 865 | usAfsgaguAfuaacCfuUfcCfauuucs | 854 | UAGAGUAUAACCUUCCAUUU | 879 | gsasaaugGfaAfgGfuuauacucu | 874 | GAAAUGGAAGGUUAUACUC |
| 002050 | | | gsa | | CGA | | a | | UA |
| SRS- | 1357 | 866 | usAfsgaguAfuaacCfuUfcCfauuccs | 855 | UAGAGUAUAACCUUCCAUUC | 880 | gsgsaaugGfaAfgGfuuauacucu | 875 | GGAAUGGAAGGUUAUACUC |
| 002051 | | | gsa | | CGA | | a | | UA |
| SRS- | 1357 | 867 | usAfsgag(T-T) | 856 | UAGAGTAUAACCUUCCAUUU | 878 | asasaaugGfaAfgGfuuauacucua | 873 | AAAAUGGAAGGUUAUACUC |
| 002052 | | | AfuaacCfuUfcCfauuuusgsa | | UGA | | | | UA |
| SRS- | 1357 | 868 | usAfsgaguAf(T-T) | 857 | UAGAGUATAACCUUCCAUUU | 878 | asasaaugGfaAfgGfuuauacucua | 873 | AAAAUGGAAGGUUAUACUC |
| 002053 | | | aacCfuUfcCfauuuusgsa | | UGA | | | | UA |

Note:
the target position is relative to human transcript NM_014495.4; the corresponding sequence of a certain SEQ ID NO. is located on its right column of the same row.
“A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “al” refers to 2-Amino-2′-O-methyladenosine-3′-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “u3” refers to 2′-O-methyl-2-thiouridine-3′-phosphate; “U3f” refers to 2′-fluoro-2-thiouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate; “(T-T)” refers to acyclic L-threoninol nucleic acid-thymine-3′-phosphate; “(T-A)” refers to acyclic L-threoninol nucleic acid-adenine-3′-phosphate; “(T-NAc)” refers to acyclic N-acetyl L-threoninol abasic nucleic acid-3′phosphate; “(dAB)” refers to 1′,2′-Dideoxyribose-3′phosphate; Each modified sense sequence is coupled to a targeting moiety (e.g., GalNAc or galactose) at its 3′ end via a linker (e.g., Formula V′, V““, V““““, or V““““““).

TABLE 4
siRNAs Selected for In Vivo NHP Screening

| | | | | | | SEQ | Passenger/Sense | | |
|---|---|---|---|---|---|---|---|---|---|
| Duplex | Target | SEQ ID | Guide/Antisense Structure Code | SEQ ID | Guide/Antisense Base | ID | Structure | SEQ ID | Passenger/Sense Base |
| ID | Position | NO. | (5′-3′) | NO. | Sequence (5′-3′) | NO. | Code (5′-3′) | NO. | Sequence (5′-3′) |
| SRS- | 1101 | 313 | usUfsaguuGfguuuCfgUfgAfuuucc | 113 | UUAGUUGGUUUCGUGAU | 713 | gsgsaaauCfaCfgAfaaccaacu | 513 | GGAAAUCACGAAACCAAC |
| 000656 | | | scsa | | UUCCCA | | aa | | UAA |
| SRS- | 1358 | 346 | usUfsagagUfauaaCfcUfuCfcauuu | 146 | UUAGAGUAUAACCUUCC | 746 | asasauggAfaGfgUfuauacucu | 546 | AAAUGGAAGGUUAUACUC |
| 000576 | | | susg | | AUUUUG | | aa | | UAA |
| SRS- | 1384 | 353 | usAfsuggaUfcaacAfuUfuUfgguug | 153 | UAUGGAUCAACAUUUUG | 753 | csasaccaAfaAfuGfuugaucca | 553 | CAACCAAAAUGUUGAUCC |
| 000577 | | | sasu | | GUUGAU | | ua | | AUA |
| SRS- | 1496 | 358 | usUfsaaggAfuuuaAfuAfcCfagauu | 158 | UUAAGGAUUUAAUACCA | 758 | asasucugGfuAfuUfaaauccuu | 558 | AAUCUGGUAUUAAAUCCU |
| 000578 | | | sasu | | GAUUAU | | aa | | UAA |
| SRS- | 1012 | 308 | usAfsuuagAfuugcUfuCfaCfuaugg | 108 | UAUUAGAUUGCUUCACU | 708 | cscsauagUfgAfaGfcaaucuaa | 508 | CCAUAGUGAAGCAAUCUA |
| 000655 | | | sasg | | AUGGAG | | ua | | AUA |
| SRS- | 1103 | 314 | usUfsauagUfugguUfuCfgUfgauuu | 114 | UUAUAGUUGGUUUCGUG | 714 | asasaucaCfgAfaAfccaacuau | 514 | AAAUCACGAAACCAACUA |
| 000657 | | | scsc | | AUUUCC | | aa | | UAA |
| SRS- | 1357 | 345 | usAfsgaguAfuaacCfuUfcCfauuuu | 145 | UAGAGUAUAACCUUCCA | 745 | asasaaugGfaAfgGfuuauacuc | 545 | AAAAUGGAAGGUUAUACU |
| 000658 | | | sgsa | | UUUUGA | | ua | | CUA |

Note:
the target position is relative to human transcript NM_014495.4; the corresponding sequence of a certain SEQ ID NO. is located on its right column of the same row.
“A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “dU” refers to 2′-deoxyuridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to thymidine-3′-phosphate; “(invAb)” refers to inverted abasic deoxyribonucleotide; “s” refers to 3′-phosphorothioate; Each modified sense sequence is coupled to a targeting moeity (e.g, GalNAc or galactose) at its 3′ end via linker (e.g, Formula V′, V“, V“, V“).

TABLE 5
Results of In vitro efficacy of siRNAs targeting ANGPTL3

| | | | | | | 10 μM screen | | | 0.5 μM screen | | |
|---|---|---|---|---|---|---|---|---|---|---|---|
| | | | | | | 10 uM | | | 0.5 uM | | |
| Sequence Info | | | | | | % | | | % | | |
| | SEQ | | SED | | | Residual | | | Residual | | |
| Duplex | ID | | ID | | Transcript | Target | SD quad- | 10 μM | Target | SD quad- | 0.5 μM |
| ID | NO. | Sense Sequence | NO. | Antisense Sequence | Position | mRNA | ruplicates | Rank | mRNA | ruplicates | Rank |
| SRS- | 601 | asasaaauGfuUfcAfca | 201 | usGfscuuaAfuuguGf | 44 | 64.5 | 5.6 | 51 | 70.7 | 6.8 | 57 |
| 000362 | | auuaagca | | aAfcAfuuuuusasu | | | | | | | |
| SRS- | 602 | asasuguuCfaCfaAfuu | 202 | usGfsgagcUfuaauUfg | 47 | 50.0 | 3.3 | 34 | 50.8 | 6.9 | 29 |
| 000363 | | aagcucca | | UfgAfacauususu | | | | | | | |
| SRS- | 603 | usgsuucaCfaAfuUfaa | 203 | usAfsaggaGfcuuaAfu | 49 | 28.7 | 3.8 | 4 | 33.2 | 2.6 | 4 |
| 000364 | | gcuccuua | | UfgUfgaacasusu | | | | | | | |
| SRS- | 604 | uscsacaaUfuAfaGfcu | 204 | usAfsagaaGfgagcUfu | 52 | 83.4 | 3.0 | 99 | 86.3 | 7.8 | 102 |
| 000365 | | ccuucuua | | AfaUfugugasasc | | | | | | | |
| SRS- | 605 | asasuuaaGfcUfcCfuu | 205 | usUfsaaaaAfgaagGfa | 56 | 31.1 | 1.9 | 9 | 37.5 | 4.7 | 9 |
| 000366 | | cuuuuuaa | | GfcUfuaauusgsu | | | | | | | |
| SRS- | 606 | asusuaagCfuCfcUfuc | 206 | usAfsuaaaAfagaaGfg | 57 | 29.7 | 3.2 | 5 | 29.3 | 1.5 | 1 |
| 000367 | | uuuuuaua | | AfgCfuuaaususg | | | | | | | |
| SRS- | 607 | ususuuauUfgUfuCfcu | 207 | usUfsaacuAfgaggAfa | 71 | 30.3 | 1.8 | 7 | 40.1 | 1.1 | 16 |
| 000368 | | cuaguuaa | | CfaAfuaaaasasg | | | | | | | |
| SRS- | 608 | ususuauuGfuUfcCfuc | 208 | usAfsuaacUfagagGfa | 72 | 35.2 | 3.1 | 13 | 38.7 | 3.3 | 12 |
| 000369 | | uaguuaua | | AfcAfauaaasasa | | | | | | | |
| SRS- | 609 | ususguucCfuCfuAfgu | 209 | usGfsgaaaUfaacuAfg | 76 | 70.5 | 8.0 | 66 | 72.7 | 5.6 | 63 |
| 000370 | | uauuucca | | AfgGfaacaasusa | | | | | | | |
| SRS- | 610 | asuscaagAfuUfuGfcu | 210 | usCfsuaacAfuagcAfa | 149 | 68.8 | 7.2 | 60 | 71.5 | 5.3 | 59 |
| 000371 | | auguuaga | | AfuCfuugaususu | | | | | | | |
| SRS- | 611 | csasagauUfuGfcUfau | 211 | usGfsucuaAfcauaGfc | 151 | 43.6 | 2.5 | 26 | 60.3 | 5.4 | 41 |
| 000372 | | guuagaca | | AfaAfucuugsasu | | | | | | | |
| SRS- | 612 | asasgauuUfgCfuAfug | 212 | usCfsgucuAfacauAfg | 152 | 66.2 | 3.9 | 54 | 67.7 | 2.1 | 52 |
| 000373 | | uuagacga | | CfaAfaucuusgsa | | | | | | | |
| SRS- | 613 | asgsauuuGfcUfaUfgu | 213 | usUfscgucUfaacaUfa | 153 | 88.2 | 8.5 | 112 | 76.3 | 10.2 | 71 |
| 000374 | | uagacgaa | | GfcAfaaucususg | | | | | | | |
| SRS- | 614 | gsasuuugCfuAfuGfu | 214 | usAfsucguCfuaacAfu | 154 | 37.2 | 0.9 | 16 | 39.2 | 3.1 | 13 |
| 000375 | | uagacgaua | | AfgCfaaaucsusu | | | | | | | |
| SRS- | 615 | asusuugcUfaUfgUfua | 215 | usCfsaucgUfcuaaCfa | 155 | 67.5 | 5.5 | 57 | 57.1 | 1.3 | 37 |
| 000376 | | gacgauga | | UfaGfcaaauscsu | | | | | | | |
| SRS- | 616 | ususugcuAfuGfuUfa | 216 | usAfscaucGfucuaAfc | 156 | 75.6 | 6.7 | 78 | 71.0 | 3.5 | 58 |
| 000377 | | gacgaugua | | AfuAfgcaaasusc | | | | | | | |
| SRS- | 617 | ususgcuaUfgUfuAfga | 217 | usUfsacauCfgucuAfa | 157 | 72.4 | 3.2 | 73 | 70.4 | 3.2 | 56 |
| 000378 | | cgauguaa | | CfaUfagcaasasu | | | | | | | |
| SRS- | 618 | gscsuaugUfuAfgAfcg | 218 | usUfsuuacAfucguCfu | 159 | 34.7 | 2.8 | 12 | 39.2 | 3.9 | 14 |
| 000379 | | auguaaaa | | AfaCfauagcsasa | | | | | | | |
| SRS- | 619 | csusauguUfaGfaCfga | 219 | usUfsuuuaCfaucgUfc | 160 | 62.8 | 10.6 | 45 | 66.1 | 4.3 | 50 |
| 000380 | | uguaaaaa | | UfaAfcauagscsa | | | | | | | |
| SRS- | 620 | usasuguuAfgAfcGfau | 220 | usUfsuuuuAfcaucGf | 161 | 33.9 | 5.5 | 11 | 38.2 | 3.0 | 11 |
| 000381 | | guaaaaaa | | uCfuAfacauasgsc | | | | | | | |
| SRS- | 621 | usgsuuagAfcGfaUfgu | 221 | usAfsauuuUfuacaUfc | 163 | 41.6 | 5.1 | 22 | 49.2 | 2.5 | 27 |
| 000382 | | aaaaauua | | GfuCfuaacasusa | | | | | | | |
| SRS- | 622 | ususagacGfaUfgUfaa | 222 | usAfsaaauUfuuuaCfa | 165 | 23.5 | 0.3 | 2 | 36.8 | 5.4 | 7 |
| 000383 | | aaauuuua | | UfcGfucuaascsa | | | | | | | |
| SRS- | 623 | asasauuuUfaGfcCfaa | 223 | usGfsaggcCfauugGfc | 178 | 114.2 | 9.2 | 164 | 114.0 | 6.7 | 191 |
| 000384 | | uggccuca | | UfaAfaauuususu | | | | | | | |
| SRS- | 624 | asasuuuuAfgCfcAfau | 224 | usGfsgaggCfcauuGfg | 179 | 102.4 | 14.5 | 144 | 104.7 | 12.3 | 175 |
| 000385 | | ggccucca | | CfuAfaaauususu | | | | | | | |
| SRS- | 625 | ususagccAfaUfgGfcc | 225 | usUfsgaagGfaggcCfa | 183 | 84.3 | 9.8 | 101 | 93.6 | 11.9 | 133 |
| 000386 | | uccuucaa | | UfuGfgcuaasasa | | | | | | | |
| SRS- | 626 | cscsaaugGfcCfuCfcu | 226 | usCfsaacuGfaaggAfg | 187 | 97.6 | 10.6 | 133 | 112.7 | 14.8 | 190 |
| 000387 | | ucaguuga | | GfcCfauuggscsu | | | | | | | |
| SRS- | 627 | csasauggCfcUfcCfuu | 227 | usCfscaacUfgaagGfa | 188 | 104.7 | 11.1 | 149 | 106.2 | 12.1 | 178 |
| 000388 | | caguugga | | GfgCfcauugsgsc | | | | | | | |
| SRS- | 628 | asasuggcCfuCfcUfuc | 228 | usCfsccaaCfugaaGfg | 189 | 103.3 | 4.8 | 145 | 108.1 | 10.4 | 182 |
| 000389 | | aguuggga | | AfgGfccauusgsg | | | | | | | |
| SRS- | 629 | csusccuuCfaGfuUfgg | 229 | usCfscaugUfcccaAfc | 195 | 109.8 | 15.9 | 155 | 122.8 | 22.2 | 194 |
| 000390 | | gacaugga | | UfgAfaggagsgsc | | | | | | | |
| SRS- | 630 | csusucagUfuGfgGfac | 230 | usAfsgaccAfugucCfc | 198 | 89.0 | 9.5 | 117 | 92.9 | 11.3 | 127 |
| 000391 | | auggucua | | AfaCfugaagsgsa | | | | | | | |
| SRS- | 631 | asgsuuggGfaCfaUfgg | 231 | usUfsuuaaGfaccaUfg | 202 | 77.9 | 5.0 | 82 | 97.2 | 7.2 | 146 |
| 000392 | | ucuuaaaa | | UfcCfcaacusgsa | | | | | | | |
| SRS- | 632 | ususgggaCfaUfgGfuc | 232 | usUfscuuuAfagacCfa | 204 | 64.7 | 5.1 | 52 | 91.1 | 4.5 | 121 |
| 000393 | | uuaaagaa | | UfgUfcccaascsu | | | | | | | |
| SRS- | 633 | gsgsgacaUfgGfuCfuu | 233 | usAfsgucuUfuaagAfc | 206 | 82.9 | 7.3 | 97 | 99.3 | 7.3 | 153 |
| 000394 | | aaagacua | | CfaUfgucccsasa | | | | | | | |
| SRS- | 634 | gsgsacauGfgUfcUfua | 234 | usAfsagucUfuuaaGfa | 207 | 58.0 | 2.0 | 38 | 84.4 | 7.6 | 98 |
| 000395 | | aagacuua | | CfcAfugucescsa | | | | | | | |
| SRS- | 635 | gsascaugGfuCfuUfaa | 235 | usAfsaaguCfuuuaAfg | 208 | 70.3 | 5.0 | 65 | 84.6 | 3.0 | 99 |
| 000396 | | agacuuua | | AfcCfaugucsesc | | | | | | | |
| SRS- | 636 | ascsauggUfcUfuAfaa | 236 | usCfsaaagUfcuuuAfa | 209 | 79.2 | 5.2 | 87 | 98.6 | 4.6 | 149 |
| 000397 | | gacuuuga | | GfaCfcauguscsc | | | | | | | |
| SRS- | 637 | csasugguCfuUfaAfag | 237 | usAfscaaaGfucuuUfa | 210 | 78.5 | 3.3 | 84 | 86.9 | 8.4 | 103 |
| 000398 | | acuuugua | | AfgAfccaugsusc | | | | | | | |
| SRS- | 638 | ususaaagAfcUfuUfgu | 238 | usCfsuuauGfgacaAfa | 217 | 88.3 | 8.2 | 113 | 94.4 | 3.1 | 138 |
| 000399 | | ccauaaga | | GfuCfuuuaasgsa | | | | | | | |
| SRS- | 639 | ascsgaagGfgCfcAfaa | 239 | usUfscauuAfauuuGf | 237 | 80.7 | 6.6 | 92 | 94.5 | 7.5 | 139 |
| 000400 | | uuaaugaa | | gCfcCfuucguscsu | | | | | | | |
| SRS- | 640 | ascsucaaCfaUfaUfuu | 240 | usAfscugaUfcaaaUfa | 269 | 51.0 | 15.4 | 35 | 52.8 | 14.3 | 35 |
| 000401 | | gaucagua | | UfgUfugagususu | | | | | | | |
| SRS- | 641 | ascsauauUfuGfaUfca | 241 | usAfsaaagAfcugaUfc | 274 | 30.1 | 1.8 | 6 | 35.7 | 1.5 | 6 |
| 000402 | | gucuuuua | | AfaAfuaugususg | | | | | | | |
| SRS- | 642 | gsasucagUfcUfuUfuu | 242 | usAfsgaucAfuaaaAfa | 282 | 35.4 | 5.2 | 14 | 50.7 | 1.5 | 28 |
| 000403 | | augaucua | | GfaCfugaucsasa | | | | | | | |
| SRS- | 643 | asuscaguCfuUfuUfua | 243 | usUfsagauCfauaaAfa | 283 | 20.8 | 1.5 | 1 | 31.1 | 2.0 | 2 |
| 000404 | | ugaucuaa | | AfgAfcugauscsa | | | | | | | |
| SRS- | 644 | ascsauauAfaAfcUfac | 244 | usUfsugacUfuguaGf | 354 | 41.2 | 2.2 | 20 | 45.5 | 4.2 | 21 |
| 000405 | | aagucaaa | | uUfuAfuaugusasg | | | | | | | |
| SRS- | 645 | uscsacuuGfaAfcUfca | 245 | usUfsuugaGfuugaGf | 396 | 39.8 | 3.2 | 19 | 46.4 | 4.6 | 23 |
| 000406 | | acucaaaa | | uUfcAfagugascsa | | | | | | | |
| SRS- | 646 | csasaaacUfuGfaAfag | 246 | usUfsaggaGfgcuuUfc | 412 | 58.2 | 6.5 | 41 | 59.4 | 7.1 | 40 |
| 000407 | | ccuccuaa | | AfaGfuuuugsasg | | | | | | | |
| SRS- | 647 | asasauauUfuAfgAfag | 247 | usAfsguugCfucuuCf | 462 | 78.5 | 7.2 | 86 | 80.2 | 14.1 | 89 |
| 000408 | | agcaacua | | uAfaAfuauuuscsa | | | | | | | |
| SRS- | 648 | asgsaagaGfcAfaCfua | 248 | usAfsguuaGfuuagUf | 470 | 115.5 | 5.4 | 170 | 102.6 | 8.9 | 167 |
| 000409 | | acuaacua | | uGfcUfcuucusasa | | | | | | | |
| SRS- | 649 | asasgagcAfaCfuAfac | 249 | usUfsaaguUfaguuAf | 472 | 73.4 | 6.0 | 74 | 79.9 | 9.0 | 88 |
| 000410 | | uaacuuaa | | gUfuGfcucuuscsu | | | | | | | |
| SRS- | 650 | asgsagcaAfcUfaAfcu | 250 | usUfsuaagUfuaguUfa | 473 | 43.4 | 3.3 | 25 | 46.0 | 2.3 | 22 |
| 000411 | | aacuuaaa | | GfuUfgcucususc | | | | | | | |
| SRS- | 651 | gsasgcaaCfuAfaCfua | 251 | usAfsuuaaGfuuagUf | 474 | 52.8 | 3.9 | 37 | 60.5 | 10.9 | 42 |
| 000412 | | acuuaaua | | uAfgUfugcucsusu | | | | | | | |
| SRS- | 652 | usasacuuAfaUfuCfaa | 252 | usGfsuugaUfuuugAf | 485 | 58.1 | 6.6 | 39 | 67.2 | 6.1 | 51 |
| 000413 | | aaucaaca | | aUfuAfaguuasgsu | | | | | | | |
| SRS- | 653 | ascsccagAfaGfuAfac | 253 | usAfsagugAfaguuAf | 520 | 42.5 | 0.6 | 24 | 58.4 | 4.1 | 38 |
| 000414 | | uucacuua | | cUfuCfugggusgsu | | | | | | | |
| SRS- | 654 | cscscagaAfgUfaAfcu | 254 | usUfsaaguGfaaguUfa | 521 | 47.3 | 4.8 | 33 | 53.4 | 2.1 | 36 |
| 000415 | | ucacuuaa | | CfuUfcugggsusg | | | | | | | |
| SRS- | 655 | asasaaacAfaGfaUfaa | 255 | usGfsaugcUfauuaUfc | 553 | 63.4 | 1.4 | 47 | 65.8 | 3.1 | 48 |
| 000416 | | uagcauca | | UfuGfuuuuuscsu | | | | | | | |
| SRS- | 656 | asasacaaGfaUfaAfua | 256 | usUfsugauGfcuauUfa | 555 | 25.8 | 1.3 | 3 | 31.4 | 1.1 | 3 |
| 000417 | | gcaucaaa | | UfcUfuguuususu | | | | | | | |
| SRS- | 657 | asgsauaaUfaGfcAfuc | 257 | usGfsgucuUfugauGf | 560 | 73.9 | 3.9 | 75 | 74.3 | 3.2 | 66 |
| 000418 | | aaagacca | | cUfaUfuaucususg | | | | | | | |
| SRS- | 658 | asusaauaGfcAfuCfaa | 258 | usAfsagguCfuuugAf | 562 | 93.1 | 3.1 | 126 | 88.1 | 6.6 | 109 |
| 000419 | | agaccuua | | uGfcUfauuauscsu | | | | | | | |
| SRS- | 659 | csasauuaAfaCfcAfac | 259 | usCfsuaugCfuguuGf | 609 | 99.8 | 10.0 | 139 | 100.7 | 11.7 | 161 |
| 000420 | | agcauaga | | gUfuUfaauugsusu | | | | | | | |
| SRS- | 660 | asasccaaCfaGfcAfua | 260 | usAfsuuugAfcuauGf | 615 | 36.9 | 3.2 | 15 | 48.7 | 2.3 | 26 |
| 000421 | | gucaaaua | | cUfgUfugguususa | | | | | | | |
| SRS- | 661 | asgsccaaGfaGfcAfcc | 261 | usAfsguucUfugguGf | 703 | 67.3 | 4.7 | 56 | 63.6 | 2.5 | 45 |
| 000422 | | aagaacua | | cUfcUfuggcususg | | | | | | | |
| SRS- | 662 | uscsagacCfcAfgCfaa | 262 | usUfsugagAfguugCf | 832 | 92.0 | 6.3 | 122 | 93.6 | 0.6 | 134 |
| 000423 | | cucucaaa | | uGfgGfucugasusg | | | | | | | |
| SRS- | 663 | csasgaccCfaGfcAfac | 263 | usCfsuugaGfaguuGfc | 833 | 82.4 | 4.1 | 95 | 97.5 | 7.7 | 148 |
| 000424 | | ucucaaga | | UfgGfgucugsasu | | | | | | | |
| SRS- | 664 | asgsacccAfgCfaAfcu | 264 | usAfscuugAfgaguUf | 834 | 93.6 | 3.2 | 127 | 101.1 | 11.8 | 163 |
| 000425 | | cucaagua | | gCfuGfggucusgsa | | | | | | | |
| SRS- | 665 | asgscaacUfcUfcAfag | 265 | usUfsgaaaAfacuuGfa | 840 | 79.3 | 5.7 | 89 | 78.2 | 7.0 | 80 |
| 000426 | | uuuuucaa | | GfaGfuugcusgsg | | | | | | | |
| SRS- | 666 | ususauauCfaGfgUfag | 266 | usCfscaugGfacuaCfc | 874 | 108.3 | 5.8 | 153 | 99.9 | 8.9 | 157 |
| 000427 | | uccaugga | | UfgAfuauaascsa | | | | | | | |
| SRS- | 667 | usasucagGfuAfgUfcc | 267 | usUfsguccAfuggaCfu | 877 | 92.1 | 4.6 | 123 | 88.9 | 3.8 | 113 |
| 000428 | | auggacaa | | AfcCfugauasusa | | | | | | | |
| SRS- | 668 | asuscaggUfaGfuCfca | 268 | usAfsugucCfauggAfc | 878 | 119.0 | 17.7 | 175 | 91.2 | 7.5 | 122 |
| 000429 | | uggacaua | | UfaCfcugausasu | | | | | | | |
| SRS- | 669 | uscsagguAfgUfcCfau | 269 | usAfsauguCfcaugGfa | 879 | 125.5 | 8.1 | 184 | 98.7 | 9.2 | 150 |
| 000430 | | ggacauua | | CfuAfccugasusa | | | | | | | |
| SRS- | 670 | asgsuccaUfgGfaCfau | 270 | usUfsgaauUfaaugUfc | 885 | 136.9 | 9.4 | 190 | 99.0 | 4.9 | 151 |
| 000431 | | uaauucaa | | CfaUfggacusasc | | | | | | | |
| SRS- | 671 | gsusccauGfgAfcAfuu | 271 | usUfsugaaUfuaauGfu | 886 | 72.2 | 7.3 | 71 | 61.9 | 4.4 | 43 |
| 000432 | | aauucaaa | | CfcAfuggacsusa | | | | | | | |
| SRS- | 672 | uscscaugGfaCfaUfua | 272 | usGfsuugaAfuuaaUf | 887 | 70.5 | 5.9 | 67 | 63.8 | 6.7 | 46 |
| 000238 | | auucaaca | | gUfcCfauggascsu | | | | | | | |
| SRS- | 673 | gsgsacauUfaAfuUfca | 273 | usUfscgauGfuugaAf | 892 | 85.2 | 8.2 | 105 | 83.4 | 4.6 | 96 |
| 000433 | | acaucgaa | | uUfaAfuguccsasu | | | | | | | |
| SRS- | 674 | ascsauuaAfuUfcAfac | 274 | usAfsuucgAfuguuGf | 894 | 81.5 | 1.3 | 93 | 76.6 | 6.2 | 72 |
| 000434 | | aucgaaua | | aAfuUfaauguscsc | | | | | | | |
| SRS- | 675 | csasuuaaUfuCfaAfca | 275 | usUfsauucGfauguUf | 895 | 71.4 | 7.8 | 70 | 77.1 | 4.6 | 74 |
| 000435 | | ucgaauaa | | gAfaUfuaaugsusc | | | | | | | |
| SRS- | 676 | ususaauuCfaAfcAfuc | 276 | usUfscuauUfcgauGfu | 897 | 67.0 | 10.7 | 55 | 75.4 | 6.1 | 70 |
| 000436 | | gaauagaa | | UfgAfauuaasusg | | | | | | | |
| SRS- | 677 | usasauucAfaCfaUfcg | 277 | usAfsucuaUfucgaUfg | 898 | 58.6 | 7.0 | 42 | 79.8 | 5.6 | 87 |
| 000437 | | aauagaua | | UfuGfaauuasasu | | | | | | | |
| SRS- | 678 | asasuucaAfcAfuCfga | 278 | usCfsaucuAfuucgAfu | 899 | 82.4 | 8.6 | 96 | 91.0 | 7.6 | 119 |
| 000438 | | auagauga | | GfuUfgaauusasa | | | | | | | |
| SRS- | 679 | uscsaacaUfcGfaAfua | 279 | usAfsuccaUfcuauUfc | 902 | 75.5 | 12.0 | 77 | 85.9 | 4.9 | 101 |
| 000439 | | gauggaua | | GfaUfguugasasu | | | | | | | |
| SRS- | 680 | csasacauCfgAfaUfag | 280 | usGfsauceAfucuaUfu | 903 | 72.3 | 12.8 | 72 | 73.9 | 6.7 | 64 |
| 000440 | | auggauca | | CfgAfuguugsasa | | | | | | | |
| SRS- | 681 | asascaucGfaAfuAfga | 281 | usUfsgaucCfaucuAfu | 904 | 70.6 | 7.3 | 69 | 79.4 | 4.4 | 86 |
| 000441 | | uggaucaa | | UfcGfauguusgsa | | | | | | | |
| SRS- | 682 | ascsaucgAfaUfaGfau | 282 | usGfsugauCfcaucUfa | 905 | 86.6 | 6.1 | 109 | 88.9 | 2.2 | 112 |
| 000442 | | ggaucaca | | UfuCfgaugususg | | | | | | | |
| SRS- | 683 | uscsgaauAfgAfuGfga | 283 | usUfsuuguGfauccAf | 908 | 89.5 | 5.6 | 119 | 88.5 | 4.6 | 110 |
| 000443 | | ucacaaaa | | uCfuAfuucgasusg | | | | | | | |
| SRS- | 684 | asusagauGfgAfuCfac | 284 | usAfsaguuUfugugAf | 912 | 64.2 | 5.8 | 50 | 71.9 | 6.3 | 60 |
| 000444 | | aaaacuua | | uCfcAfucuaususc | | | | | | | |
| SRS- | 685 | gsasuggaUfcAfcAfaa | 285 | usUfsugaaGfuuuuGf | 915 | 118.4 | 10.5 | 174 | 107.0 | 11.7 | 180 |
| 000445 | | acuucaaa | | uGfaUfccaucsusa | | | | | | | |
| SRS- | 686 | ascsaaaaCfuUfcAfau | 286 | usAfscguuUfcauuGfa | 923 | 97.7 | 11.7 | 134 | 90.3 | 8.2 | 115 |
| 000446 | | gaaacgua | | AfgUfuuugusgsa | | | | | | | |
| SRS- | 687 | asasaacuUfcAfaUfga | 287 | usCfscacgUfuucaUfu | 925 | 117.8 | 10.0 | 172 | 111.8 | 7.1 | 188 |
| 000447 | | aacgugga | | GfaAfguuuusgsu | | | | | | | |
| SRS- | 688 | asasacuuCfaAfuGfaa | 288 | usCfsccacGfuuucAfu | 926 | 113.6 | 15.2 | 162 | 107.5 | 11.5 | 181 |
| 000448 | | acguggga | | UfgAfaguuususg | | | | | | | |
| SRS- | 689 | csusucaaUfgAfaAfcg | 289 | usUfscuccCfacguUfu | 929 | 123.4 | 12.5 | 181 | 116.9 | 18.8 | 192 |
| 000449 | | ugggagaa | | CfaUfugaagsusu | | | | | | | |
| SRS- | 690 | ususcaauGfaAfaCfgu | 290 | usUfsucucCfcacgUfu | 930 | 124.6 | 13.4 | 183 | 111.2 | 9.7 | 187 |
| 000450 | | gggagaaa | | UfcAfuugaasgsu | | | | | | | |
| SRS- | 691 | uscsaaugAfaAfcGfug | 291 | usGfsuucuCfccacGfu | 931 | 97.2 | 9.9 | 132 | 94.3 | 11.5 | 137 |
| 000451 | | ggagaaca | | UfuCfauugasasg | | | | | | | |
| SRS- | 692 | asasugaaAfcGfuGfgg | 292 | usUfsaguuCfucccAfc | 933 | 115.5 | 13.9 | 169 | 106.2 | 9.0 | 177 |
| 000452 | | agaacuaa | | GfuUfucauusgsa | | | | | | | |
| SRS- | 693 | asusgaaaCfgUfgGfga | 293 | usGfsuaguUfcuccCfa | 934 | 105.1 | 6.6 | 150 | 109.6 | 8.1 | 185 |
| 000453 | | gaacuaca | | CfgUfuucaususg | | | | | | | |
| SRS- | 694 | gsasaacgUfgGfgAfga | 294 | usUfsuguaGfuucuCfc | 936 | 112.0 | 9.4 | 158 | 111.2 | 9.2 | 186 |
| 000454 | | acuacaaa | | CfaCfguuucsasu | | | | | | | |
| SRS- | 695 | asasacguGfgGfaGfaa | 295 | usUfsuuguAfguucUf | 937 | 107.7 | 5.5 | 152 | 87.5 | 9.7 | 105 |
| 000455 | | cuacaaaa | | cCfcAfcguuuscsa | | | | | | | |
| SRS- | 696 | usgsggagAfaCfuAfca | 296 | usCfscauaUfuuguAfg | 942 | 68.0 | 4.8 | 58 | 79.2 | 12.1 | 85 |
| 000456 | | aauaugga | | UfuCfucccascsg | | | | | | | |
| SRS- | 697 | usgsggagGfcUfuGfau | 297 | usAfsuucuCfcaucAfa | 965 | 79.2 | 1.9 | 88 | 87.9 | 4.8 | 108 |
| 000457 | | ggagaaua | | GfcCfucccasasa | | | | | | | |
| SRS- | 698 | gsgsgaggCfuUfgAfu | 298 | usAfsauucUfccauCfa | 966 | 43.8 | 1.7 | 28 | 51.7 | 3.7 | 32 |
| 000458 | | ggagaauua | | AfgCfcuccesasa | | | | | | | |
| SRS- | 699 | gsgsgccuAfgAfgAfag | 299 | usAfsguauAfucuuCf | 992 | 88.6 | 2.4 | 115 | 90.5 | 9.4 | 117 |
| 000459 | | auauacua | | uCfuAfggcccsasa | | | | | | | |
| SRS- | 700 | gscscuagAfgAfaGfau | 300 | usGfsgaguAfuaucUf | 994 | 83.3 | 3.9 | 98 | 81.3 | 4.2 | 91 |
| 000460 | | auacucca | | uCfuCfuaggescsc | | | | | | | |
| SRS- | 701 | asgsagaaGfaUfaUfac | 301 | usCfsuaugGfaguaUfa | 998 | 69.3 | 4.1 | 62 | 65.0 | 5.7 | 47 |
| 000461 | | uccauaga | | UfcUfucucusasg | | | | | | | |
| SRS- | 702 | gsasgaagAfuAfuAfcu | 302 | usAfscuauGfgaguAf | 999 | 87.2 | 18.0 | 110 | 81.0 | 5.8 | 90 |
| 000462 | | ccauagua | | uAfuCfuucucsusa | | | | | | | |
| SRS- | 703 | asgsaagaUfaUfaCfuc | 303 | usCfsacuaUfggagUfa | 1000 | 82.3 | 5.7 | 94 | 77.1 | 3.2 | 75 |
| 000463 | | cauaguga | | UfaUfcuucusesu | | | | | | | |
| SRS- | 704 | gsasagauAfuAfcUfcc | 304 | usUfscacuAfuggaGfu | 1001 | 78.5 | 5.8 | 85 | 78.1 | 8.1 | 79 |
| 000464 | | auagugaa | | AfuAfucuucsusc | | | | | | | |
| SRS- | 705 | asasgauaUfaCfuCfca | 305 | usUfsucacUfauggAfg | 1002 | 110.5 | 9.5 | 156 | 118.3 | 6.0 | 193 |
| 000465 | | uagugaaa | | UfaUfaucuuscsu | | | | | | | |
| SRS- | 706 | usasuacuCfcAfuAfgu | 306 | usUfsugcuUfcacuAfu | 1006 | 113.1 | 14.3 | 161 | 104.6 | 17.2 | 174 |
| 000466 | | gaagcaaa | | GfgAfguauasusc | | | | | | | |
| SRS- | 707 | asusacucCfaUfaGfug | 307 | usAfsuugcUfucacUfa | 1007 | 44.7 | 4.2 | 31 | 44.7 | 14.3 | 20 |
| 000467 | | aagcaaua | | UfgGfaguausasu | | | | | | | |
| SRS- | 708 | cscsauagUfgAfaGfca | 308 | usAfsuuagAfuugcUf | 1012 | 41.3 | 2.0 | 21 | 63.0 | 35.1 | 44 |
| 000468 | | aucuaaua | | uCfaCfuauggsasg | | | | | | | |
| SRS- | 709 | asusagugAfaGfcAfau | 309 | usUfsaauuAfgauuGfc | 1014 | 33.9 | 3.7 | 10 | 37.6 | 5.6 | 10 |
| 000469 | | cuaauuaa | | UfuCfacuausgsg | | | | | | | |
| SRS- | 710 | ususuuacUfuGfgGfaa | 310 | usUfscgugAfuuucCfc | 1092 | 76.1 | 1.4 | 79 | 70.0 | 9.7 | 55 |
| 000470 | | aucacgaa | | AfaGfuaaaasasg | | | | | | | |
| SRS- | 711 | ususgggaAfaUfcAfcg | 311 | usUfsugguUfucguGf | 1098 | 38.9 | 1.9 | 18 | 43.6 | 5.5 | 19 |
| 000471 | | aaaccaaa | | aUfuUfcccaasgsu | | | | | | | |
| SRS- | 712 | gsgsgaaaUfcAfcGfaa | 312 | usAfsguugGfuuucGf | 1100 | 101.5 | 9.6 | 142 | 93.6 | 10.0 | 135 |
| 000472 | | accaacua | | uGfaUfuucccsasa | | | | | | | |
| SRS- | 713 | gsgsaaauCfaCfgAfaa | 313 | usUfsaguuGfguuuCf | 1101 | 44.4 | 1.4 | 30 | 43.5 | 6.4 | 18 |
| 000473 | | ccaacuaa | | gUfgAfuuucescsa | | | | | | | |
| SRS- | 714 | asasaucaCfgAfaAfcc | 314 | usUfsauagUfugguUf | 1103 | 43.6 | 3.8 | 27 | 39.6 | 1.9 | 15 |
| 000474 | | aacuauaa | | uCfgUfgauuuscsc | | | | | | | |
| SRS- | 715 | asasucacGfaAfaCfca | 315 | usGfsuauaGfuuggUf | 1104 | 84.5 | 7.1 | 102 | 75.1 | 14.0 | 69 |
| 000475 | | acuauaca | | uUfcGfugauususc | | | | | | | |
| SRS- | 716 | asuscacgAfaAfcCfaa | 316 | usCfsguauAfguugGf | 1105 | 138.9 | 15.1 | 192 | 112.4 | 11.9 | 189 |
| 000476 | | cuauacga | | uUfuCfgugaususu | | | | | | | |
| SRS- | 717 | uscsacgaAfaCfcAfac | 317 | usGfscguaUfaguuGf | 1106 | 136.8 | 12.1 | 189 | 103.0 | 6.5 | 170 |
| 000477 | | uauacgca | | gUfuUfcgugasusu | | | | | | | |
| SRS- | 718 | csasegaaAfcCfaAfcu | 318 | usAfsgcguAfuaguUf | 1107 | 121.9 | 8.2 | 179 | 95.5 | 7.8 | 144 |
| 000478 | | auacgcua | | gGfuUfucgugsasu | | | | | | | |
| SRS- | 719 | gsasaaccAfaCfuAfua | 319 | usUfsguagCfguauAf | 1110 | 134.1 | 17.1 | 188 | 91.0 | 11.5 | 120 |
| 000479 | | cgcuacaa | | gUfuGfguuucsgsu | | | | | | | |
| SRS- | 720 | usgscgauUfaCfuGfgc | 320 | usGfsgacaUfugccAfg | 1136 | 128.9 | 12.7 | 185 | 93.1 | 3.4 | 129 |
| 000480 | | aaugucca | | UfaAfucgcasasc | | | | | | | |
| SRS- | 721 | gscsgauuAfcUfgGfca | 321 | usGfsggacAfuugcCfa | 1137 | 139.5 | 14.8 | 194 | 96.5 | 6.5 | 145 |
| 000481 | | auguccca | | GfuAfaucgcsasa | | | | | | | |
| SRS- | 722 | csgsauuaCfuGfgCfaa | 322 | usGfsgggaCfauugCfc | 1138 | 139.5 | 9.1 | 193 | 94.8 | 20.5 | 140 |
| 000482 | | ugucccca | | AfgUfaaucgscsa | | | | | | | |
| SRS- | 723 | gsasuuacUfgGfcAfau | 323 | usUfsggggAfcauuGf | 1139 | 120.0 | 15.5 | 177 | 90.2 | 10.2 | 114 |
| 000483 | | guccccaa | | cCfaGfuaaucsgsc | | | | | | | |
| SRS- | 724 | ususacugGfcAfaUfgu | 324 | usAfsuuggGfgacaUf | 1141 | 130.9 | 2.9 | 187 | 100.2 | 17.4 | 159 |
| 000484 | | ccccaaua | | uGfcCfaguaasusc | | | | | | | |
| SRS- | 725 | gscsaaugUfcCfcCfaa | 325 | usGfsauugCfauugGf | 1147 | 111.0 | 9.1 | 157 | 109.0 | 7.6 | 184 |
| 000485 | | ugcaauca | | gGfaCfauugcscsa | | | | | | | |
| SRS- | 726 | csasauguCfcCfcAfau | 326 | usGfsgauuGfcauuGf | 1148 | 112.1 | 8.5 | 159 | 105.7 | 5.7 | 176 |
| 000486 | | gcaaucca | | gGfgAfcauugscsc | | | | | | | |
| SRS- | 727 | asusguccCfcAfaUfgc | 327 | usCfsgggaUfugcaUfu | 1150 | 146.4 | 22.6 | 195 | 99.9 | 16.4 | 156 |
| 000487 | | aaucccga | | GfgGfgacaususg | | | | | | | |
| SRS- | 728 | cscsccaaUfgCfaAfuc | 328 | usUfsuuccGfggauUf | 1154 | 104.5 | 15.0 | 148 | 78.4 | 18.5 | 81 |
| 000488 | | ccggaaaa | | gCfaUfuggggsasc | | | | | | | |
| SRS- | 729 | csasaugcAfaUfcCfcg | 329 | usUfsguuuUfccggGf | 1157 | 88.8 | 9.2 | 116 | 90.8 | 17.0 | 118 |
| 000489 | | gaaaacaa | | aUfuGfcauugsgsg | | | | | | | |
| SRS- | 730 | asasugcaAfuCfcCfgg | 330 | usUfsuguuUfuccgGf | 1158 | 138.3 | 50.1 | 191 | 77.9 | 31.1 | 78 |
| 000490 | | aaaacaaa | | gAfuUfgcauusgsg | | | | | | | |
| SRS- | 731 | asusgcaaUfcCfcGfga | 331 | usUfsuuguUfuuccGf | 1159 | 108.7 | 16.6 | 154 | 94.2 | 8.9 | 136 |
| 000491 | | aaacaaaa | | gGfaUfugcaususg | | | | | | | |
| SRS- | 732 | usgscaauCfcCfgGfaa | 332 | usCfsuuugUfuuucCf | 1160 | 112.4 | 8.5 | 160 | 82.0 | 19.7 | 92 |
| 000492 | | aacaaaga | | gGfgAfuugcasusu | | | | | | | |
| SRS- | 733 | gscsaaucCfcGfgAfaa | 333 | usUfscuuuGfuuuuCf | 1161 | 115.4 | 4.9 | 168 | 104.6 | 15.8 | 173 |
| 000493 | | acaaagaa | | cGfgGfauugcsasu | | | | | | | |
| SRS- | 734 | csasauccCfgGfaAfaa | 334 | usAfsucuuUfguuuUf | 1162 | 175.8 | 33.1 | 196 | 100.7 | 12.2 | 160 |
| 000494 | | caaagaua | | cCfgGfgauugscsa | | | | | | | |
| SRS- | 735 | gsasuuugGfuGfuUfu | 335 | usCfsaaguAfgaaaAfc | 1179 | 61.8 | 2.6 | 43 | 72.6 | 13.9 | 61 |
| 000495 | | ucuacuuga | | AfcCfaaaucsusu | | | | | | | |
| SRS- | 736 | asusuuggUfgUfuUfu | 336 | usCfscaagUfagaaAfa | 1180 | 78.2 | 3.3 | 83 | 88.5 | 3.2 | 111 |
| 000496 | | cuacuugga | | CfaCfcaaauscsu | | | | | | | |
| SRS- | 737 | ususugguGfuUfuUfc | 337 | usCfsccaaGfuagaAfa | 1181 | 88.4 | 21.1 | 114 | 99.4 | 13.1 | 154 |
| 000497 | | uacuuggga | | AfcAfccaaasusc | | | | | | | |
| SRS- | 738 | usgsuuuuCfuAfcUfu | 338 | usGfsugauCfccaaGfu | 1186 | 61.9 | 14.3 | 44 | 66.0 | 4.7 | 49 |
| 000498 | | gggaucaca | | AfgAfaaacascsc | | | | | | | |
| SRS- | 739 | ususucuaCfuUfgGfga | 339 | usUfsuuguGfauccCfa | 1189 | 86.3 | 13.1 | 108 | 93.4 | 7.4 | 131 |
| 000499 | | ucacaaaa | | AfgUfagaaasasc | | | | | | | |
| SRS- | 740 | ususcuacUfuGfgGfau | 340 | usCfsuuugUfgaucCfc | 1190 | 101.7 | 35.2 | 143 | 78.7 | 3.5 | 83 |
| 000500 | | cacaaaga | | AfaGfuagaasasa | | | | | | | |
| SRS- | 741 | csusacuuGfgGfaUfca | 341 | usUfsgcuuUfgugaUf | 1192 | 93.0 | 2.4 | 125 | 92.8 | 14.2 | 126 |
| 000501 | | caaagcaa | | cCfcAfaguagsasa | | | | | | | |
| SRS- | 742 | usascuugGfgAfuCfac | 342 | usUfsugcuUfugugAf | 1193 | 89.4 | 33.3 | 118 | 68.8 | 14.0 | 54 |
| 000502 | | aaagcaaa | | uCfcCfaaguasgsa | | | | | | | |
| SRS- | 743 | asasugguAfaAfuAfua | 343 | usGfsguuuGfuuauAf | 1287 | 96.7 | 1.7 | 130 | 101.2 | 36.7 | 164 |
| 000503 | | acaaacca | | uUfuAfccauususa | | | | | | | |
| SRS- | 744 | gsasagucUfcAfaAfau | 344 | usAfsccuuCfcauuUfu | 1349 | 107.6 | 11.1 | 151 | 108.9 | 5.3 | 183 |
| 000504 | | ggaaggua | | GfaGfacuucscsa | | | | | | | |
| SRS- | 745 | asasaaugGfaAfgGfuu | 345 | usAfsgaguAfuaacCfu | 1357 | 41.9 | 2.4 | 23 | 48.4 | 10.8 | 25 |
| 000505 | | auacucua | | UfcCfauuuusgsa | | | | | | | |
| SRS- | 746 | asasauggAfaGfgUfua | 346 | usUfsagagUfauaaCfc | 1358 | 31.1 | 6.0 | 8 | 34.5 | 14.1 | 5 |
| 000506 | | uacucuaa | | UfuCfcauuususg | | | | | | | |
| SRS- | 747 | asasuggaAfgGfuUfau | 347 | usAfsuagaGfuauaAfc | 1359 | 63.3 | 6.3 | 46 | 52.3 | 4.9 | 34 |
| 000507 | | acucuaua | | CfuUfccauususu | | | | | | | |
| SRS- | 748 | gsasagguUfaUfaCfuc | 348 | usUfsuuuaUfagagUfa | 1363 | 46.0 | 2.3 | 32 | 51.9 | 12.8 | 33 |
| 000508 | | uauaaaaa | | UfaAfccuucscsa | | | | | | | |
| SRS- | 749 | asgsguuaUfaCfuCfua | 349 | usGfsauuuUfauagAf | 1365 | 63.5 | 3.8 | 48 | 47.4 | 15.6 | 24 |
| 000509 | | uaaaauca | | gUfaUfaaccususc | | | | | | | |
| SRS- | 750 | asasucaaCfcAfaAfau | 350 | usGfsaucaAfcauuUfu | 1381 | 116.8 | 14.2 | 171 | 78.7 | 7.1 | 84 |
| 000510 | | guugauca | | GfgUfugauususu | | | | | | | |
| SRS- | 751 | asuscaacCfaAfaAfug | 351 | usGfsgaucAfacauUfu | 1382 | 65.2 | 1.7 | 53 | 50.9 | 5.8 | 30 |
| 000511 | | uugaucca | | UfgGfuugaususu | | | | | | | |
| SRS- | 752 | uscsaaccAfaAfaUfgu | 352 | usUfsggauCfaacaUfu | 1383 | 86.1 | 8.0 | 107 | 74.4 | 10.0 | 67 |
| 000512 | | ugauccaa | | UfuGfguugasusu | | | | | | | |
| SRS- | 753 | csasaccaAfaAfuGfuu | 353 | usAfsuggaUfcaacAfu | 1384 | 44.0 | 2.6 | 29 | 37.3 | 7.2 | 8 |
| 000513 | | gauccaua | | UfuUfgguugsasu | | | | | | | |
| SRS- | 754 | ascscaaaAfuGfuUfga | 354 | usGfsgaugGfaucaAfc | 1386 | 124.3 | 7.8 | 182 | 101.3 | 10.7 | 165 |
| 000514 | | uccaucca | | AfuUfuuggususg | | | | | | | |
| SRS- | 755 | cscsaaaaUfgUfuGfau | 355 | usUfsggauGfgaucAfa | 1387 | 69.5 | 10.7 | 63 | 78.5 | 9.1 | 82 |
| 000515 | | ccauccaa | | CfaUfuuuggsusu | | | | | | | |
| SRS- | 756 | asasccucAfuUfcCfaa | 356 | usCfsauuaAfcuugGfa | 1467 | 99.2 | 9.7 | 137 | 94.9 | 10.3 | 141 |
| 000516 | | guuaauga | | AfuGfagguusasa | | | | | | | |
| SRS- | 757 | asusaaucUfgGfuAfuu | 357 | usAfsggauUfuaauAfc | 1494 | 51.4 | 6.4 | 36 | 59.3 | 8.5 | 39 |
| 000517 | | aaauccua | | CfaGfauuaususa | | | | | | | |
| SRS- | 758 | asasucugGfuAfuUfaa | 358 | usUfsaaggAfuuuaAf | 1496 | 38.2 | 5.4 | 17 | 40.3 | 7.9 | 17 |
| 000518 | | auccuuaa | | uAfcCfagauusasu | | | | | | | |
| SRS- | 759 | uscsugguAfuUfaAfau | 359 | usCfsuuaaGfgauuUfa | 1498 | 74.2 | 7.4 | 76 | 74.9 | 10.5 | 68 |
| 000519 | | ccuuaaga | | AfuAfccagasusu | | | | | | | |
| SRS- | 760 | asusuuaaGfaUfuAfaa | 360 | usUfsuguaUfguuuAf | 1563 | 58.1 | 10.7 | 40 | 51.6 | 6.9 | 31 |
| 000520 | | cauacaaa | | aUfcUfuaaausasg | | | | | | | |
| SRS- | 761 | usasagauUfaAfaCfau | 361 | usUfsgauuGfuaugUf | 1566 | 63.9 | 6.5 | 49 | 72.6 | 17.8 | 62 |
| 000521 | | acaaucaa | | uUfaAfucuuasasa | | | | | | | |
| SRS- | 762 | uscsacauAfaCfcUfua | 362 | usUfsauucUfuuaaGfg | 1583 | 69.1 | 11.0 | 61 | 67.9 | 8.3 | 53 |
| 000522 | | aagaauaa | | UfuAfugugasusu | | | | | | | |
| SRS- | 763 | usgsugauGfuGfgGfaa | 363 | usAfsaauuGfauucCfc | 1654 | 69.7 | 7.4 | 64 | 82.0 | 14.7 | 93 |
| 000523 | | ucaauuua | | AfcAfucacasasa | | | | | | | |
| SRS- | 764 | gsgsgaauCfaAfuUfuu | 364 | usAfsccauCfuaaaAfu | 1662 | 103.3 | 13.1 | 146 | 93.3 | 3.7 | 130 |
| 000524 | | agauggua | | UfgAfuucccsasc | | | | | | | |
| SRS- | 765 | asgsgcauCfaUfaUfga | 365 | usUfsauuaGfcucaUfa | 1761 | 68.3 | 3.5 | 59 | 74.3 | 5.5 | 65 |
| 000525 | | gcuaauaa | | UfgAfugccususu | | | | | | | |
| SRS- | 766 | asuscauaUfgAfgCfua | 366 | usGfsugauAfuuagCf | 1765 | 87.8 | 9.0 | 111 | 95.4 | 9.3 | 142 |
| 000526 | | auaucaca | | uCfaUfaugausgsc | | | | | | | |
| SRS- | 767 | uscsauauGfaGfcUfaa | 367 | usUfsgugaUfauuaGfc | 1766 | 84.9 | 11.5 | 103 | 87.7 | 8.0 | 107 |
| 000527 | | uaucacaa | | UfcAfuaugasusg | | | | | | | |
| SRS- | 768 | gsusuaaaAfcUfcUfaa | 368 | usGfsucaaGfuuuaGfa | 1816 | 90.0 | 10.8 | 120 | 87.4 | 8.9 | 104 |
| 000528 | | acuugaca | | GfuUfuuaacsasa | | | | | | | |
| SRS- | 769 | usasaaacUfcUfaAfac | 369 | usUfsagucAfaguuUfa | 1818 | 70.6 | 11.2 | 68 | 77.7 | 7.6 | 77 |
| 000529 | | uugacuaa | | GfaGfuuuuasasc | | | | | | | |
| SRS- | 770 | ascsucuaAfaCfuUfga | 370 | usUfsauuuAfgucaAf | 1822 | 79.5 | 3.5 | 90 | 77.3 | 3.8 | 76 |
| 000530 | | cuaaauaa | | gUfuUfagagususu | | | | | | | |
| SRS- | 771 | asuscgucAfgCfaCfag | 371 | usAfscauaCfucugUfg | 1900 | 114.2 | 12.3 | 165 | 103.8 | 19.8 | 171 |
| 000531 | | aguaugua | | CfuGfacgaususu | | | | | | | |
| SRS- | 772 | gsascuacUfaAfgUfca | 372 | usGfsucaaUfgugaCfu | 2074 | 100.8 | 8.6 | 140 | 87.6 | 10.4 | 106 |
| 000532 | | cauugaca | | UfaGfuagucsasu | | | | | | | |
| SRS- | 773 | ascsaugaGfgUfaUfca | 373 | usGfsguauAfgugaUf | 2098 | 114.4 | 6.4 | 166 | 155.9 | 21.4 | 196 |
| 000533 | | cuauacca | | aCfcUfcaugususa | | | | | | | |
| SRS- | 774 | usgsgcauCfgAfgUfua | 374 | usUfsaaacUfuuaaCfu | 2231 | 77.2 | 4.5 | 81 | 83.7 | 13.8 | 97 |
| 000534 | | aaguuuaa | | CfgAfugccascsa | | | | | | | |
| SRS- | 775 | gsgscaucGfaGfuUfaa | 375 | usAfsuaaaCfuuuaAfc | 2232 | 77.2 | 2.2 | 80 | 84.9 | 6.7 | 100 |
| 000535 | | aguuuaua | | UfcGfaugccsasc | | | | | | | |
| SRS- | 776 | usasaaguUfuAfuAfuu | 376 | usUfsagggGfaaauAfu | 2242 | 79.9 | 4.7 | 91 | 101.4 | 13.5 | 166 |
| 000536 | | uccecuaa | | AfaAfcuuuasasc | | | | | | | |
| SRS- | 777 | ususaaacCfcAfuUfug | 377 | usCfscuuuAfacaaAfu | 2560 | 96.3 | 8.8 | 129 | 99.5 | 6.6 | 155 |
| 000537 | | uuaaagga | | GfgGfuuuaasusu | | | | | | | |
| SRS- | 778 | asusuuguUfaAfaGfga | 378 | usCfsacuaUfauccUfu | 2568 | 92.4 | 9.9 | 124 | 92.8 | 2.9 | 125 |
| 000538 | | uauaguga | | UfaAfcaaausgsg | | | | | | | |
| SRS- | 779 | ususuguuAfaAfgGfa | 379 | usGfscacuAfuaucCfu | 2569 | 84.1 | 16.1 | 100 | 99.1 | 6.0 | 152 |
| 000539 | | uauagugca | | UfuAfacaaasusg | | | | | | | |
| SRS- | 780 | usgsuuaaAfgGfaUfau | 380 | usGfsggcaCfuauaUfc | 2571 | 97.0 | 5.5 | 131 | 76.8 | 19.4 | 73 |
| 000540 | | agugccca | | CfuUfuaacasasa | | | | | | | |
| SRS- | 781 | gsusuaaaGfgAfuAfua | 381 | usUfsgggcAfcuauAf | 2572 | 85.0 | 6.0 | 104 | 83.0 | 5.4 | 94 |
| 000541 | | gugcccaa | | uCfcUfuuaacsasa | | | | | | | |
| SRS- | 782 | asasggauAfuAfgUfgc | 382 | usAfsacuuGfggcaCfu | 2576 | 99.4 | 2.0 | 138 | 93.5 | 11.5 | 132 |
| 000542 | | ccaaguua | | AfuAfuccuususa | | | | | | | |
| SRS- | 783 | asgsgauaUfaGfuGfcc | 383 | usUfsaacuUfgggcAfc | 2577 | 91.5 | 3.5 | 121 | 92.9 | 7.4 | 128 |
| 000543 | | caaguuaa | | UfaUfauccususu | | | | | | | |
| SRS- | 784 | gsgsauauAfgUfgCfcc | 384 | usAfsuaacUfugggCfa | 2578 | 99.0 | 7.4 | 136 | 92.5 | 7.2 | 124 |
| 000544 | | aaguuaua | | CfuAfuaucesusu | | | | | | | |
| SRS- | 785 | gsasuauaGfuGfcCfca | 385 | usUfsauaaCfuuggGfc | 2579 | 98.8 | 8.1 | 135 | 97.3 | 8.8 | 147 |
| 000545 | | aguuauaa | | AfcUfauaucscsu | | | | | | | |
| SRS- | 786 | asusggugAfcCfuAfcc | 386 | usUfsgacaAfagguAfg | 2598 | 101.5 | 14.2 | 141 | 106.7 | 8.7 | 179 |
| 000546 | | uuugucaa | | GfuCfaccausasu | | | | | | | |
| SRS- | 787 | usgsgugaCfcUfaCfcu | 387 | usUfsugacAfaaggUfa | 2599 | 96.0 | 5.6 | 128 | 100.7 | 14.1 | 162 |
| 000547 | | uugucaaa | | GfgUfcaccasusa | | | | | | | |
| SRS- | 788 | gsgsugacCfuAfcCfuu | 388 | usAfsuugaCfaaagGfu | 2600 | 85.7 | 8.5 | 106 | 83.1 | 7.6 | 95 |
| 000548 | | ugucaaua | | AfgGfucaccsasu | | | | | | | |
| SRS- | 789 | asasuuauCfcAfaUfau | 389 | usGfsacauGfuauaUfu | 2640 | 114.0 | 5.8 | 163 | 102.8 | 15.6 | 169 |
| 000549 | | acauguca | | GfgAfuaauususg | | | | | | | |
| SRS- | 790 | asusuuucAfgGfaCfca | 390 | usUfsagucUfguggUf | 2772 | 103.8 | 13.4 | 147 | 91.4 | 21.6 | 123 |
| 000550 | | cagacuaa | | cCfuGfaaaaususa | | | | | | | |
| SRS- | 791 | ususcaggAfcCfaCfag | 391 | usGfscuuaGfucugUf | 2775 | 130.4 | 12.7 | 186 | 104.2 | 11.4 | 172 |
| 000551 | | acuaagca | | gGfuCfcugaasasa | | | | | | | |
| SRS- | 792 | asgsgaccAfcAfgAfcu | 392 | usAfscagcUfuaguCfu | 2778 | 115.0 | 10.9 | 167 | 90.5 | 7.3 | 116 |
| 000552 | | aagcugua | | GfuGfguccusgsa | | | | | | | |
| SRS- | 793 | ususuuuuUfaGfgGfcc | 393 | usGfsuauuCfuggcCfc | 2813 | 122.1 | 13.9 | 180 | 139.2 | 42.1 | 195 |
| 000553 | | agaauaca | | UfaAfaaaaasusc | | | | | | | |
| SRS- | 794 | ususuuagGfgCfcAfga | 394 | usUfsugguAfuucuGf | 2816 | 118.3 | 14.9 | 173 | 100.0 | 15.3 | 158 |
| 000554 | | auaccaaa | | gCfcCfuaaaasasa | | | | | | | |
| SRS- | 795 | ususuaggGfcCfaGfaa | 395 | usUfsuuggUfauucUf | 2817 | 120.6 | 12.2 | 178 | 102.6 | 11.0 | 168 |
| 000555 | | uaccaaaa | | gGfcCfcuaaasasa | | | | | | | |
| SRS- | 796 | ususuaauAfuAfuGfag | 396 | usGfsaagcAfacucAfu | 2892 | 119.4 | 5.3 | 176 | 95.4 | 12.1 | 143 |
| 000556 | | uugcuuca | | AfuAfuuaaasusa | | | | | | | |

TABLE 7
Resuts of Dose Response In vitro of siRNAs targeting ANGPTL3

| | | SEQ | | SEQ | | IC₅₀ | IC₈₀ | MV | SD |
|---|---|---|---|---|---|---|---|---|---|
| Duplex | Transcript | ID | | ID | | @ | @ | max | max |
| ID | Position | NO. | Antisense strand sequence | NO. | Sense strand sequence | [μM] | [μM] | KD | KD |
| SRS- | 47 | 202 | usGfsgagcUfuaauUfgUfgAfacauu | 602 | asasuguuCfaCfaAfuuaagcu | 0.007 | 2.813 | 81 | 2 |
| 000363 | | | susu | | cca | | | | |
| SRS- | 49 | 203 | usAfsaggaGfcuuaAfuUfgUfgaaca | 603 | usgsuucaCfaAfuUfaagcucc | 0.020 | n/a | 70 | 2 |
| 000364 | | | susu | | uua | | | | |
| SRS- | 56 | 205 | usUfsaaaaAfgaagGfaGfcUfuaauu | 605 | asasuuaaGfcUfcCfuucuuuu | 0.005 | n/a | 78 | 1 |
| 000366 | | | sgsu | | uaa | | | | |
| SRS- | 71 | 207 | usUfsaacuAfgaggAfaCfaAfuaaaas | 607 | ususuuauUfgUfuCfcucuagu | 0.224 | n/a | 60 | 2 |
| 000368 | | | asg | | uaa | | | | |
| SRS- | 72 | 208 | usAfsuaacUfagagGfaAfcAfauaaas | 608 | ususuauuGfuUfcCfucuaguu | 0.019 | n/a | 70 | 3 |
| 000369 | | | asa | | aua | | | | |
| SRS- | 149 | 210 | usCfsuaacAfuagcAfaAfuCfuugau | 610 | asuscaagAfuUfuGfcuauguu | 0.728 | n/a | 58 | 2 |
| 000371 | | | susu | | aga | | | | |
| SRS- | 151 | 211 | usGfsucuaAfcauaGfcAfaAfucuug | 611 | csasagauUfuGfcUfauguuag | 0.283 | n/a | 61 | 2 |
| 000372 | | | sasu | | aca | | | | |
| SRS- | 154 | 214 | usAfsucguCfuaacAfuAfgCfaaauc | 614 | gsasuuugCfuAfuGfuuagacg | 0.854 | n/a | 54 | 1 |
| 000375 | | | susu | | aua | | | | |
| SRS- | 155 | 215 | usCfsaucgUfcuaaCfaUfaGfcaaaus | 615 | asusuugcUfaUfgUfuagacga | 1.461 | n/a | 59 | 2 |
| 000376 | | | csu | | uga | | | | |
| SRS- | 159 | 218 | usUfsuuacAfucguCfuAfaCfauagc | 618 | gscsuaugUfuAfgAfcgaugua | 0.047 | n/a | 75 | 1 |
| 000379 | | | sasa | | aaa | | | | |
| SRS- | 161 | 220 | usUfsuuuuAfcaucGfuCfuAfacaua | 620 | usasuguuAfgAfcGfauguaaa | 0.288 | n/a | 66 | 4 |
| 000381 | | | sgsc | | aaa | | | | |
| SRS- | 163 | 221 | usAfsauuuUfuacaUfcGfuCfuaaca | 621 | usgsuuagAfcGfaUfguaaaaa | 1.486 | n/a | 51 | 3 |
| 000382 | | | susa | | uua | | | | |
| SRS- | 165 | 222 | usAfsaaauUfuuuaCfaUfcGfucuaa | 622 | ususagacGfaUfgUfaaaaauu | 0.009 | 1.735 | 82 | 4 |
| 000383 | | | scsa | | uua | | | | |
| SRS- | 204 | 232 | usUfscuuuAfagacCfaUfgUfcccaas | 632 | ususgggaCfaUfgGfucuuaaa | 48.262 | n/a | 38 | 2 |
| 000393 | | | csu | | gaa | | | | |
| SRS- | 207 | 234 | usAfsagucUfuuaaGfaCfcAfugucc | 634 | gsgsacauGfgUfcUfuaaagac | n/a | n/a | 37 | 4 |
| 000395 | | | scsa | | uua | | | | |
| SRS- | 282 | 242 | usAfsgaucAfuaaaAfaGfaCfugauc | 642 | gsasucagUfcUfuUfuuaugau | 2.848 | n/a | 54 | 4 |
| 000403 | | | sasa | | cua | | | | |
| SRS- | 283 | 243 | usUfsagauCfauaaAfaAfgAfcugau | 643 | asuscaguCfuUfuUfuaugauc | 0.078 | n/a | 66 | 4 |
| 000404 | | | scsa | | uaa | | | | |
| SRS- | 354 | 244 | usUfsugacUfuguaGfuUfuAfuaugu | 644 | ascsauauAfaAfcUfacaaguc | 0.006 | n/a | 78 | 2 |
| 000405 | | | sasg | | aaa | | | | |
| SRS- | 396 | 245 | usUfsuugaGfuugaGfuUfcAfaguga | 645 | uscsacuuGfaAfcUfcaacuca | 0.010 | n/a | 77 | 3 |
| 000406 | | | scsa | | aaa | | | | |
| SRS- | 412 | 246 | usUfsaggaGfgcuuUfcAfaGfuuuug | 646 | csasaaacUfuGfaAfagccucc | 0.014 | 30.020 | 77 | 2 |
| 000407 | | | sasg | | uaa | | | | |
| SRS- | 473 | 250 | usUfsuaagUfuaguUfaGfuUfgcucu | 650 | asgsagcaAfcUfaAfcuaacuu | 0.014 | 2.834 | 82 | 2 |
| 000411 | | | susc | | aaa | | | | |
| SRS- | 474 | 251 | usAfsuuaaGfuuagUfuAfgUfugcuc | 651 | gsasgcaaCfuAfaCfuaacuua | 5.837 | n/a | 53 | 4 |
| 000412 | | | susu | | aua | | | | |
| SRS- | 485 | 252 | usGfsuugaUfuuugAfaUfuAfaguua | 652 | usasacuuAfaUfuCfaaaauca | 0.042 | n/a | 68 | 1 |
| 000413 | | | sgsu | | aca | | | | |
| SRS- | 520 | 253 | usAfsagugAfaguuAfcUfuCfugggu | 653 | ascsccagAfaGfuAfacuucac | 0.050 | n/a | 70 | 1 |
| 000414 | | | sgsu | | uua | | | | |
| SRS- | 521 | 254 | usUfsaaguGfaaguUfaCfuUfcuggg | 654 | cscscagaAfgUfaAfcuucacu | 0.563 | n/a | 53 | 15 |
| 000415 | | | susg | | uaa | | | | |
| SRS- | 553 | 255 | usGfsaugcUfauuaUfcUfuGfuuuuu | 655 | asasaaacAfaGfaUfaauagcau | 18.689 | n/a | 49 | 9 |
| 000416 | | | scsu | | ca | | | | |
| SRS- | 555 | 256 | usUfsugauGfcuauUfaUfcUfuguuu | 656 | asasacaaGfaUfaAfuagcauca | 0.023 | n/a | 73 | 3 |
| 000417 | | | susu | | aa | | | | |
| SRS- | 615 | 260 | usAfsuuugAfcuauGfcUfgUfuggu | 660 | asasccaaCfaGfcAfuagucaaa | 0.479 | n/a | 66 | 5 |
| 000421 | | | ususa | | ua | | | | |
| SRS- | 897 | 276 | usUfscuauUfcgauGfuUfgAfauuaa | 676 | ususaauuCfaAfcAfucgaaua | n/a | n/a | 11 | 18 |
| 000436 | | | susg | | gaa | | | | |
| SRS- | 898 | 277 | usAfsucuaUfucgaUfgUfuGfaauua | 677 | usasauucAfaCfaUfcgaauag | n/a | n/a | 30 | 11 |
| 000437 | | | sasu | | aua | | | | |
| SRS- | 942 | 296 | usCfscauaUfuuguAfgUfuCfuccca | 696 | usgsggagAfaCfuAfcaaauau | n/a | n/a | 21 | 24 |
| 000456 | | | scsg | | gga | | | | |
| SRS- | 966 | 298 | usAfsauucUfccauCfaAfgCfcucccs | 698 | gsgsgaggCfuUfgAfuggagaa | n/a | n/a | 51 | 6 |
| 000458 | | | asa | | uua | | | | |
| SRS- | 1007 | 307 | usAfsuugcUfucacUfaUfgGfaguau | 707 | asusacucCfaUfaGfugaagca | 0.728 | n/a | 61 | 3 |
| 000467 | | | sasu | | aua | | | | |
| SRS- | 1012 | 308 | usAfsuuagAfuugcUfuCfaCfuaugg | 708 | cscsauagUfgAfaGfcaaucua | 0.373 | n/a | 66 | 6 |
| 000468 | | | sasg | | aua | | | | |
| SRS- | 1014 | 309 | usUfsaauuAfgauuGfcUfuCfacuau | 709 | asusagugAfaGfcAfaucuaau | 0.115 | n/a | 70 | 2 |
| 000469 | | | sgsg | | uaa | | | | |
| SRS- | 1098 | 311 | usUfsugguUfucguGfaUfuUfcccaa | 711 | ususgggaAfaUfcAfcgaaacc | 4.966 | n/a | 60 | 6 |
| 000471 | | | sgsu | | aaa | | | | |
| SRS- | 1101 | 313 | usUfsaguuGfguuuCfgUfgAfuuucc | 713 | gsgsaaauCfaCfgAfaaccaacu | 0.025 | n/a | 70 | 6 |
| 000473 | | | scsa | | aa | | | | |
| SRS- | 1103 | 314 | usUfsauagUfugguUfuCfgUfgauuu | 714 | asasaucaCfgAfaAfccaacuau | 0.303 | n/a | 59 | 4 |
| 000474 | | | scSc | | aa | | | | |
| SRS- | 1179 | 335 | usCfsaaguAfgaaaAfcAfcCfaaaucs | 735 | gsasuuugGfuGfuUfuucuacu | 2.674 | n/a | 55 | 9 |
| 000495 | | | usu | | uga | | | | |
| SRS- | 1357 | 345 | usAfsgaguAfuaacCfuUfcCfauuuu | 745 | asasaaugGfaAfgGfuuauacu | 0.100 | n/a | 70 | 5 |
| 000505 | | | sgsa | | cua | | | | |
| SRS- | 1358 | 346 | usUfsagagUfauaaCfcUfuCfcauuu | 746 | asasauggAfaGfgUfuauacuc | 0.013 | 10.634 | 78 | 2 |
| 000506 | | | susg | | uaa | | | | |
| SRS- | 1363 | 348 | usUfsuuuaUfagagUfaUfaAfccuuc | 748 | gsasagguUfaUfaCfucuauaa | 0.265 | n/a | 66 | 4 |
| 000508 | | | scsa | | aaa | | | | |
| SRS- | 1365 | 349 | usGfsauuuUfauagAfgUfaUfaaccu | 749 | asgsguuaUfaCfuCfuauaaaa | n/a | n/a | 40 | 10 |
| 000509 | | | susc | | uca | | | | |
| SRS- | 1384 | 353 | usAfsuggaUfcaacAfuUfuUfgguug | 753 | csasaccaAfaAfuGfuugaucc | 0.030 | n/a | 68 | 6 |
| 000513 | | | sasu | | aua | | | | |
| SRS- | 1494 | 357 | usAfsggauUfuaauAfcCfaGfauuau | 757 | asusaaucUfgGfuAfuuaaauc | 0.305 | 54.336 | 67 | 5 |
| 000517 | | | susa | | cua | | | | |
| SRS- | 1496 | 358 | usUfsaaggAfuuuaAfuAfcCfagauu | 758 | asasucugGfuAfuUfaaauccu | 0.058 | 86.253 | 78 | 4 |
| 000518 | | | sasu | | uaa | | | | |
| SRS- | 1563 | 360 | usUfsuguaUfguuuAfaUfcUfuaaau | 760 | asusuuaaGfaUfuAfaacauac | 159.734 | n/a | 46 | 4 |
| 000520 | | | sasg | | aaa | | | | |
| SRS- | 1566 | 361 | usUfsgauuGfuaugUfuUfaAfucuua | 761 | usasagauUfaAfaCfauacaau | 0.629 | n/a | 56 | 5 |
| 000521 | | | sasa | | caa | | | | |

TABLE 8
Results of % change in serum hANGPTL3 protein levels
Percent Change in Serum hANGPTL3 Concentration from Day −4 Baseline

| | | | SRS- | | SRS- | | SRS- | | SRS- | | SRS- | | SRS- | | SRS- | |
|---|---|---|---|---|---|---|---|---|---|---|---|---|---|---|---|---|
| Study | Vehicle | | 000651 | | 000652 | | 000653 | | 000654 | | 000656 | | 000658 | | 000659 | |
| Day | Avg | SEM | Avg | SEM | Avg | SEM | Avg | SEM | Avg | SEM | Avg | SEM | Avg | SEM | Avg | SEM |
| −4 | 0.0 | 0.0 | 0.0 | 0.0 | 0.0 | 0.0 | 0.0 | 0.0 | 0.0 | 0.0 | 0.0 | 0.0 | 0.0 | 0.0 | 0.0 | 0.0 |
| 7 | 25.5 | 15.5 | −54.7 | 11.9 | 52.9 | 19.9 | 33.7 | 10.8 | −11.6 | 18.4 | −57.8 | 7.1 | −67.0 | 6.3 | −35.4 | 10.8 |
| 14 | −9.7 | 3.1 | −62.3 | 8.4 | 19.5 | 19.5 | 23.4 | 21.1 | 12.9 | 16.2 | −63.7 | 4.9 | −53.4 | 13.3 | 52.1 | 9.0 |
| 21 | 23.3 | 9.7 | −43.7 | 12.8 | 2.4 | 11.7 | 24.3 | 9.2 | 10.2 | 20.5 | −51.5 | 6.6 | −61.1 | 6.3 | 22.0 | 10.4 |
| 28 | 3.1 | 17.4 | −43.5 | 19.0 | −21.9 | 6.5 | 4.1 | 11.2 | −5.6 | 10.4 | −46.4 | 9.3 | −51.7 | 7.5 | 43.4 | 19.0 |
| 35 | −13.3 | 7.4 | −42.8 | 18.8 | −32.3 | 9.0 | 6.4 | 9.1 | −13.9 | 10.8 | −40.4 | 8.7 | −49.7 | 9.3 | 15.1 | 13.9 |
| 42 | 0.8 | 6.3 | −36.9 | 15.2 | −31.1 | 11.8 | 5.4 | 7.4 | 0.1 | 12.6 | −36.0 | 6.7 | −34.5 | 2.7 | 2.7 | 12.5 |
| 49 | −0.7 | 6.9 | −14.1 | 21.6 | −16.5 | 11.7 | 13.6 | 5.5 | 20.0 | 29.0 | −32.8 | 15.5 | −27.9 | 8.4 | 5.3 | 8.4 |
| 56 | −8.0 | 5.4 | −13.7 | 4.3 | 2.2 | 21.3 | 31.1 | 15.1 | −1.9 | 15.0 | −8.7 | 11.3 | −24.6 | 7.0 | 18.1 | 3.7 |
| 63 | 28.9 | 24.1 | 47.4 | 59.3 | −5.1 | 25.1 | 41.2 | 15.2 | 19.1 | 21.7 | −10.0 | 11.8 | −18.5 | 4.4 | 21.0 | 6.6 |

TABLE 9
Results of % change in serum hANGPTL3 protein levels
Percent Change in Serum hANGPTL3 Concentration from Day −4 Baseline

| | | | SRS- | | SRS- | | SRS- | | SRS- | | SRS- | | SRS- | |
|---|---|---|---|---|---|---|---|---|---|---|---|---|---|---|
| Study | Vehicle | | 000559 | | 000569 | | 000566 | | 000570 | | 000576 | | 000571 | |
| Day | Avg | SEM | Avg | SEM | Avg | SEM | Avg | SEM | Avg | SEM | Avg | SEM | Avg | SEM |
| −4 | 0.0 | 0.0 | 0.0 | 0.0 | 0.0 | 0.0 | 0.0 | 0.0 | 0.0 | 0.0 | 0.0 | 0.0 | 0.0 | 0.0 |
| 7 | −3.7 | 10.3 | 3.0 | 11.0 | −26.1 | 7.0 | −20.5 | 10.9 | −24.7 | 7.1 | −58.6 | 9.0 | −37.0 | 7.1 |
| 14 | −9.8 | 10.7 | −0.9 | 9.3 | −32.8 | 5.0 | −19.8 | 11.4 | −22.6 | 7.7 | −64.9 | 5.1 | −24.7 | 3.7 |
| 21 | −7.2 | 12.8 | −2.0 | 17.3 | 4.9 | 8.1 | 9.5 | 14.7 | −2.4 | 12.0 | −59.2 | 5.4 | −21.2 | 8.5 |
| 28 | −18.1 | 8.2 | −22.3 | 6.9 | −26.4 | 7.0 | −24.5 | 11.7 | −34.1 | 3.5 | −58.2 | 4.5 | −33.4 | 7.1 |
| 35 | −6.4 | 12.7 | −6.1 | 6.3 | 2.7 | 6.8 | −16.4 | 13.0 | −13.5 | 5.0 | −43.4 | 5.7 | −22.8 | 8.6 |
| 42 | 24.1 | 12.2 | 13.2 | 10.8 | 12.2 | 10.0 | −5.2 | 15.6 | −2.4 | 11.2 | −37.5 | 7.1 | −13.5 | 9.2 |
| 49 | −5.6 | 9.2 | −3.6 | 10.5 | −28.8 | 6.4 | −6.6 | 17.7 | 5.0 | 12.4 | −42.8 | 11.7 | −39.2 | 7.0 |
| 56 | −15.4 | 7.9 | −9.9 | 11.4 | −9.7 | 10.9 | −18.8 | 12.9 | −12.5 | 6.3 | −22.3 | 9.8 | −17.5 | 7.0 |
| 63 | 1.3 | 9.2 | 7.4 | 7.6 | 5.5 | 12.8 | −1.0 | 11.9 | −14.3 | 13.0 | −25.9 | 9.1 | −11.0 | 7.2 |

| | | | | |
|---|---|---|---|---|
| | SRS- | SRS- | SRS- | SRS- |
| Study | 000561 | 000572 | 000577 | 000651 |

| Day | Avg | SEM | Avg | SEM | Avg | SEM | Avg | SEM |
|---|---|---|---|---|---|---|---|---|
| −4 | 0.0 | 0.0 | 0.0 | 0.0 | 0.0 | 0.0 | 0.0 | 0.0 |
| 7 | −26.1 | 11.4 | −0.1 | 13.4 | −54.8 | 7.4 | −49.5 | 10.2 |
| 14 | −6.6 | 7.2 | −35.0 | 7.4 | −71.0 | 6.4 | −67.4 | 5.9 |
| 21 | 23.9 | 12.2 | −25.1 | 8.2 | −63.3 | 5.7 | −61.2 | 7.4 |
| 28 | −35.1 | 10.9 | −8.5 | 10.3 | −55.9 | 5.9 | −51.9 | 7.9 |
| 35 | 0.2 | 11.7 | −9.9 | 8.5 | −58.3 | 8.0 | −39.2 | 11.1 |
| 42 | 15.4 | 16.3 | 10.4 | 13.3 | −34.5 | 11.8 | −28.7 | 12.0 |
| 49 | 5.9 | 14.9 | 10.6 | 19.1 | −50.7 | 7.7 | −24.2 | 15.4 |
| 56 | −3.4 | 12.1 | −1.9 | 11.1 | −43.0 | 9.1 | −20.5 | 9.3 |
| 63 | 4.0 | 16.1 | −4.9 | 8.4 | −39.0 | 9.1 | −17.1 | 9.4 |

TABLE 10
Results of % change in serum hANGPTL3 protein levels
Percent Change in Serum hANGPTL3 Concentration from Day −4 Baseline

| | | | SRS- | | SRS- | | SRS- | | SRS- | | SRS- | |
|---|---|---|---|---|---|---|---|---|---|---|---|---|
| Study | Vehicle | | 000563 | | 000578 | | 000568 | | 000575 | | 000560 | |
| Day | Avg | SEM | Avg | SEM | Avg | SEM | Avg | SEM | Avg | SEM | Avg | SEM |
| −4 | 0.0 | 0.0 | 0.0 | 0.0 | 0.0 | 0.0 | 0.0 | 0.0 | 0.0 | 0.0 | 0.0 | 0.0 |
| 7 | −29.0 | 14.5 | −32.2 | 11.3 | −77.1 | 2.0 | −53.5 | 7.7 | −72.7 | 2.2 | −34.4 | 6.7 |
| 14 | −24.5 | 15.0 | −30.0 | 11.0 | −75.5 | 2.8 | −56.8 | 4.1 | −78.8 | 2.6 | −36.3 | 8.0 |
| 21 | −37.7 | 13.8 | −29.0 | 8.5 | −69.8 | 6.8 | −47.2 | 8.6 | −73.7 | 3.6 | −25.3 | 9.6 |
| 28 | −12.0 | 21.1 | −21.1 | 9.6 | −47.2 | 7.6 | −43.1 | 6.7 | −69.9 | 4.4 | −25.1 | 10.2 |
| | SRS- | | SRS- | | SRS- | | SRS- | | SRS- | | SRS- | |
| Study | 000650 | | 000564 | | 000657 | | 000661 | | 000655 | | 000651 | |
| Day | Avg | SEM | Avg | SEM | Avg | SEM | Avg | SEM | Avg | SEM | Avg | SEM |
| −4 | 0.0 | 0.0 | 0.0 | 0.0 | 0.0 | 0.0 | 0.0 | 0.0 | 0.0 | 0.0 | 0.0 | 0.0 |
| 7 | −57.5 | 4.4 | −39.1 | 5.1 | −74.6 | 2.3 | −56.0 | 3.9 | −67.4 | 2.0 | −75.1 | 1.9 |
| 14 | −57.7 | 5.6 | −29.9 | 3.5 | −83.0 | 1.8 | −72.4 | 3.4 | −80.8 | 2.1 | −81.4 | 2.0 |
| 21 | −56.0 | 3.3 | −28.9 | 6.5 | −79.5 | 2.0 | −60.7 | 2.6 | −77.4 | 2.0 | −71.3 | 3.8 |
| 28 | −61.6 | 2.9 | −24.8 | 5.8 | −65.7 | 4.3 | −49.4 | 3.6 | −70.2 | 2.5 | −60.2 | 6.0 |

TABLE 11
Results of % change in serum hANGPTL3 protein levels
Percent Change in Serum hANGPTL3 Concentration Relative to Vehicle

| | | | SRS- | | SRS- | | SRS- | | SRS- | | SRS- | |
|---|---|---|---|---|---|---|---|---|---|---|---|---|
| Study | Vehicle | | 001803 | | 001804 | | 001805 | | 001806 | | 001807 | |
| Day | Avg | SEM | Avg | SEM | Avg | SEM | Avg | SEM | Avg | SEM | Avg | SEM |
| −4 | 0.0 | 8.4 | −3.6 | 8.9 | −2.2 | 6.0 | −2.5 | 6.2 | −2.7 | 6.0 | −2.7 | 6.0 |
| 7 | 0.0 | 4.0 | −4.6 | 9.9 | −44.7 | 7.3 | −44.0 | 4.0 | −33.0 | 6.5 | −69.1 | 3.4 |
| 14 | 0.0 | 6.7 | −13.3 | 6.6 | −43.3 | 5.7 | −52.5 | 2.9 | −20.7 | 3.4 | −61.8 | 3.3 |
| 21 | 0.0 | 11.3 | −8.3 | 9.7 | −50.2 | 6.9 | −41.9 | 2.9 | −23.0 | 11.5 | −69.3 | 1.2 |
| 28 | 0.0 | 6.0 | 2.3 | 10.0 | −41.3 | 17.6 | −41.4 | 3.7 | −13.4 | 6.8 | −63.0 | 4.1 |
| 35 | 0.0 | 3.6 | −21.1 | 6.9 | −18.9 | 18.6 | −35.4 | 5.1 | −13.4 | 3.4 | −56.9 | 2.4 |
| 42 | 0.0 | 4.8 | −9.4 | 4.1 | −43.0 | 6.0 | −43.7 | 4.6 | −27.1 | 2.7 | −55.3 | 3.5 |
| | SRS- | | SRS- | | SRS- | | SRS- | | SRS- | | SRS- | |
| Study | 001808 | | 001809 | | 001810 | | 001811 | | 001812 | | 000651 | |
| Day | Avg | SEM | Avg | SEM | Avg | SEM | Avg | SEM | Avg | SEM | Avg | SEM |
| −4 | −3.2 | 6.0 | −4.3 | 5.8 | −4.8 | 5.3 | −5.4 | 5.1 | −4.8 | 5.2 | −3.5 | 4.1 |
| 7 | −68.4 | 4.5 | −34.0 | 1.9 | −80.5 | 2.7 | −37.3 | 7.4 | −46.7 | 4.9 | −71.3 | 3.4 |
| 14 | −70.3 | 3.1 | −40.6 | 5.0 | −76.3 | 7.1 | −12.7 | 7.0 | −10.2 | 9.2 | | |
| 21 | −64.8 | 2.8 | −46.2 | 3.4 | −81.2 | 2.5 | −53.2 | 4.5 | −54.9 | 1.6 | −72.9 | 2.4 |
| 28 | −48.9 | 6.0 | −39.4 | 4.8 | −69.2 | 5.7 | −41.9 | 2.2 | −41.0 | 4.9 | −69.1 | 2.5 |
| 35 | −52.4 | 1.9 | −35.5 | 5.8 | −58.2 | 3.8 | −33.3 | 5.7 | −37.3 | 2.0 | −57.9 | 2.5 |
| 42 | −38.8 | 2.1 | −37.7 | 4.2 | −57.4 | 3.6 | −42.0 | 4.8 | −33.6 | 2.5 | −55.7 | 2.2 |

TABLE 12
Results of % change in serum ANGPTL3 protein levels in cynomolgus monkeys
% Change in Serum ANGPTL3 Relative to Day 1 Following Single
2 mg/kg Dose in Cynomolgus Monkeys

| Duplex | | | Study Day | | | | | | | | | | |
|---|---|---|---|---|---|---|---|---|---|---|---|---|---|
| ID | Dose | Statistic | 1 | 4 | 8 | 11 | 15 | 22 | 29 | 36 | 43 | 50 | 57 |
| Saline | 2 mg/kg | Mean | 0.0 | 9.4 | −11.9 | −4.0 | −38.9 | 12.4 | 18.6 | −11.6 | −14.5 | 1.3 | −30.8 |
| | | SD | 0.0 | 30.6 | 26.8 | 8.8 | 13.8 | 9.5 | 19.9 | 12.0 | 12.2 | 27.5 | 13.2 |
| | | N | 4 | 4 | 4 | 4 | 4 | 4 | 4 | 4 | 4 | 4 | 4 |
| SRS- | 2 mg/kg | Mean | 0.0 | −37.8 | −56.5 | −74.3 | −78.4 | −71.9 | −76.8 | −75.0 | −77.9 | −72.5 | −78.7 |
| 001704 | | SD | 0.0 | 34.3 | 14.9 | 9.8 | 10.1 | 15.0 | 13.2 | 9.9 | 9.0 | 12.2 | 8.6 |
| | | N | 4 | 4 | 4 | 4 | 4 | 4 | 4 | 4 | 4 | 4 | 4 |
| SRS- | 2 mg/kg | Mean | 0.0 | −35.3 | −47.5 | −65.3 | −72.8 | −56.4 | −61.8 | −65.2 | −67.6 | −54.6 | −75.1 |
| 000656 | | SD | 0.0 | 9.3 | 9.0 | 4.0 | 7.8 | 8.6 | 8.6 | 9.4 | 9.7 | 6.0 | 7.6 |
| | | N | 4 | 4 | 4 | 4 | 4 | 4 | 4 | 4 | 4 | 4 | 4 |
| SRS- | 2 mg/kg | Mean | 0.0 | −62.5 | −74.6 | −82.6 | −87.7 | −86.5 | −87.2 | −87.0 | −87.9 | −80.3 | −85.0 |
| 000658 | | SD | 0.0 | 11.5 | 7.6 | 7.0 | 4.2 | 2.9 | 5.9 | 4.0 | 5.0 | 8.9 | 9.2 |
| | | N | 4 | 4 | 4 | 4 | 4 | 4 | 4 | 4 | 4 | 4 | 4 |
| SRS- | 2 mg/kg | Mean | 0.0 | −35.5 | −53.5 | −69.9 | −75.9 | −67.1 | −75.4 | −71.8 | −74.9 | −63.8 | −72.8 |
| 000576 | | SD | 0.0 | 22.2 | 13.8 | 10.1 | 8.0 | 12.0 | 9.0 | 10.8 | 9.6 | 13.3 | 11.0 |
| | | N | 4 | 4 | 4 | 4 | 4 | 4 | 4 | 4 | 4 | 4 | 4 |
| SRS- | 2 mg/kg | Mean | 0.0 | −32.6 | −66.9 | −69.4 | −76.3 | −66.0 | −72.8 | −64.9 | −76.5 | −64.5 | −77.8 |
| 000577 | | SD | 0.0 | 40.0 | 12.6 | 11.9 | 8.3 | 15.5 | 19.1 | 18.9 | 7.5 | 14.2 | 16.7 |
| | | N | 4 | 4 | 4 | 4 | 4 | 4 | 4 | 4 | 4 | 4 | 4 |
| SRS- | 2 mg/kg | Mean | 0.0 | 6.0 | −9.6 | −39.1 | −37.7 | −33.9 | −24.7 | −40.0 | −42.7 | — | — |
| 000578 | | SD | 0.0 | 50.7 | 19.8 | 4.4 | 16.9 | 18.1 | 25.3 | 19.5 | 11.1 | — | — |
| | | N | 4 | 4 | 4 | 4 | 4 | 4 | 4 | 4 | 4 | — | — |
| SRS- | 2 mg/kg | Mean | 0.0 | −3.8 | −51.8 | −58.1 | −64.6 | −63.3 | −57.8 | −64.4 | −67.2 | −52.7 | −66.3 |
| 000657 | | SD | 0.0 | 22.3 | 9.1 | 11.0 | 5.2 | 12.2 | 8.5 | 5.3 | 6.7 | 15.9 | 9.6 |
| | | N | 4 | 4 | 4 | 4 | 4 | 4 | 4 | 4 | 4 | 4 | 4 |
| SRS- | 2 mg/kg | Mean | 0.0 | −13.5 | −52.5 | −59.4 | −63.8 | −65.0 | −58.9 | −58.1 | −61.5 | −48.1 | −64.9 |
| 000655 | | SD | 0.0 | 22.3 | 14.6 | 18.5 | 16.7 | 15.6 | 19.4 | 21.0 | 9.5 | 25.6 | 16.4 |
| | | N | 4 | 4 | 4 | 4 | 4 | 4 | 4 | 4 | 4 | 4 | 4 |

| Duplex | | | Study Day | | | | | | | | | |
|---|---|---|---|---|---|---|---|---|---|---|---|---|
| ID | Dose | Statistic | 64 | 71 | 78 | 85 | 92 | 99 | 113 | 127 | 141 | 155 |
| Saline | 2 mg/kg | Mean | −12.7 | −13.3 | −18.6 | −5.7 | 1.9 | −5.8 | — | — | — | — |
| | | SD | 6.8 | 24.4 | 11.0 | 29.8 | 13.9 | 31.4 | — | — | — | — |
| | | N | 4 | 4 | 4 | 4 | 4 | 4 | — | — | — | — |
| SRS- | 2 mg/kg | Mean | −70.0 | −72.1 | −67.9 | −66.5 | −63.0 | −58.0 | −47.8 | −45.1 | −31.4 | −43.0 |
| 001704 | | SD | 14.9 | 13.0 | 13.5 | 13.4 | 14.2 | 16.8 | 27.2 | 22.7 | 38.0 | 31.7 |
| | | N | 4 | 4 | 4 | 4 | 4 | 4 | 4 | 4 | 4 | 4 |
| SRS- | 2 mg/kg | Mean | −60.2 | −56.7 | −49.8 | −44.4 | −42.6 | −42.8 | −45.2 | −41.3 | −26.5 | −17.8 |
| 000656 | | SD | 3.2 | 17.2 | 11.4 | 13.8 | 12.4 | 11.4 | 8.3 | 5.7 | 15.0 | 31.1 |
| | | N | 4 | 4 | 4 | 4 | 4 | 4 | 4 | 4 | 4 | 4 |
| SRS- | 2 mg/kg | Mean | −81.9 | −81.7 | −76.4 | −77.5 | −68.5 | −71.2 | −60.6 | −59.4 | −57.5 | −51.2 |
| 000658 | | SD | 8.0 | 4.7 | 9.4 | 11.3 | 13.4 | 11.4 | 9.1 | 16.5 | 10.9 | 13.6 |
| | | N | 4 | 4 | 4 | 4 | 4 | 4 | 4 | 4 | 4 | 4 |
| SRS- | 2 mg/kg | Mean | −61.7 | −64.6 | −60.1 | −56.7 | −49.2 | −50.2 | −23.9 | −26.6 | −10.1 | −25.1 |
| 000576 | | SD | 18.3 | 16.7 | 14.8 | 20.6 | 25.8 | 19.8 | 29.7 | 33.1 | 31.3 | 20.3 |
| | | N | 4 | 4 | 4 | 4 | 4 | 4 | 4 | 4 | 4 | 4 |
| SRS- | 2 mg/kg | Mean | −70.4 | −64.7 | −55.1 | −60.0 | −64.4 | −67.3 | −45.7 | −57.0 | −45.2 | −39.0 |
| 000577 | | SD | 14.5 | 20.8 | 19.3 | 16.3 | 14.4 | 17.2 | 28.9 | 9.7 | 28.7 | 27.0 |
| | | N | 4 | 4 | 4 | 4 | 4 | 4 | 4 | 4 | 4 | 4 |
| SRS- | 2 mg/kg | Mean | — | — | — | — | — | — | — | — | — | — |
| 000578 | | SD | — | — | — | — | — | — | — | — | — | — |
| | | N | — | — | — | — | — | — | — | — | — | — |
| SRS- | 2 mg/kg | Mean | −59.3 | −67.7 | −59.0 | −60.6 | −31.7 | −33.9 | −26.0 | −6.4 | −8.2 | 16.1 |
| 000657 | | SD | 13.6 | 6.2 | 13.5 | 10.8 | 28.6 | 26.1 | 32.3 | 63.5 | 35.6 | 75.8 |
| | | N | 4 | 4 | 4 | 4 | 4 | 4 | 4 | 4 | 4 | 4 |
| SRS- | 2 mg/kg | Mean | −59.4 | −68.7 | −55.7 | −59.5 | −33.3 | −43.3 | −49.0 | −43.2 | −30.6 | −42.2 |
| 000655 | | SD | 18.2 | 13.9 | 16.0 | 13.9 | 29.2 | 21.3 | 17.2 | 14.7 | 19.3 | 13.7 |
| | | N | 4 | 4 | 4 | 4 | 4 | 4 | 4 | 4 | 4 | 4 |

TABLE 13
Results of % change in serum hANGPTL3 protein levels
Percent Change in Serum hANGPTL3 Concentration Relative to Vehicle

| | | | SRS- | | SRS- | | SRS- | | SRS- | | SRS- | | SRS- | |
|---|---|---|---|---|---|---|---|---|---|---|---|---|---|---|
| Study | PBS | | 000658 | | 002032 | | 002024 | | 002045 | | 002052 | | 002047 | |
| Day | Avg | SEM | Avg | SEM | Avg | SEM | Avg | SEM | Avg | SEM | Avg | SEM | Avg | SEM |
| −4 | 0 | 8.8 | −0.3 | 8.7 | −0.6 | 8.0 | −0.7 | 9.8 | −0.2 | 7.7 | −0.8 | 7.9 | −0.2 | 10.3 |
| 7 | 0 | 9.5 | −74.4 | 0.7 | −55.1 | 4.3 | −18.9 | 4.6 | −25.9 | 7.6 | −39.0 | 5.4 | −56.6 | 1.7 |
| 14 | 0 | 3.9 | −69.4 | 3.5 | −27.9 | 5.8 | −9.3 | 5.8 | 1.3 | 2.9 | −21.1 | 5.6 | −40.2 | 3.3 |
| 21 | 0 | 19.9 | −71.5 | 3.3 | −32.7 | 4.4 | −15.1 | 8.5 | −20.3 | 6.9 | −27.1 | 8.5 | −50.0 | 2.8 |
| 28 | 0 | 13.7 | −42.2 | 10.9 | −5.0 | 5.9 | 1.7 | 8.5 | 6.0 | 11.3 | −12.0 | 7.1 | −22.5 | 4.8 |
| 35 | 0 | 10.4 | −21.4 | 10.0 | 2.5 | 8.9 | 23.8 | 7.5 | 33.5 | 12.1 | 8.1 | 7.0 | −9.9 | 5.1 |

| | SRS- | | SRS- | | SRS- | | SRS- | | SRS- | | SRS- | |
|---|---|---|---|---|---|---|---|---|---|---|---|---|
| Study | 002053 | | 002034 | | 002028 | | 002049 | | 002050 | | 002051 | |
| Day | Avg | SEM | Avg | SEM | Avg | SEM | Avg | SEM | Avg | SEM | Avg | SEM |
| −4 | −0.2 | 9.5 | −0.8 | 8.3 | 0.0 | 10.5 | −0.1 | 10.7 | 0.0 | 10.4 | −1.1 | 12.3 |
| 7 | −33.2 | 6.2 | −58.4 | 1.8 | −33.2 | 5.3 | −56.2 | 2.8 | −73.0 | 2.9 | −65.2 | 1.5 |
| 14 | 1.6 | 8.6 | −25.2 | 4.4 | 3.0 | 8.8 | −27.0 | 4.1 | −81.6 | 4.3 | −53.9 | 2.6 |
| 21 | −38.4 | 5.0 | −52.5 | 3.2 | −27.8 | 4.5 | −32.9 | 5.6 | −76.7 | 2.3 | −71.4 | 2.1 |
| 28 | −16.5 | 11.8 | −12.0 | 4.7 | 24.6 | 9.2 | 2.8 | 9.5 | −58.1 | 1.3 | −53.2 | 1.8 |
| 35 | 13.5 | 10.0 | 35.8 | 32.5 | 49.1 | 11.2 | 5.8 | 7.5 | −56.7 | 5.8 | −36.2 | 3.7 |

What is claimed is:

1. A polynucleic acid molecule for modulating an expression of Angiopoietin-like protein 3 (ANGPTL3) gene, wherein the polynucleic acid molecule comprises a nucleic acid sequence in Table 1, Table 2, Table 3, or Table 4.

2. The polynucleic acid molecule of claim 1, wherein the polynucleic acid molecule is a single-stranded nucleic acid molecule.

3. The polynucleic acid molecule of claim 2, wherein the single-stranded nucleic acid molecule comprises at least 14, 15, 16, 17, 18 consecutive nucleotides that are complementary to a nucleic acid sequence selected from SEQ ID NOs: 401-596, 819-828, and 870-875, with no more than 1, 2, 3, 4 mismatches.

4. The polynucleic acid molecule of any one claims 2-3, wherein the single-stranded nucleic acid molecule comprises a sequence that is at least 80%, at least 85%, at least 90%, or at least 95% complementary to a nucleic acid sequence selected from SEQ ID NOs: 401-596, 819-828, and 870-875.

5. The polynucleic acid molecule of claim 1, wherein the polynucleic acid molecule is a double-stranded nucleic acid molecule comprising a passenger strand and a guide strand.

6. The polynucleic acid molecule of claim 5, wherein the passenger strand comprises a nucleic acid sequence that is at least 80%, at least 85%, at least 90%, or at least 95% identical to a nucleic acid sequence selected from SEQ ID NOs: 401-596, 819-828, and 870-875.

7. The polynucleic acid molecule of any one of claims 5-6, wherein the guide strand comprises a nucleic acid sequence that is at least 80%, at least 85%, at least 90%, or at least 95% identical to a nucleic acid sequence selected from SEQ ID NOs: 1-196, 797-806, and 850-857.

8. The polynucleic acid molecule of any one of claims 5-7, wherein the passenger strand comprises a nucleic acid sequence comprising at least 14, 15, 16, 17, 18, 19, or 20 consecutive sequences of a sequence selected from SEQ ID NOs: 401-596, 819-828, and 870-875 with no more than 1, 2, 3, or 4 mismatches.

9. The polynucleic acid molecule of any one of claims 5-8, wherein the guide strand comprises a nucleic acid sequence comprising at least 14, 15, 16, 17, 18, 19, 20, 21, or 22 consecutive sequences of a sequence selected from SEQ ID NOs: SEQ ID NOs: 1-196, 797-806, and 850-857 with no more than 1, 2, 3, or 4 mismatches.

10. The polynucleic acid molecule of any one of claims 5-9, wherein the passenger strand comprises a nucleic acid sequence selected from SEQ ID NOs: 401-596, 819-828, and 870-875 and the guide strand comprises a nucleic acid sequence selected from SEQ ID NOs: 1-196, 797-806, and 850-857.

11. The polynucleic acid molecule of claim 5, wherein the passenger strand comprises a nucleic acid sequence that is at least 90%, or at least 95% identical to a nucleic acid sequence selected from Table 3 (SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875).

12. The polynucleic acid molecule of any one of claims 5 or 11, wherein the guide strand comprises a nucleic acid sequence that is at least 90%, or at least 95% identical to a nucleic acid sequence selected from Table 3 (SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857.

13. The polynucleic acid molecule of any one of claims 5, 11, or 12, wherein the passenger strand comprises a nucleic acid sequence selected from Table 3 (SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875) and the guide strand comprises a sequence selected from a nucleic acid sequence selected from SEQ ID NOs: Table 3 (SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857).

14. The polynucleic acid molecule of any one of claims 1-13, wherein the polynucleic acid molecule comprises (1) a 2′-fluoro modified nucleotide; (2) a 2′-O-methyl modified nucleotide; (3) 2′-deoxy modified nucleotide; or (4) a modified intemucleotide linkage.

15. The polynucleic acid molecule of any one of claims 1-14, wherein the polynucleic acid molecule comprises at least two consecutive modified intemucleotide linkages at the 5′ end.

16. The polynucleic acid molecule of any one of claims 5-15, wherein the guide strand comprises at least two intemucleotide linkages among 3 intemucleotide linkages at the 3′end substituted with modified intemucleotide linkages.

17. The polynucleic acid molecule of any one of claims 5-16, wherein the guide strand comprises 5′-nNfnnnNfnNfNfnnnnNfnNfnnnnnnn-3′, 5′-nNfnnnNfnnnnnnnNfnNfnnnnnnn-3′, 5′-nNfnnnnNfnnnnNfnNfnnnnnnnnn-3′, 5′-nNfnnnnNfnnnnNfnNfnNfnnnnnnn-3′, or 5′-nNfnnnnnnnnnNfnNfnNfnnnnnnn-3′, wherein “Nf” stands for a 2′-fluoro modified nucleotide, and wherein “n” stands for a 2′-O-methyl modified nucleotide.

18. The polynucleic acid molecule of any one of claims 5-16, wherein the passenger strand comprises 5′-nnnnnnNfnNfnNfnnnnnnnnnn-3′, 5′-nnnnnnNfnNfNfNfmrnnnnnnnn-3′, or 5′-nnnnnnnnNfNfNfnnnnnnnnnn-3′, wherein “Nf” stands for a 2′-fluoro modified nucleotide, and wherein “n” stands for a 2′-O-methyl modified nucleotide.

19. The polynucleic acid molecule of any one of claims 5-16, wherein the passenger strand comprises 5′-NfnNfnNfnNfnNfNfNfnNfnNfnNfnNfnNf-3′, wherein the guide strand comprises 5′-nNfnNfnNfnNfnNfnnnNfnNfnNfnNfnnn-3′, wherein “Nf” stands for a 2′-fluoro modified nucleotide, and wherein “n” stands for a 2′-O-methyl modified nucleotide.

20. The polynucleic acid molecule of any one of claims 5-16, wherein the passenger strand comprises 5′-nnnnnnNfnNfNfNfmrnnnnnnnn-3′, wherein the guide strand comprises 5′-nNfnnnNfnNfNfnnnnNfnNfnnnnnnn-3′, wherein “Nf” stands for a 2′-fluoro modified nucleotide, and wherein “n” stands for a 2′-O-methyl modified nucleotide.

21. The polynucleic acid molecule of any one of claims 5-16, wherein the passenger strand comprises 5′-nnnnnnnnNfnNfnnnnnnnnn-3′, wherein the guide strand comprises 5′-nNfnnnnnnnnnNfnNfnnnnnnnnn-3′, wherein “Nf” stands for a 2′-fluoro modified nucleotide, and wherein “n” stands for a 2′-O-methyl modified nucleotide.

22. The polynucleic acid molecule of any one of claims 5-16, wherein the passenger strand comprises 5′-nnnnnnNfnNfnNfnnnnnnnnnn-3′, wherein the guide strand comprises 5′-nNfnnnnnnnnnNfnNfnNfnnnnnnn-3′, wherein “Nf” stands for a 2′-fluoro modified nucleotide, and wherein “n” stands for a 2′-O-methyl modified nucleotide.

23. The polynucleic acid molecule of any one of claims 5-16, wherein the passenger strand comprises 5′-nnnnnnNfnNfnNfnnnnnnnnnn-3′, wherein the guide strand comprises 5′-nNfnnnnNfnnnnNfnNfnNfnnnnnnn-3′, wherein “Nf” stands for a 2′-fluoro modified nucleotide, and wherein “n” stands for a 2′-O-methyl modified nucleotide.

24. The polynucleic acid molecule of any one of claims 14-23, wherein the modified intemucleotide linkage is a phosphorothioate intemucleotide linkage.

25. The polynucleic acid molecule of claim 24, wherein the modified intemucleotide linkage comprises a stereochemically enriched phosphorothioate intemucleotide linkage.

26. The polynucleic acid molecule of any one of claims 5-25, wherein the guide strand comprises a nucleotide analogue selected from a group consisting of acyclic L-threoninol nucleic acid-thymine-3′-phosphate (T-T), acyclic L-threoninol nucleic acid-adenine-3′-phosphate (T-A), acyclic N-acetyl L-threoninol abasic nucleic acid-3′phosphate (T-NAc), and 1′,2′-Dideoxyribose-3′phosphate (dAB).

27. The polynucleic acid molecule of claim 26, wherein the nucleotide analogue is located at a seed region of the guide strand (positions 2-8) from the 5′ end.

28. The polynucleic acid molecule of any one of claims 14-27, wherein the passenger strand comprises a nucleic acid sequence that is at least 80%, at least 85%, at least 90%, or at least 95% identical to a nucleic acid sequence selected from SEQ ID NOs: 601-796, 830-839, and 876-881.

29. The polynucleic acid molecule of any one of claims 14-28, wherein the guide strand comprises a nucleic acid sequence that is at least 80%, at least 85%, at least 90%, or at least 95% identical to a nucleic acid sequence selected from SEQ ID NOs: 201-396, 808-817, and 858-868.

30. The polynucleic acid molecule of any one of claims 14-29, wherein the passenger strand comprises a nucleic acid sequence selected from SEQ ID NOs: 601-796, 830-839, and 876-881, and the guide strand comprises a nucleic acid sequence selected from SEQ ID NOs: 201-396, 808-817, and 858-868.

31. The polynucleic acid molecule of any one of claims 1-30, wherein the polynucleic acid molecule is 19-25 nucleotides in length.

32. The polynucleic acid molecule of any one of claims 1-30, wherein the polynucleic acid molecule is 21-23 nucleotides in length.

33. A polynucleic acid molecule for modulating expression of Angiopoietin-like protein 3 (ANGPTL3) gene, wherein the polynucleic acid molecule comprises:
(a) a guide strand comprising the nucleotide sequence selected from SEQ ID NOs: 5, 7, 8, 11, 18, 20, 22, 40, 43, 44, 45, 46, 50, 52, 53, 56, 108, 109, 113, 114, 145, 146, 148, 153, 157, 158, 797-806, and 850-857, and a passenger strand comprising the nucleotide sequence selected from SEQ ID NOs: 405, 407, 408, 411, 418, 420, 422, 440, 443-446, 450, 452, 453, 456, 508, 509, 513, 514, 545, 546, 548, 553, 557, 558, 819-828, and 870-875;
(b) a guide strand comprising the nucleotide sequence selected from SEQ ID NOs: 205, 207, 208, 211, 218, 220, 222, 240, 243-246, 250, 252, 253, 256, 308, 309, 313, 314, 345, 346, 348, 353, 357, 358, 808-817, and 858-868, and a passenger strand comprising the nucleotide sequence selected from SEQ ID NOs: 605, 607, 608, 611, 618, 620, 622, 640, 643, 644, 645, 646, 650, 652, 653, 656, 708, 709, 713, 714, 745, 746, 748, 753, 757, 758, 830-839, and 876-881;
(c) a guide strand comprising the nucleotide sequence of usUfsaguuGfguuuCfgUfgAfuuuccscsa (SEQ ID NO: 313) and a passenger strand comprising the nucleotide sequence of gsgsaaauCfaCfgAfaaccaacuaa (SEQ ID NO: 713);
(d) a guide strand comprising the nucleotide sequence of usUfsagagUfauaaCfcUfuCfcauuususg (SEQ ID NO: 346) and a passenger strand comprising the nucleotide sequence of asasauggAfaGfgUfuauacucuaa (SEQ ID NO: 746);
(e) a guide strand comprising the nucleotide sequence of usAfsuggaUfcaacAfuUfuUfgguugsasu (SEQ ID NO: 353) and a passenger strand comprising the nucleotide sequence of csasaccaAfaAfuGfuugauccaua (SEQ ID NO: 753);
(f) a guide strand comprising the nucleotide sequence of usUfsaaggAfuuuaAfuAfcCfagauusasu (SEQ ID NO: 358) and a passenger strand comprising the nucleotide sequence of asasucugGfuAfuUfaaauccuuaa (SEQ ID NO: 758);
(g) a guide strand comprising the nucleotide sequence of usAfsuuagAfuugcUfuCfaCfuauggsasg (SEQ ID NO: 308) and a passenger strand comprising the nucleotide sequence of cscsauagUfgAfaGfcaaucuaaua (SEQ ID NO: 708);
(h) a guide strand comprising the nucleotide sequence of usUfsauagUfugguUfuCfgUfgauuuscsc (SEQ ID NO: 314) and a passenger strand comprising the nucleotide sequence of asasaucaCfgAfaAfccaacuauaa (SEQ ID NO: 714);
(i) a guide strand comprising the nucleotide sequence of usAfsgaguAfuaacCfuUfcCfauuuusgsa (SEQ ID NO: 345) and a passenger strand comprising the nucleotide sequence of asasaaugGfaAfgGfuuauacucua (SEQ ID NO: 745);
(j) a guide strand comprising the nucleotide sequence of usUfsaauuAfgauuGfcUfuCfacuausgsg (SEQ ID NO: 309) and a passenger strand comprising the nucleotide sequence of asusagugAfaGfcAfaucuaauuaa (SEQ ID NO: 709);
(k) a guide strand comprising the nucleotide sequence of usAfsauuaGfauugCfuUfcAfcuaugsgsa (SEQ ID NO: 815) and a passenger strand comprising the nucleotide sequence of csasuaguGfaAfgCfaaucuaauua (SEQ ID NO: 837);
(1) a guide strand comprising the nucleotide sequence of usUfsucauUfgaagUfuUfuGfugaucscsa (SEQ ID NO: 812) and a passenger strand comprising the nucleotide sequence of gsasucacAfaAfaCfuucaaugaaa (SEQ ID NO: 834);
(m) a guide strand comprising the nucleotide sequence of usAfsuugcUfucacUfaUfgGfaguausasu (SEQ ID NO: 813) and a passenger strand comprising the nucleotide sequence of asusacucCfaUfaGfugaagcaaua (SEQ ID NO: 835);
(n) a guide strand comprising the nucleotide sequence of usAfsgaguAfuaacCfuUfcCfauuucsgsa (SEQ ID NO: 865) and a passenger strand comprising the nucleotide sequence of gsasaaugGfaAfgGfuuauacucua (SEQ ID NO: 879); or
(o) a guide strand comprising the nucleotide sequence of usAfsgaguAfuaacCfuUfcCfauuccsgsa (SEQ ID NO: 866) and a passenger strand comprising the nucleotide sequence of gsgsaaugGfaAfgGfuuauacucua (SEQ ID NO: 880),
where smaller case “n” stands for 2′-O-methyl modified nucleotide, upper case followed with an “f” (i.e., “Nf”) stands for 2′-fluoro modified nucleotide, and “s” stands for 3′-phosphorothioate.

34. A polynucleic acid molecule conjugate for modulating an expression of Angiopoietin-like protein 3 (ANGPTL3) gene, wherein the polynucleic acid molecule conjugate comprises a polynucleic acid molecule of any one of claims 1-33 and an asialoglycoprotein receptor targeting moiety.

35. The polynucleic acid molecule conjugate of claim 34, wherein the polynucleic acid molecule and the asialoglycoprotein receptor targeting moiety is coupled via a linker.

36. The polynucleic acid molecule conjugate of claim 35, wherein the linker comprises formula (IV) below, 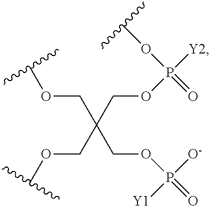
wherein at least one of Y1 and Y2 is a nucleotide in the polynucleic acid molecule.

37. The polynucleic acid molecule conjugate of claim 36, wherein the Y1 is the last nucleotide on the 3′ end of the passenger strand of the polynucleic acid molecule.

38. The polynucleic acid molecule conjugate of claim 36, wherein the Y1 and Y2 are two consecutive nucleotides in the polynucleic acid molecule.

39. The polynucleic acid molecule conjugate of any one of claims 34-38, wherein the asialoglycoprotein receptor targeting moiety comprises N-Acetylgalactosamine (GalNAc).

40. The polynucleic acid molecule conjugate of any one of claims 34-39, wherein the linker and the asialoglycoprotein receptor targeting moiety with the last nucleotide on the 3′ end of the passenger strand of the polynucleic acid molecule are shown in: 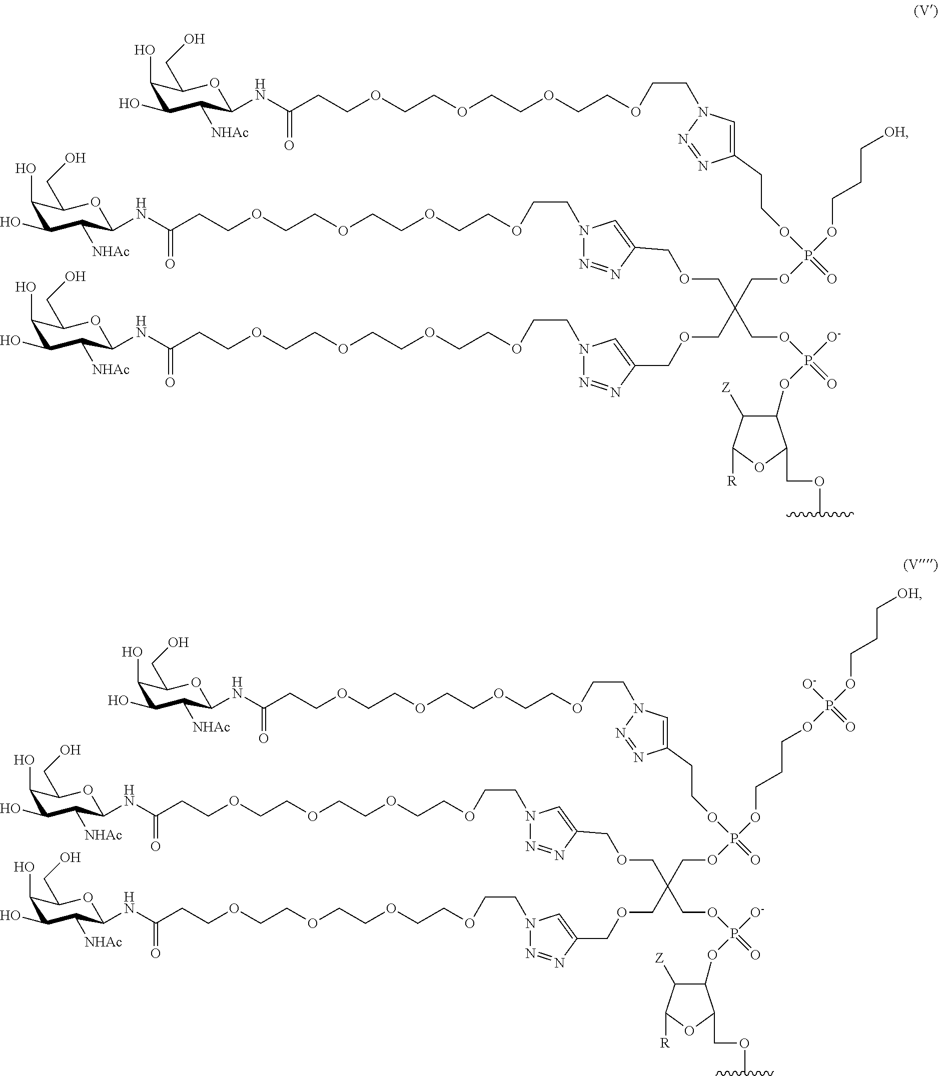 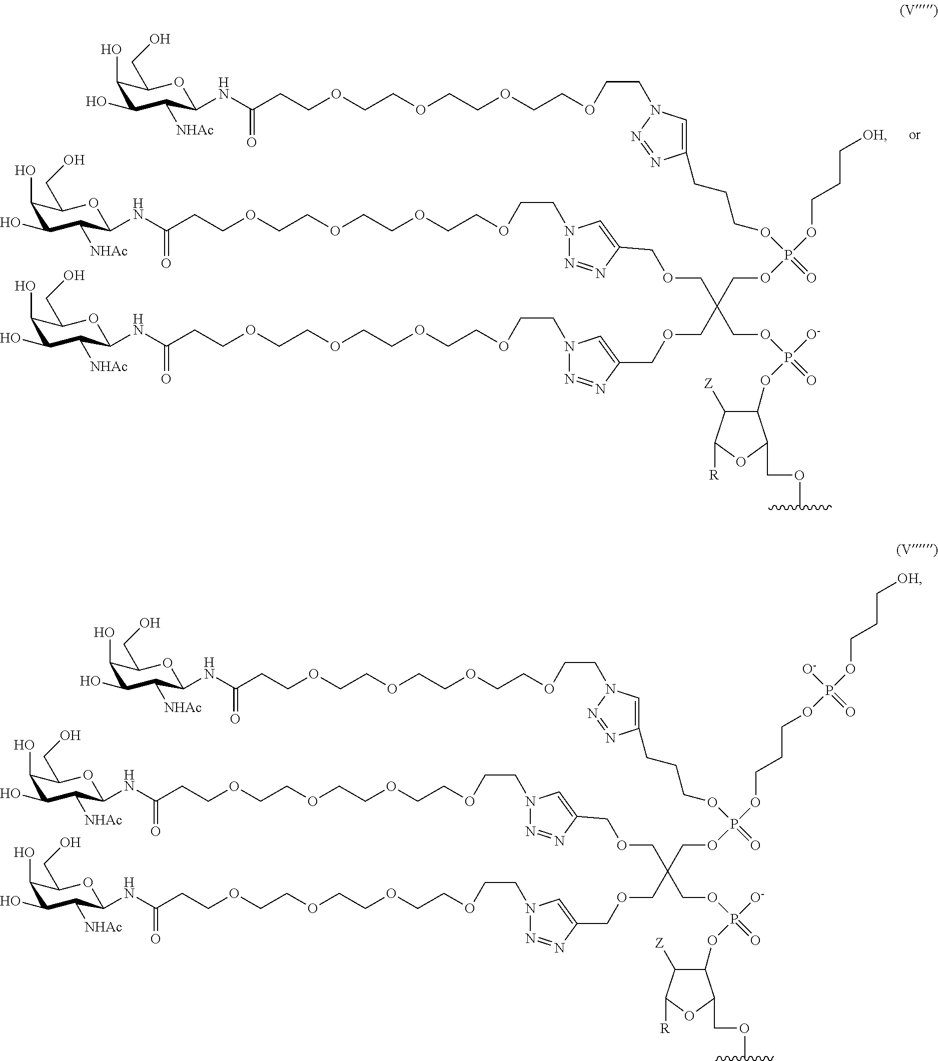
wherein Z in formula (V′), (V″″), (V″″′) or (V″″″) is —H, —OH, —O-Methyl, —F, or —O— methoxyethyl, and R in formula (V′), (V″″), (V″″′), or (V″″″) is adenine, uracil, guanine, cytosine, thymine, abasic, or others.

41. A pharmaceutical composition comprising a polynucleic acid molecule of any one of claims 1-33 or a polynucleic acid molecule conjugate of any one of claims 34-40, and a pharmaceutically acceptable excipient.

42. The pharmaceutical composition of claim 41, wherein the pharmaceutical composition is formulated as a nanoparticle formulation.

43. The pharmaceutical composition of any one of claim 41 or claim 42, wherein the pharmaceutical composition is formulated for parenteral, oral, intranasal, buccal, rectal, transdermal, intravenous, subcutaneous, or intrathecal administration.

44. A method of modulating an mRNA expression of Angiopoietin-like protein 3 (ANGPTL3) gene in a subject, comprising: administering to the subject a polynucleic acid molecule of any one of claims 1-33 or a polynucleic acid molecule conjugate of any one of claims 34-40, or a pharmaceutical composition of any one of claims 41-43, thereby modulating the mRNA expression of ANGPTL3 gene in the subject.

45. A method of preventing, alleviating, or treating ANGPTL3-associated disease or symptom thereof in a subject in need thereof, comprising: administering to the subject a polynucleic acid molecule of any one of claims 1-33 or a polynucleic acid molecule conjugate of any one of claims 34-40, or a pharmaceutical composition of any one of claims 41-43, thereby preventing, alleviating, or treating ANGPTL3-associated disease or symptom thereof in the subject.

46. The method of claim 45, wherein the ANGPTL3-associated disease or symptom thereof comprises hyperlipidermia.

47. The method of claim 45, wherein the ANGPTL3-associated disease or symptom thereof comprises atherosclerosis, coronary heart disease, or vascular disease.
